(19) United States
(12) Patent Application Publication
(10) Pub. No.: US 2026/0224760 A1
(43) Pub. Date: Aug. 6, 2026

(54) SOMATOSTATIN BINDING COMPOSITIONS AND METHODS OF USE THEREOF

(71) Applicant: Ratio Therapeutics, Inc., Boston, MA (US)

(72) Inventors: Shashikanth Ponnala, Tenafly, NJ (US); Pradeep K. Singh, Ashland, MA (US); Stephen DiMagno, Garden City, NY (US); John W. Babich, Brightwaters, NY (US)

(21) Appl. No.: 19/375,714

(22) Filed: Oct. 31, 2025

Related U.S. Application Data
(60) Provisional application No. 63/715,164, filed on Nov. 1, 2024.

(51) Int. Cl. A61K 51/08; A61P 35/00

(52) U.S. Cl. CPC A61K 51/083; A61K 51/088; A61P 35/00

(57) ABSTRACT

Disclosed are compounds represented by the following structural formula (I):

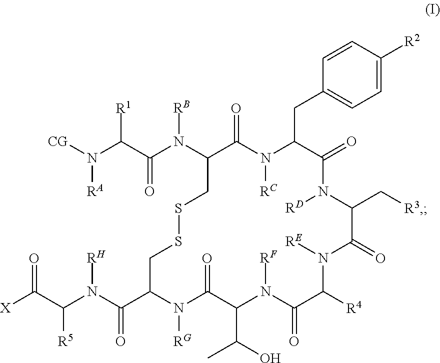

or a pharmaceutically acceptable salt thereof. The variables of structural formula (I) are described herein. The compound of the invention can be attached to chelating groups for radionuclide binding and are therefore suitable for radioimaging and/or radiotherapy applications, for example the disclosed compounds can be radiolabeled with a positron emitter such as ¹⁸F, ⁶⁸Ga or ⁶⁴Cu, and used for positron emission tomography (PET). Alternatively, the compounds can be radiolabeled with an alpha particle emitter such as ²²⁵Ac, a beta particle emitter such as ⁶⁷Cu or ¹⁷⁷Lu, or an Auger electron emitter (e.g. ¹¹¹In, ⁶⁷Ga, ⁹⁹mTc, ¹⁹⁵mPt, ¹²⁵I, ¹²³I and ¹⁶¹Tb) for use as a radiotherapeutic.

### RELATED APPLICATIONS

This application claims priority to U.S. Provisional Patent Application No. 63/715,164, filed Nov. 1, 2024, the contents of which are herein incorporated by reference.

### FIELD OF THE INVENTION

The present technology relates to targeted therapy agents, more particularly polypeptides useful in the treatment of disease. For example, the compositions described herein may be used as radiopharmaceutical agents, or conjugated to drug/toxin conjugates, useful for the treatment of somatostatin receptor positive cancers.

### BACKGROUND OF THE INVENTION

The class of somatostatin receptors (SSTRs) consists of five members (SSTR1, SSTR2, SSTR2, SSTR4, SSTR5), which are widely expressed in different tissues in the body including nervous, pituitary, kidney, lung, and immune cells. Their natural ligand is the neuropeptide somatostatin (SST), which occurs in two active isoforms, SST-14 and SST-28. In combination with their receptors, both isoforms act as inhibitory hormones. An important physiological function of the SSTR/SST axis is, for example, the inhibition of the release of growth hormones. SSTRs, particularly the SSTR subtype 2, are found highly expressed in many neoplastic cells and in tumoral blood vessels. Overexpression of SSTRs, in particular SSTR2, has been found in various neuroendocrine tumors, as well as other tumors such as breast, ovarian, and lung cancer. Targeting of the SSTR2 for drug delivery has been accomplished by using stabilized, cyclic somatostatin analogs such as octreotate, octreotide, and lanreotide. For example, covalently attaching a DOTA chelator to octreotide (DOTATATE, also known as DOTA-(Tyr3)-octreotate) has made it possible to target delivery of radionuclides to tumor cells expressing somatostatin receptors. ¹⁷⁷Lu DOTATATE therapy is a form of peptide receptor radionuclide therapy (PRRT) which targets somatostatin receptors and is a form of targeted drug delivery.

The clinical use of Lutathera (¹⁷⁷Lu-DOTATATE) for treating SSTR2-positive neuroendocrine tumors (NETs), is associated with significant kidney uptake, which can lead to nephrotoxicity. To mitigate this, patients are currently administered an infusion of amino acids to reduce kidney uptake, a procedure known to cause severe nausea and vomiting, making it difficult for patients to tolerate. There is therefore a critical need for SSTR2 binding drug conjugates with reduced kidney uptake and increased tumor to kidney uptake ratios.

### SUMMARY OF THE INVENTION

Disclosed herein are bifunctional compounds that exhibit strong binding to SSTR2 (see Example 3) and demonstrate improved tumor-to-kidney ratios (see Example 4 and FIGS. 1-9), achieved through decreased kidney uptake, increased tumor uptake, or a combination of both. As shown in FIGS. 10-12, the compounds of the present disclosure exhibit similar or increased efficacy compared to the state of the art compounds, DOTATATE and DOTA-JR11, at equivalent doses in a mouse survival study. The favorable pharmacokinetic properties of the compounds, including reduced “wash-out” effects, contribute to their improved targeting capabilities while minimizing off-target tissue accumulation. The compounds of the invention can be chelated with radionuclides and are therefore suitable for radioimaging and/or radiotherapy applications. For example, the disclosed compounds can be radiolabeled with a positron emitter such as ⁶⁸Ga or ⁶⁴Cu, and used for positron emission tomography (PET). Alternatively, the compounds can be radiolabeled with an alpha particle emitter such as ²²⁵Ac, a beta particle emitter such as ⁶⁷Cu or ¹⁷⁷Lu, or an Auger electron emitter (e.g., ¹¹¹In, ⁶⁷Ga, ⁹⁹mTc, ¹⁹⁵mPt, ¹²⁵I, ¹²³I and ¹⁶¹Tb). Alternatively, the compounds are attached to a cytotoxic agent for targeted delivery of the cytotoxic agent to tumors, for example conjugated to gemcitabine or doxycycline, or a venom. Likewise, the compounds can be conjugated to compounds having physiological effects, such as TLR agonists to stimulate the immune response of a recipient. A key advantage of the disclosed compounds is a marked reduction in kidney uptake and/or increase in tumor retention that leads to very favorable tumor to kidney ratios.

One embodiment of the invention is a compound represented by structural formula (I):

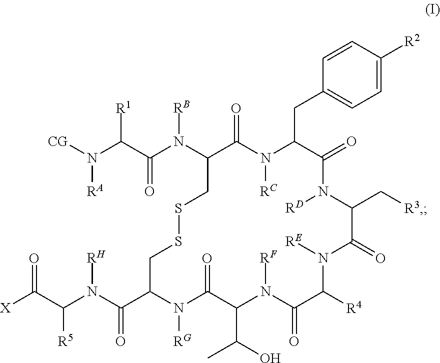

or a pharmaceutically acceptable salt thereof, wherein:
CG is a chelating group, an optical dye or fluorophore, a cytotoxic agent, or an immune stimulant;
X is OH or NH₂
R^{A}, R^{B}, R^{C}, R^{D}, R^{E}, R^{F}, R^{G}, and R^{H} are each independently selected from hydrogen and C₁₋₄ alkyl;
R¹ is C₁₋₆ alkyl, or phenyl, wherein said C₁₋₆ alkyl represented by R¹ is substituted with one R¹⁰;
   R¹⁰ is independently selected from phenyl and naphthyl, wherein said phenyl or naphthyl represented by R¹⁰ is optionally substituted with one or more groups selected from halogen;
R² is OH or

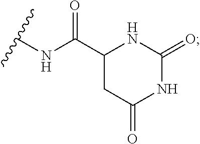 [SMILES: *NC(=O)C1CC(=O)NC(=O)N1]

R³ is

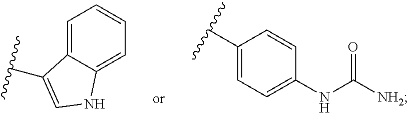 [SMILES: *c1c[nH]c2ccccc12 ; *c1ccc(NC(N)=O)cc1]

R⁴ is C₁₋₆ alkyl-NR^{4a}R^{4b} or C₁₋₆ alkyl-NHC(═NH)NH₂, wherein said C₁₋₆ alkyl in the group represented by R⁴ is optionally substituted with one or more halogen or C₁₋₃ alkyl;
R^{4a} is hydrogen or C₁₋₃ alkyl and R^{4b} is hydrogen, C₁₋₃ alkyl, C(O)C₁₋₃ alkyl and C(O)C₁₋₃ haloalkyl, provided that R^{4a} and R^{4b} are not both hydrogen;
R⁵ is C₁₋₆ alkyl or C₁₋₆ aralkyl, wherein the alkyl represented by R⁵ or the aryl portion of the aralkyl represented by R⁵ are independently substituted with one or more halogen, —OH, or C₁₋₆ alkoxy.

Another embodiment of the invention is a pharmaceutical composition comprising: i) a compound disclosed herein or a pharmaceutically acceptable salt thereof; and ii) a pharmaceutically acceptable carrier or diluent. For compounds comprising a chelating group, the chelating group is preferably chelated with a radionuclide. In another embodiment, the pharmaceutical composition comprises a compound disclosed herein that is chelated to a first radionuclide, metal ion or metal-halogen ion (e.g. ¹⁷⁷Lu) and further comprises the compound disclosed herein that is chelated to a second radionuclide, metal ion, or metal-halogen ion (e.g. ²²⁵Ac), wherein the first and second radionuclide, metal ion, or metal-halogen ion are different.

Another embodiment of the invention is a method of treating a subject with diseased tissue that expresses somatostatin receptors. The diseased tissue in one aspect can be a cancer. In another aspect, the diseased tissue can be another somatostatin receptor-expressing disease. The method comprises administering an effective amount of the compound disclosed herein or pharmaceutically acceptable salt to the subject. Preferably, the compound used for therapy comprises a cytotoxic agent, such as a chelating group having a radionuclide that emits beta, alpha, Auger or other cytotoxic radiation which can kill the diseased tissue. In an alternative embodiment, the method comprises administering an effective amount of a compound disclosed herein in combination with a second anti-cancer therapeutic agent.

Yet another embodiment of the invention is a method of treating a disease in a subject, wherein the disease is characterized by the expression of somatostatin receptors. The disease can be a cancer. The method comprises administering an effective amount of the compound disclosed herein or pharmaceutically acceptable salt to the subject. Preferably, the compound used for therapy comprises a cytotoxic agent, such as a chelating group having a radionuclide that emits beta, alpha, Auger or other cytotoxic radiation which can kill the diseased tissue. In an alternative embodiment, the method comprises administering an effective amount of a compound disclosed herein in combination with a second anti-cancer therapeutic agent.

Yet another embodiment of the invention is a method of imaging a region in a subject having or suspected of having a cancer or disease which expresses SSTR2, comprising:
(i) administering to the subject a diagnostically effective amount of a compound disclosed herein, or pharmaceutically acceptable salt thereof, or a pharmaceutical composition thereof,
(ii) exposing a region in the subject to an imaging device, the region suspected of having diseased tissue; and
(iii) obtaining an image of diseased tissue in the region.

Yet another embodiment of the invention is a method of imaging a region in a subject having or suspected of having a cancer or disease which expresses SSTR2, comprising:
(i) administering to the subject a diagnostically effective amount of a compound disclosed herein, or pharmaceutically acceptable salt thereof, or a pharmaceutical composition disclosed herein;
(ii) exposing a region in the subject to an imaging device, the region suspected of having diseased tissue; and
(iii) obtaining an image of the region suspected of having diseased tissue.

Preferably, the compound used for imaging comprises a chelating group having a radionuclide that emits gamma-rays or positrons or other detectible radiation. In another aspect, the compound comprises an optical dye or a fluorophore, the emissions of which can be detected.

Yet another embodiment of the invention is a method of imaging tumors. The method comprises:
(i) administering to a subject a compound disclosed herein, or a pharmaceutically acceptable salt thereof, or a pharmaceutical composition thereof, in an amount effective to contact and bind to the tumor and/or surrounding tissue;
(ii) irradiating the tumor and/or surrounding tissue at a wavelength absorbed by the bound compound; and
(iii) detecting a signal from the irradiated bound compound, thereby imaging the tumor and/or surrounding tissue.

Preferably, the compound used for imaging comprises a chelating group having a radionuclide that emits gamma-rays or positrons, or an optical dye or a fluorophore, or other detectible radiation.

Still another embodiment of the invention is a method of treating diseased tissue. The method comprises:
(i) administering to a subject a compound disclosed herein, or a pharmaceutically acceptable salt thereof, or a pharmaceutical composition thereof, in an amount effective to contact and bind to the diseased tissue; and
(ii) using the compound as a fiducial, irradiating the region of the bound compound with one or more doses of external beam radiation, thereby treating the diseased tissue with radiation.

Preferably, the compound used for the fiducial comprises a chelating group having a radionuclide that emits gamma-rays or positrons, or an optical dye or a fluorophore, or other detectible radiation.

Even still another embodiment of the invention is a method of treating diseased tissue.

### The Method Comprises:

(i) administering to a subject a compound disclosed herein, or a pharmaceutically acceptable salt thereof, or a pharmaceutical composition thereof, in an amount effective to contact and bind to the diseased tissue; and
(ii) using the compound as a fiducial for guided surgery applications, to resect the region of the diseased tissue, thereby excising the diseased tissue.

Preferably, the compound used for the fiducial comprises a chelating group having a radionuclide that emits gamma-rays or positrons, or an optical dye or a fluorophore, or other detectible radiation.

### BRIEF DESCRIPTION OF THE FIGURES

FIG. 1 shows the percent change in kidney uptake and tumor to kidney ratio of ¹⁷⁷Lu-Compound 2 compared to ¹⁷⁷Lu-Compound 2-comp.

FIG. 2 shows the percent change in kidney uptake and tumor to kidney ratio of ¹⁷⁷Lu-Compound 6 compared to ¹⁷⁷Lu-Compound 6-comp.

FIG. 3 shows the percent change in kidney uptake and tumor to kidney ratio of ¹⁷⁷Lu-Compound 8 compared to ¹⁷⁷Lu-Compound 8-comp.

FIG. 4 shows the percent change in kidney uptake and tumor to kidney ratio of ¹⁷⁷Lu-Compound 9 compared to ¹⁷⁷Lu-Compound 9-comp.

FIG. 5 shows the percent change in kidney uptake and tumor to kidney ratio of ¹⁷⁷Lu-Compound 10 compared to ¹⁷⁷Lu-Compound 10-comp.

FIG. 6 shows the percent change in kidney uptake and tumor to kidney ratio of ¹⁷⁷Lu-Compound 11 compared to ¹⁷⁷Lu-Compound 11-comp.

FIG. 7 shows the percent change in kidney uptake and tumor to kidney ratio of ¹⁷⁷Lu-Compound 12 compared to ¹⁷⁷Lu-Compound 12-comp.

FIG. 8 shows the percent change in kidney uptake and tumor to kidney ratio of ¹⁷⁷Lu-Compound 13 compared to ¹⁷⁷Lu-Compound 13-comp.

FIG. 9 shows time integrated activity coefficients (TIACs) for the tumor and kidney for ¹⁷⁷Lu-DOTATATE, ¹⁷⁷Lu-DOTA-JR11, ¹⁷⁷Lu-Compound 24, and ¹⁷⁷Lu-Compound 25.

FIGS. 10A and 10B shows comparative mouse survival studies between ¹⁷⁷Lu-DOTA-JR11, ¹⁷⁷Lu-DOTATATE, and ¹⁷⁷Lu-Compound 24 at a dosage of 7.5 MBq.

FIGS. 11A and 11B shows comparative mouse survival studies between ¹⁷⁷Lu-DOTA-JR11, ¹⁷⁷Lu-DOTATATE, and ¹⁷⁷Lu-Compound 24 at a dosage of 15 MBq.

FIGS. 12A and 12B shows comparative mouse survival studies between ¹⁷⁷Lu-DOTA-JR11, ¹⁷⁷Lu-DOTATATE, and ¹⁷⁷Lu-Compound 24 at a dosage of 30 MBq.

### DETAILED DESCRIPTION

Disclosed herein are a series of compounds that bind with high affinity to somatostatin receptor 2 (SSTR2). In some embodiments, the compounds deliver a payload to a tissue expressing SSTR2. Compounds of the invention are described herein below.

A first embodiment of the invention is a compound represented by structural formula (I) or a pharmaceutically acceptable salt thereof. The definitions of the variables in structural formula (I) are provided above in the Summary of the Invention.

A second embodiment of the invention is a compound represented by structural formula (I), or a pharmaceutically acceptable salt thereof, wherein the compound is represented by structural formula (IIa), (IIb), or (IIc):

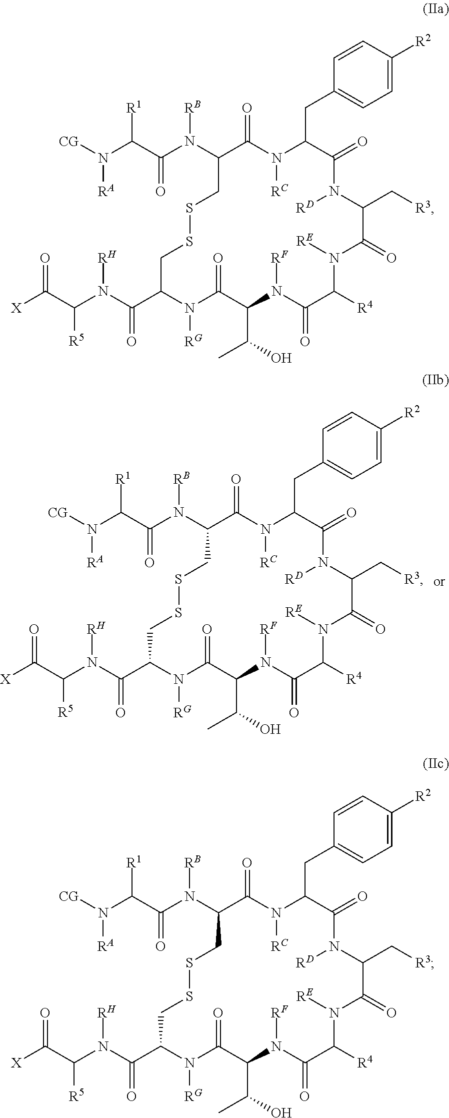

wherein the variables are as defined in the first embodiment.

A third embodiment of the invention is a compound represented by structural formula (I), (IIa), (IIb), or (IIc), or a pharmaceutically acceptable salt thereof, wherein the compound is represented by structural formula (IIIa), (IIIb), or (IIIc):

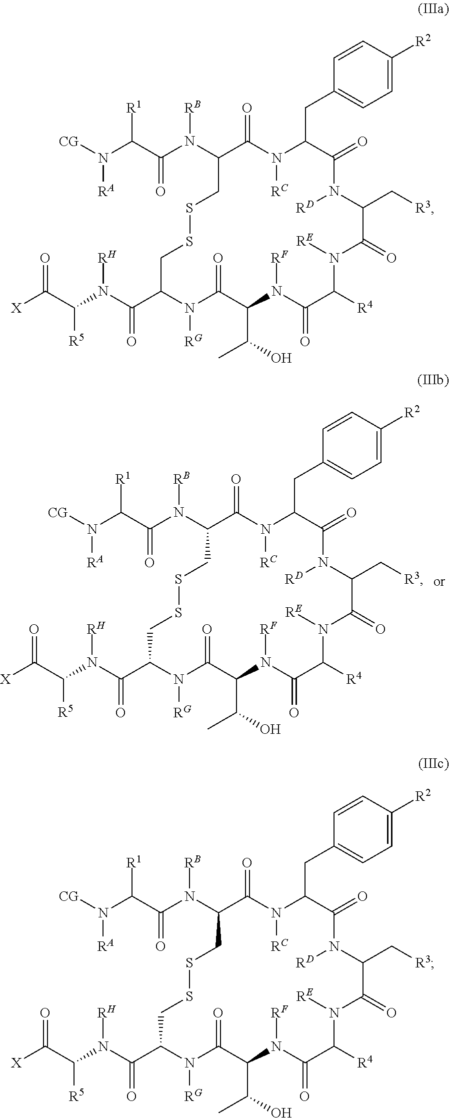

wherein the variables are as defined in the first embodiment.

A fourth embodiment of the invention is a compound represented by structural formula (I), (IIa), (IIb), or (IIc), or a pharmaceutically acceptable salt thereof, wherein the compound is represented by structural formula (IVa), (IVb), or (IVc):

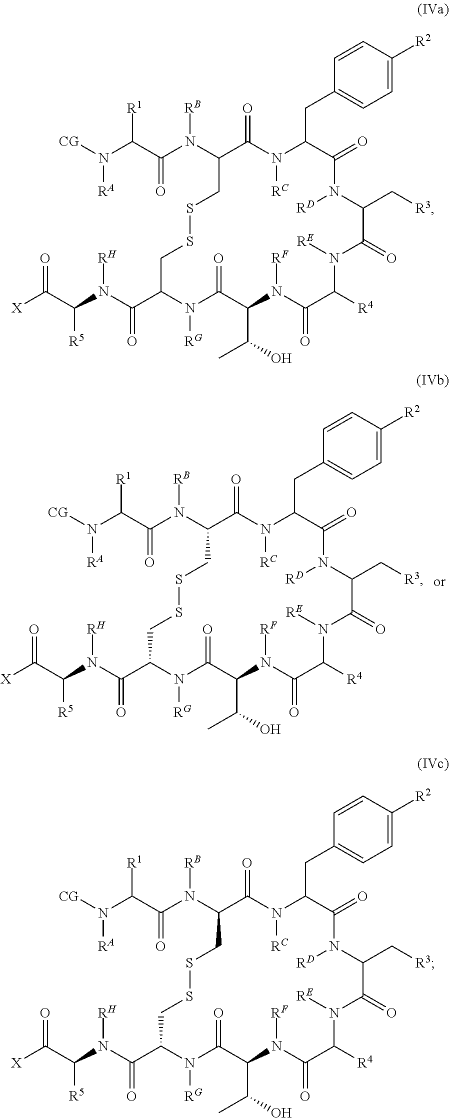

wherein the variables are as defined in the first embodiment.

A fifth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R⁴ is C₁₋₄ alkyl-NR^{4a}R^{4b}, and the remainder of the variables are as defined in the first embodiment.

A sixth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R^{4a} is hydrogen and R^{4b} is C₁₋₃ alkyl, and the remainder of the variables are as described in the first or fifth embodiment.

A seventh embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R^{4a} is hydrogen and R^{4b} is CH₃, and the remainder of the variables are as described in the first or fifth embodiment.

An eighth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R¹ is C₁₋₃ alkyl substituted with one R¹⁰, and the remainder of the variables are as described in the first, fifth, sixth, or seventh embodiment.

A ninth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R¹⁰ is phenyl or naphthyl, wherein said phenyl is optionally substituted with one to five halogen, and the remainder of the variables are as described in first, fifth, sixth, seventh, or eighth embodiment.

A tenth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R¹ is selected from the group consisting of

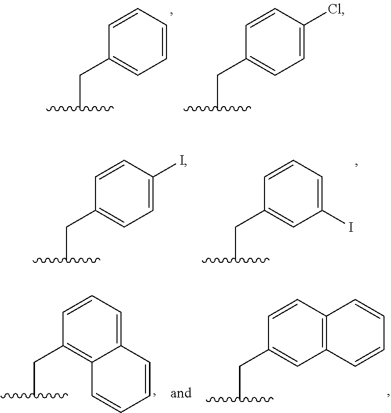

and the remainder of the variables are as described in the first, fifth, sixth, seventh, eighth, or ninth embodiment.

An eleventh embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R¹ is selected from the group consisting of

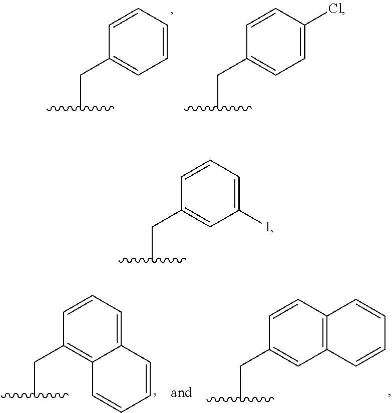 [SMILES: *Cc1ccccc1 ; *Cc1ccc(Cl)cc1 ; *Cc1cccc(I)c1 ; *Cc1cccc2ccccc12 ; *Cc1ccc2ccccc2c1]

and the remainder of the variables are as described in the first, fifth, sixth, seventh, eighth, ninth, or tenth embodiment.

A twelfth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R² is OH, and the remainder of the variables are as described in the first, fifth, sixth, seventh, eighth, ninth, tenth, or eleventh embodiment.

A thirteenth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R² is

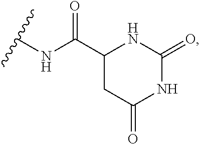 [SMILES: *NC(=O)C1CC(=O)NC(=O)N1]

and the remainder of the variables are as described in the first, fifth, sixth, seventh, eighth, ninth, tenth, or eleventh embodiment.

A fourteenth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R³ is

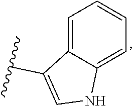 [SMILES: *c1c[nH]c2ccccc12]

and the remainder of the variables are as described in the first, fifth, sixth, seventh, eighth, ninth, tenth, eleventh, twelfth, or thirteenth embodiment.

A fifteenth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R³ is

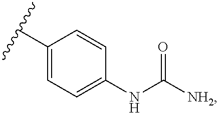 [SMILES: *c1ccc(NC(N)=O)cc1]

and the remainder of the variables are as described in the first, fifth, sixth, seventh, eighth, ninth, tenth, eleventh, twelfth, or thirteenth embodiment.

A sixteenth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R⁵ is C₁₋₃ alkyl or C₁₋₃ aralkyl, wherein the alkyl represented by R⁵ is substituted with —OH and the aryl portion of the aralkyl represented by R⁵ is substituted with one F, Cl, or —OH, and the remainder of the variables are as described in the first, fifth, sixth, seventh, eighth, ninth, tenth, eleventh, twelfth, thirteenth, fourteenth, or fifteenth embodiment.

A seventeenth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R⁵ is

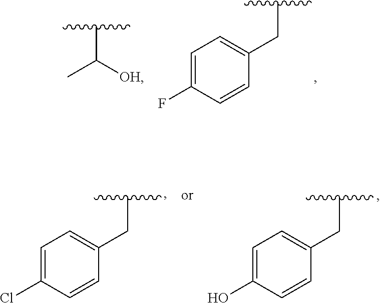

and the remainder of the variables are as described in the first, fifth, sixth, seventh, eighth, ninth, tenth, eleventh, twelfth, thirteenth, fourteenth, fifteenth, or sixteenth embodiment.

An alternative seventeenth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R⁵ is

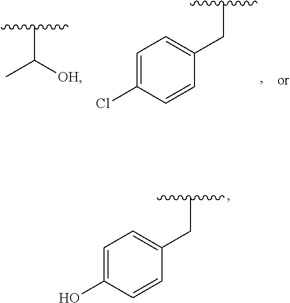

and the remainder of the variables are as described in the first, fifth, sixth, seventh, eighth, ninth, tenth, eleventh, twelfth, thirteenth, fourteenth, fifteenth, or sixteenth embodiment.

An eighteenth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein R^{A}, R^{B}, R^{C}, R^{D}, R^{E}, R^{F}, R^{G}, and R^{H} are each independently selected from hydrogen or CH₃, and the remainder of the variables are as described in the first, fifth, sixth, seventh, eighth, ninth, tenth, eleventh, twelfth, thirteenth, fourteenth, fifteenth, sixteenth, or seventeenth embodiment.

A nineteenth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein and R^{E} is independently selected from hydrogen or CH₃ and R^{A}, R^{B}, R^{C}, R^{D}, R^{F}, R^{G}, and R^{H} are each hydrogen, and the remainder of the variables are as described in the first, fifth, sixth, seventh, eighth, ninth, tenth, eleventh, twelfth, thirteenth, fourteenth, fifteenth, sixteenth, seventeenth, or eighteenth embodiment.

A twentieth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein CG comprises or is a fluorophore or an optical dye, and the remainder of the variables are as described in the first, fifth, sixth, seventh, eighth, ninth, tenth, eleventh, twelfth, thirteenth, fourteenth, fifteenth, sixteenth, seventeenth, eighteenth, or nineteenth embodiment.

A twenty-first embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein the fluorophore is

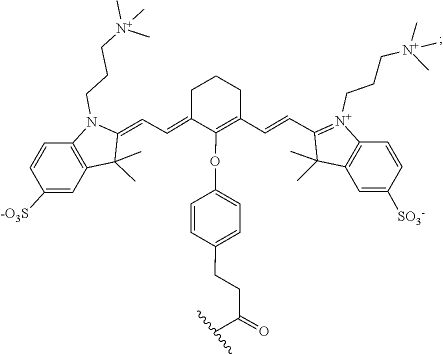

and the optical dye is selected from the group consisting of: a carbocyanine, indocarbocyanine, oxacarbocyanine, thiacarbocyanine, merocyanine, polymethine, coumarin, rhodamine, xanthene, fluorescein, Borodipyrromethane (BODIPY), VivoTag-680, VivoTag-S750, AlexaFluor dyes (e.g., AlexaFluor660, AlexaFluor680, AlexaFluor700, AlexaFluor750, AlexaFluor790) and DylightFluor dyes, and the remainder of the variables are as described in the twentieth embodiment.

A twenty-second embodiment of the invention is a compound represented by structural formula (I), (IIa), (IIb), (IIc), (IIIa), (IIIb), (IIIc), (IVa), (IVb), or (IVc), or a pharmaceutical acceptable salt thereof, wherein CG is a chelating group that is the residue of a chelating agent, and the remainder of the variables are as described in the first, fifth, sixth, seventh, eighth, ninth, tenth, eleventh, twelfth, thirteenth, fourteenth, fifteenth, sixteenth, seventeenth, eighteenth, or nineteenth embodiment.

A twenty-third embodiment of the invention is a compound represented by structural formula (I), (IIa), (IIb), (IIc), (IIIa), (IIIb), (IIIc), (IVa), (IVb), or (IVc), or a pharmaceutical acceptable salt thereof, wherein the chelating group is the residue of a chelating agent selected from 1,4,7-triazacyclononane-1,4,7-triacetic acid (NOTA), p-SCN-Bn-NOTA, NODAGA (2-(4,7-bis(carboxymethyl)-1,4,7-triazonin-1-yl)pentanedioic acid), 1,4,7,10-tetraazacyclododecane-1,4,7,10-tetraacetic acid (DOTA), DOTAGA (2-(4,7,10-tris(carboxymethyl)-1,4,7,10-tetraazacyclododecan-1-yl)pentanedioic acid), p-SCN-Bn-DOTA (also known as 2B-DOTA-NCS),PIP-DOTA, diethylenetriaminepentaacetic acid (DTPA), PIP-DTPA, AZEP-DTPA, ethylenediamine tetraacetic acid (EDTA), triethylenetetraamine-N,N,N′,N″,N′″,N′″-hexa-acetic acid (TTHA), 7-[2-(bis-carboxymethylamino)-ethyl]-4,10-bis-carboxymethyl- 1,4,7,10-tetraaza-cyclododec-1-yl-acetic acid (DEPA), 2,2′,2″-(10-(2-(bis(carboxymethyl)amino)-5-(4-isothiocyanatophenyl) pentyl)-1,4,7,10-tetraazacyclododecane-1,4,7-triyl)triacetic acid (3p-C-DEPA-NCS), NETA, {4-carboxymethyl-7-[2-(carboxymethylamino)-ethyl]-perhydro-1,4,7-triazonin-1-yl}-acetic acid (NPTA), diacetylpyridinebis(benzoylhydrazone), 1,4,7,10,13,16-hexaazacyclooctadecane N,N′,N″,N′″,N″″,N′″″-hexaaceticacid (HEHA), octadentate terephthalamide ligands, 2,2′-(4-(2-(bis(carboxymethyl)amino)-5-(4-isothiocyanatophenyl)pentyl)-10-(2-(bis(carboxymethyl)amino)ethyl)-1,4,7,10-tetraazacyclododecane-1,7-diyl)diacetic acid, N,N′-bis[(6-carboxy-2-pyridyl)methyl]-4,13-diaza-18-crown-6 (H2macropa), 6-((16-((6-carboxypyridin-2-yl)methyl)-1,4,10,13-tetraoxa-7,16-diazacyclooctadecan-7-yl)methyl)-4-isocyanatopicolinic acid (macropa-NCO), 6-((16-((6-carboxypyridin-2-yl)methyl)-1,4,10,13-tetraoxa-7,16-diazacyclooctadecan-7-yl)methyl)-4-isothiocyanatopicolinic acid (macropa-NCS), 3,9-carboxymethyl-6-(2-methoxy-5-isothiocyanatophenyl)carboxymethyl-3,6,9,15-tetraazabicyclo-[9.3.1]pentadeca-1(15),11,13-triene and 2-[4,7,10-tris(2-amino-2-oxoethyl)-1,4,7,10-tetrazacyclododec-1-yl]acetamide (TCMC or DOTAM), and the remainder of the variables are as described in the twenty-second embodiment.

An alternative twenty-third embodiment of the invention is a compound represented by structural formula (I), (IIa), (IIb), (IIc), (IIIa), (IIIb), (IIIc), (IVa), (IVb), or (IVc), or a pharmaceutical acceptable salt thereof, wherein the chelating group is the residue of a chelating agent selected from 1,4,7-triazacyclononane-1,4,7-triacetic acid (NOTA), p-SCN-Bn-NOTA, 1,4,7,10-tetraazacyclododecane-1,4,7,10-tetraacetic acid (DOTA), p-SCN-Bn-DOTA (also known as 2B-DOTA-NCS),PIP-DOTA, diethylenetriaminepentaacetic acid (DTPA), PIP-DTPA, AZEP-DTPA, ethylenediamine tetraacetic acid (EDTA), triethylenetetraamine-N,N,N′,N″,N′″,N′″-hexa-acetic acid (TTHA), 7-[2-(bis-carboxymethylamino)-ethyl]-4,10-bis-carboxymethyl-1,4,7,10-tetraaza-cyclododec-1-yl-acetic acid (DEPA), 2,2′,2″-(10-(2-(bis(carboxymethyl)amino)-5-(4-isothiocyanatophenyl) pentyl)-1,4,7,10-tetraazacyclododecane-1,4,7-triyl)triacetic acid (3p-C-DEPA-NCS), NETA,{4-carboxymethyl-7-[2-(carboxymethylamino)-ethyl]-perhydro-1,4,7-triazonin-1-yl}-acetic acid (NPTA), diacetylpyridinebis(benzoylhydrazone), 1,4,7,10,13,16-hexaazacyclooctadecane N,N′,N″,N′″,N″″,N′″″-hexaaceticacid (HEHA), octadentate terephthalamide ligands, 2,2′-(4-(2-(bis(carboxymethyl)amino)-5-(4-isothiocyanatophenyl)pentyl)-10-(2 -(bis(carboxymethyl)amino)ethyl)-1,4,7,10-tetraazacyclododecane-1,7-diyl)diacetic acid, N,N′-bis[(6-carboxy-2-pyridyl)methyl]-4,13-diaza-18-crown-6 (H2macropa), 6-((16-((6-carboxypyridin-2-yl)methyl)-1,4,10,13-tetraoxa-7,16-diazacyclooctadecan-7-yl)methyl)-4-isocyanatopicolinic acid (macropa-NCO), 6-((16-((6-carboxypyridin-2-yl)methyl)-1,4,10,13-tetraoxa-7,16-diazacyclooctadecan-7-yl)methyl)-4-isothiocyanatopicolinic acid (macropa-NCS), 3,9-carboxymethyl-6-(2-methoxy-5-isothiocyanatophenyl)carboxymethyl-3,6,9,15-tetraazabicyclo-[9.3.1]pentadeca-1(15),11,13-triene and 2-[4,7,10-tris(2-amino-2-oxoethyl)-1,4,7,10-tetrazacyclododec-1-yl]acetamide (TCMC or DOTAM), and the remainder of the variables are as described in the twenty-second embodiment

A twenty-fourth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein the chelating group comprises or is
i) represented by the structural formula

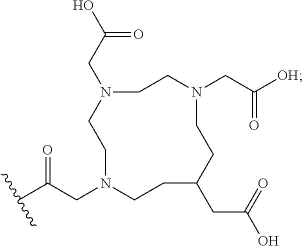 [SMILES: *C(=O)CN1CCC(CC(=O)O)CCN(CC(=O)O)CCN(CC(=O)O)CC1]

ii) represented by one of the following structural formulae

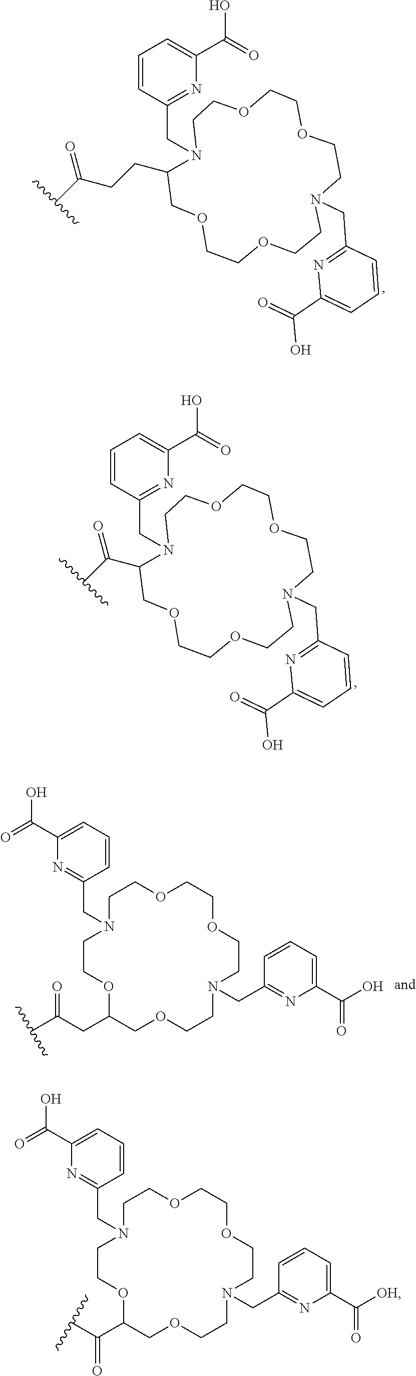 [SMILES: *C(=O)CCC1COCCOCCN(Cc2cccc(C(=O)O)n2)CCOCCOCCN1Cc1cccc(C(=O)O)n1 ; *C(=O)C1COCCOCCN(Cc2cccc(C(=O)O)n2)CCOCCOCCN1Cc1cccc(C(=O)O)n1 ; *C(=O)CC1COCCN(Cc2cccc(C(=O)O)n2)CCOCCOCCN(Cc2cccc(C(=O)O)n2)CCO1 ; *C(=O)C1COCCN(Cc2cccc(C(=O)O)n2)CCOCCOCCN(Cc2cccc(C(=O)O)n2)CCO1]

and the remainder of the variables are as described in the twenty-third embodiment.

An alternative twenty-fourth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein the chelating group comprises or is
i) represented by the following structural formula

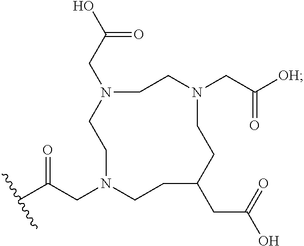 [SMILES: *C(=O)CN1CCC(CC(=O)O)CCN(CC(=O)O)CCN(CC(=O)O)CC1]

or
ii) represented by one of the following structural formulae

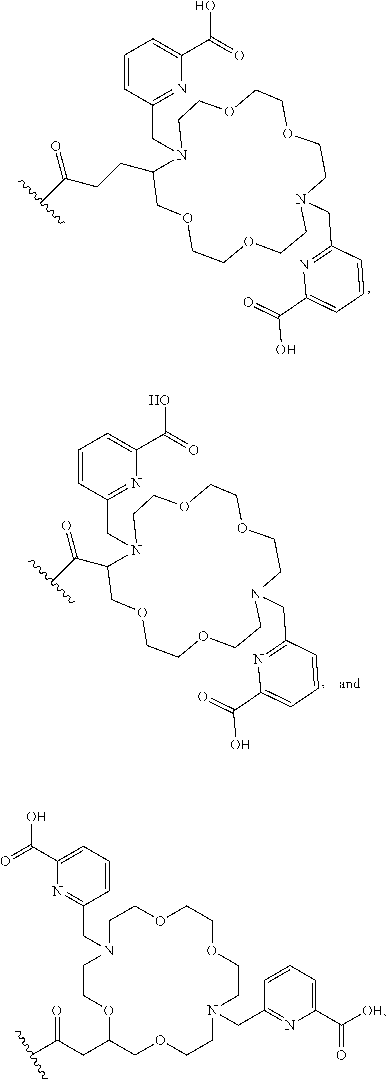 [SMILES: *C(=O)CCC1COCCOCCN(Cc2cccc(C(=O)O)n2)CCOCCOCCN1Cc1cccc(C(=O)O)n1 ; *C(=O)C1COCCOCCN(Cc2cccc(C(=O)O)n2)CCOCCOCCN1Cc1cccc(C(=O)O)n1 ; *C(=O)CC1COCCN(Cc2cccc(C(=O)O)n2)CCOCCOCCN(Cc2cccc(C(=O)O)n2)CCO1]

and the remainder of the variables are as described in the twenty-third embodiment.

A twenty-fifth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein the chelating group comprises or is represented by the structural formula

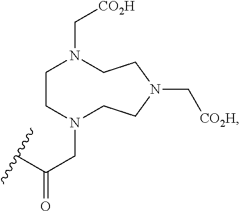 [SMILES: *C(=O)CN1CCN(CC(=O)O)CCN(CC(=O)O)CC1]

and the remainder of the variables are as described in the twenty-third embodiment.

A twenty-sixth embodiment of the invention is a compound represented by structural formula (I), (IIa), (IIb), (IIc), (IIIa), (IIIb), (IIIc), (IVa), (IVb), or (IVc), or a pharmaceutically acceptable salt thereof, wherein the chelating group comprises or is a siderophore, and the remainder of the variables are as described in the first, fifth, sixth, seventh, eighth, ninth, tenth, eleventh, twelfth, thirteenth, fourteenth, fifteenth, sixteenth, seventeenth, eighteenth, or twenty-second embodiment.

A twenty-seventh embodiment of the invention is a compound represented by structural formula (I), (IIa), (IIb), and (IIc), or a pharmaceutically acceptable salt thereof, wherein the compound is represented by structural formula (Va), (Vb), or (Vc):

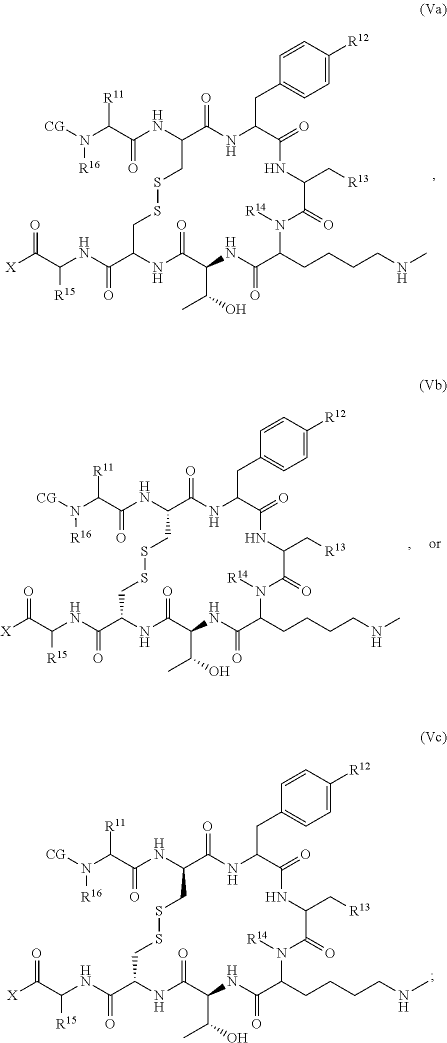

wherein:
X is OH or NH₂;
CG comprises or is a chelating group selected from:

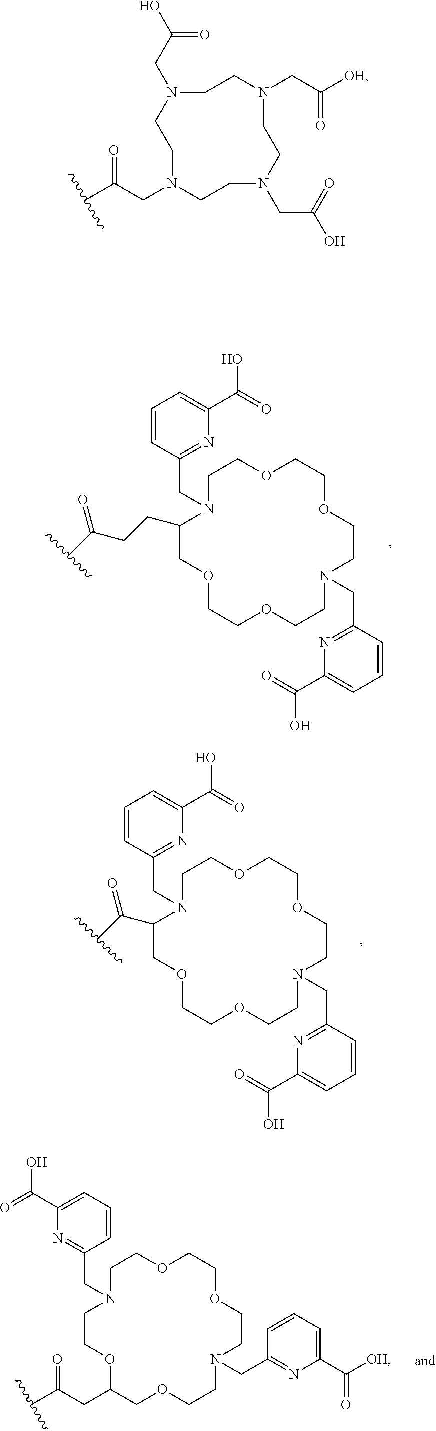 [SMILES: *C(=O)CN1CCN(CC(=O)O)CCN(CC(=O)O)CCN(CC(=O)O)CC1 ; *C(=O)CCC1COCCOCCN(Cc2cccc(C(=O)O)n2)CCOCCOCCN1Cc1cccc(C(=O)O)n1 ; *C(=O)C1COCCOCCN(Cc2cccc(C(=O)O)n2)CCOCCOCCN1Cc1cccc(C(=O)O)n1 ; *C(=O)CC1COCCN(Cc2cccc(C(=O)O)n2)CCOCCOCCN(Cc2cccc(C(=O)O)n2)CCO1]

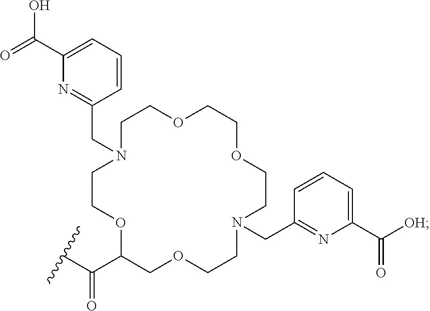 [SMILES: *C(=O)C1COCCN(Cc2cccc(C(=O)O)n2)CCOCCOCCN(Cc2cccc(C(=O)O)n2)CCO1]

R¹¹ is selected from

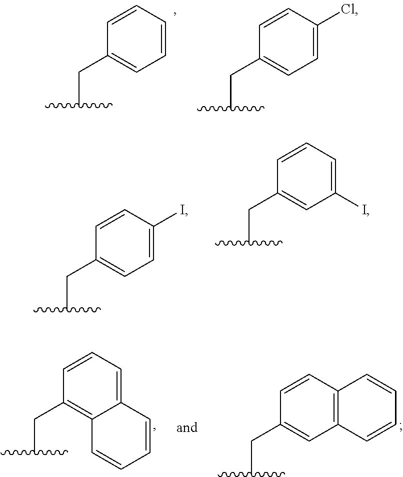 [SMILES: *Cc1ccccc1 ; *Cc1ccc(Cl)cc1 ; *Cc1ccc(I)cc1 ; *Cc1cccc(I)c1 ; *Cc1cccc2ccccc12 ; *Cc1ccc2ccccc2c1]

R¹² is OH or

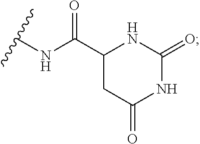 [SMILES: *NC(=O)C1CC(=O)NC(=O)N1]

R¹³ is

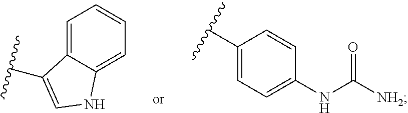 [SMILES: *c1c[nH]c2ccccc12 ; *c1ccc(NC(N)=O)cc1]

R¹⁴ and R¹⁶ are each independently H or CH₃; and R¹⁵ is

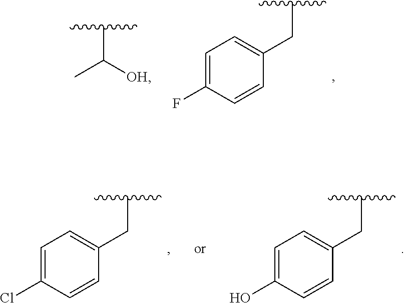

A twenty-eighth embodiment of the invention is a compound represented by structural formula (I), (IIa), (IIb), (IIc), (IVa), (IVb), (IVc), (Va), (Vb), or (Vc), or a pharmaceutically acceptable salt thereof, wherein the compound is represented by structural formula (VIa), (VIb), or (VIc):

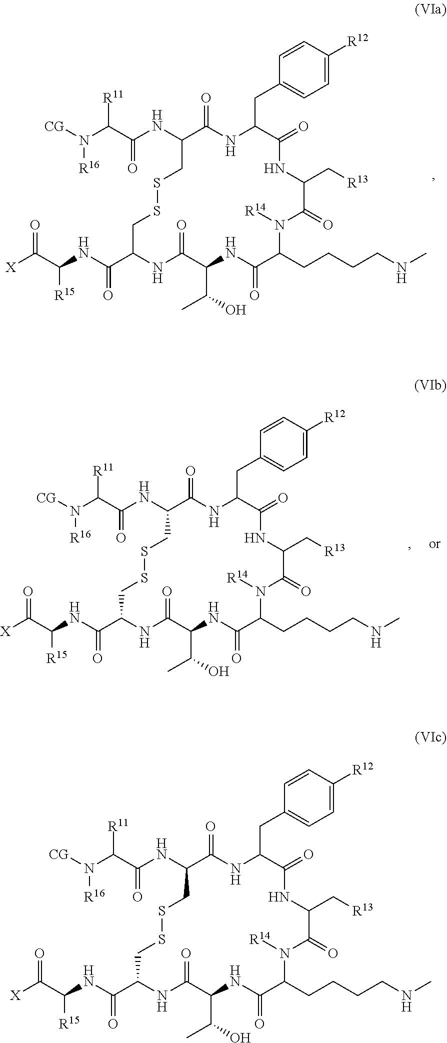

wherein the variables are as defined in the twenty-seventh embodiment.

A twenty-ninth embodiment of the invention is a compound represented by structural formula (I), (IIa), (IIb), (IIc), (IIIa), (IIIb), (IIIc), (Va), (Vb), or (Vc), or a pharmaceutically acceptable salt thereof, wherein the compound is represented by structural formula (VIIa), (VIIb), or (VIIc):

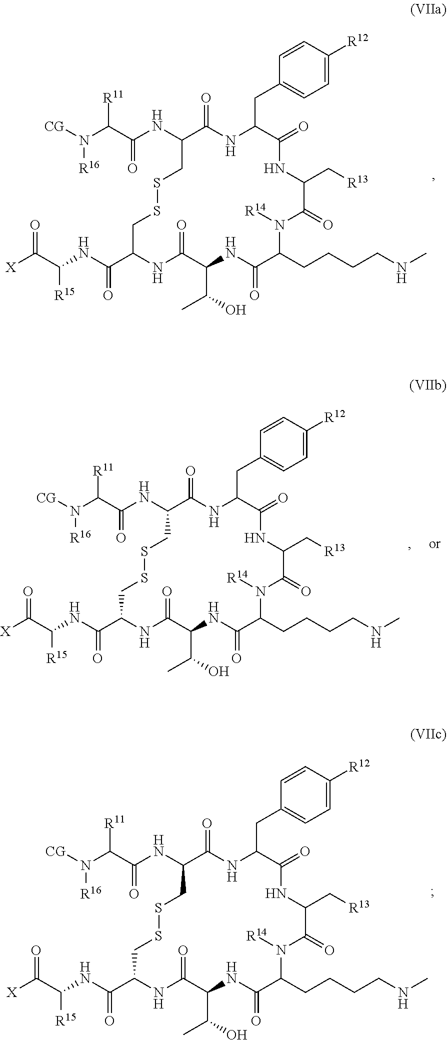

wherein the variables are as defined in the twenty-seventh embodiment.

A thirtieth embodiment of the invention is a compound represented by structural formula (I), (IIa), (IIb), (IIc), (IVa), (IVb), (IVc), (Va), (Vb), (Vc), (VIa), (VIb), or (VIc) or a pharmaceutically acceptable salt thereof, wherein the compound is represented by structural formula (VIIIa), (VIIIb), or (VIIIc).

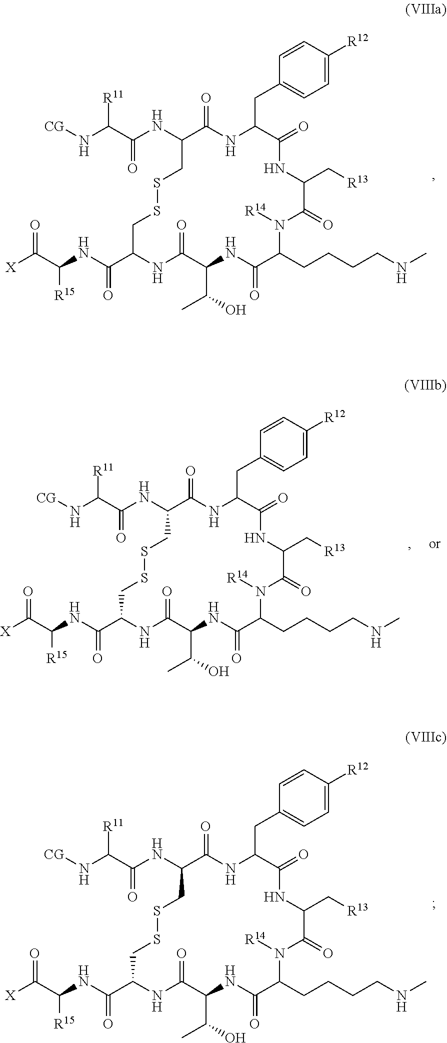

wherein:
X is OH or NH₂;
CG is a chelating group selected from:

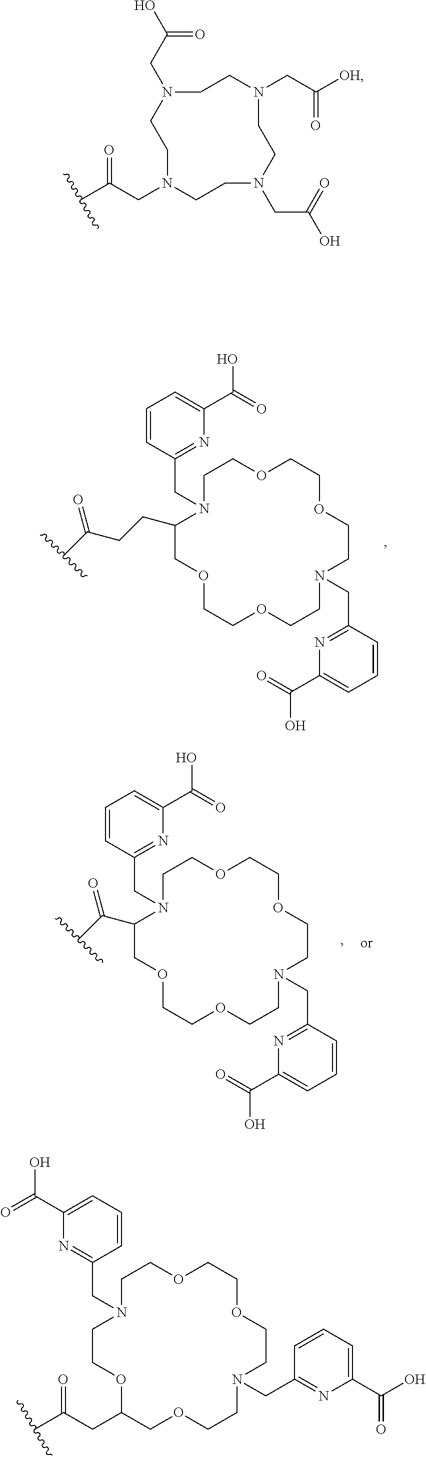 [SMILES: *C(=O)CN1CCN(CC(=O)O)CCN(CC(=O)O)CCN(CC(=O)O)CC1 ; *C(=O)CCC1COCCOCCN(Cc2cccc(C(=O)O)n2)CCOCCOCCN1Cc1cccc(C(=O)O)n1 ; *C(=O)C1COCCOCCN(Cc2cccc(C(=O)O)n2)CCOCCOCCN1Cc1cccc(C(=O)O)n1 ; *C(=O)CC1COCCN(Cc2cccc(C(=O)O)n2)CCOCCOCCN(Cc2cccc(C(=O)O)n2)CCO1]

R¹¹ is selected from

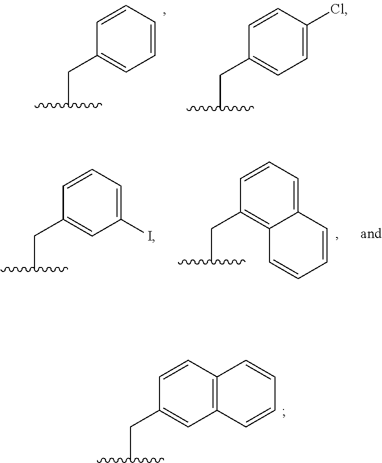 [SMILES: *Cc1ccccc1 ; *Cc1ccc(Cl)cc1 ; *Cc1cccc(I)c1 ; *Cc1cccc2ccccc12 ; *Cc1ccc2ccccc2c1]

R¹² is OH or

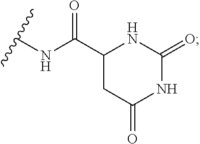 [SMILES: *NC(=O)C1CC(=O)NC(=O)N1]

R¹³ is

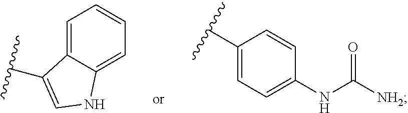 [SMILES: *c1c[nH]c2ccccc12 ; *c1ccc(NC(N)=O)cc1]

R¹⁴ is H or CH₃; and
R¹⁵ is

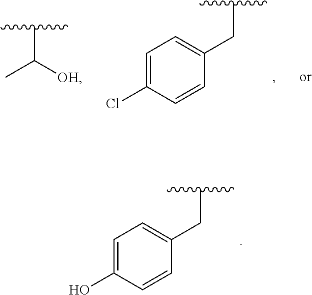

A thirty-first embodiment of the invention is a compound represented by structural formula (I), (IIa), (IIb), (IIc), (IIIa), (IIIb), (IIIc), (Va), (Vb), (Vc), (VIIa), (VIIb), or (VIIc), or a pharmaceutically acceptable salt thereof, wherein the compound is represented by structural formula (IXa), (IXb), or (IXc):

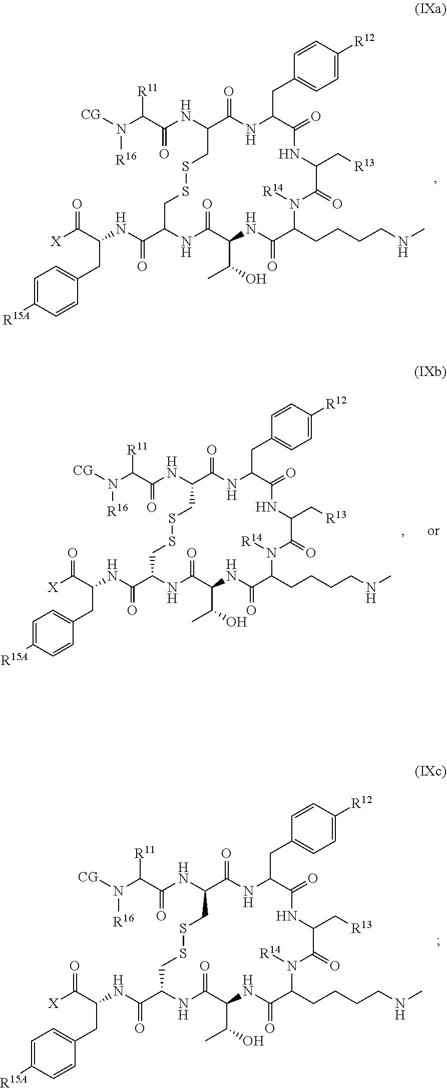

wherein:
X is OH or NH₂;
CG is a chelating group selected from:

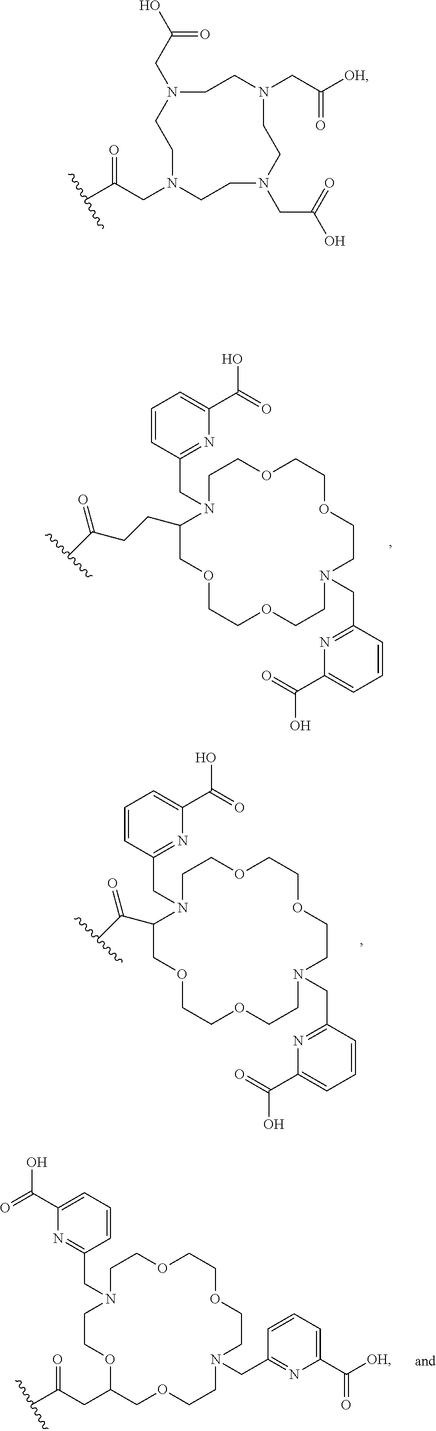 [SMILES: *C(=O)CN1CCN(CC(=O)O)CCN(CC(=O)O)CCN(CC(=O)O)CC1 ; *C(=O)CCC1COCCOCCN(Cc2cccc(C(=O)O)n2)CCOCCOCCN1Cc1cccc(C(=O)O)n1 ; *C(=O)C1COCCOCCN(Cc2cccc(C(=O)O)n2)CCOCCOCCN1Cc1cccc(C(=O)O)n1 ; *C(=O)CC1COCCN(Cc2cccc(C(=O)O)n2)CCOCCOCCN(Cc2cccc(C(=O)O)n2)CCO1]

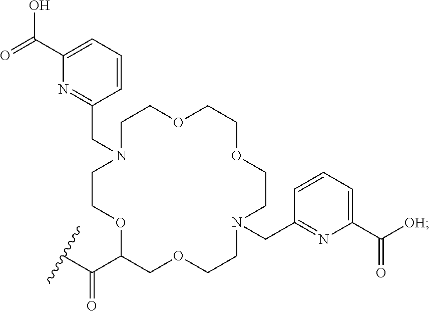 [SMILES: *C(=O)C1COCCN(Cc2cccc(C(=O)O)n2)CCOCCOCCN(Cc2cccc(C(=O)O)n2)CCO1]

R¹¹ is selected from

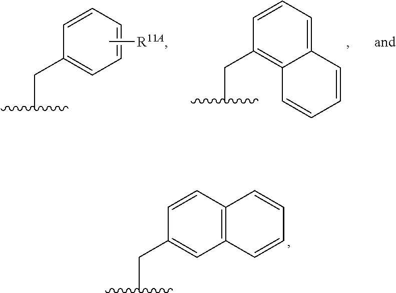

wherein R^{11A} is H or halogen;
R¹² is OH or

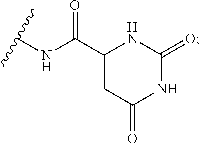 [SMILES: *NC(=O)C1CC(=O)NC(=O)N1]

R¹³ is

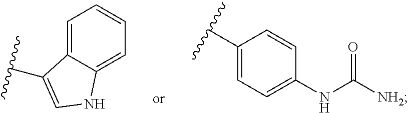 [SMILES: *c1c[nH]c2ccccc12 ; *c1ccc(NC(N)=O)cc1]

R¹⁴ and R¹⁶ are each independently H or CH₃; and
R^{15A} is Cl, OH, or F.

A thirty-second embodiment of the invention is a compound represented by structural formula (IXa), (IXb), or (IXc), or a pharmaceutically acceptable salt thereof, wherein X is NH₂ and the remainder of the variables are as described in the thirty-first embodiment.

A thirty-third embodiment of the invention is a compound represented by structural formula (IXa), (IXb), or (IXc), or a pharmaceutically acceptable salt thereof, wherein R¹³ is

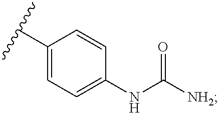 [SMILES: *c1ccc(NC(N)=O)cc1]

and the remainder of the variables are as described in the thirty-first or thirty-second embodiment.

A thirty-fourth embodiment of the invention is a compound represented by structural formula (IXa), (IXb), or (IXc), or a pharmaceutically acceptable salt thereof, wherein:
CG is a chelating group selected from:

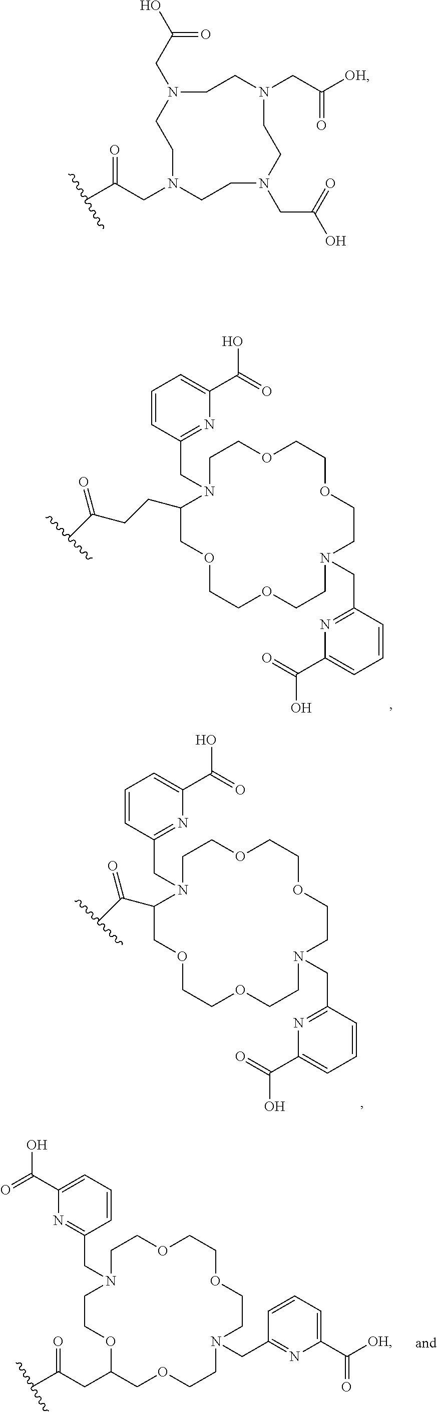 [SMILES: *C(=O)CN1CCN(CC(=O)O)CCN(CC(=O)O)CCN(CC(=O)O)CC1 ; *C(=O)CCC1COCCOCCN(Cc2cccc(C(=O)O)n2)CCOCCOCCN1Cc1cccc(C(=O)O)n1 ; *C(=O)C1COCCOCCN(Cc2cccc(C(=O)O)n2)CCOCCOCCN1Cc1cccc(C(=O)O)n1 ; *C(=O)CC1COCCN(Cc2cccc(C(=O)O)n2)CCOCCOCCN(Cc2cccc(C(=O)O)n2)CCO1]

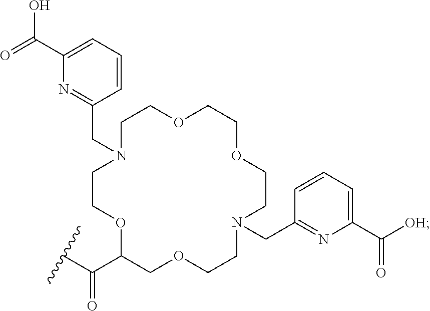 [SMILES: *C(=O)C1COCCN(Cc2cccc(C(=O)O)n2)CCOCCOCCN(Cc2cccc(C(=O)O)n2)CCO1]

R¹¹ is selected from

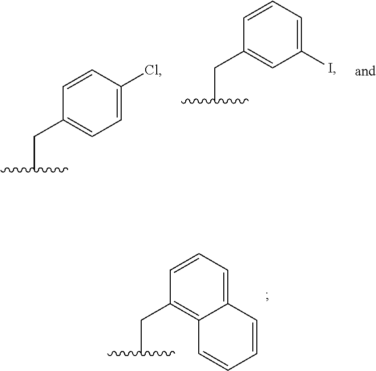 [SMILES: *Cc1ccc(Cl)cc1 ; *Cc1cccc(I)c1 ; *Cc1cccc2ccccc12]

R¹² is OH

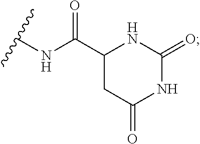 [SMILES: *NC(=O)C1CC(=O)NC(=O)N1]

R¹³ is

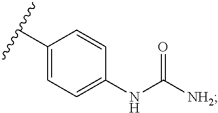 [SMILES: *c1ccc(NC(N)=O)cc1]

R¹⁴ is H or CH₃ and R¹⁶ are H; and
R^{15A} is Cl, OH, or F, and the remainder of the variables are as described in the thirty-first, thirty-second, or thirty-third embodiment.

A thirty-fifth embodiment of the invention is a compound represented by structural formula (IXa), (IXb), (IXc), or a pharmaceutically acceptable salt thereof, wherein CG is a chelating group selected from:

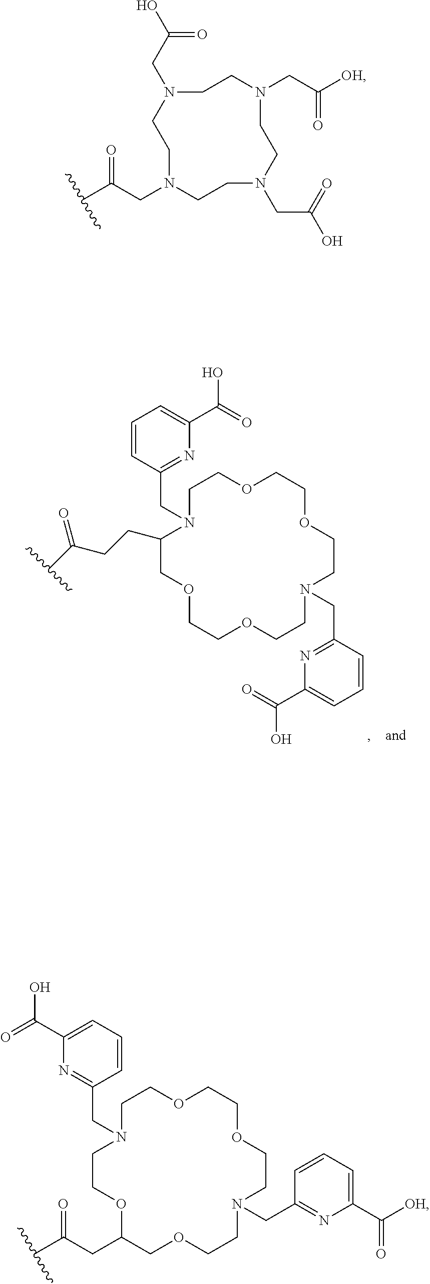 [SMILES: *C(=O)CN1CCN(CC(=O)O)CCN(CC(=O)O)CCN(CC(=O)O)CC1 ; *C(=O)CCC1COCCOCCN(Cc2cccc(C(=O)O)n2)CCOCCOCCN1Cc1cccc(C(=O)O)n1 ; *C(=O)CC1COCCN(Cc2cccc(C(=O)O)n2)CCOCCOCCN(Cc2cccc(C(=O)O)n2)CCO1]

and the remainder of the variables are as described in the thirty-first, thirty-second, thirty-third, or thirty-fourth embodiment.

A thirty-sixth embodiment of the invention is a compound represented by structural formula (IXa), (IX), (IX), or a pharmaceutically acceptable salt thereof, wherein CG is a chelating group selected from

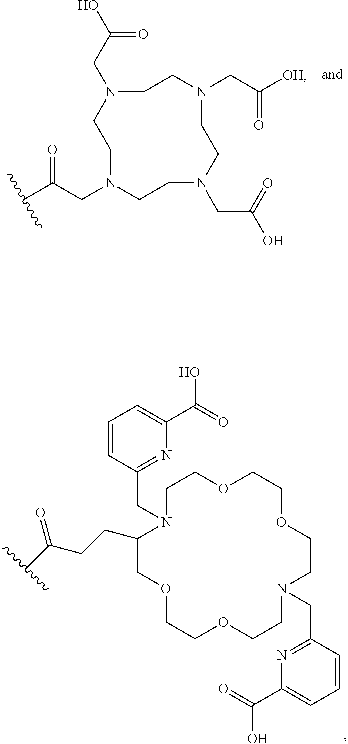 [SMILES: *C(=O)CN1CCN(CC(=O)O)CCN(CC(=O)O)CCN(CC(=O)O)CC1 ; *C(=O)CCC1COCCOCCN(Cc2cccc(C(=O)O)n2)CCOCCOCCN1Cc1cccc(C(=O)O)n1]

and the remainder of the variables are as described in the thirty-first, thirty-second, thirty-third, or thirty-fourth embodiment.

Also, included in the invention are the compounds whose preparation is described in the Exemplification and shown in the Figures, both pharmaceutically acceptable salts thereof and the neutral form. For those compounds comprising a chelating group, chelation with a radionuclide is also included in the invention.

Also included in the invention are the compounds shown in Table 1 below, both pharmaceutically acceptable salts thereof and the neutral form. Chelation with a radionuclide is also included in the invention.

TABLE 1
Compounds of the Invention

| Compound | |
|---|---|
| No. | Structure |
| 1 | 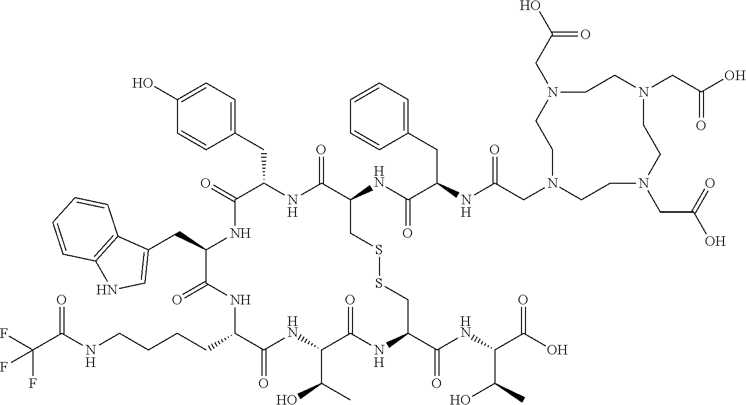 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCNC(=O)C(F)(F)F)C(=O)N[C@@H]([C@@H](C)O)C(=O)N1)C(=O)O] |
| 1a | 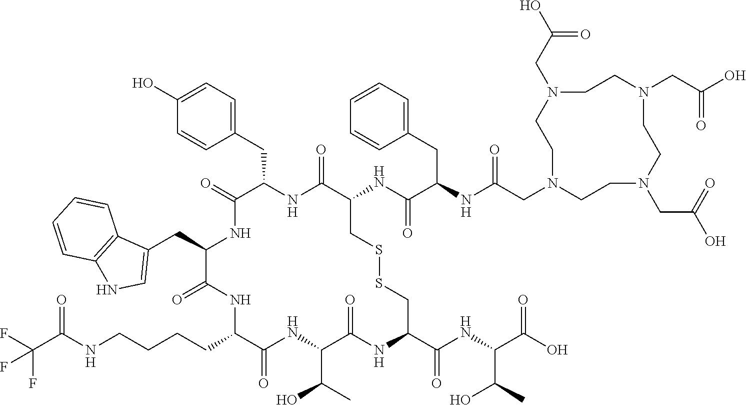 [SMILES: C[C@@H](O)[C@H](NC(=O)[C@@H]1CSSC[C@@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCNC(=O)C(F)(F)F)C(=O)N[C@@H]([C@@H](C)O)C(=O)N1)C(=O)O] |
| 2 | 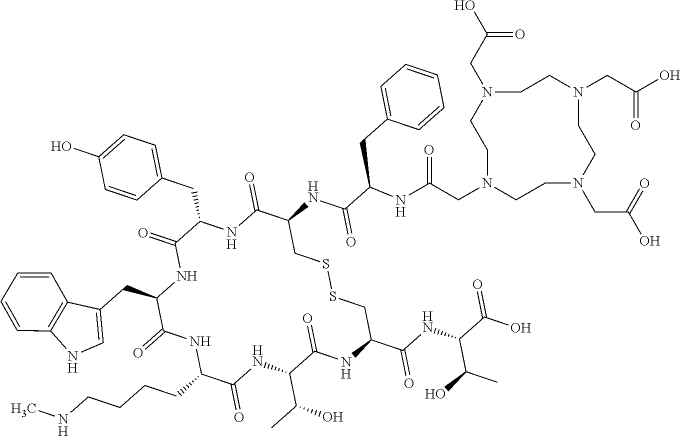 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 2a | 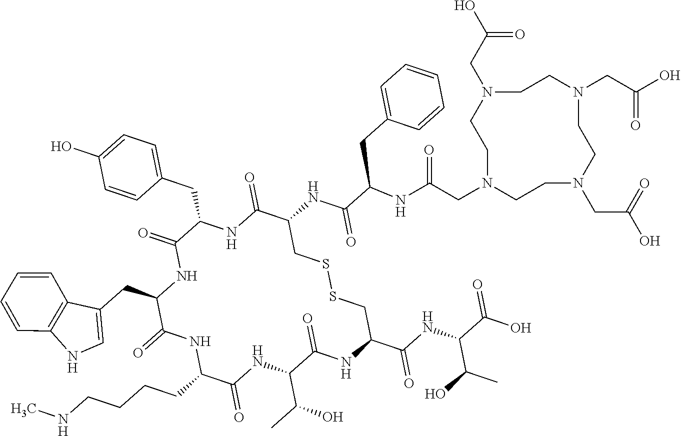 [SMILES: CNCCCC[C@@H]1NC(=O)[C@@H](Cc2c[nH]c3ccccc23)NC(=O)[C@H](Cc2ccc(O)cc2)NC(=O)[C@H](NC(=O)[C@@H](Cc2ccccc2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 3 | 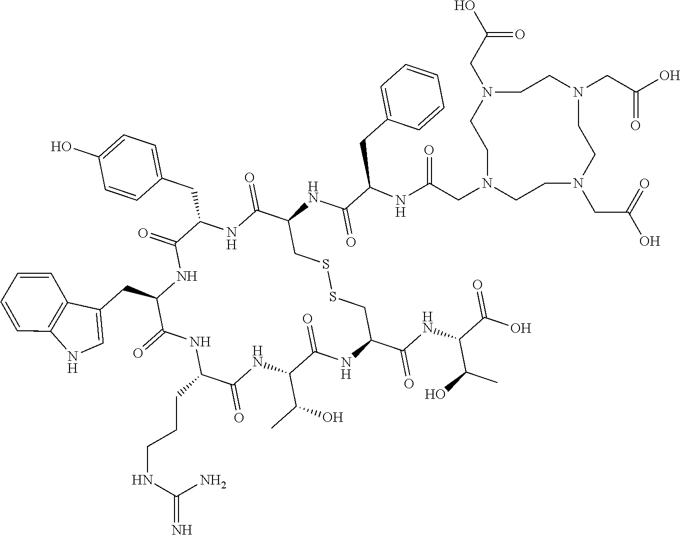 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCNC(=N)N)C(=O)N[C@@H]([C@@H](C)O)C(=O)N1)C(=O)O] |
| 3a | 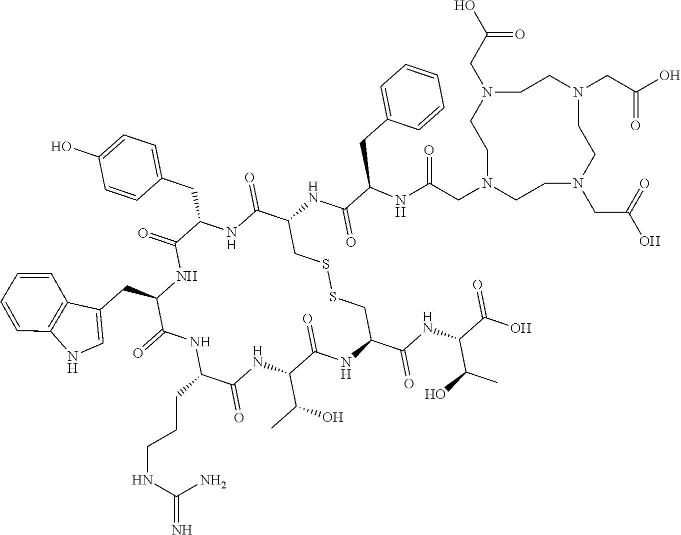 [SMILES: C[C@@H](O)[C@H](NC(=O)[C@@H]1CSSC[C@@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCNC(=N)N)C(=O)N[C@@H]([C@@H](C)O)C(=O)N1)C(=O)O] |
| 4 | 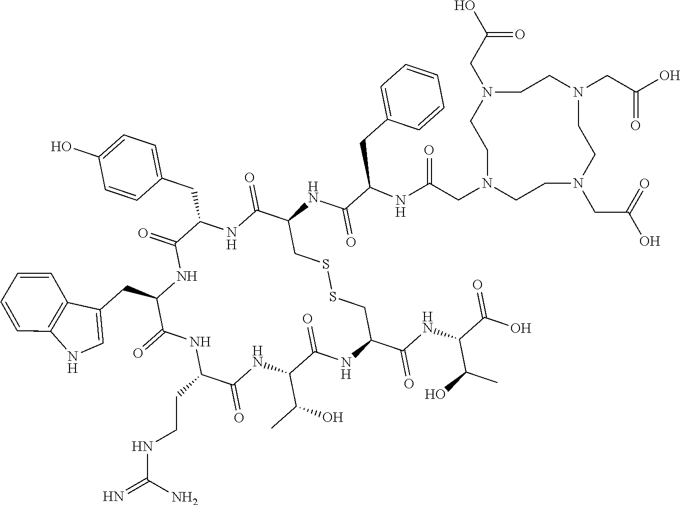 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCNC(=N)N)C(=O)N[C@@H]([C@@H](C)O)C(=O)N1)C(=O)O] |
| 4a | 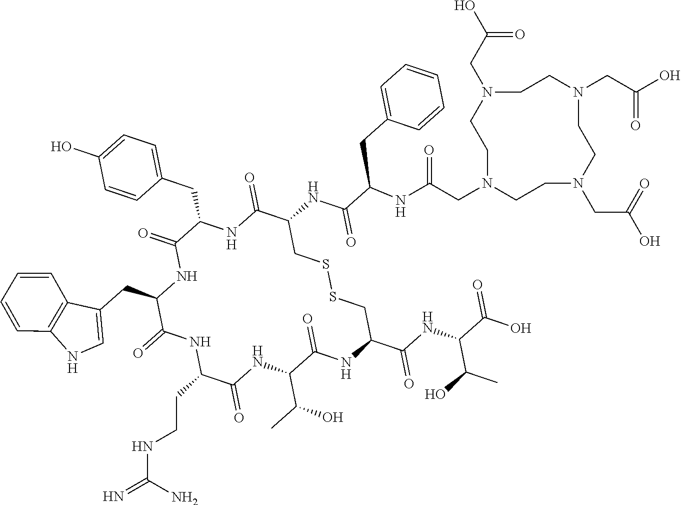 [SMILES: C[C@@H](O)[C@H](NC(=O)[C@@H]1CSSC[C@@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCNC(=N)N)C(=O)N[C@@H]([C@@H](C)O)C(=O)N1)C(=O)O] |
| 5 | 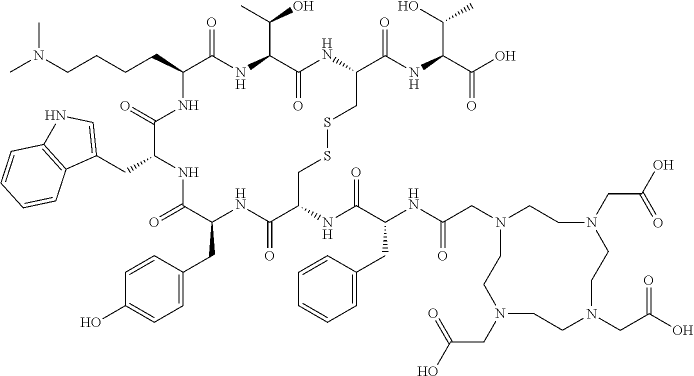 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN(C)C)C(=O)N[C@@H]([C@@H](C)O)C(=O)N1)C(=O)O] |
| 5a | 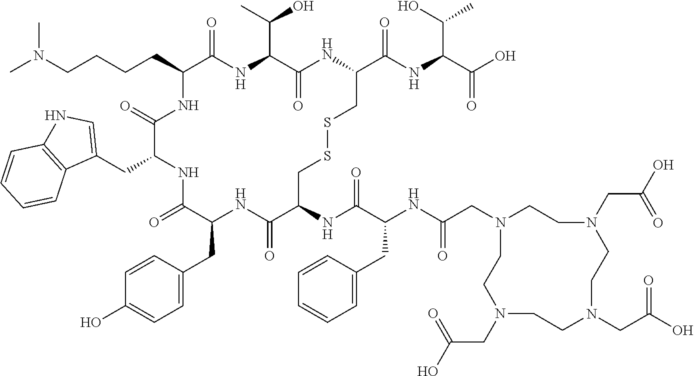 [SMILES: C[C@@H](O)[C@H](NC(=O)[C@@H]1CSSC[C@@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN(C)C)C(=O)N[C@@H]([C@@H](C)O)C(=O)N1)C(=O)O] |
| 6 | 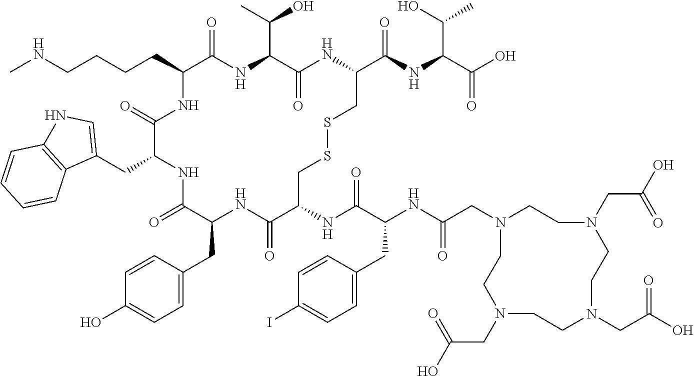 |
| 6a | 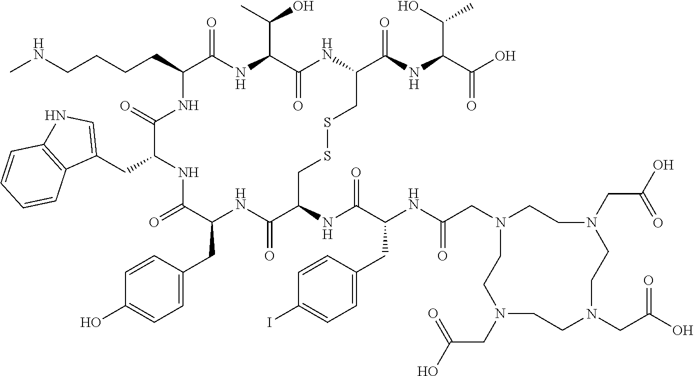 |
| 7 | 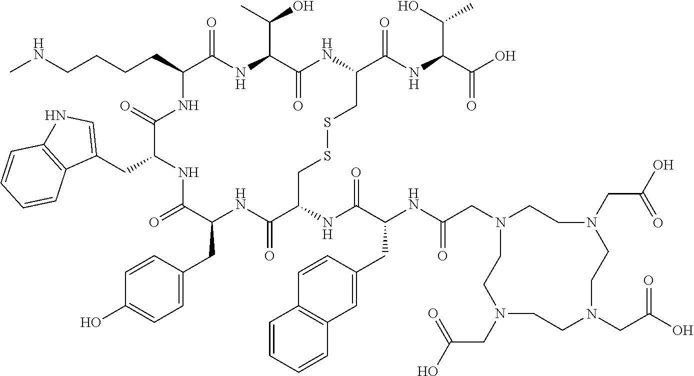 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccc3ccccc3c2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 7a | 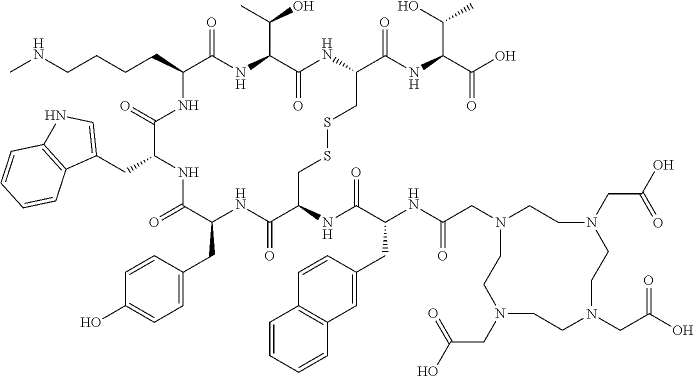 [SMILES: CNCCCC[C@@H]1NC(=O)[C@@H](Cc2c[nH]c3ccccc23)NC(=O)[C@H](Cc2ccc(O)cc2)NC(=O)[C@H](NC(=O)[C@@H](Cc2ccc3ccccc3c2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 8 | 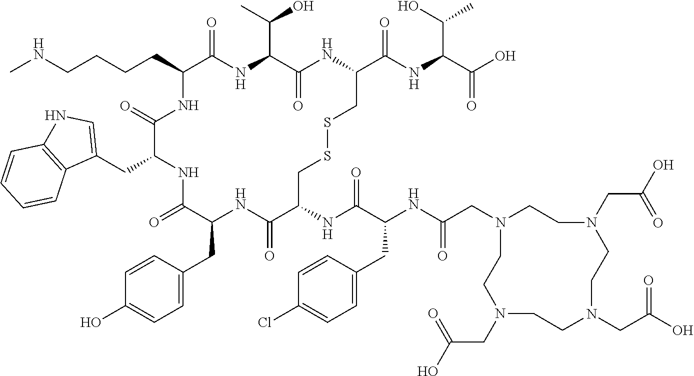 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccc(Cl)cc2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 8a | 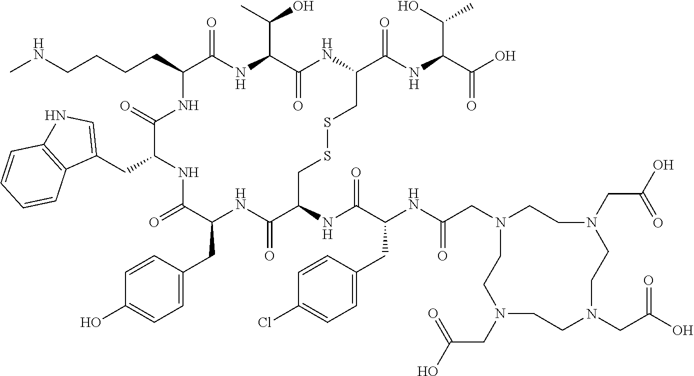 [SMILES: CNCCCC[C@@H]1NC(=O)[C@@H](Cc2c[nH]c3ccccc23)NC(=O)[C@H](Cc2ccc(O)cc2)NC(=O)[C@H](NC(=O)[C@@H](Cc2ccc(Cl)cc2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 9 | 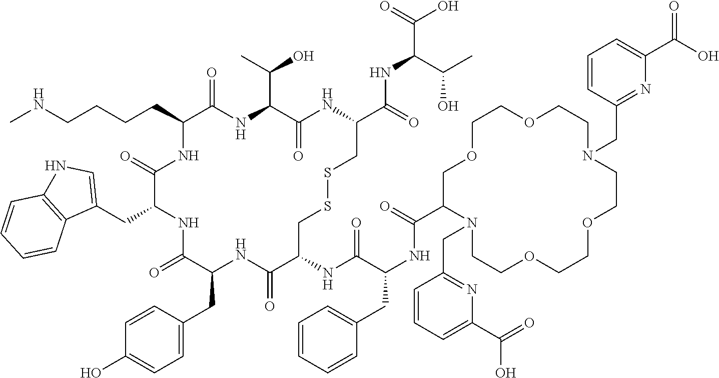 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)C2COCCOCCN(Cc3cccc(C(=O)O)n3)CCOCCOCCN2Cc2cccc(C(=O)O)n2)CSSC[C@@H](C(=O)N[C@@H](C(=O)O)[C@H](C)O)NC(=O)[C@H]([C@@H](C)O)NC1=O] |
| 9a | 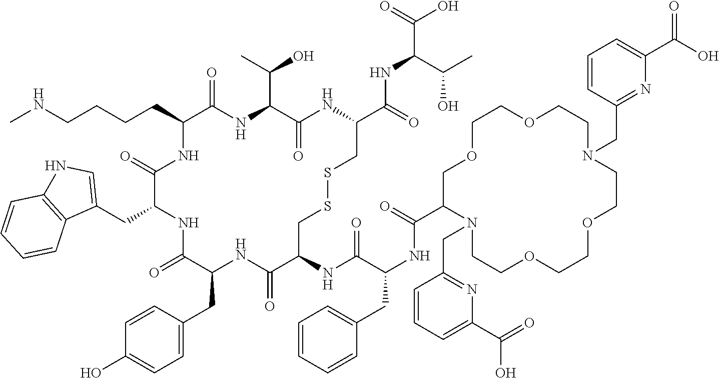 [SMILES: CNCCCC[C@@H]1NC(=O)[C@@H](Cc2c[nH]c3ccccc23)NC(=O)[C@H](Cc2ccc(O)cc2)NC(=O)[C@H](NC(=O)[C@@H](Cc2ccccc2)NC(=O)C2COCCOCCN(Cc3cccc(C(=O)O)n3)CCOCCOCCN2Cc2cccc(C(=O)O)n2)CSSC[C@@H](C(=O)N[C@@H](C(=O)O)[C@H](C)O)NC(=O)[C@H]([C@@H](C)O)NC1=O] |
| 10 | 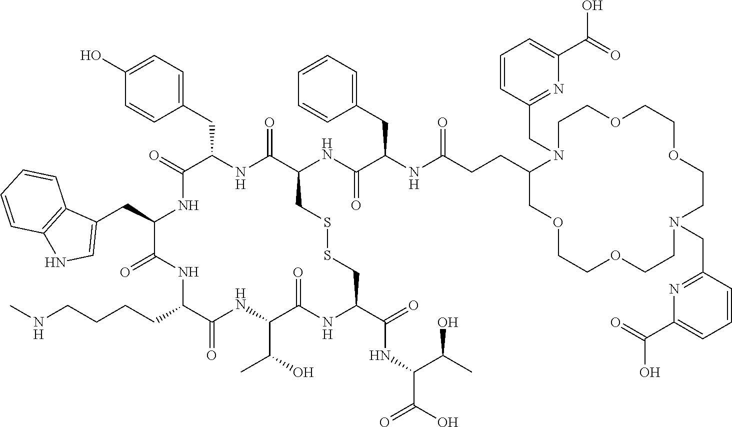 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)CCC2COCCOCCN(Cc3cccc(C(=O)O)n3)CCOCCOCCN2Cc2cccc(C(=O)O)n2)CSSC[C@@H](C(=O)N[C@@H](C(=O)O)[C@H](C)O)NC(=O)[C@H]([C@@H](C)O)NC1=O] |
| 10a | 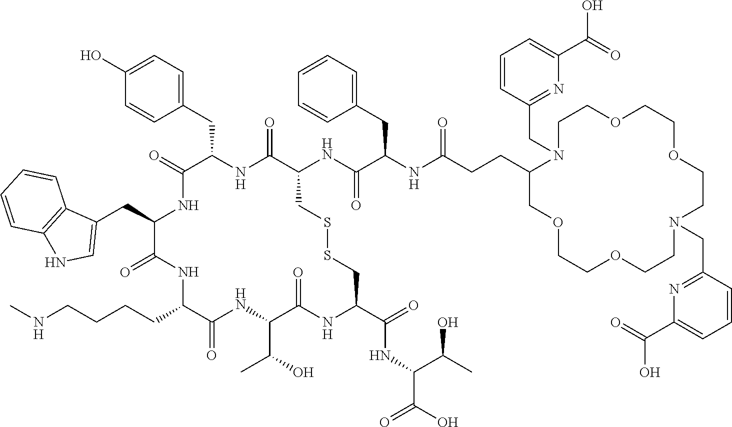 [SMILES: CNCCCC[C@@H]1NC(=O)[C@@H](Cc2c[nH]c3ccccc23)NC(=O)[C@H](Cc2ccc(O)cc2)NC(=O)[C@H](NC(=O)[C@@H](Cc2ccccc2)NC(=O)CCC2COCCOCCN(Cc3cccc(C(=O)O)n3)CCOCCOCCN2Cc2cccc(C(=O)O)n2)CSSC[C@@H](C(=O)N[C@@H](C(=O)O)[C@H](C)O)NC(=O)[C@H]([C@@H](C)O)NC1=O] |
| 11 | 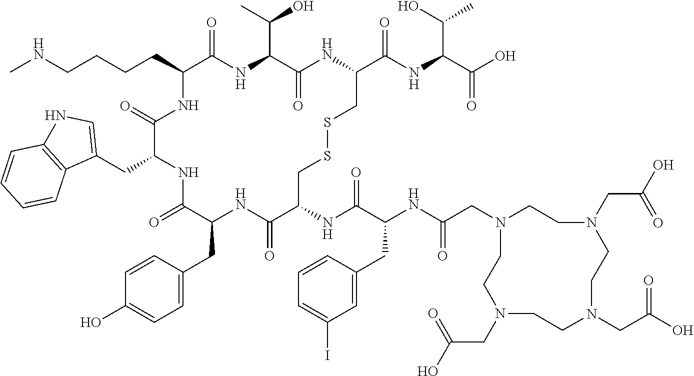 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2cccc(I)c2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 11a | 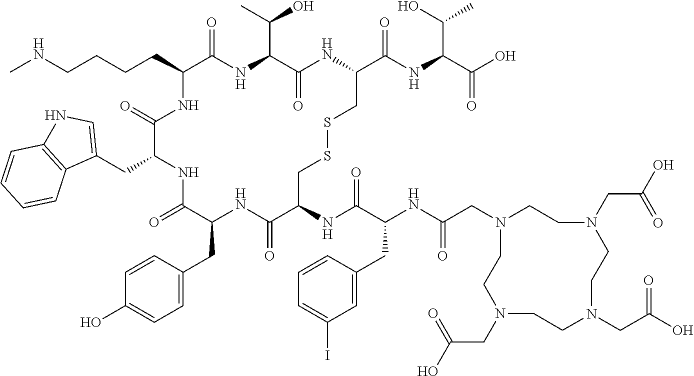 [SMILES: CNCCCC[C@@H]1NC(=O)[C@@H](Cc2c[nH]c3ccccc23)NC(=O)[C@H](Cc2ccc(O)cc2)NC(=O)[C@H](NC(=O)[C@@H](Cc2cccc(I)c2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 12 | 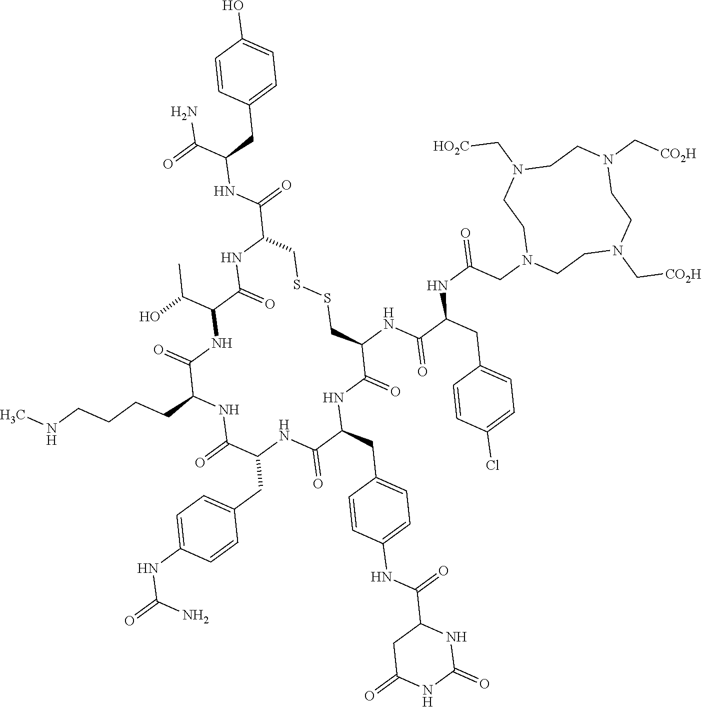 |
| 12a | 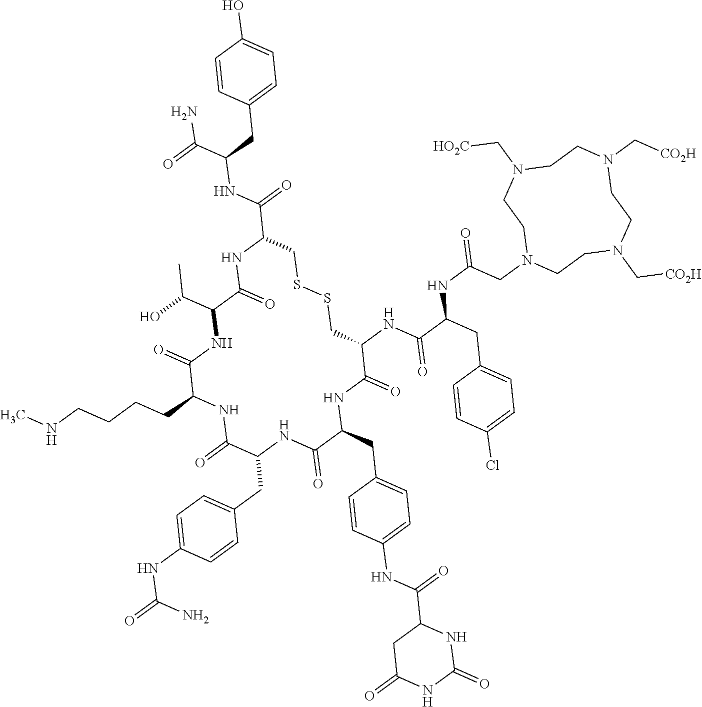 |
| 13 | 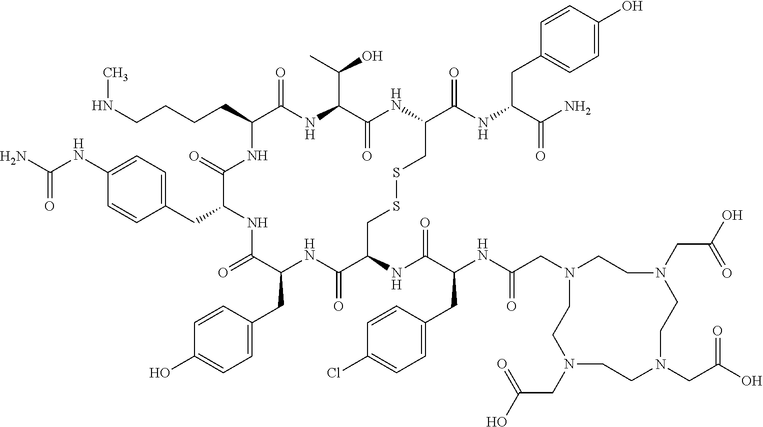 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2ccc(Cl)cc2)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |
| 13a | 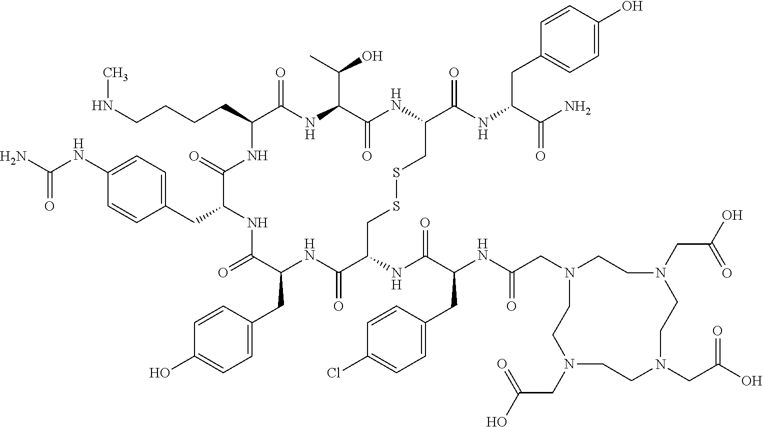 [SMILES: CNCCCC[C@@H]1NC(=O)[C@@H](Cc2ccc(NC(N)=O)cc2)NC(=O)[C@H](Cc2ccc(O)cc2)NC(=O)[C@@H](NC(=O)[C@H](Cc2ccc(Cl)cc2)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |
| 14 | 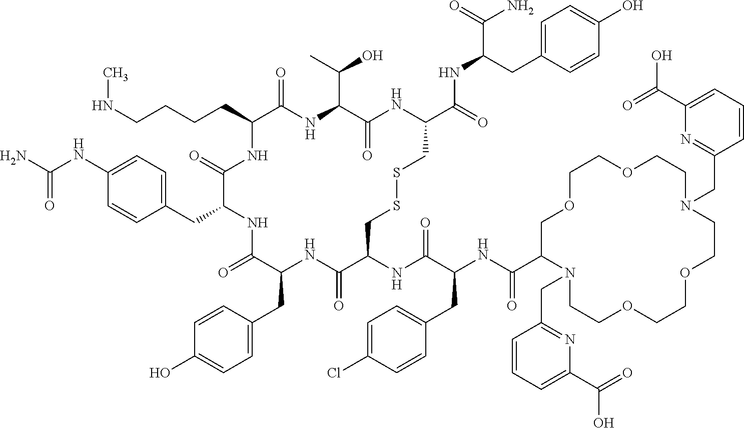 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2ccc(Cl)cc2)NC(=O)C2COCCOCCN(Cc3cccc(C(=O)O)n3)CCOCCOCCN2Cc2cccc(C(=O)O)n2)CSSC[C@@H](C(=O)N[C@H](Cc2ccc(O)cc2)C(N)=O)NC(=O)[C@H]([C@@H](C)O)NC1=O] |
| 14a | 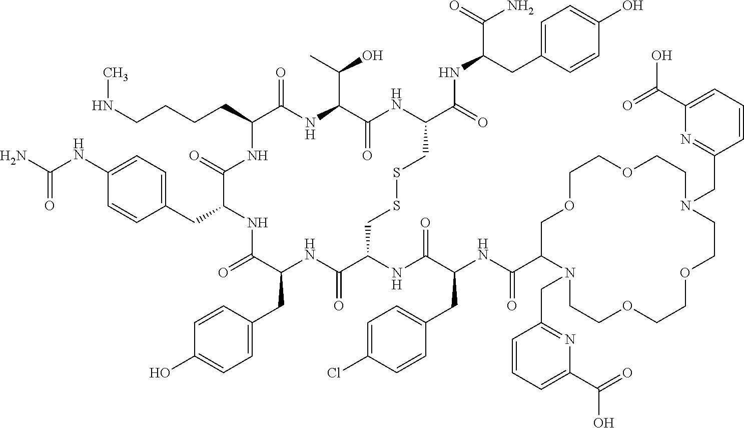 [SMILES: CNCCCC[C@@H]1NC(=O)[C@@H](Cc2ccc(NC(N)=O)cc2)NC(=O)[C@H](Cc2ccc(O)cc2)NC(=O)[C@@H](NC(=O)[C@H](Cc2ccc(Cl)cc2)NC(=O)C2COCCOCCN(Cc3cccc(C(=O)O)n3)CCOCCOCCN2Cc2cccc(C(=O)O)n2)CSSC[C@@H](C(=O)N[C@H](Cc2ccc(O)cc2)C(N)=O)NC(=O)[C@H]([C@@H](C)O)NC1=O] |
| 15 | 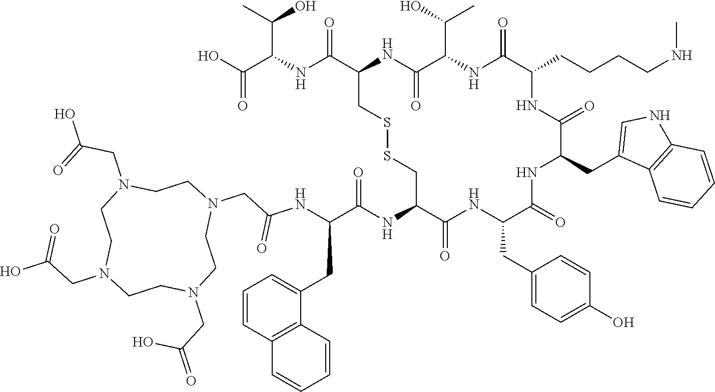 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2cccc3ccccc23)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 15a | 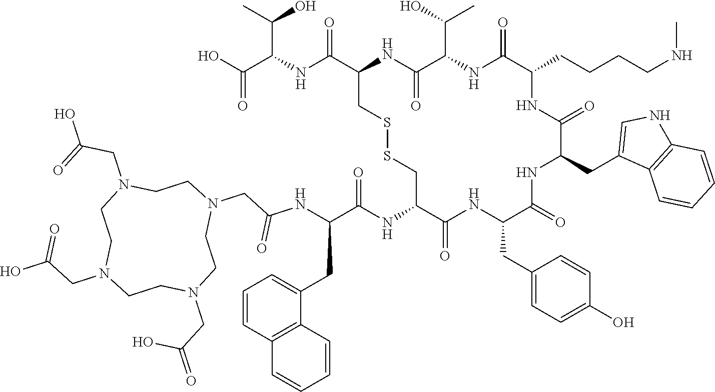 [SMILES: CNCCCC[C@@H]1NC(=O)[C@@H](Cc2c[nH]c3ccccc23)NC(=O)[C@H](Cc2ccc(O)cc2)NC(=O)[C@H](NC(=O)[C@@H](Cc2cccc3ccccc23)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 16 | 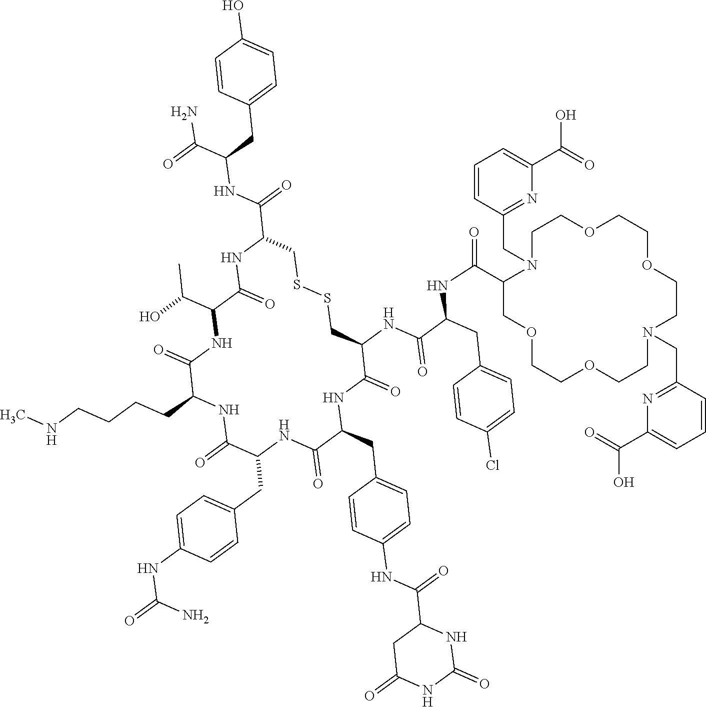 |
| 16a | 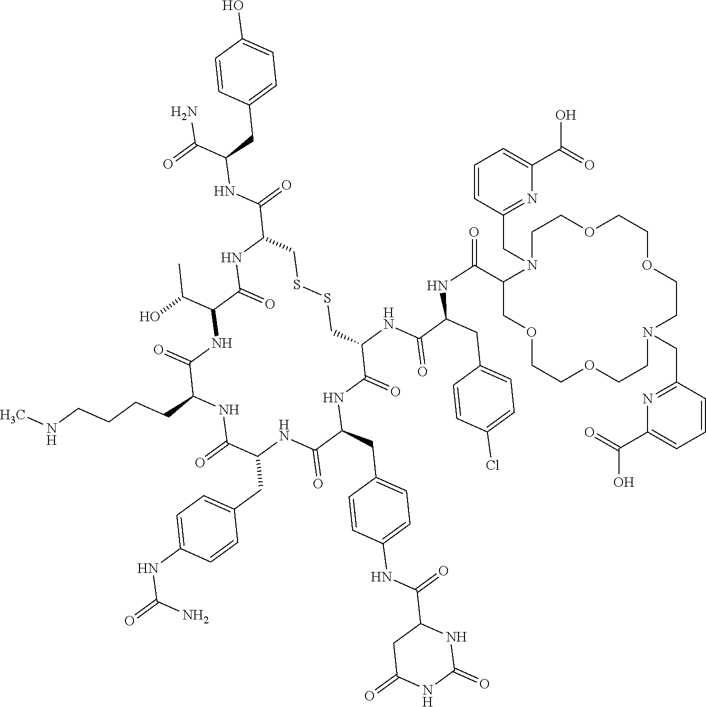 |
| 17 | 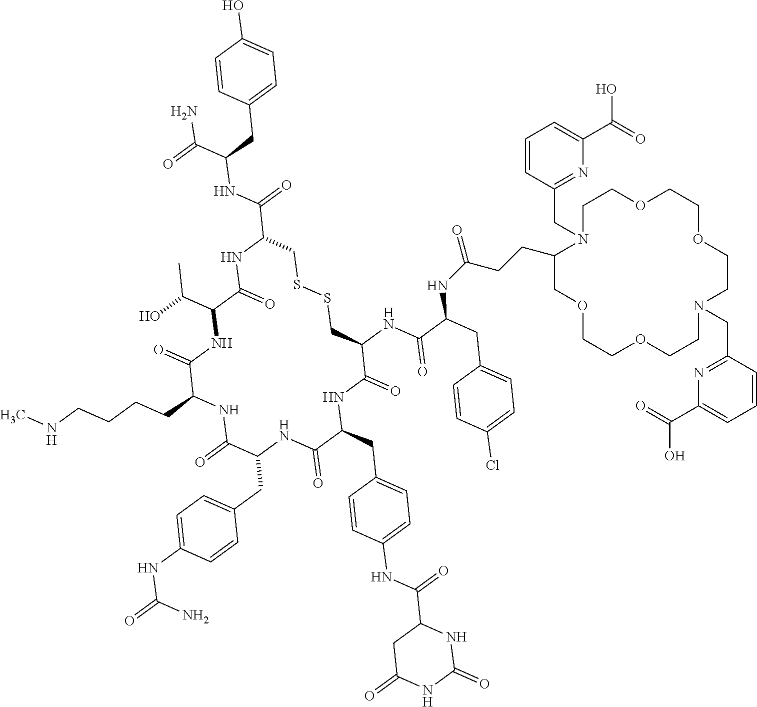 |
| 17a | 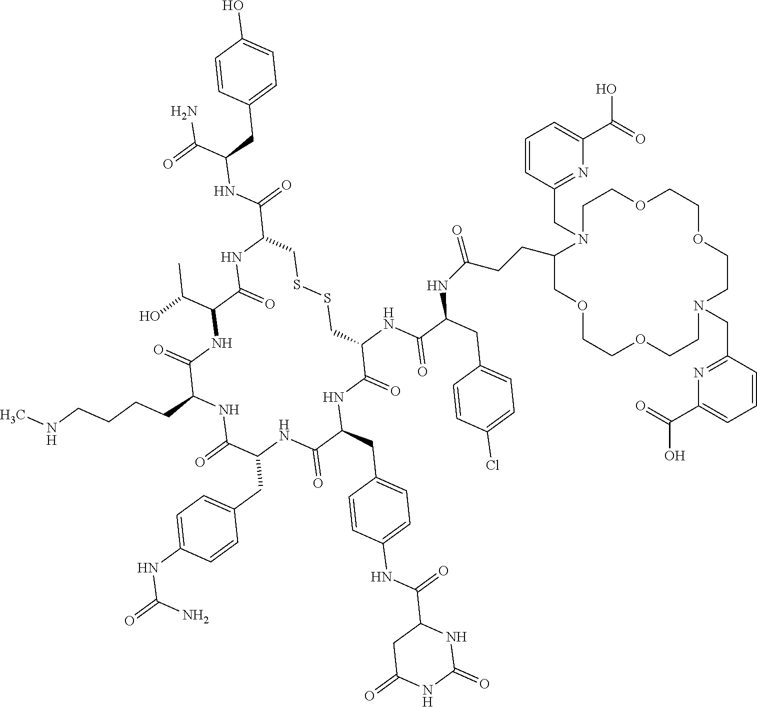 |
| 18 | 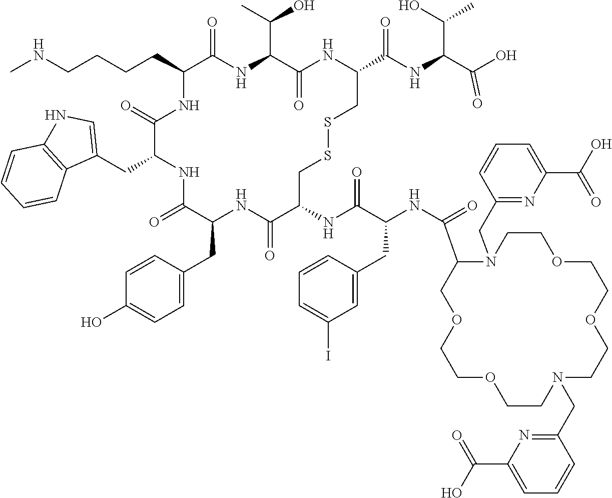 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2cccc(I)c2)NC(=O)C2COCCOCCN(Cc3cccc(C(=O)O)n3)CCOCCOCCN2Cc2cccc(C(=O)O)n2)CSSC[C@@H](C(=O)N[C@H](C(=O)O)[C@@H](C)O)NC(=O)[C@H]([C@@H](C)O)NC1=O] |
| 18a | 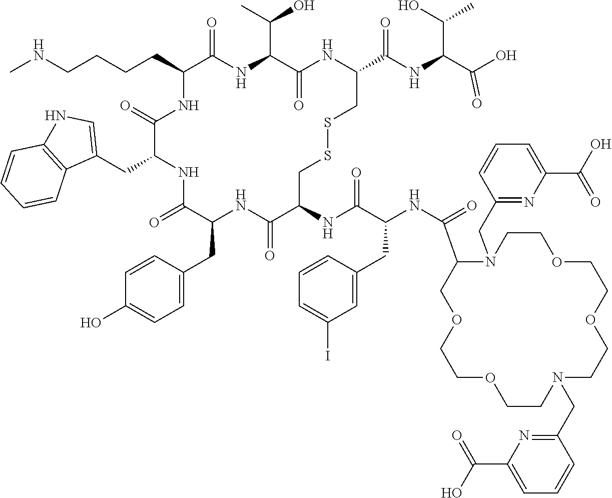 [SMILES: CNCCCC[C@@H]1NC(=O)[C@@H](Cc2c[nH]c3ccccc23)NC(=O)[C@H](Cc2ccc(O)cc2)NC(=O)[C@H](NC(=O)[C@@H](Cc2cccc(I)c2)NC(=O)C2COCCOCCN(Cc3cccc(C(=O)O)n3)CCOCCOCCN2Cc2cccc(C(=O)O)n2)CSSC[C@@H](C(=O)N[C@H](C(=O)O)[C@@H](C)O)NC(=O)[C@H]([C@@H](C)O)NC1=O] |
| 19 | 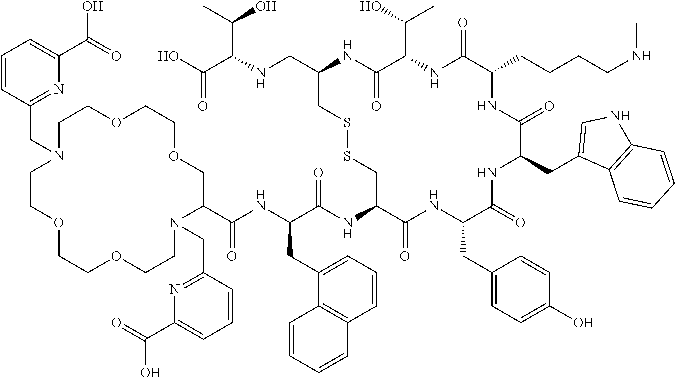 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2cccc3ccccc23)NC(=O)C2COCCOCCN(Cc3cccc(C(=O)O)n3)CCOCCOCCN2Cc2cccc(C(=O)O)n2)CSSC[C@@H](CN[C@H](C(=O)O)[C@@H](C)O)NC(=O)[C@H]([C@@H](C)O)NC1=O] |
| 19a | 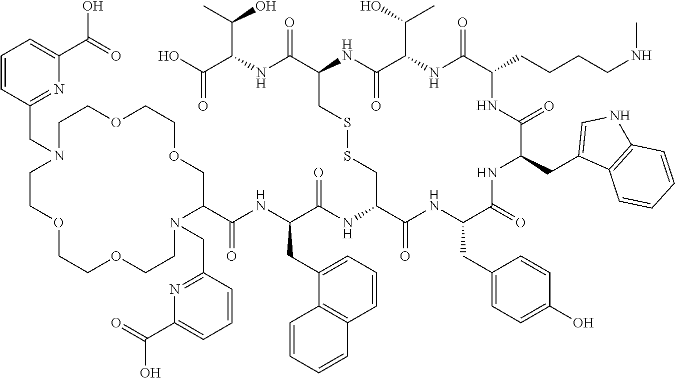 [SMILES: CNCCCC[C@@H]1NC(=O)[C@@H](Cc2c[nH]c3ccccc23)NC(=O)[C@H](Cc2ccc(O)cc2)NC(=O)[C@H](NC(=O)[C@@H](Cc2cccc3ccccc23)NC(=O)C2COCCOCCN(Cc3cccc(C(=O)O)n3)CCOCCOCCN2Cc2cccc(C(=O)O)n2)CSSC[C@@H](C(=O)N[C@H](C(=O)O)[C@@H](C)O)NC(=O)[C@H]([C@@H](C)O)NC1=O] |
| 20 | 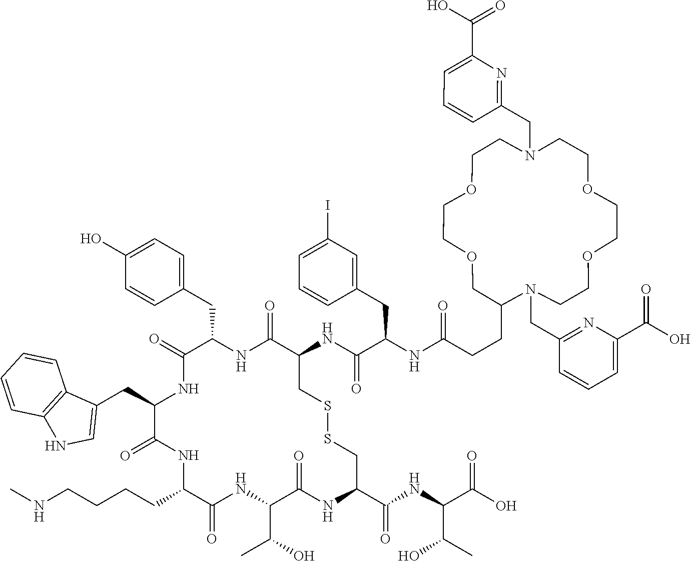 |
| 20a | 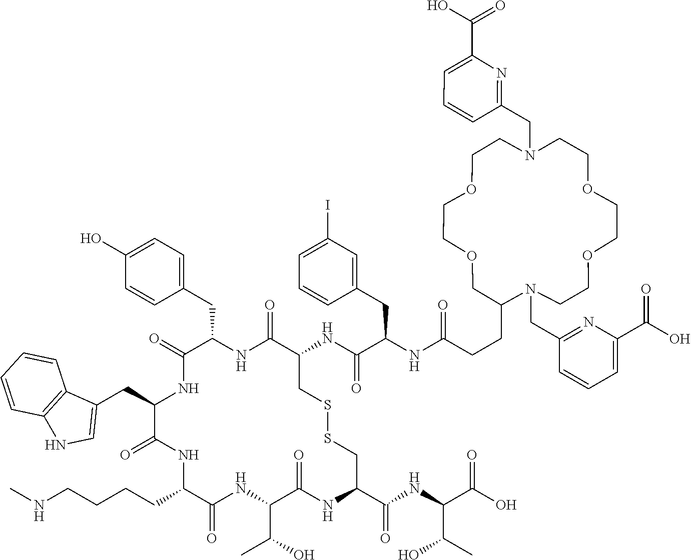 |
| 21 | 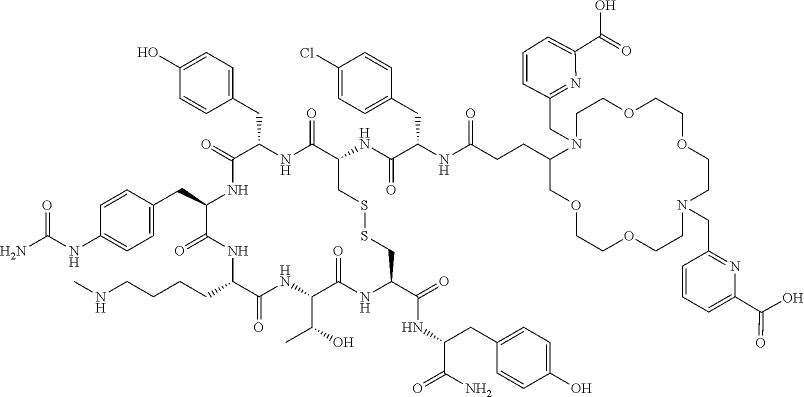 |
| 21a | 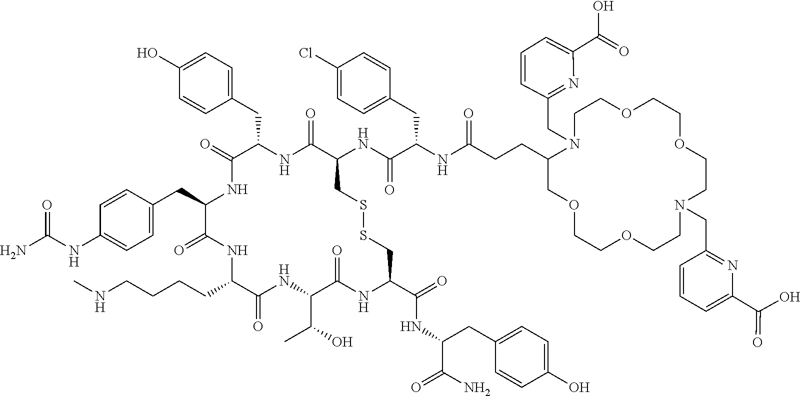 |
| 22 | 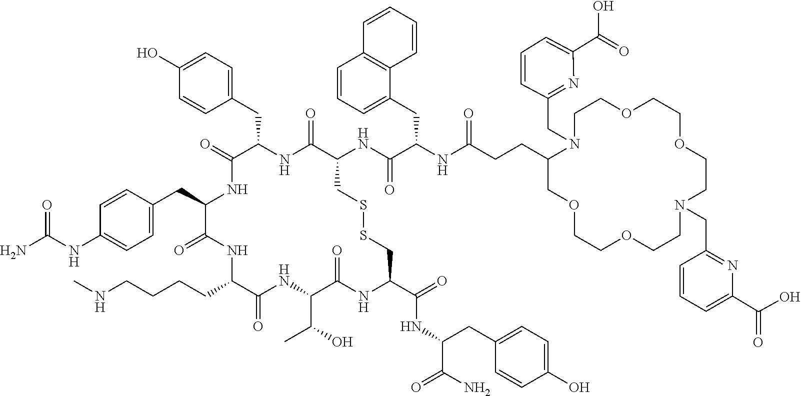 |
| 22a | 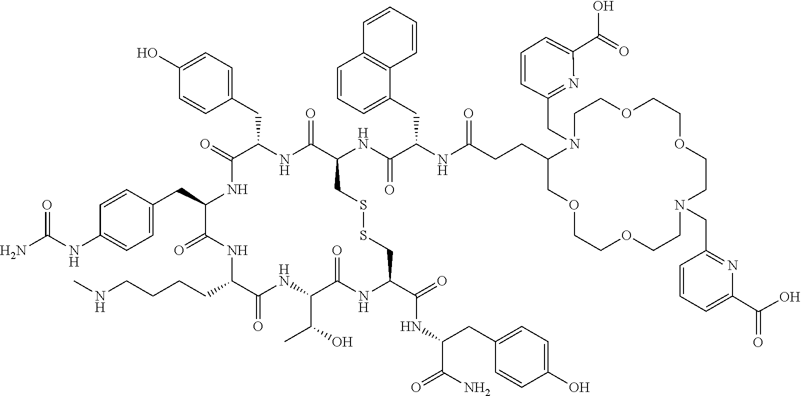 |
| 23 | 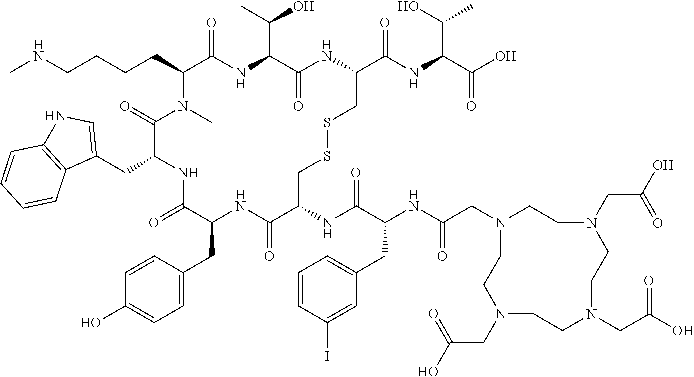 [SMILES: CNCCCC[C@H]1C(=O)N[C@@H]([C@@H](C)O)C(=O)N[C@H](C(=O)N[C@H](C(=O)O)[C@@H](C)O)CSSC[C@H](NC(=O)[C@@H](Cc2cccc(I)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N1C] |
| 23a | 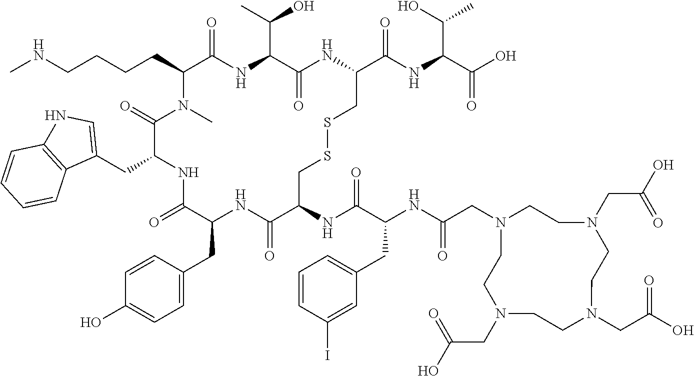 [SMILES: CNCCCC[C@H]1C(=O)N[C@@H]([C@@H](C)O)C(=O)N[C@H](C(=O)N[C@H](C(=O)O)[C@@H](C)O)CSSC[C@@H](NC(=O)[C@@H](Cc2cccc(I)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N1C] |
| 24 | 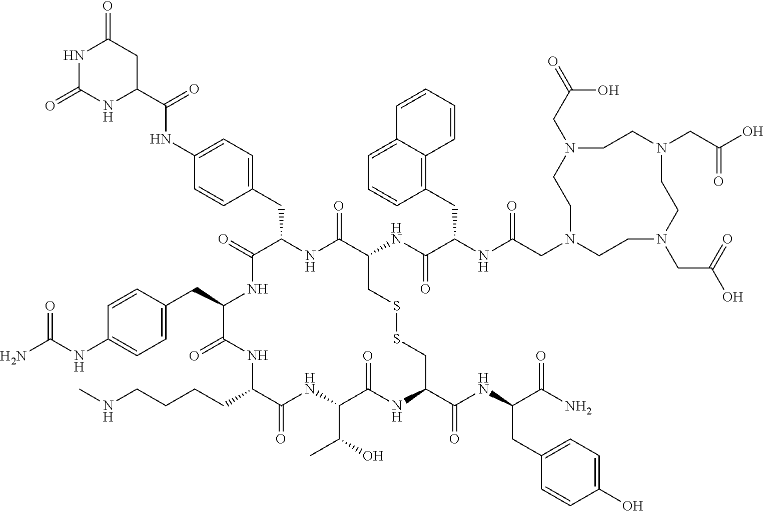 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2cccc3ccccc23)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |
| 24a | 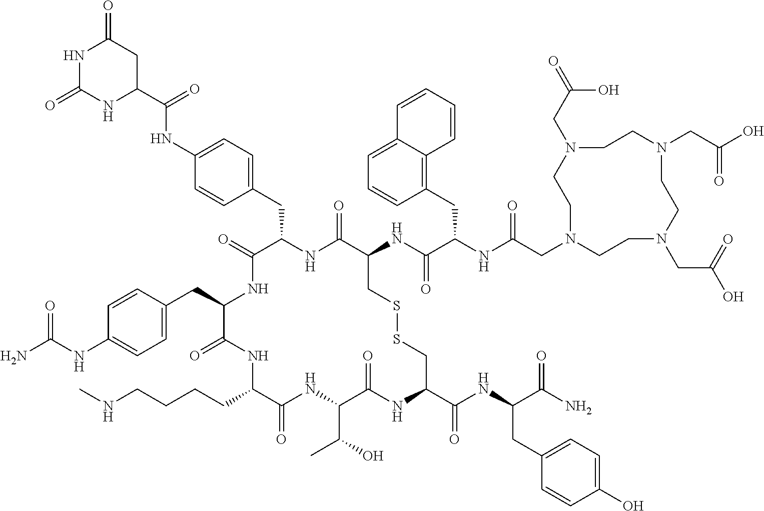 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@@H](NC(=O)[C@H](Cc2cccc3ccccc23)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |
| 25 | 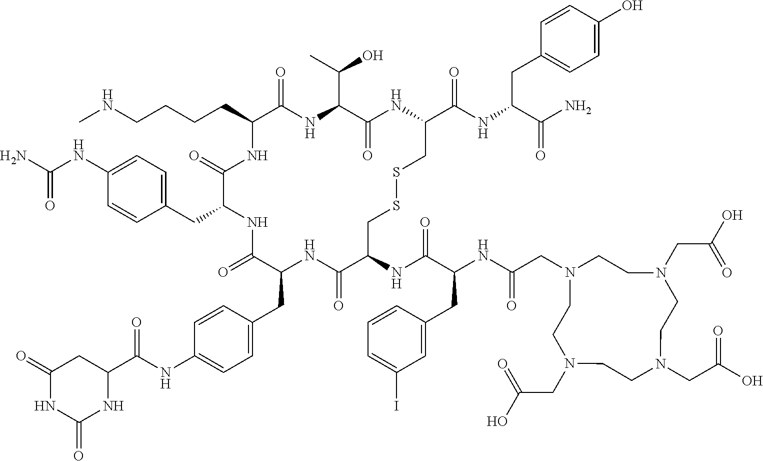 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2cccc(I)c2)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |
| 25a | 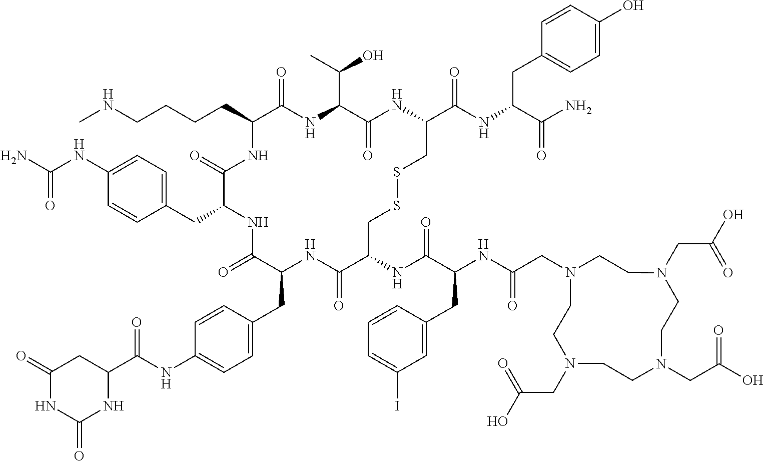 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@@H](NC(=O)[C@H](Cc2cccc(I)c2)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |
| 26 | 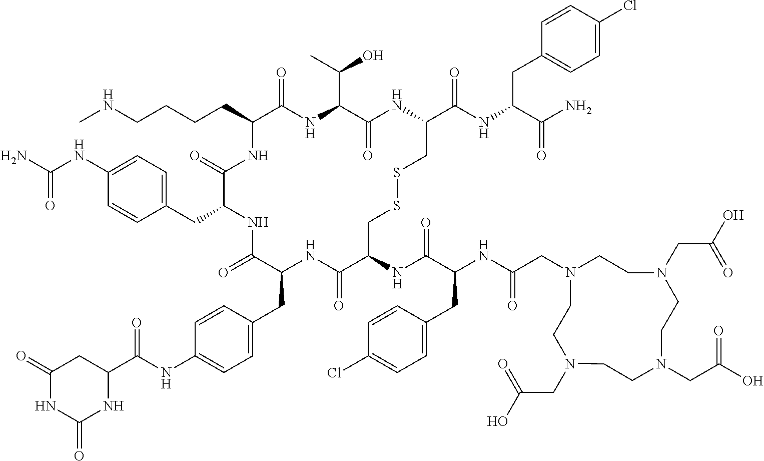 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2ccc(Cl)cc2)NC(=O)CN2CCN(CC(=O)O)CCN(CC(=O)O)CCN(CC(=O)O)CC2)CSSC[C@@H](C(=O)N[C@H](Cc2ccc(Cl)cc2)C(N)=O)NC(=O)[C@H]([C@@H](C)O)NC1=O] |
| 26a | 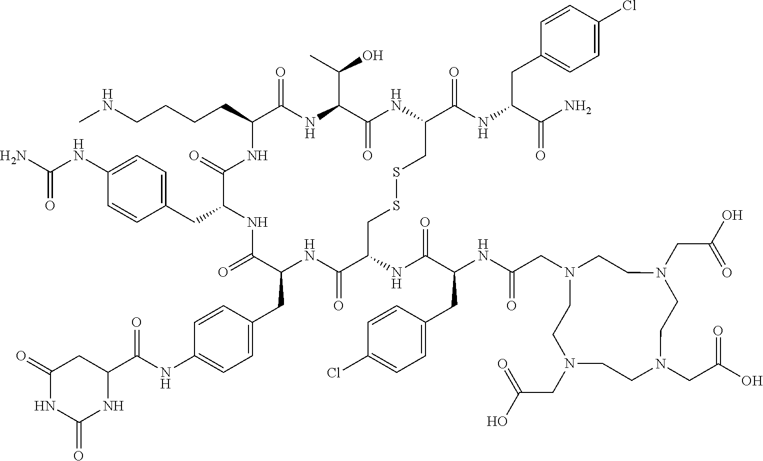 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@@H](NC(=O)[C@H](Cc2ccc(Cl)cc2)NC(=O)CN2CCN(CC(=O)O)CCN(CC(=O)O)CCN(CC(=O)O)CC2)CSSC[C@@H](C(=O)N[C@H](Cc2ccc(Cl)cc2)C(N)=O)NC(=O)[C@H]([C@@H](C)O)NC1=O] |
| 27 | 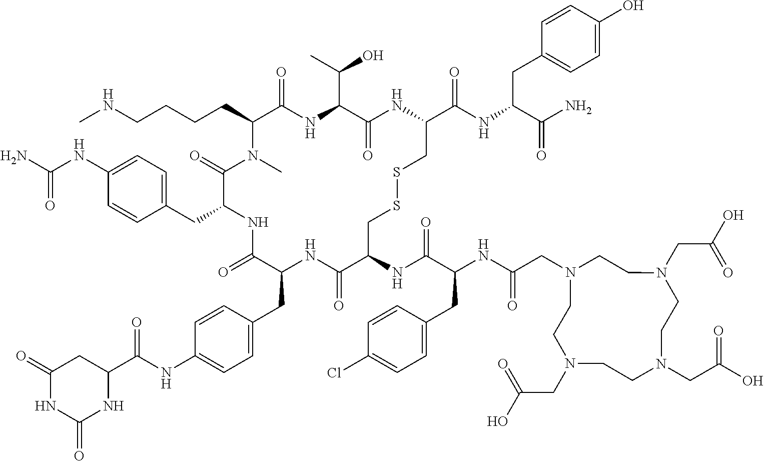 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)CN2CCN(CC(=O)O)CCN(CC(=O)O)CCN(CC(=O)O)CC2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| 27a | 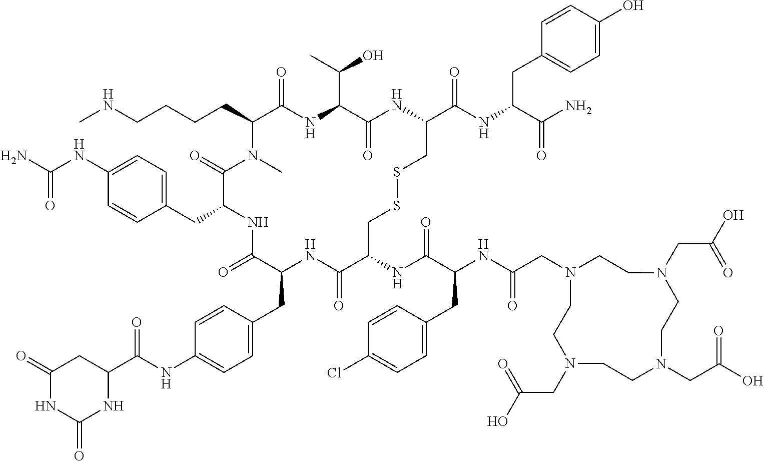 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@H](NC(=O)[C@H](Cc2ccc(Cl)cc2)NC(=O)CN2CCN(CC(=O)O)CCN(CC(=O)O)CCN(CC(=O)O)CC2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| 28 | 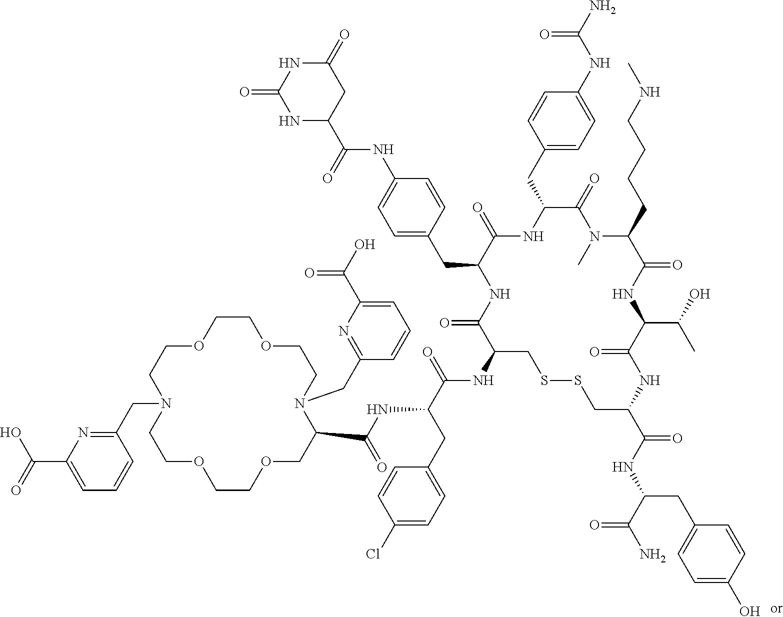 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)[C@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| | 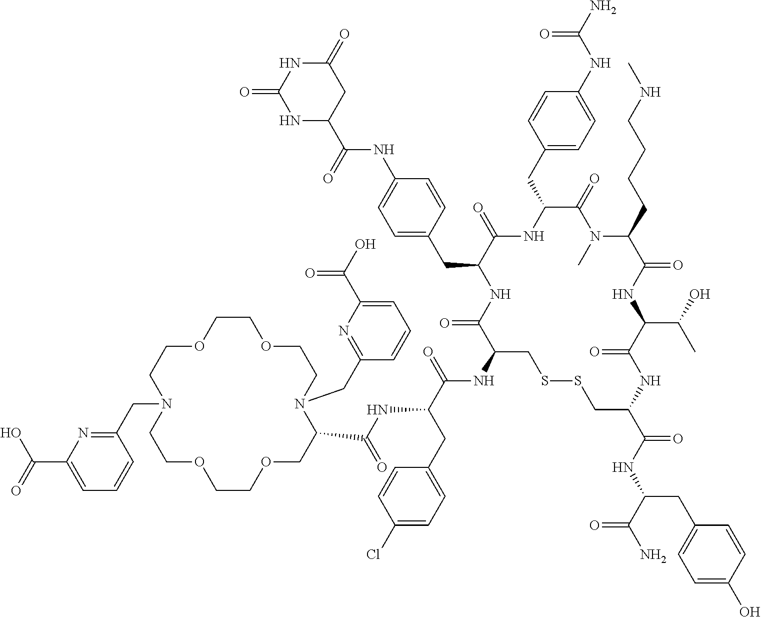 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)[C@@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| 28a | 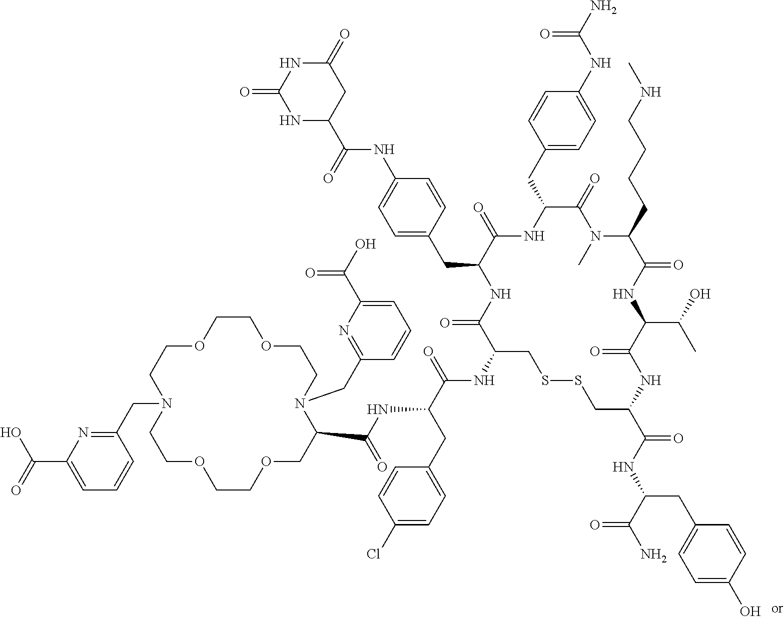 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@H](NC(=O)[C@H](Cc2ccc(Cl)cc2)NC(=O)[C@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| | 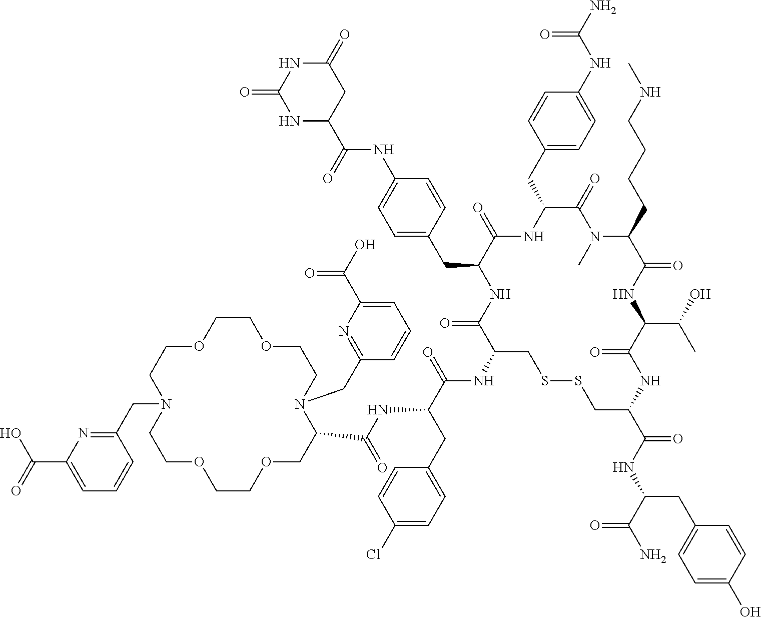 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@H](NC(=O)[C@H](Cc2ccc(Cl)cc2)NC(=O)[C@@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| 29 | 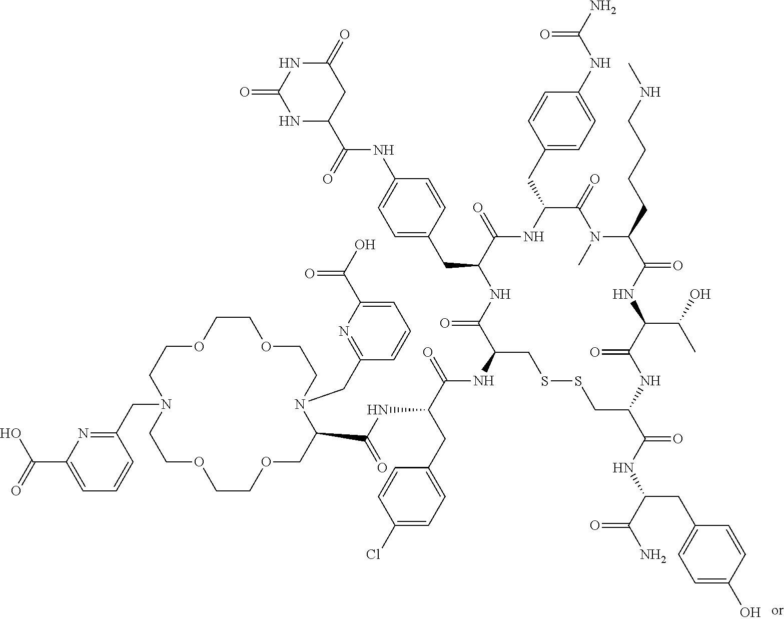 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)[C@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| | 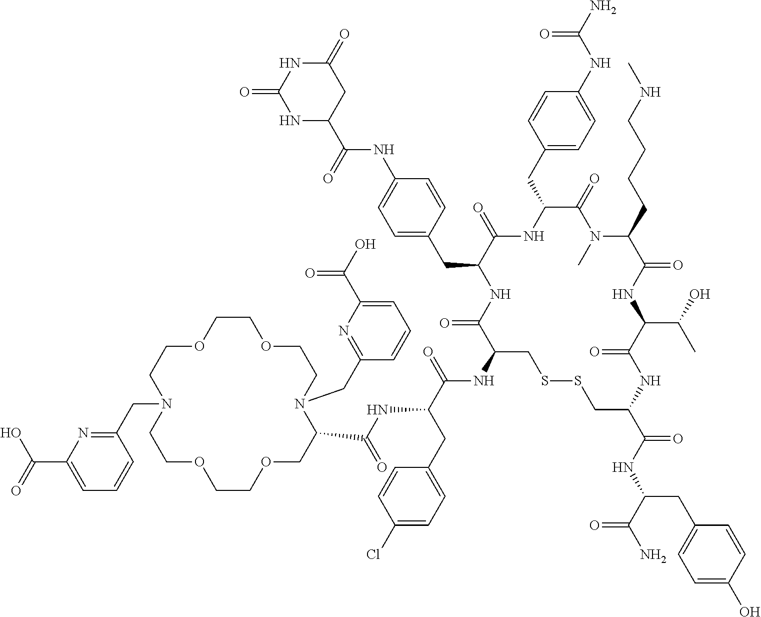 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)[C@@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| 29a | 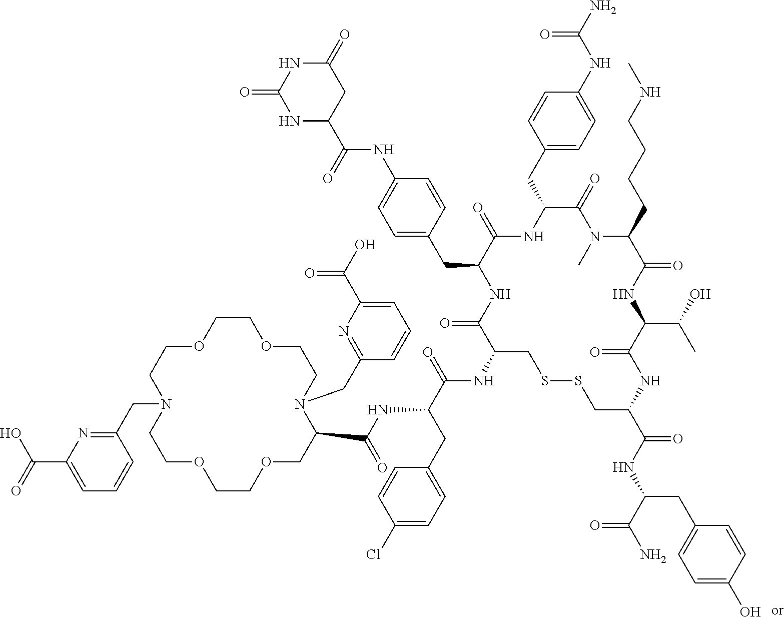 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@H](NC(=O)[C@H](Cc2ccc(Cl)cc2)NC(=O)[C@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| | 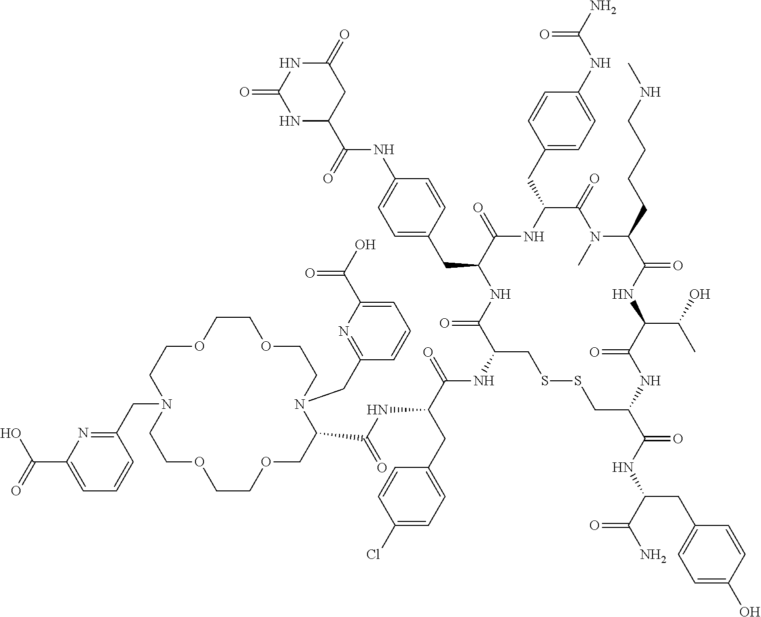 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@H](NC(=O)[C@H](Cc2ccc(Cl)cc2)NC(=O)[C@@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| 30 | 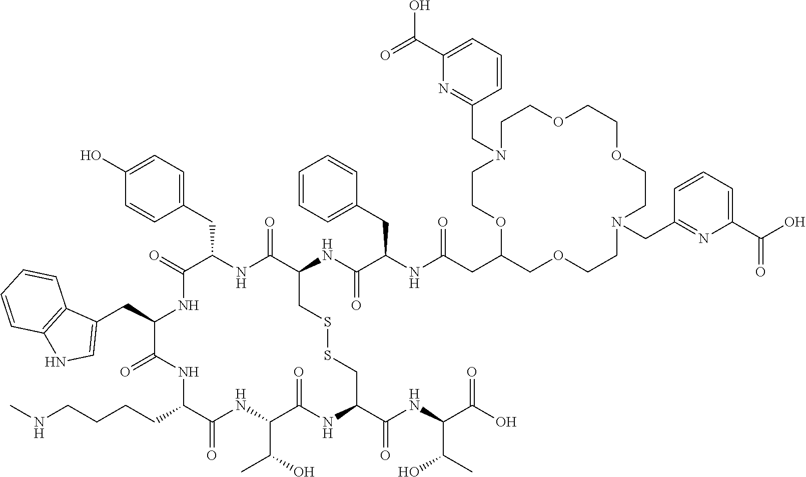 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)CC2COCCN(Cc3cccc(C(=O)O)n3)CCOCCOCCN(Cc3cccc(C(=O)O)n3)CCO2)CSSC[C@@H](C(=O)N[C@@H](C(=O)O)[C@H](C)O)NC(=O)[C@H]([C@@H](C)O)NC1=O] |
| 30a | 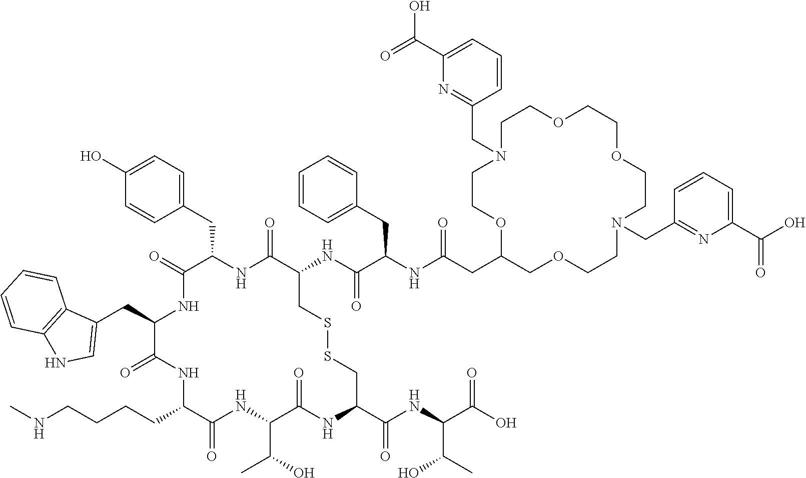 [SMILES: CNCCCC[C@@H]1NC(=O)[C@@H](Cc2c[nH]c3ccccc23)NC(=O)[C@H](Cc2ccc(O)cc2)NC(=O)[C@H](NC(=O)[C@@H](Cc2ccccc2)NC(=O)CC2COCCN(Cc3cccc(C(=O)O)n3)CCOCCOCCN(Cc3cccc(C(=O)O)n3)CCO2)CSSC[C@@H](C(=O)N[C@@H](C(=O)O)[C@H](C)O)NC(=O)[C@H]([C@@H](C)O)NC1=O] |
| 31 | 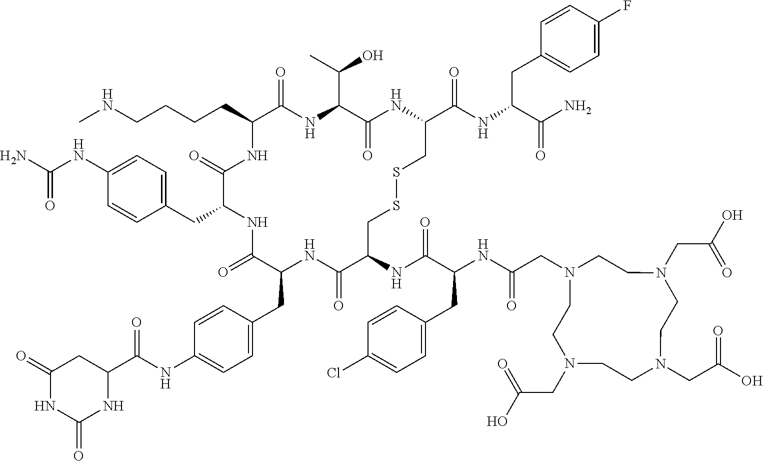 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2ccc(Cl)cc2)NC(=O)CN2CCN(CC(=O)O)CCN(CC(=O)O)CCN(CC(=O)O)CC2)CSSC[C@@H](C(=O)N[C@H](Cc2ccc(F)cc2)C(N)=O)NC(=O)[C@H]([C@@H](C)O)NC1=O] |
| 31a | 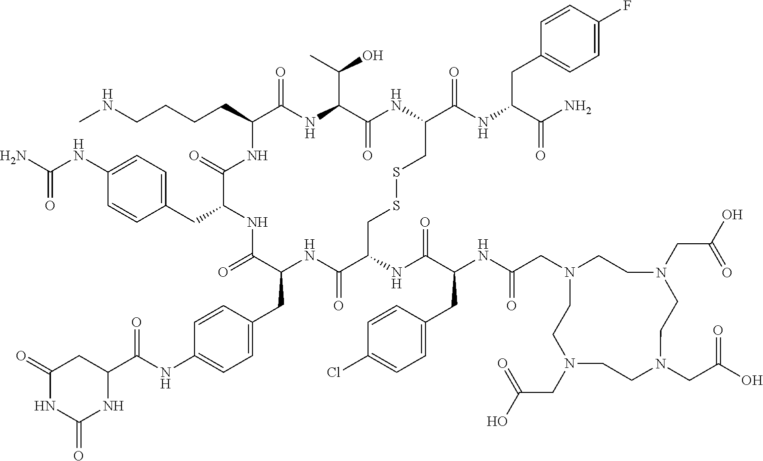 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@@H](NC(=O)[C@H](Cc2ccc(Cl)cc2)NC(=O)CN2CCN(CC(=O)O)CCN(CC(=O)O)CCN(CC(=O)O)CC2)CSSC[C@@H](C(=O)N[C@H](Cc2ccc(F)cc2)C(N)=O)NC(=O)[C@H]([C@@H](C)O)NC1=O] |
| 32 | 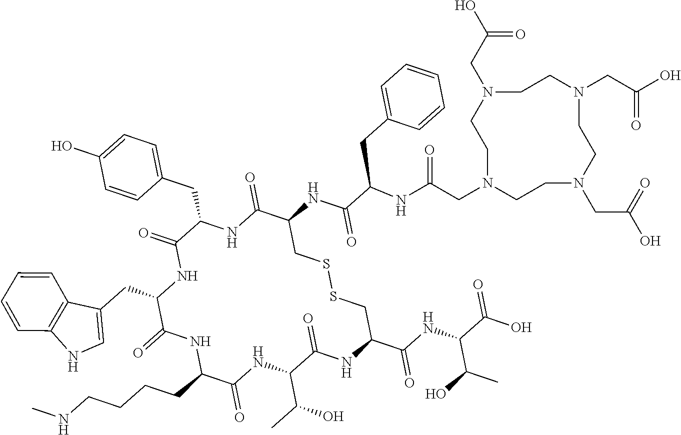 [SMILES: CNCCCC[C@H]1NC(=O)[C@H](Cc2c[nH]c3ccccc23)NC(=O)[C@H](Cc2ccc(O)cc2)NC(=O)[C@@H](NC(=O)[C@@H](Cc2ccccc2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 32a | 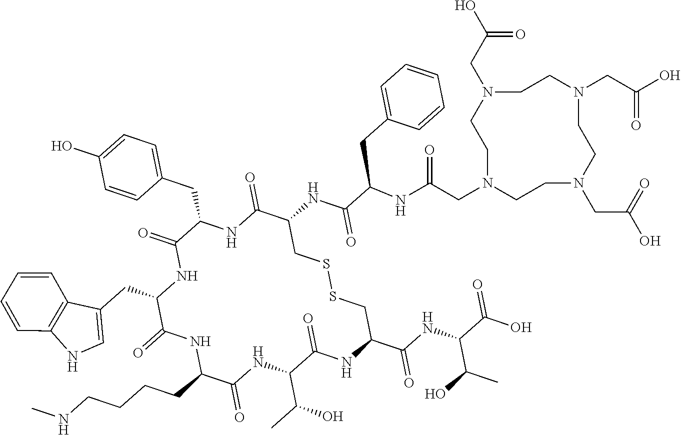 [SMILES: CNCCCC[C@H]1NC(=O)[C@H](Cc2c[nH]c3ccccc23)NC(=O)[C@H](Cc2ccc(O)cc2)NC(=O)[C@H](NC(=O)[C@@H](Cc2ccccc2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 33 | 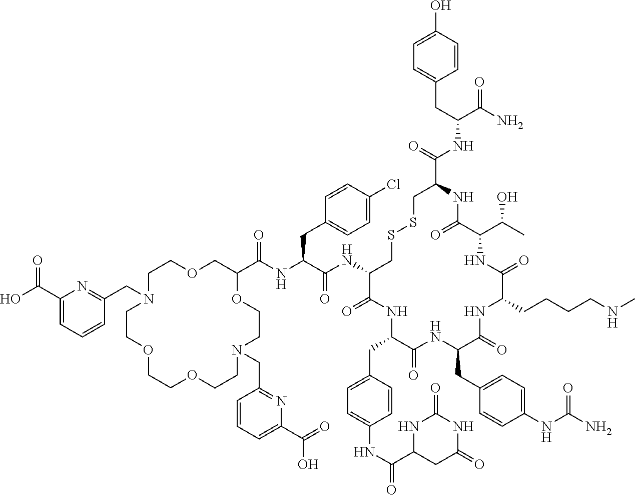 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2ccc(Cl)cc2)NC(=O)C2COCCN(Cc3cccc(C(=O)O)n3)CCOCCOCCN(Cc3cccc(C(=O)O)n3)CCO2)CSSC[C@@H](C(=O)N[C@H](Cc2ccc(O)cc2)C(N)=O)NC(=O)[C@H]([C@@H](C)O)NC1=O] |
| 33a | 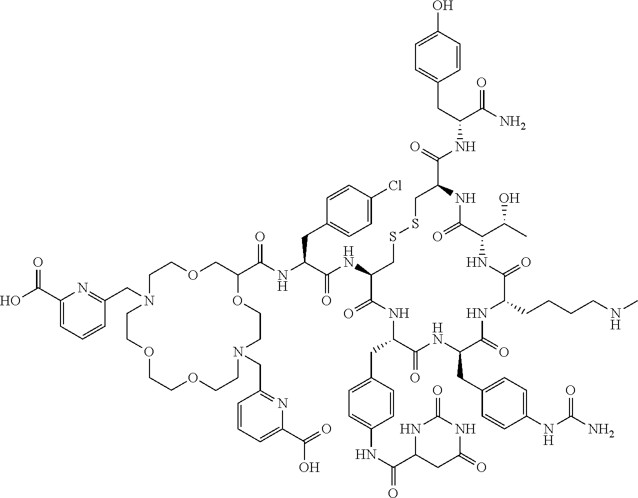 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@@H](NC(=O)[C@H](Cc2ccc(Cl)cc2)NC(=O)C2COCCN(Cc3cccc(C(=O)O)n3)CCOCCOCCN(Cc3cccc(C(=O)O)n3)CCO2)CSSC[C@@H](C(=O)N[C@H](Cc2ccc(O)cc2)C(N)=O)NC(=O)[C@H]([C@@H](C)O)NC1=O] |
| 34 | 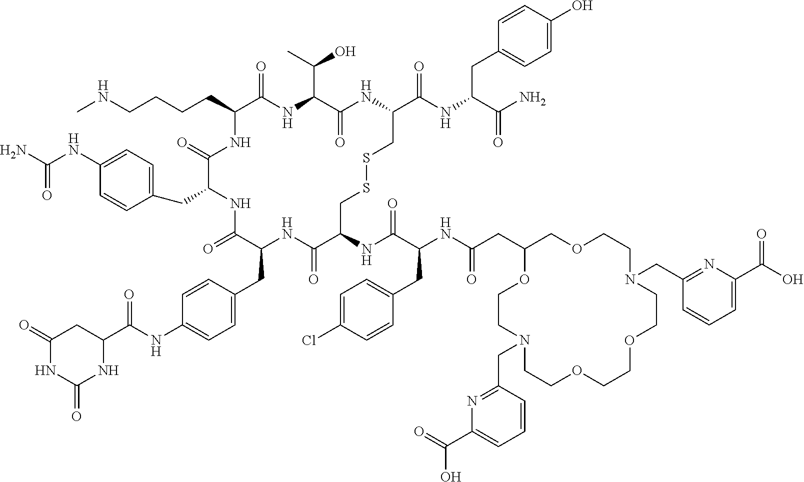 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2ccc(Cl)cc2)NC(=O)CC2COCCN(Cc3cccc(C(=O)O)n3)CCOCCOCCN(Cc3cccc(C(=O)O)n3)CCO2)CSSC[C@@H](C(=O)N[C@H](Cc2ccc(O)cc2)C(N)=O)NC(=O)[C@H]([C@@H](C)O)NC1=O] |
| 34a | 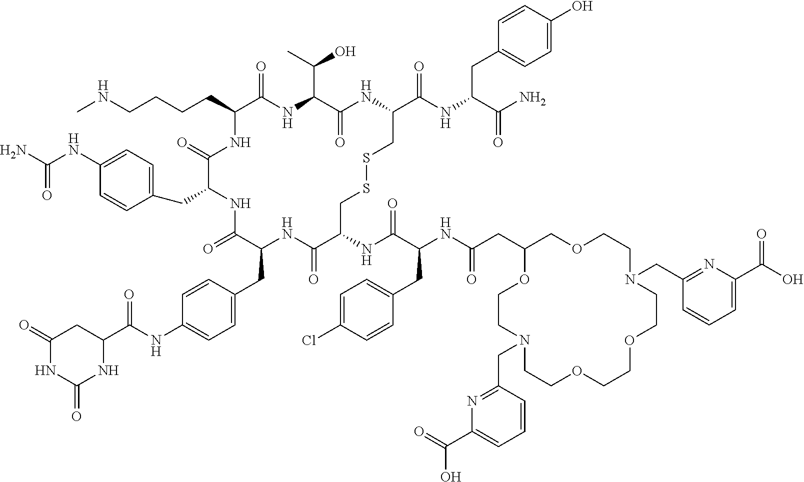 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@@H](NC(=O)[C@H](Cc2ccc(Cl)cc2)NC(=O)CC2COCCN(Cc3cccc(C(=O)O)n3)CCOCCOCCN(Cc3cccc(C(=O)O)n3)CCO2)CSSC[C@@H](C(=O)N[C@H](Cc2ccc(O)cc2)C(N)=O)NC(=O)[C@H]([C@@H](C)O)NC1=O] |
| 35 | 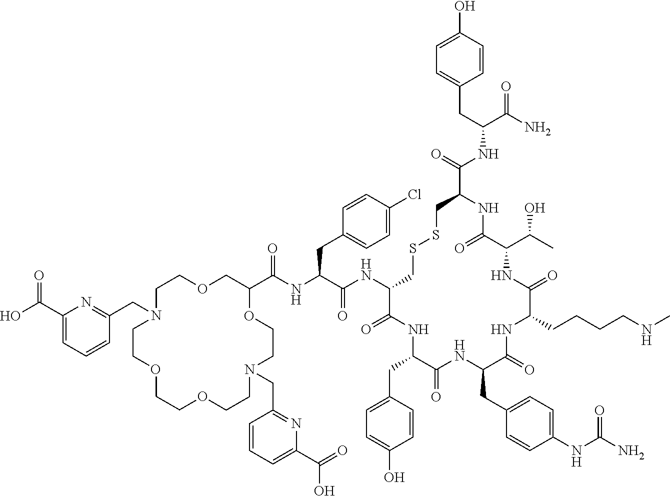 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2ccc(Cl)cc2)NC(=O)C2COCCN(Cc3cccc(C(=O)O)n3)CCOCCOCCN(Cc3cccc(C(=O)O)n3)CCO2)CSSC[C@@H](C(=O)N[C@H](Cc2ccc(O)cc2)C(N)=O)NC(=O)[C@H]([C@@H](C)O)NC1=O] |
| 35a | 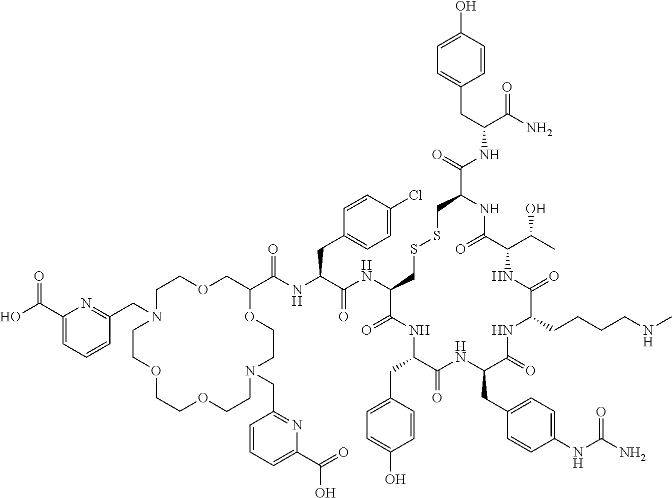 [SMILES: CNCCCC[C@@H]1NC(=O)[C@@H](Cc2ccc(NC(N)=O)cc2)NC(=O)[C@H](Cc2ccc(O)cc2)NC(=O)[C@@H](NC(=O)[C@H](Cc2ccc(Cl)cc2)NC(=O)C2COCCN(Cc3cccc(C(=O)O)n3)CCOCCOCCN(Cc3cccc(C(=O)O)n3)CCO2)CSSC[C@@H](C(=O)N[C@H](Cc2ccc(O)cc2)C(N)=O)NC(=O)[C@H]([C@@H](C)O)NC1=O] |
| 36 | 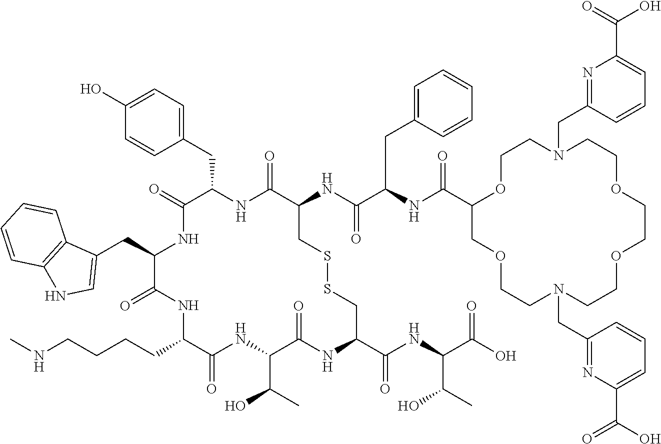 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)C2COCCN(Cc3cccc(C(=O)O)n3)CCOCCOCCN(Cc3cccc(C(=O)O)n3)CCO2)CSSC[C@@H](C(=O)N[C@@H](C(=O)O)[C@H](C)O)NC(=O)[C@H]([C@@H](C)O)NC1=O] |
| 36a | 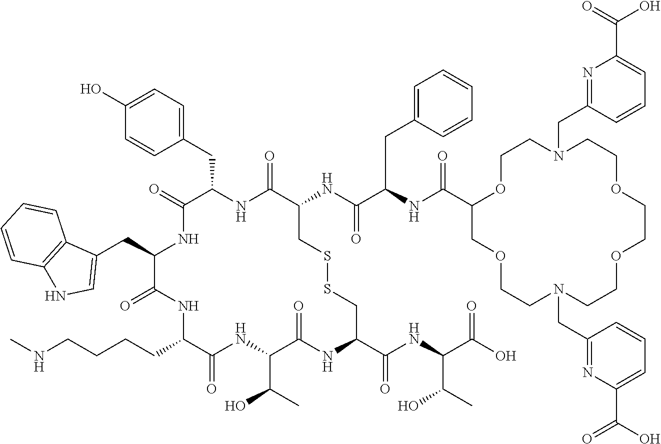 [SMILES: CNCCCC[C@@H]1NC(=O)[C@@H](Cc2c[nH]c3ccccc23)NC(=O)[C@H](Cc2ccc(O)cc2)NC(=O)[C@H](NC(=O)[C@@H](Cc2ccccc2)NC(=O)C2COCCN(Cc3cccc(C(=O)O)n3)CCOCCOCCN(Cc3cccc(C(=O)O)n3)CCO2)CSSC[C@@H](C(=O)N[C@@H](C(=O)O)[C@H](C)O)NC(=O)[C@H]([C@@H](C)O)NC1=O] |
| 37 | 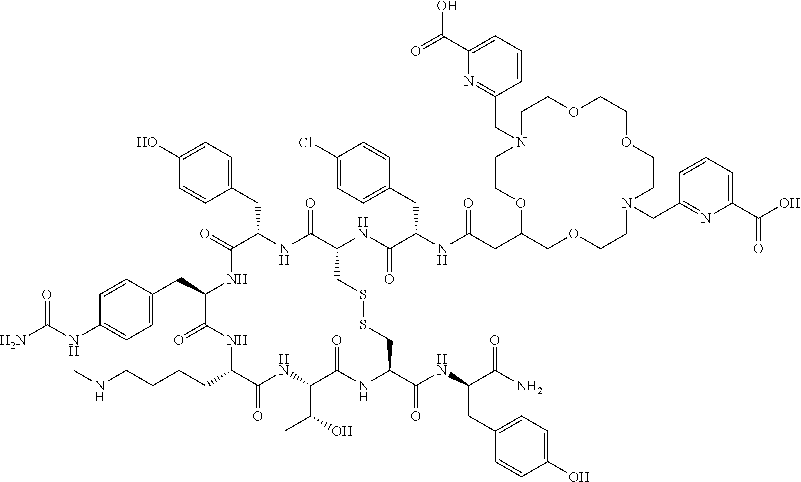 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2ccc(Cl)cc2)NC(=O)CC2COCCN(Cc3cccc(C(=O)O)n3)CCOCCOCCN(Cc3cccc(C(=O)O)n3)CCO2)CSSC[C@@H](C(=O)N[C@H](Cc2ccc(O)cc2)C(N)=O)NC(=O)[C@H]([C@@H](C)O)NC1=O] |
| 37a | 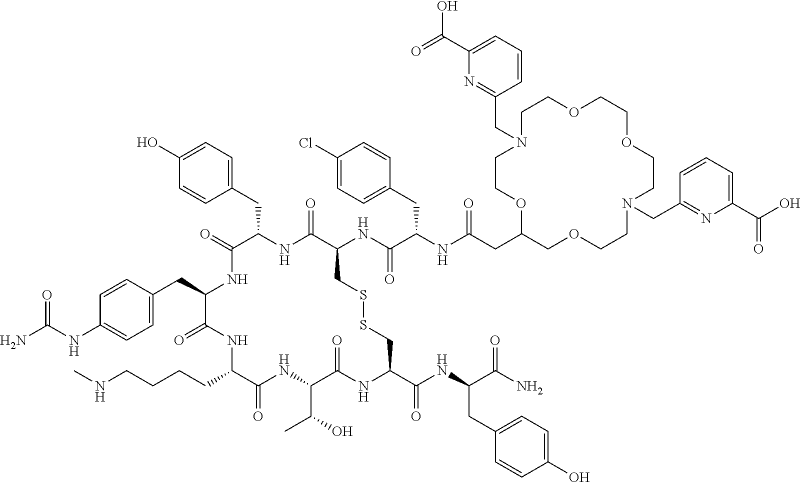 [SMILES: CNCCCC[C@@H]1NC(=O)[C@@H](Cc2ccc(NC(N)=O)cc2)NC(=O)[C@H](Cc2ccc(O)cc2)NC(=O)[C@@H](NC(=O)[C@H](Cc2ccc(Cl)cc2)NC(=O)CC2COCCN(Cc3cccc(C(=O)O)n3)CCOCCOCCN(Cc3cccc(C(=O)O)n3)CCO2)CSSC[C@@H](C(=O)N[C@H](Cc2ccc(O)cc2)C(N)=O)NC(=O)[C@H]([C@@H](C)O)NC1=O] |
| 38 | 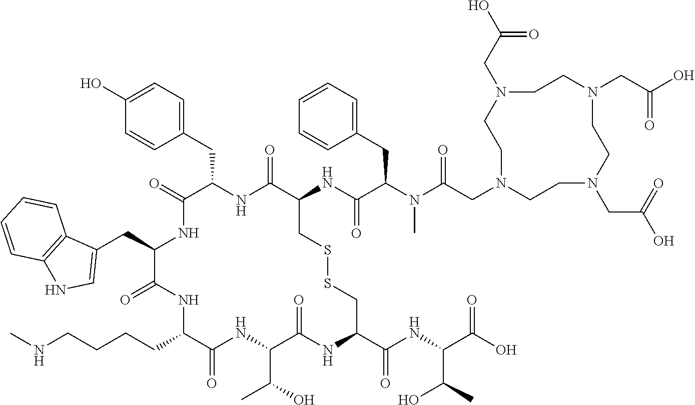 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccccc2)N(C)C(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 38a | 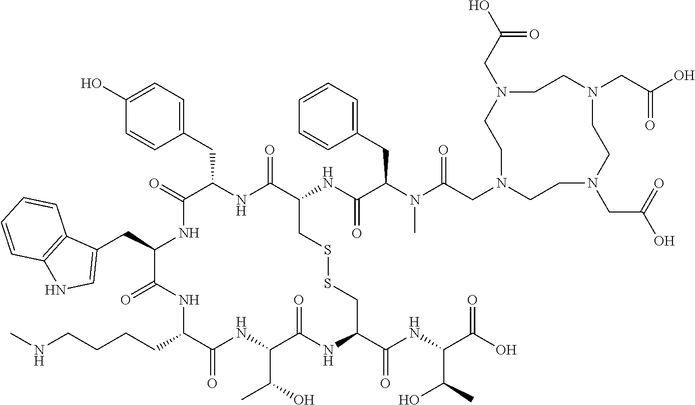 [SMILES: CNCCCC[C@@H]1NC(=O)[C@@H](Cc2c[nH]c3ccccc23)NC(=O)[C@H](Cc2ccc(O)cc2)NC(=O)[C@H](NC(=O)[C@@H](Cc2ccccc2)N(C)C(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 39 | 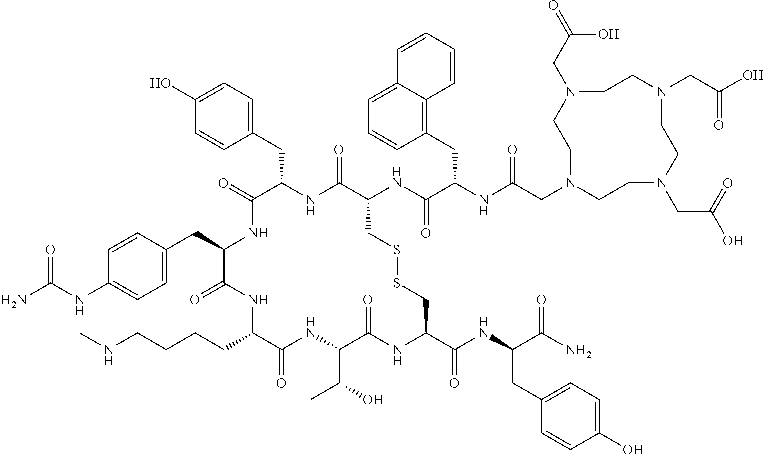 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2cccc3ccccc23)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |
| 39a | 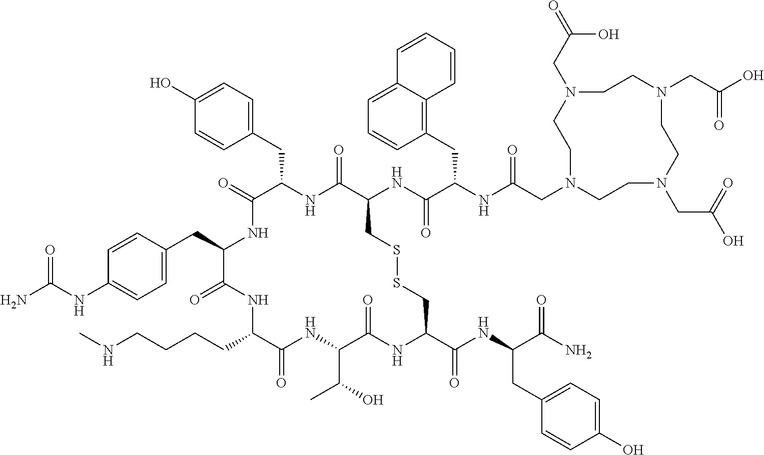 [SMILES: CNCCCC[C@@H]1NC(=O)[C@@H](Cc2ccc(NC(N)=O)cc2)NC(=O)[C@H](Cc2ccc(O)cc2)NC(=O)[C@@H](NC(=O)[C@H](Cc2cccc3ccccc23)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |
| 40 | 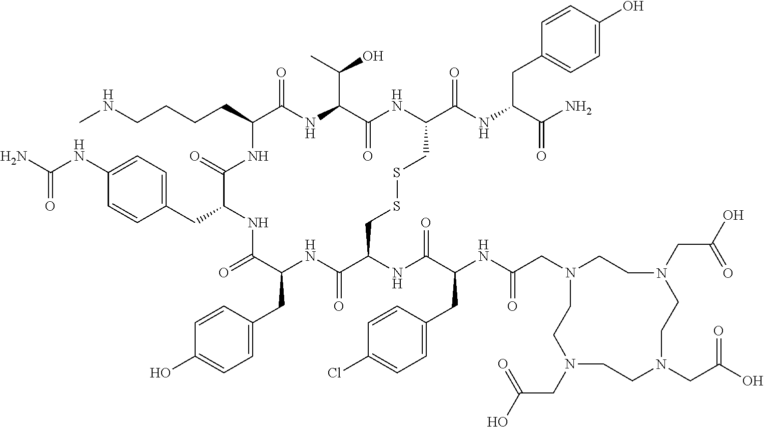 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2ccc(Cl)cc2)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |
| 40a | 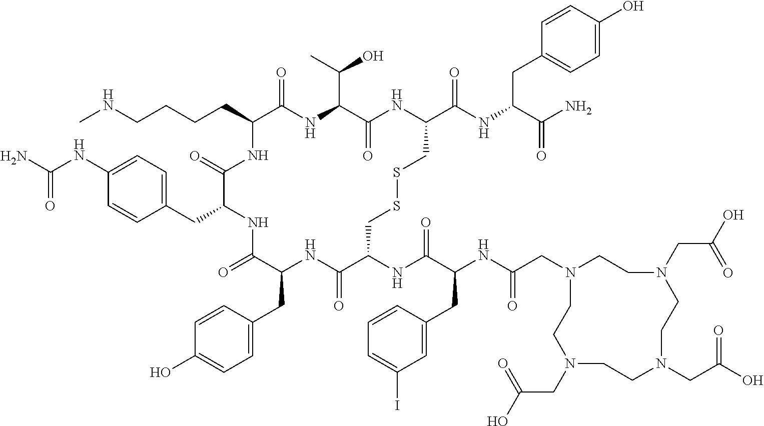 [SMILES: CNCCCC[C@@H]1NC(=O)[C@@H](Cc2ccc(NC(N)=O)cc2)NC(=O)[C@H](Cc2ccc(O)cc2)NC(=O)[C@@H](NC(=O)[C@H](Cc2cccc(I)c2)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |

The nomenclature in which a compound name is preceded by an isotope indicates that the isotope is chelated to the chelating group of the compound. For example, “[⁶⁸Ga]Compound 1” refers to Compound 1 in which its chelating group is chelated with ⁶⁸Ga.

Exemplary compounds of the invention (with their chelating group) include [⁶⁸Ga]Compound 2; [¹¹¹In]Compound 2; [¹⁶¹Tb]Compound 2; [¹⁷⁷Lu]Compound 2; [²¹²Pb]Compound 2; [²²⁵Ac]Compound 2; [⁶⁸Ga]Compound 12; [¹¹¹In]Compound 12; [¹⁶¹Tb]Compound 12; [¹⁷⁷Lu]Compound 12; [²¹²Pb]Compound 12; [²²⁵Ac]Compound 12; [¹¹¹In]Compound 17; [²¹²Pb]Compound 17; [²²⁵Ac]Compound 17; [¹¹¹In]Compound 21; [²¹²Pb]Compound 21; [²²⁵Ac]Compound 21; [⁶⁸Ga]Compound 24; [¹¹¹In]Compound 24; [¹⁶¹Tb]Compound 24; [¹⁷⁷Lu]Compound 24; [²¹²Pb]Compound 24; [²²⁵Ac]Compound 24; [⁶⁸Ga]Compound 25; [¹¹¹In]Compound 25; [¹⁶¹Tb]Compound 25; [¹⁷⁷Lu]Compound 25; [²¹²Pb]Compound 25; [²²⁵Ac]Compound 25; [⁶⁸Ga]Compound 27; [¹¹¹In]Compound 27; [¹⁶¹Tb]Compound 27; [¹⁷⁷Lu]Compound 27; [²¹²Pb]Compound 27; [²²⁵Ac]Compound 27. In another aspect, the invention is any one of the compounds in Table 1 above, wherein the chelating group of the compound is chelated with ⁶⁸Ga. In another aspect, the invention is any one of the compounds in Table 1 above, wherein the chelating group of the compound is chelated with ¹¹¹In. In another aspect, the invention is any one of the compounds in Table 1 above, wherein the chelating group of the compound is chelated with ¹⁶¹Tb. In another aspect, the invention is any one of the compounds in Table 1 above, wherein the chelating group of the compound is chelated with ¹⁷⁷Lu. In another aspect, the invention is any one of the compounds in Table 1 above, wherein the chelating group of the compound is chelated with ²¹²Pb. In another aspect, the invention is any one of the compounds in Table 1 above, wherein the chelating group of the compound is chelated with ²²⁵Ac.

Also included in the invention is a compound represented by structural formula (I), (IIa), (IIb), (IIc), (IIIa), (IIIb), (IIIc), (IVa), (IVb), (IVc), (Va), (Vb), (Vc), (VIa), (VIb), (VIc), (VIIa), (VIIb), (VIIc), (VIIIa), (VIIIb), (VIIIc), (IXa), (IXb), or (IXc), or a pharmaceutically acceptable salt thereof, described herein (e.g., in any one of the first to thirty-third embodiments), wherein the CG moiety in the structural formula is replaced with hydrogen. The compounds shown in Table 2 below, wherein the chelating group is replaced by hydrogen are also included in the invention.

TABLE 2
Compounds of the Invention

| Compound | |
|---|---|
| No. | Structure |
| 41 | 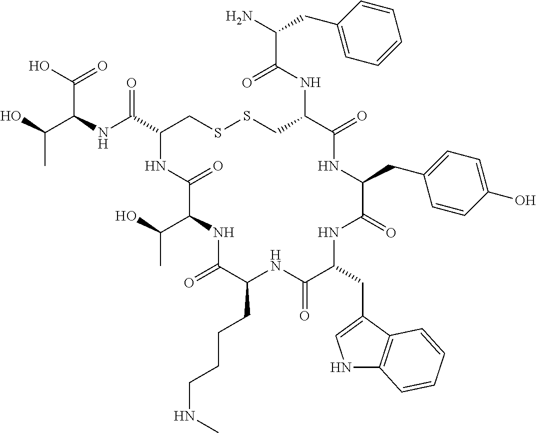 |
| 41a | 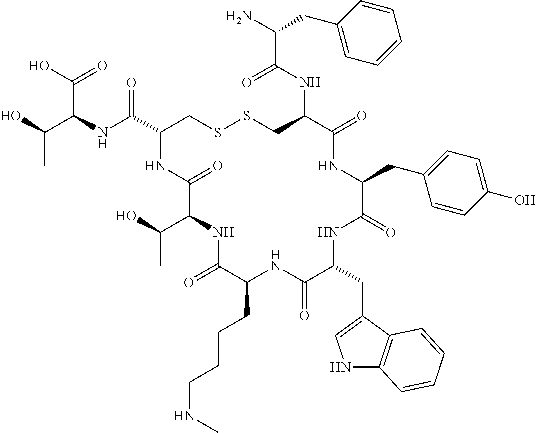 |
| 42 | 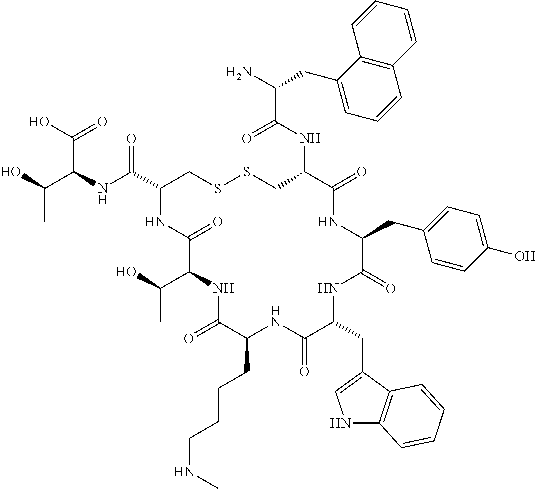 |
| 42a | 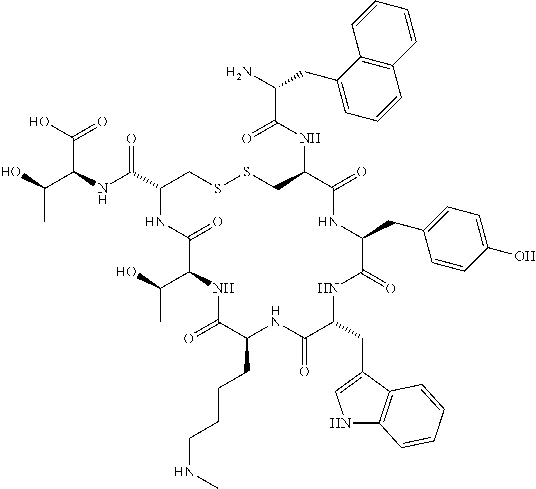 |
| 43 | 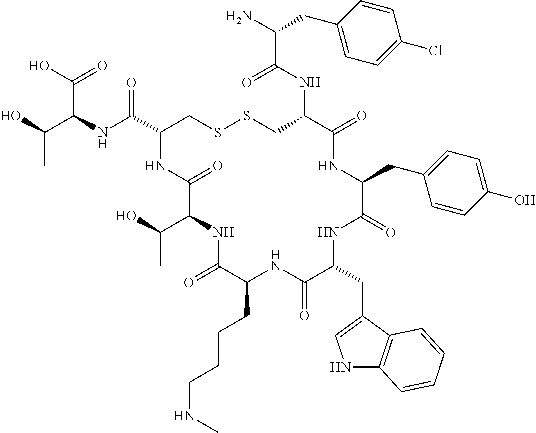 |
| 43a | 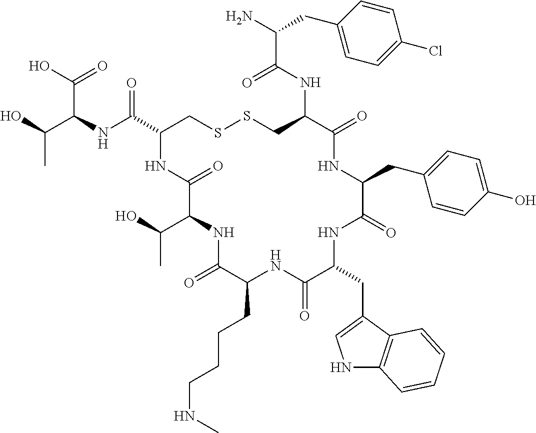 |
| 44 | 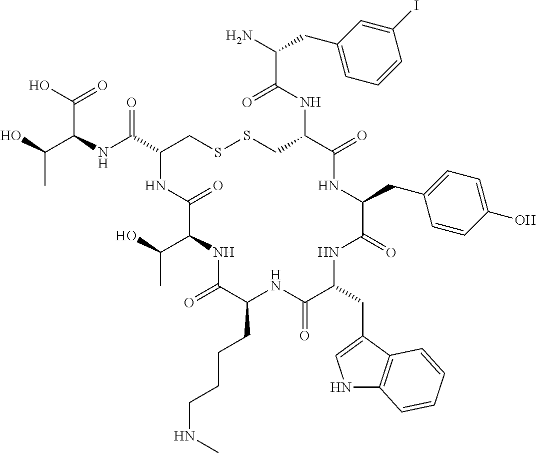 |
| 44a | 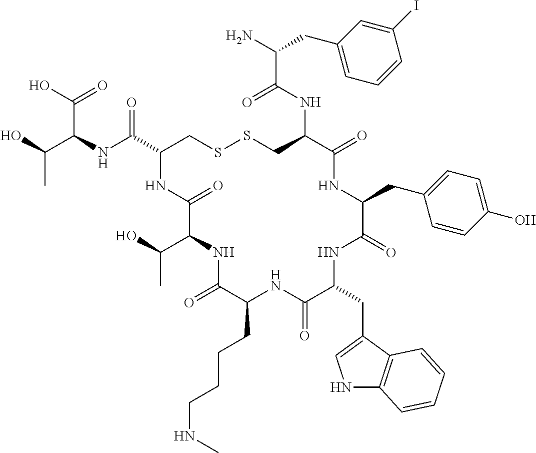 |
| 45 | 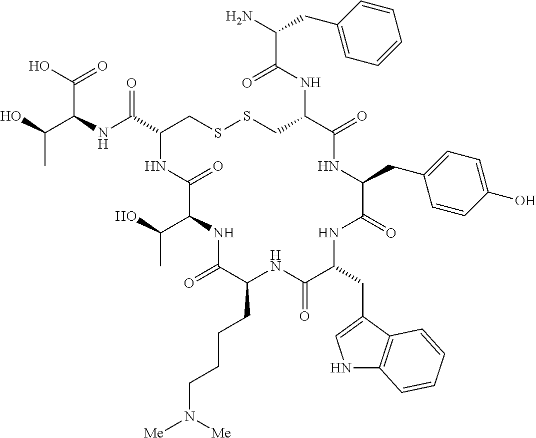 |
| 45a | 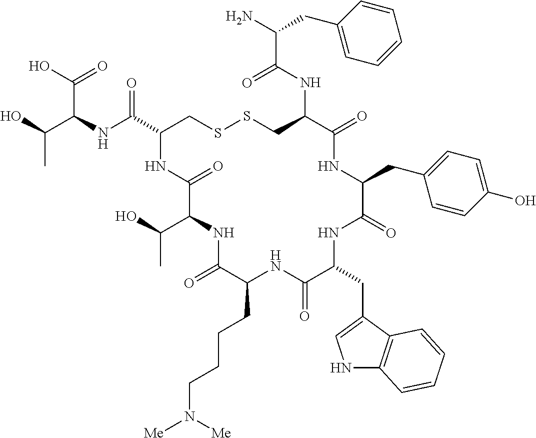 |
| 46 | 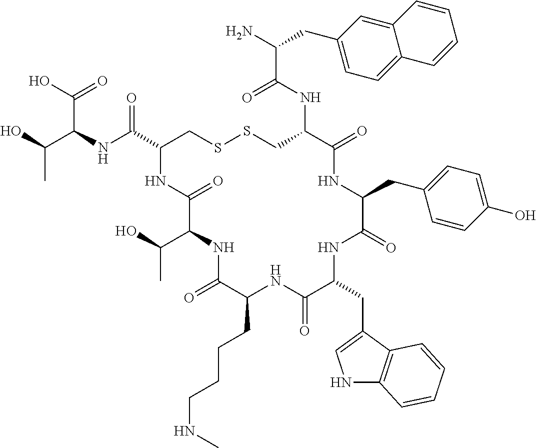 |
| 46a | 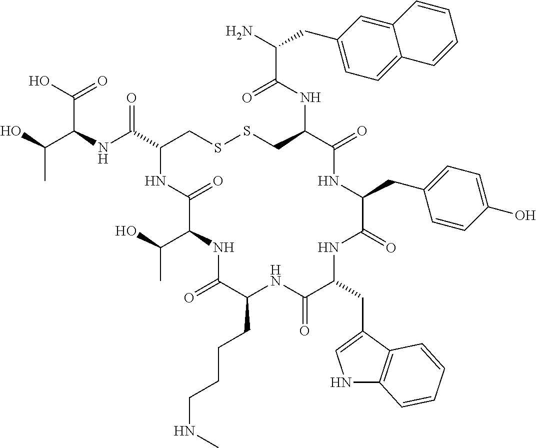 |
| 47 | 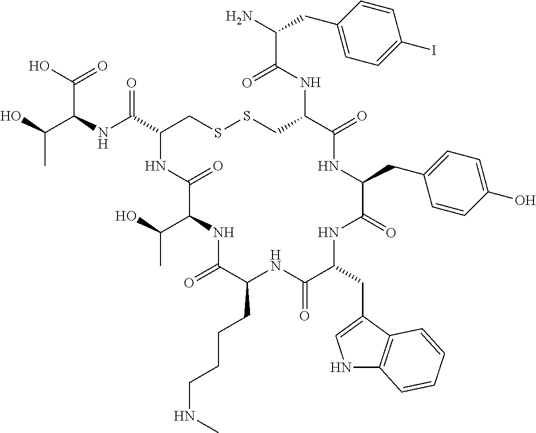 |
| 47a | 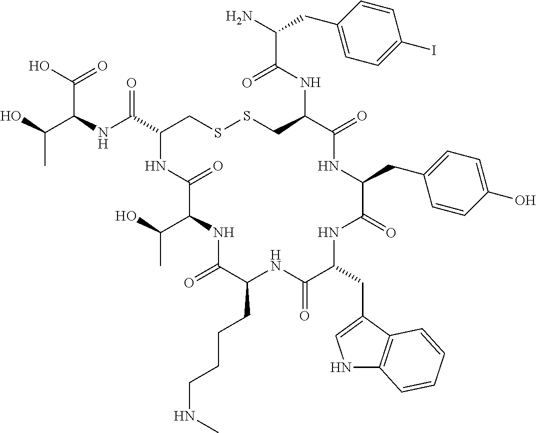 |
| 48 | 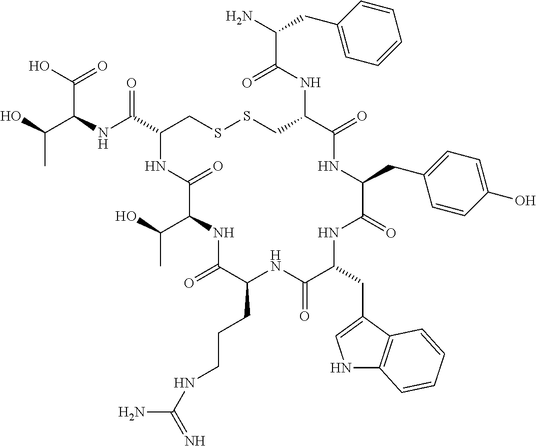 |
| 48a | 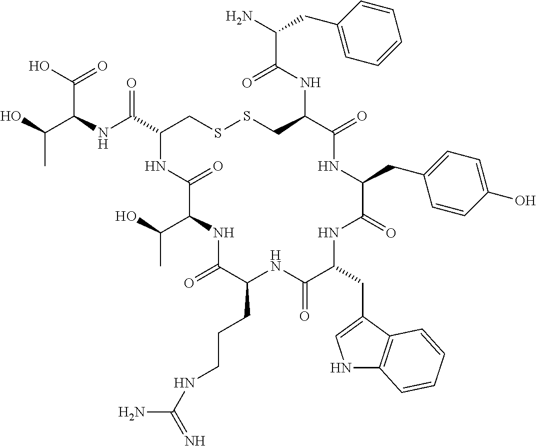 |
| 49 | 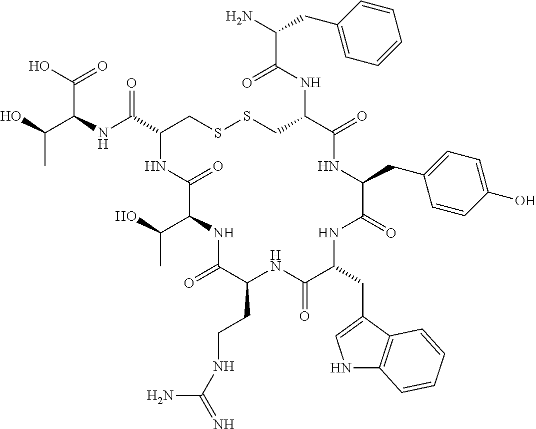 |
| 49a | 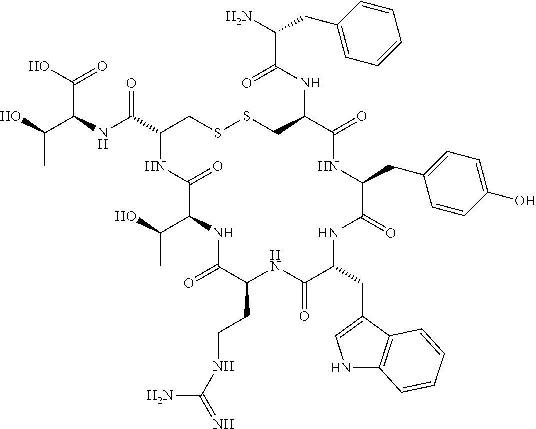 |
| 50 | 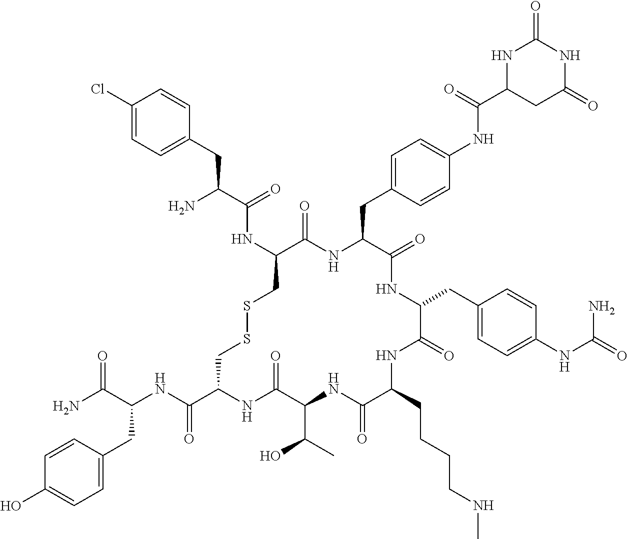 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@@H](N)Cc2ccc(Cl)cc2)CSSC[C@@H](C(=O)N[C@H](Cc2ccc(O)cc2)C(N)=O)NC(=O)[C@H]([C@@H](C)O)NC1=O] |
| 50a | 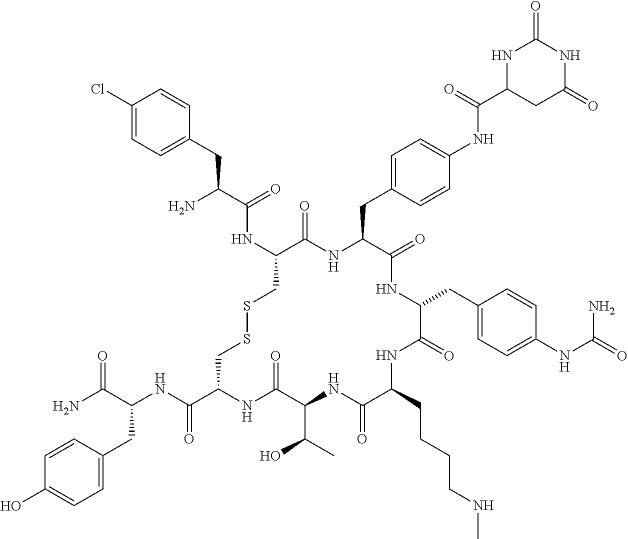 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@@H](NC(=O)[C@@H](N)Cc2ccc(Cl)cc2)CSSC[C@@H](C(=O)N[C@H](Cc2ccc(O)cc2)C(N)=O)NC(=O)[C@H]([C@@H](C)O)NC1=O] |
| 51 | 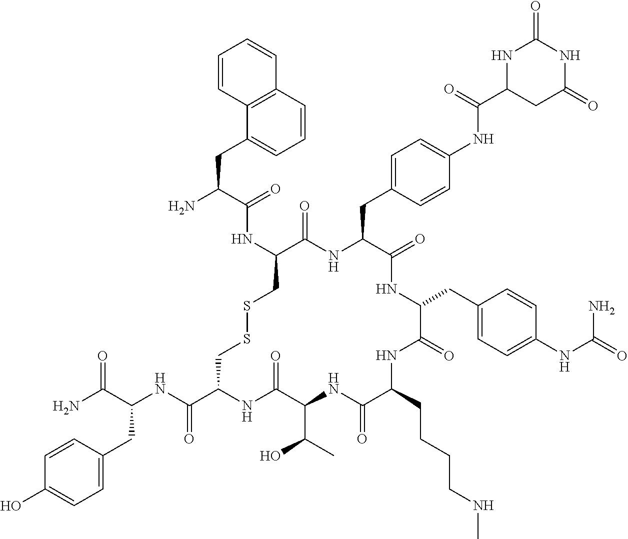 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@@H](N)Cc2cccc3ccccc23)CSSC[C@@H](C(=O)N[C@H](Cc2ccc(O)cc2)C(N)=O)NC(=O)[C@H]([C@@H](C)O)NC1=O] |
| 51a | 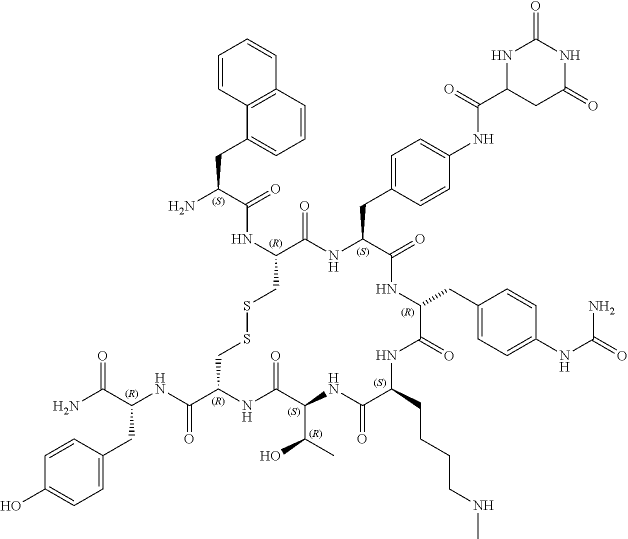 |
| 52 | 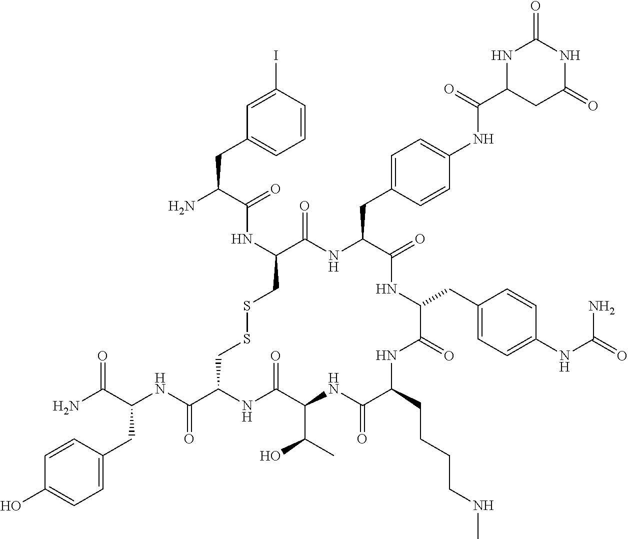 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@@H](N)Cc2cccc(I)c2)CSSC[C@@H](C(=O)N[C@H](Cc2ccc(O)cc2)C(N)=O)NC(=O)[C@H]([C@@H](C)O)NC1=O] |
| 52a | 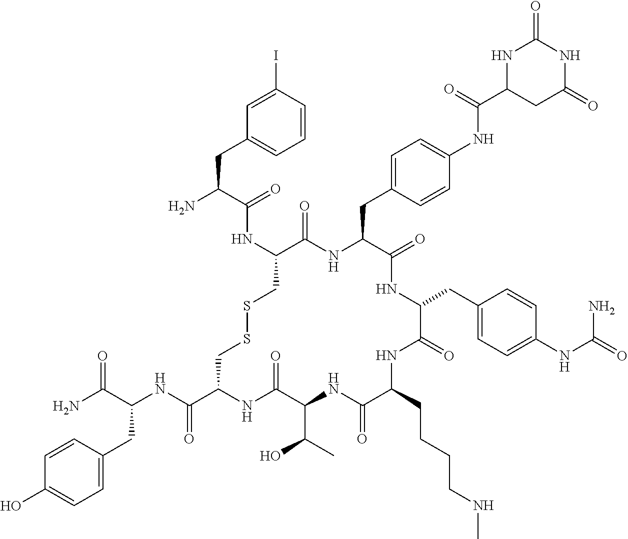 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@@H](NC(=O)[C@@H](N)Cc2cccc(I)c2)CSSC[C@@H](C(=O)N[C@H](Cc2ccc(O)cc2)C(N)=O)NC(=O)[C@H]([C@@H](C)O)NC1=O] |
| 53 | 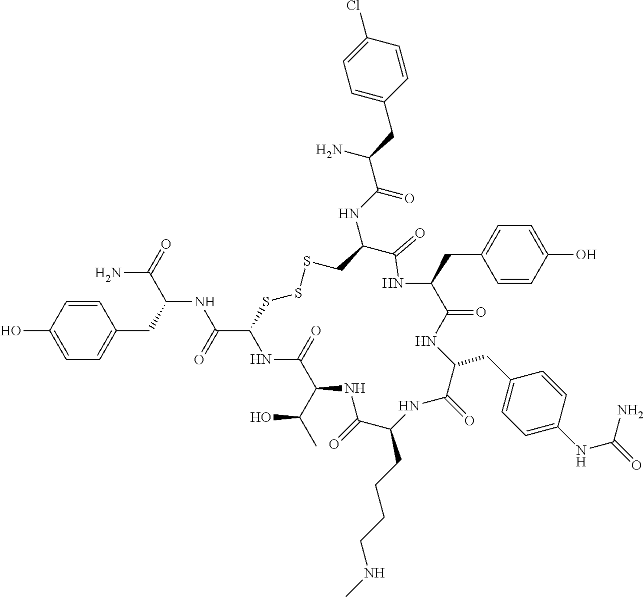 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@@H](N)Cc2ccc(Cl)cc2)CSSS[C@@H](C(=O)N[C@H](Cc2ccc(O)cc2)C(N)=O)NC(=O)[C@H]([C@@H](C)O)NC1=O] |
| 53a | 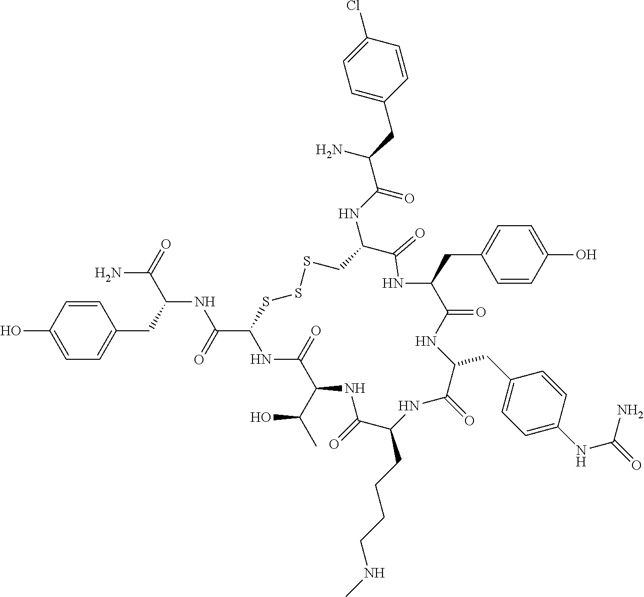 [SMILES: CNCCCC[C@@H]1NC(=O)[C@@H](Cc2ccc(NC(N)=O)cc2)NC(=O)[C@H](Cc2ccc(O)cc2)NC(=O)[C@@H](NC(=O)[C@@H](N)Cc2ccc(Cl)cc2)CSSS[C@@H](C(=O)N[C@H](Cc2ccc(O)cc2)C(N)=O)NC(=O)[C@H]([C@@H](C)O)NC1=O] |
| 54 | 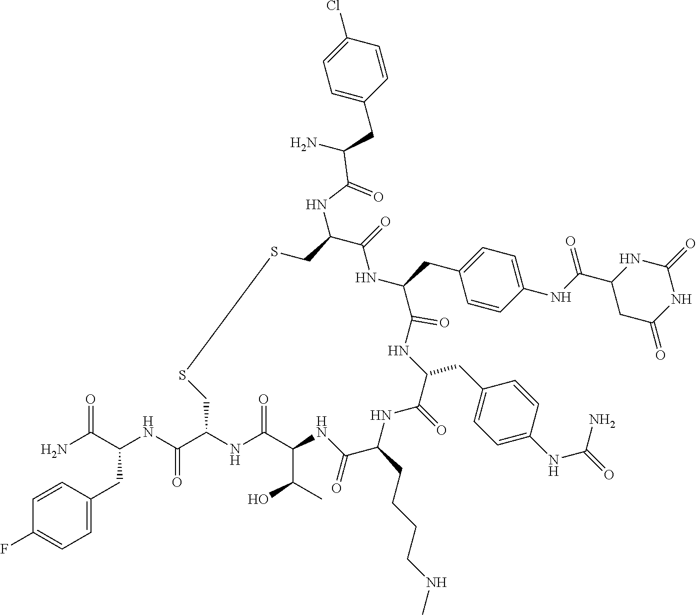 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@@H](N)Cc2ccc(Cl)cc2)CSSC[C@@H](C(=O)N[C@H](Cc2ccc(F)cc2)C(N)=O)NC(=O)[C@H]([C@@H](C)O)NC1=O] |
| 54a | 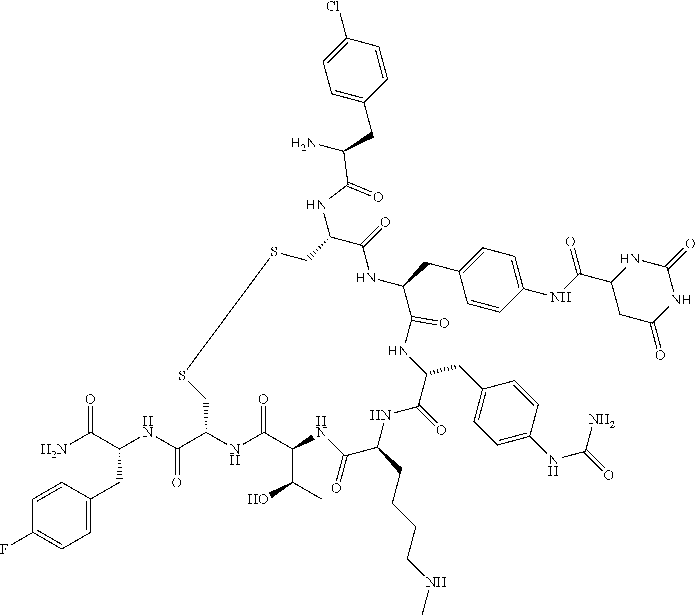 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@@H](NC(=O)[C@@H](N)Cc2ccc(Cl)cc2)CSSC[C@@H](C(=O)N[C@H](Cc2ccc(F)cc2)C(N)=O)NC(=O)[C@H]([C@@H](C)O)NC1=O] |
| 55 | 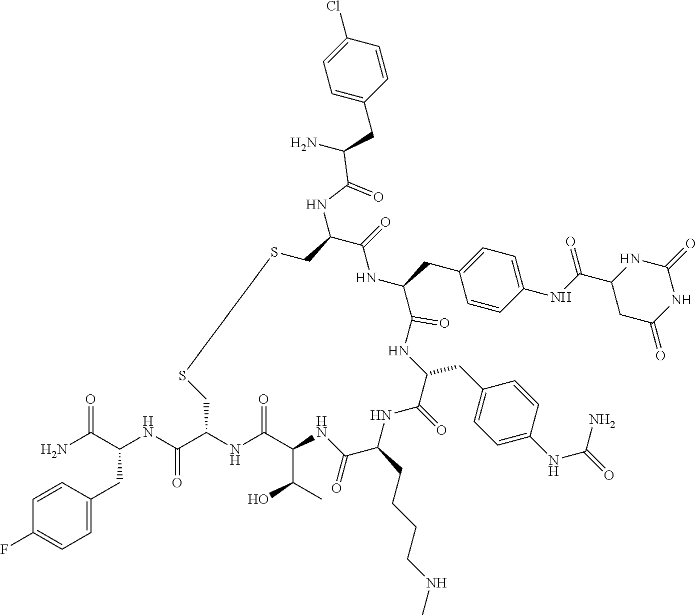 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@@H](N)Cc2ccc(Cl)cc2)CSSC[C@@H](C(=O)N[C@H](Cc2ccc(F)cc2)C(N)=O)NC(=O)[C@H]([C@@H](C)O)NC1=O] |
| 55a | 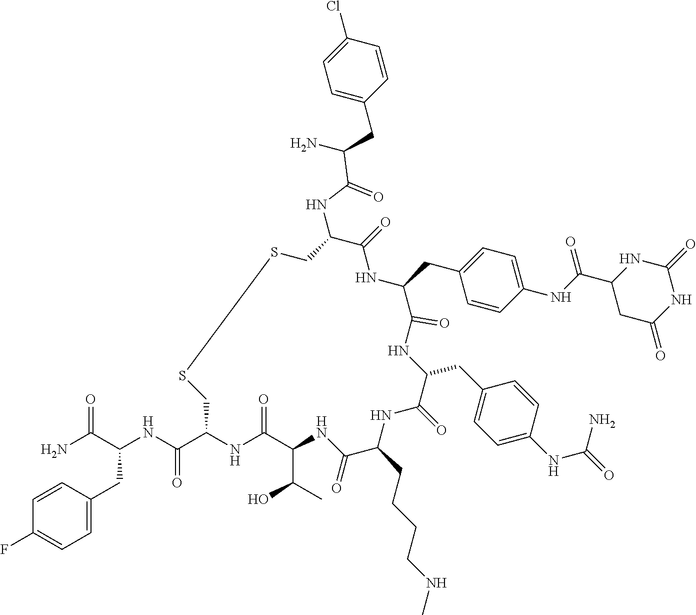 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@@H](NC(=O)[C@@H](N)Cc2ccc(Cl)cc2)CSSC[C@@H](C(=O)N[C@H](Cc2ccc(F)cc2)C(N)=O)NC(=O)[C@H]([C@@H](C)O)NC1=O] |
| 56 | 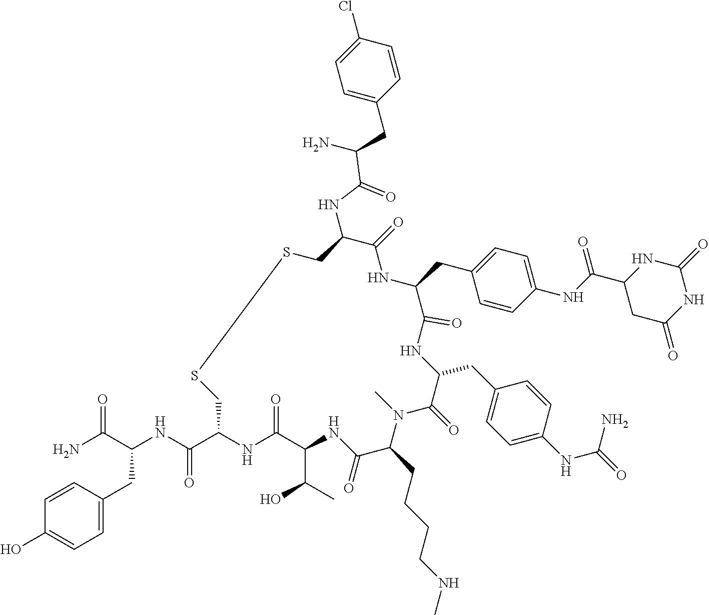 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@@H](N)Cc2ccc(Cl)cc2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| 56a | 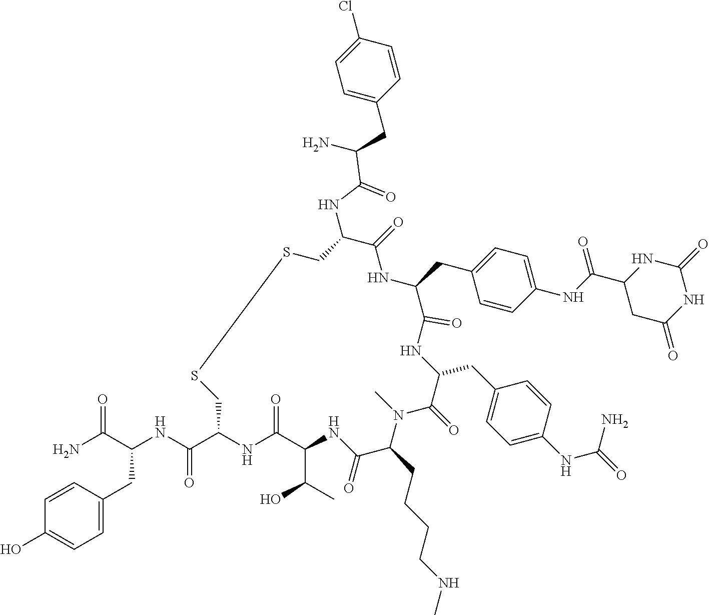 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@H](NC(=O)[C@@H](N)Cc2ccc(Cl)cc2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| 57 | 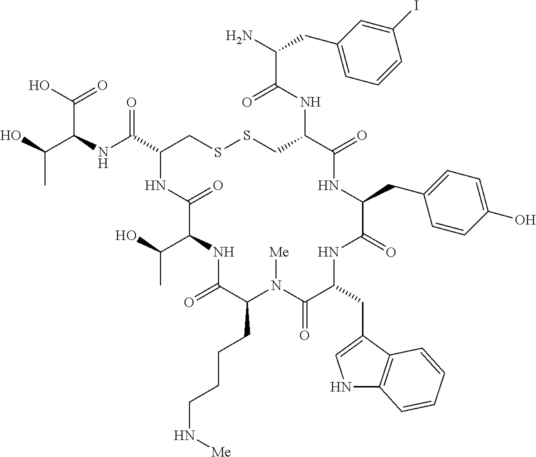 |
| 57a | 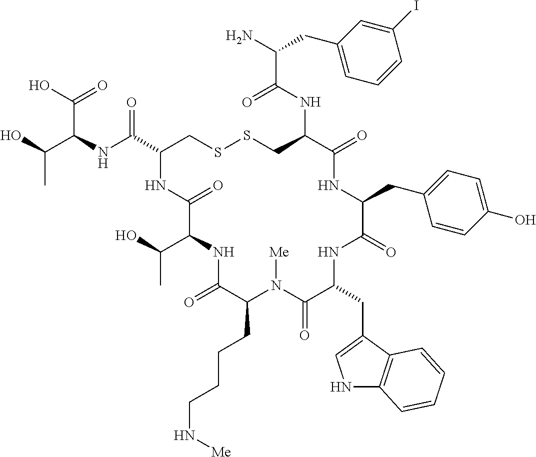 |
| 58 | 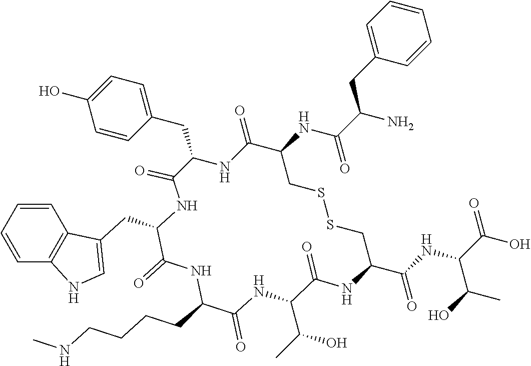 |
| 58a | 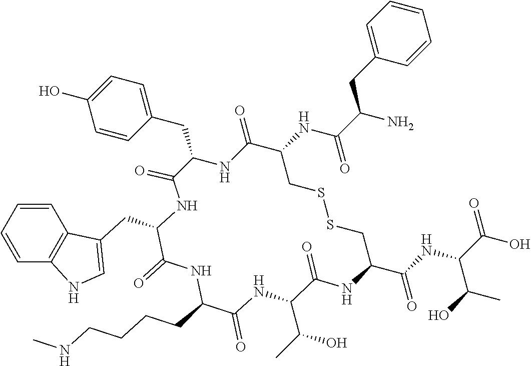 |
| 59 | 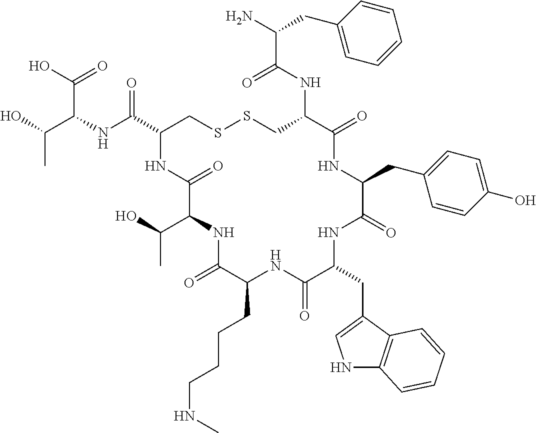 |
| 59a | 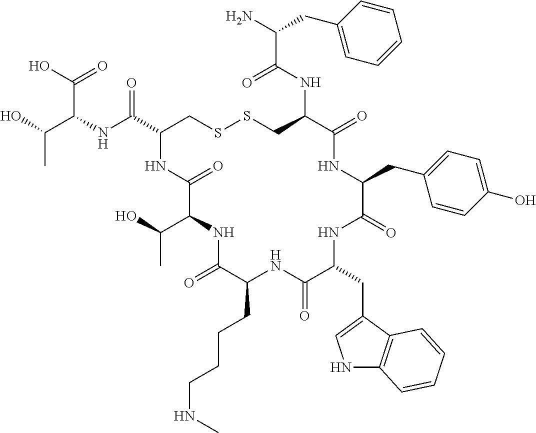 |
| 60 | 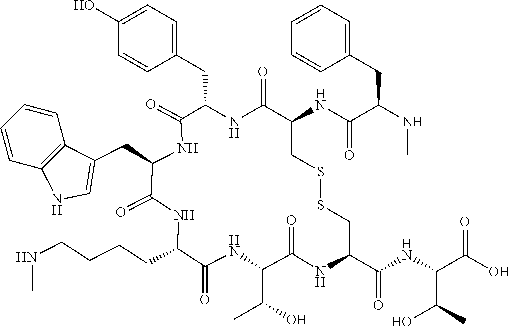 |
| 60a | 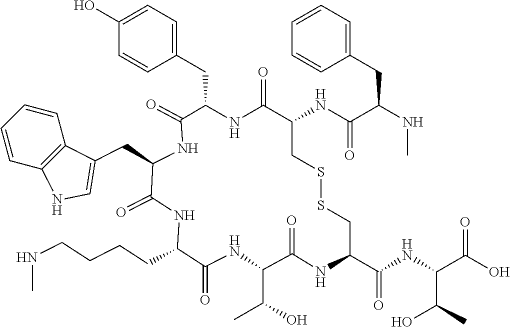 |
| 61 | 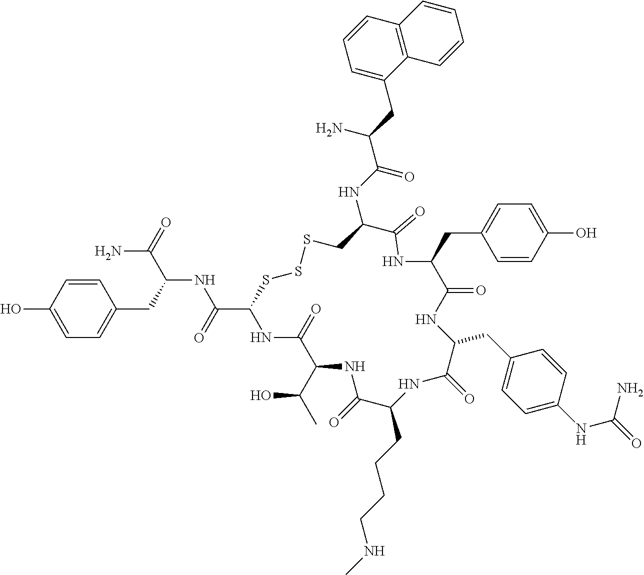 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@@H](N)Cc2cccc3ccccc23)CSSS[C@@H](C(=O)N[C@H](Cc2ccc(O)cc2)C(N)=O)NC(=O)[C@H]([C@@H](C)O)NC1=O] |
| 61a | 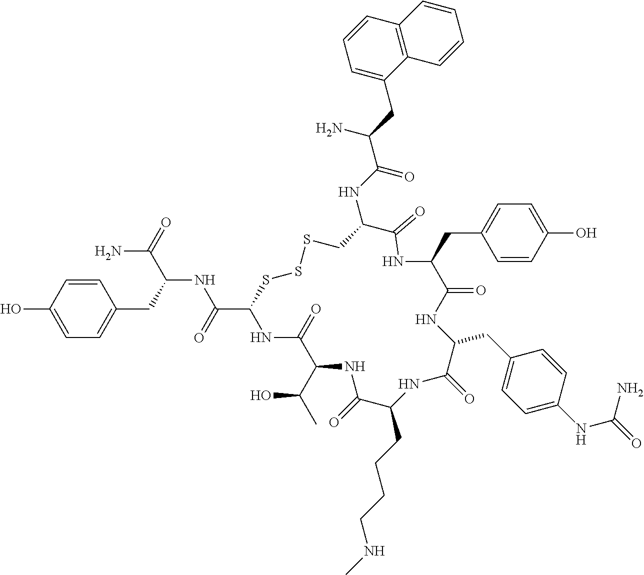 [SMILES: CNCCCC[C@@H]1NC(=O)[C@@H](Cc2ccc(NC(N)=O)cc2)NC(=O)[C@H](Cc2ccc(O)cc2)NC(=O)[C@@H](NC(=O)[C@@H](N)Cc2cccc3ccccc23)CSSS[C@@H](C(=O)N[C@H](Cc2ccc(O)cc2)C(N)=O)NC(=O)[C@H]([C@@H](C)O)NC1=O] |
| 62 | 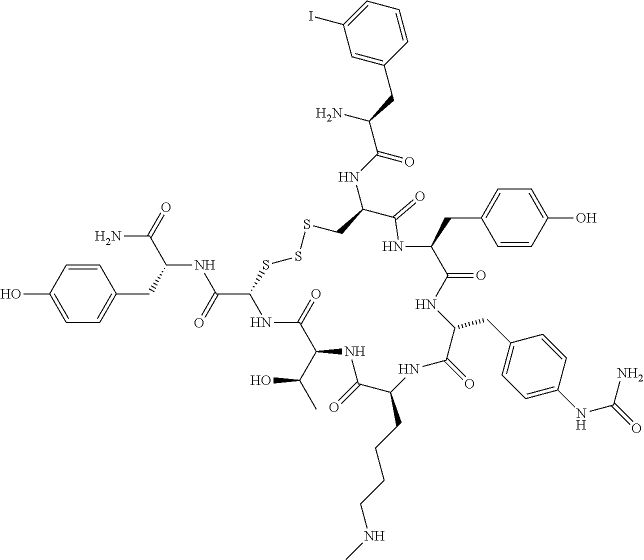 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@@H](N)Cc2cccc(I)c2)CSSS[C@@H](C(=O)N[C@H](Cc2ccc(O)cc2)C(N)=O)NC(=O)[C@H]([C@@H](C)O)NC1=O] |
| 62a | 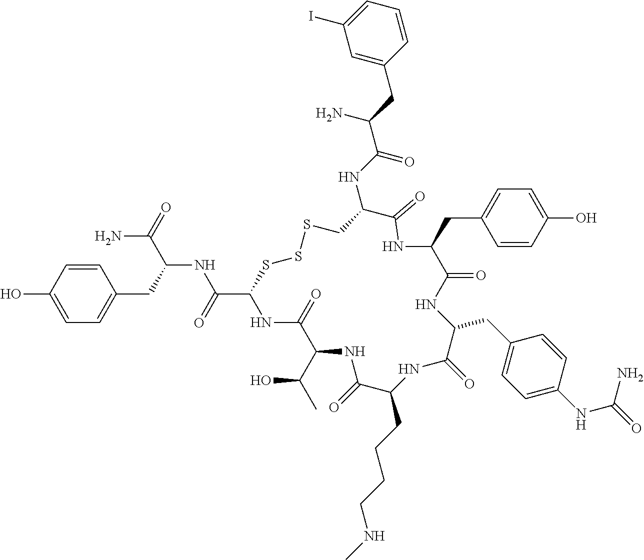 [SMILES: CNCCCC[C@@H]1NC(=O)[C@@H](Cc2ccc(NC(N)=O)cc2)NC(=O)[C@H](Cc2ccc(O)cc2)NC(=O)[C@@H](NC(=O)[C@@H](N)Cc2cccc(I)c2)CSSS[C@@H](C(=O)N[C@H](Cc2ccc(O)cc2)C(N)=O)NC(=O)[C@H]([C@@H](C)O)NC1=O] |

“Aliphatic” means a saturated or unsaturated straight-chain or branched monovalent or bivalent hydrocarbon radical. The term “aliphatic” encompasses alkyl, alkenyl, and alkynyl groups. Unless otherwise specified, an aliphatic group typically has 1 to 10 carbon atoms. “Alkyl” means a saturated aliphatic straight-chain or branched monovalent aliphatic radical. “Alkylene” refers to a bivalent alkyl group, e.g., (CH₂)ₓ wherein x is unless otherwise specified typically an integer from 1-10. Unless otherwise specified, an alkyl or alkylene group typically has 1 to 6 carbon atoms (C₁₋₆ alkyl) or (CH₂)₁₋₆, alternatively, 1 to 3 carbon atoms (C₁₋₃ alkyl or (CH₂)₁₋₃) (i.e., 1, 2 or 3).

“Haloalkyl” and “haloalkoxy” means alkyl or alkoxy, as the case may be, substituted with one or more halogen atoms. Examples of haloalkyl, include, but are not limited to, trifluoromethyl, trichloromethyl, pentafluoroethyl and the like.

“Cyclic aliphatic” means a saturated or unsaturated, monovalent or bivalent, cyclic hydrocarbon ring radical. Unless otherwise specified, a cyclic aliphatic has 3 to 8 ring carbon atoms (“C₃₋₈ cyclic aliphatic”). “Cycloalkyl” means a saturated aliphatic cyclic aliphatic. Unless otherwise specified, a cycloalkyl has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 8 ring carbon atoms (“C₅₋₈ cycloalkyl”).

“Aryl”, alone or part or a larger moiety such as “aralkyl” is an aromatic carbocyclic group such as phenyl or naphthyl. “Aryl” may refer to a radical of a monocyclic or polycyclic (e.g., bicyclic) 4n+2 aromatic ring system (e.g., having 6 or 10 π electrons shared in a cyclic array) having 6-10 ring carbon atoms and zero heteroatoms provided in the aromatic ring system (“C₆₋₁₀ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl).

“Aralkyl” refers to an alkyl group substituted with an aryl group. Unless otherwise specified, the alkyl portion of an aralkyl group has from 1-10 carbon atoms. “C₁-Cₓ aralkyl” refers to an aralkyl group in which the alkyl portion has 1 to x carbon atoms. Unless otherwise specified, x is an integer from 2 to 10.

“Heterocycle” or “heterocyclyl” refer to a non-aromatic group with 3 to 10 ring members containing 1 to 4 heteroatoms independently selected from nitrogen, oxygen, and sulfur. A “4-7 membered heterocycle” refers to a monocyclic, non-aromatic ring with 4 to 7 members containing 1 to 4 heteroatoms independently selected from nitrogen, oxygen, and sulfur. Exemplary 5-membered heterocycles include pyrrolidinyl and triazinyl. Exemplary 6-membered heterocycles include piperidinyl, morpholinyl, and piperazinyl.

Compounds having one or more chiral centers can exist in various stereoisomeric forms, i.e., each chiral center can have an R or S configuration or can be a mixture of both. Stereoisomers are compounds that differ only in their spatial arrangement. Stereoisomers include all diastereomeric and enantiomeric forms of a compound. Enantiomers are stereoisomers that are non-superimposable mirror images of each other. Diastereomers are stereoisomers having two or more chiral centers that are not identical and are not mirror images of each other.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a disclosed compound having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a disclosed compound having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

“Peak 1” or “first eluting isomer” in the Experimental section refers to an intended reaction product compound obtained from a chromatography separation/purification that elutes earlier than a second intended reaction product compound from the same preceding reaction. The second intended product compound is referred to as “peak 2” or “second eluting isomer”.

When two or more stereoisomers are depicted by their chemical names or structures, and the names or structures are connected by an “or”, one or the other of the two or more stereoisomers is intended, but not both. The enrichment of one stereoisomer relative to the other is as indicated above.

The SSTR2 targeted compounds of the present invention are useful imaging agents for diagnostic applications. For example, these can be conjugated to various metals for magnetic resonance imaging applications or conjugated to an optical dye or a fluorophore or other detectable moiety (i.e., dyes, quantum dots, etc.) for histochemistry applications and luminescence imaging applications. The compounds can also be radiolabeled and used in nuclear medicine applications. Radionuclides useful for imaging applications are referred to herein as “imaging radionuclides”. Non-limiting examples of imaging radionuclides include ¹⁸F, ⁶⁴Cu or ⁶⁸Ga, which are suitable for use in PET imaging applications, and ⁶⁷Cu or ¹⁷⁷Lu, which are typically therapeutic radionuclides but are also suitable for use in SPECT imaging applications.

The SSTR2 targeting compounds of the present invention are useful therapy compounds. Such a therapy compound includes a SSTR2 targeted compound of the invention with a suitable therapeutic moiety. For radiotherapy, the SSTR targeted compound is conjugated to a chelator, which is selected based on its suitability to hold an appropriate therapeutic radionuclide. A “therapeutic radionuclide” is a radionuclide that can be used for therapeutic purposes, e.g., for treating cancer due to their radioactive emissions, which have cytotoxic effects on targeted tissues (i.e., SSTR expressing cancers and tumor microenvironments, and malignancies). Although targeted radiotherapy has been practiced for some time using macrocyclic complexes of radionuclides, the macrocycles currently in use (e.g., DOTA) generally form complexes with many therapeutic radionuclide metals, such as actinium, radium, bismuth, astatine, lutetium, and lead isotopes among others. Instability of many known macrocyclic-containing compounds can result in some dissociation of the radionuclide from the macrocycle, and this results in a lack of selective delivery to the intended targeted tissue, which can also result in toxicity to non-targeted tissue. Alpha-emitting radionuclides such as ²²⁵Ac can provide much greater cytotoxic effects, and thus for therapy are considered substantially more potent than beta-emitting radionuclides. But this toxicity requires a chelator with increased retention of the chelated metal. U.S. Pat. No. 11,279,698 (see also, PCT/US2018/025488, and PCT/US2019/062479) describes a novel chelator (“Macropa”) and its use as a chelator for ²²⁵Ac. In some embodiments, the compounds of the invention exhibit increased circulatory residence time, reduced renal clearance, or improved tumor to non-target tissue uptake ratios. The ratio of tumor activity to kidney activity of 1 or greater may persist up to about 36 hours after administration of the radiotherapeutic, and in the case of an ²²⁵Ac based therapeutics may persist for 72, or even 128 hours, or longer, maximizing the therapeutic effects of the radiation on the target tissues.

Accordingly, an exemplary preferred SSTR targeted compound will have a chelator. Macropa is a preferred chelator for ²²⁵Ac-SSTR-targeted compounds. Exemplary structures follow, (see also, PCT/CA2021/050226).

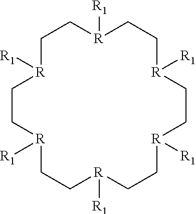

R can be O, N or S

Non-limiting examples of R1 are —CH₂CO₂H, alkyl, Targeting compound, 4-(4-isothiocyanatophenethoxy)picolinyl-2-methyl, picolinyl-2-methyl, 2-methyl pyridine, 2-methyl-4-(4-isothiocyanatophenethoxy)pyridine

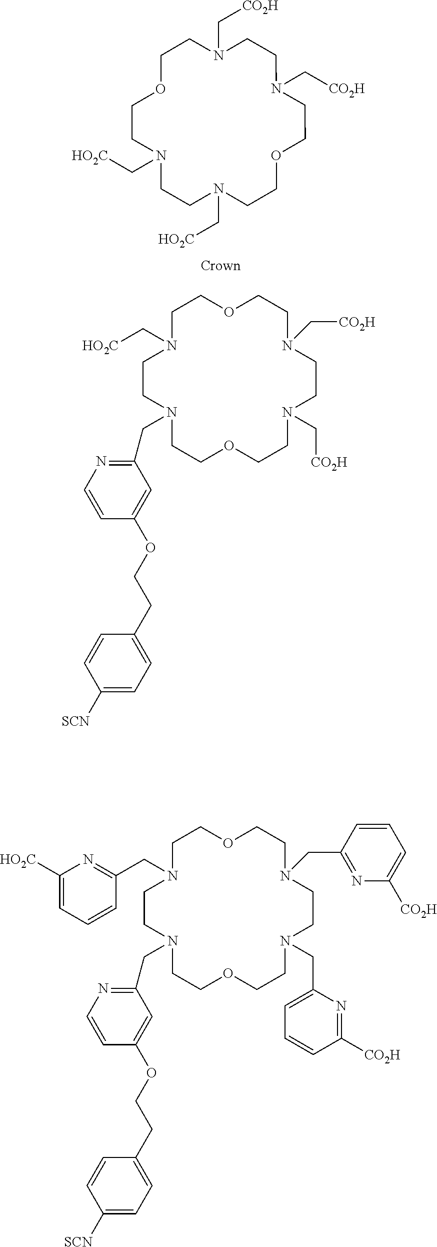

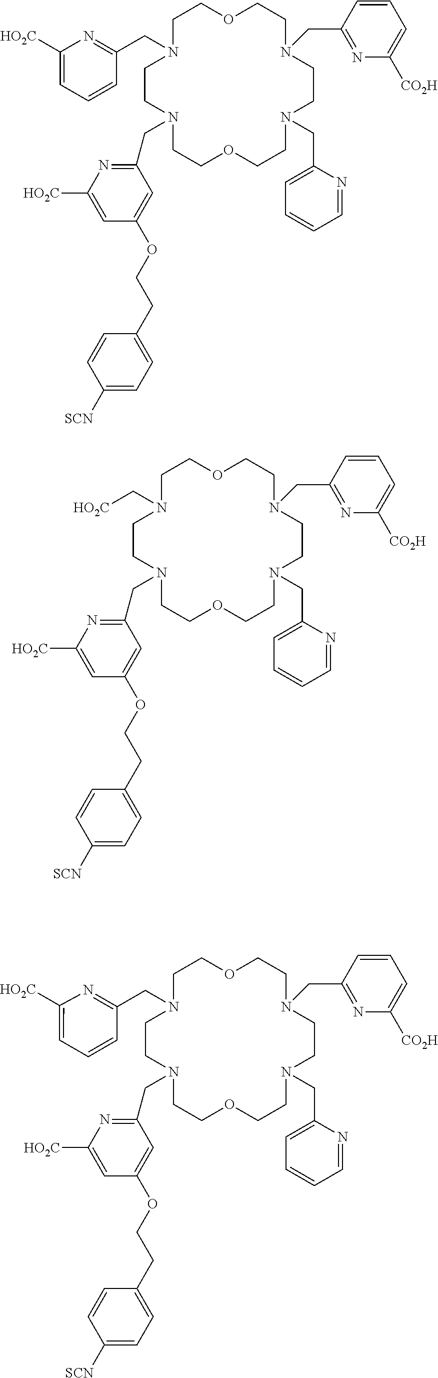

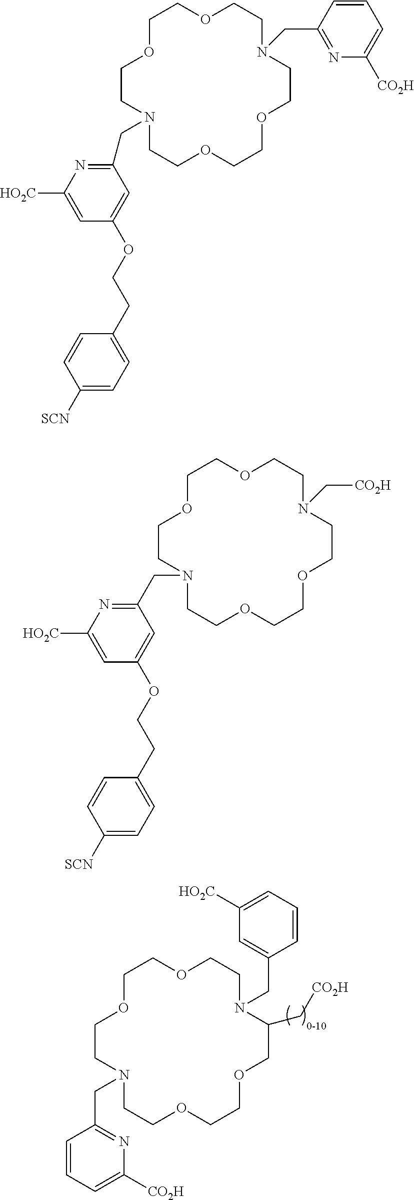

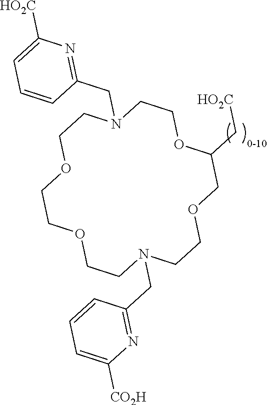

The radionuclide usable with the compounds disclosed herein depends on the application, radiation type desired and half-life as will be apparent to those of skill in the art. Exemplary radionuclides include: ¹⁷⁷Lu, ¹⁷⁵Lu, ⁴⁵Sc, ⁶⁴Cu, ⁶⁷Cu, ⁶⁸Cu, ⁶⁶Ga, ⁶⁷Ga, ⁶⁸Ga, ⁶⁹Ga, ⁷¹Ga, ⁹⁰Y, ⁸⁹Y, ⁸⁶Y, ⁸⁹Zr, ⁹⁰Yl, ⁹⁹mTc, ¹¹¹In, ¹¹³In, ¹¹⁵In, ¹³⁹La, ¹³⁴Ce, ¹³⁶Ce, ¹³⁸Ce, ¹⁴⁰Ce, ¹⁴²Ce, ¹⁵¹Eu, ¹⁵³Eu, ¹⁵²Dy, ¹⁴⁹Tb, ¹⁵⁹Tb, ¹⁶¹Tb, ¹⁵⁴Gd, ¹⁵⁵Gd, ¹⁵⁶Gd, ¹⁵⁷Gd, ¹⁵⁸Gd, ¹⁶⁰Gd, ¹⁸⁸Re, ¹⁸⁶Re, ²¹³Bi, ²¹¹At, ²¹⁷At, ²²⁷Th, ²²⁶Th, ²²⁵Ac, ²³³Ra, ¹⁵²Dy, ²¹³Bi, ²¹²Bi, ²¹¹Bi, ²⁰³Pb, ²¹²Pb, ²⁵⁵Fm, and ²³⁰U. The radionuclide of any embodiment herein may be both a therapeutic radionuclide, and a diagnostic radionuclide depending on its' decay profile. Currently preferred alpha-emitting radionuclides for therapy applications include ²²⁵Ac, ²³³Ra, and ²¹²Pb. Currently preferred beta-emitting radionuclides for therapy applications include ¹⁷⁷Lu, ⁹⁰Y, and ⁶⁷Cu.

Chelating groups and polyaza polycarboxylic macrocycles useful in the present technology include, and refer to a group that can chelate, bind or otherwise deliver a radionuclide to a therapeutic or diagnostic target. In one aspect, the chelating group comprises a macrocycle that binds the radionuclide, metal-ion, or metal-halogen ion, and optionally a linker that connects the macrocycle to the remainder of the molecule (e.g. the somatostatin binding group). A chelating group is the residue of a chelating agent after the chelating agent reacts with a nucleophilic group in a compound to form a targeted bivalent radio pharmaceutical or radio diagnostic agent that can bind and deliver a radionuclide. In the case of the disclosed compounds, the reactive group is N-terminus of the cyclic peptide or the side chain amine of a lysyl group in the penultimate precursor that reacts with the chelating agent to form the disclosed compounds. Examples of chelating agents include, but are not limited to, a covalently conjugated substituted or unsubstituted member of the following group: 1,4,7-triazacyclononane-1,4,7-triacetic acid (NOTA), p-SCN-Bn-NOTA, 1,4,7,10-tetraazacyclododecane-1,4,7,10-tetraacetic acid (DOTA), p-SCN-Bn-DOTA (also known as 2B-DOTA-NCS), PIP-DOTA, diethylenetriaminepentaacetic acid (DTPA), PIP-DTPA, AZEP-DTPA, ethylenediamine tetraacetic acid (EDTA), triethylenetetraamine-N,N,N′,N″,N′″,N′″-hexa-acetic acid (TTHA), 7-[2-(bis-carboxymethylamino)-ethyl]-4,10-bis-carboxymethyl-1,4,7,10-tetraaza-cyclododec-1-yl-acetic acid (DEPA), 2,2′,2″-(10-(2-(bis(carboxymethyl)amino)-5-(4-isothiocyanatophenyl) pentyl)-1,4,7,10-tetraazacyclododecane-1,4,7-triyl)triacetic acid (3p-C-DEPA-NCS), NETA,{4-carboxymethyl-7-[2-(carboxymethylamino)-ethyl]-perhydro-1,4,7-triazonin-1-yl}-acetic acid (NPTA), diacetylpyridinebis(benzoylhydrazone), 1,4,7,10,13,16-hexaazacyclooctadecane N,N′,N″,N′″,N″″,N′″″-hexaaceticacid (HEHA), octadentate terephthalamide ligands, 2,2′-(4-(2-(bis(carboxymethyl)amino)-5-(4-isothiocyanatophenyl)pentyl)-10-(2-(bis(carboxymethyl)amino)ethyl)-1,4,7,10-tetraazacyclododecane-1,7-diyl)diacetic acid, N,N′-bis[(6-carboxy-2-pyridyl)methyl]-4,13-diaza-18-crown-6 (H2macropa), 6-((16-((6-carboxypyridin-2-yl)methyl)-1,4,10,13-tetraoxa-7,16-diazacyclooctadecan-7-yl)methyl)-4-isothiocyanatopicolinic acid (macropa-NCS), 6-((16-((6-carboxypyridin-2-yl)methyl)-1,4,10,13-tetraoxa-7,16-diazacyclooctadecan-7-yl)methyl)-4-isocyanatopicolinic acid (macropa-NCO), 3,9-carboxymethyl-6-(2-methoxy-5-isothiocyanatophenyl)carboxymethyl-3,6,9,15-tetraazabicyclo-[9.3.1]pentadeca-1(15),11,13-triene and 2-[4,7,10-tris(2-amino-2-oxoethyl)-1,4,7,10-tetrazacyclododec-1-yl]acetamide (TCMC or DOTAM). In another example, the chelating group is the residue of a polyaza polycarboxylic macrocycle, such as

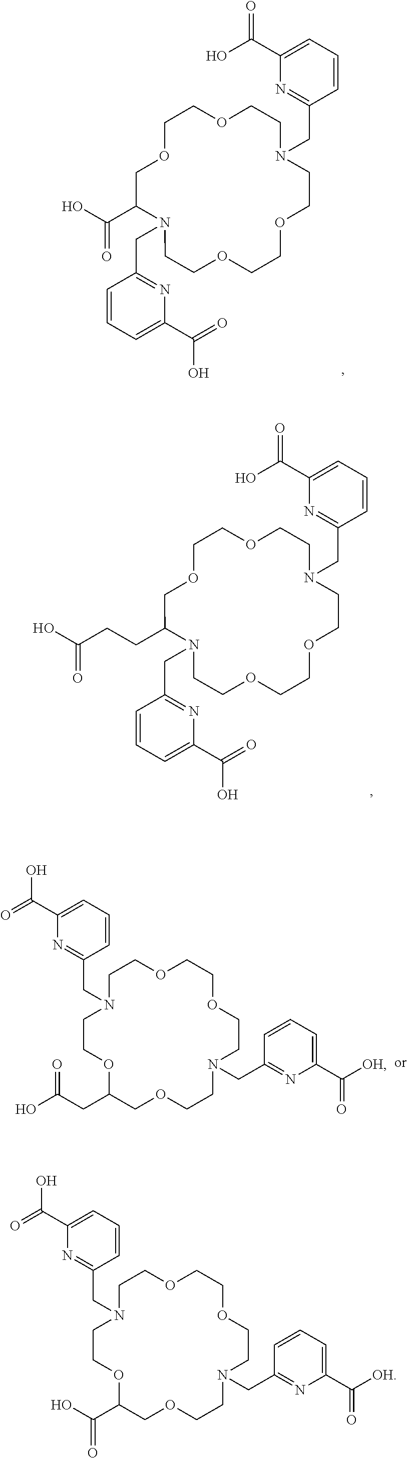 [SMILES: O=C(O)c1cccc(CN2CCOCCOCCN(Cc3cccc(C(=O)O)n3)C(C(=O)O)COCCOCC2)n1 ; O=C(O)CCC1COCCOCCN(Cc2cccc(C(=O)O)n2)CCOCCOCCN1Cc1cccc(C(=O)O)n1 ; O=C(O)CC1COCCN(Cc2cccc(C(=O)O)n2)CCOCCOCCN(Cc2cccc(C(=O)O)n2)CCO1 ; O=C(O)c1cccc(CN2CCOCCOCCN(Cc3cccc(C(=O)O)n3)CCOC(C(=O)O)COCC2)n1]

In another aspect, the chelating group is the residue of a siderophores, In one aspect, ²²⁵Ac is the radionuclide for a macropa chelating group. In another aspect, the chelating group is the residue of p-SCN-Bn-DOTA, p-SCN-Bn-NOTA, NOTA or DOTA. In another embodiment, the chelating group comprises or is a sarcophagine chelator. In another aspect, the chelating group is the residue of p-SCN-Bn-DOTA, p-SCN-Bn-NOTA, NOTA or DOTA chelated with ⁶⁸Ga.

In an alternative embodiment, the chelating group of the compounds disclosed herein may be chelated with a metal ion or metal-halogen ion. In some embodiments, the metal ion or metal-halogen ion comprises a radionuclide. In some embodiments, the metal ion or metal-halogen ion comprises a non-radioactive metal ion or metal-halogen ion. Exemplary metal ions and metal-halogen ions include: ¹⁷⁷Lu, ¹⁷⁵Lu, ⁴⁴Sc, ⁴⁵Sc, ⁴⁷Sc, ⁶⁴Cu, ⁶⁷Cu, ⁶⁸Cu, ¹⁸F (e.g., in the form of [¹⁸F]AlF²⁺)⁶⁶Ga, ⁶⁷Ga, ⁶⁸Ga, ⁶⁹Ga, ⁷¹Ga, ⁹⁰Y, ⁸⁹Y, ⁸⁶Y ⁸⁹Zr, ⁹⁰Y, ⁹⁹mTc, ¹¹¹In, ¹¹³n, ¹¹⁵In, ¹²³I, ¹²⁵I, ¹³¹I, ¹³⁹La, ¹³⁴Ce, ¹³⁶Ce, ¹³⁸Ce, ¹⁴⁰Ce, ¹⁴²Ce, ¹⁴³Ce, ¹⁵³Sm, ¹⁵¹Eu, ¹⁵³Eu, ¹⁵²Dy, ¹⁴⁹Tb, ¹⁵⁹Tb, ¹⁶¹Tb, ¹⁵⁴Gd, ¹⁵⁵Gd, ¹⁵⁶Gd, ¹⁵⁷Gd, ¹⁵⁸Gd, ¹⁶⁰Gd, ¹⁸⁸Re, ¹⁸⁶Re, ²¹³Bi, ²¹¹At, ²¹⁷At, ²²⁷Th, ²²⁶Th ²²⁵Ac, ²²³Ra, ²²⁴Ra, ²³³Ra, ¹⁵²Dy, ²¹³Bi, ²¹²Bi, ²¹¹Bi, ²⁰³Pb, ²¹²Pb, ²⁵⁵Fm, and ²³⁰U.

In another embodiment of the invention, the radionuclide is an Auger electron emitting radionuclide or a beta-minus-emitting radionuclide such as ¹⁷⁷Lu, ¹⁸⁸Re, ¹⁶¹Tb, ⁹⁰Y, and ⁶⁷Cu.

Another aspect of the invention is a metal complex comprising a compound of any one of the embodiments disclosed herein, or a pharmaceutically acceptable salt thereof, and any one of the radionuclides disclosed herein, wherein the radionuclide is complexed to the chelating group. An alternative aspect of the invention is a metal complex comprising a compound of any one of the embodiments disclosed herein, or a pharmaceutically acceptable salt thereof, and any one of the metal ions or metal-halogen ions disclosed herein, wherein the metal ion or metal-halogen ion is complexed to the chelating group.

As noted above, complexes of the disclosed compounds or pharmaceutically acceptable salts thereof may contain one or more radionuclides which are suitable for use as radio-imaging agents. Imaging methods include positron emission tomography (PET) or single photon emission computed tomography (SPECT). In an exemplary embodiment, the radionuclide is a beta-plus-emitting (i.e. a positron-emitting) radionuclide for positron-emitting tomography (PET) or gamma ray/photon emitting radionuclide for single-photon emission computerized tomography (SPECT) (e.g., ¹⁸F, ⁶⁸Ga, and ⁶⁴Cu, or ^{99m}Tc, ¹¹¹In, and ¹⁸⁶Re). Accordingly in another aspect, the invention provides for theranostic applications, i.e., methods where a subject with a cancer or a tumor is administered an effective amount of a disclosed compound (or a pharmaceutically acceptable salt thereof) having a chelator, which is complexed to an imaging radionuclide for imaging applications, and administered an effective amount of the compound complexed to a therapeutic radionuclide for treatment.

The disclosed compounds can be used to treat cancers in a subject. Cancers treatable with the disclosed compounds are cancers associated with expression of somatostatin type 2 receptors (SSTR2). In specific embodiments, the cancer which expresses SSTR2 is selected from the group consisting of pituitary tumors, neuroendocrine tumors, renal cell cancer, breast cancer, meningioma, glioma, neuroblastoma, colorectal cancer, pheochromocytoma, medullary thyroid cancer, ovarian cancer, head and/or neck cancer, gastric cancer, adrenal cancer, brain cancer, and a hematologic malignancy such as lymphoma or leukemia. In particular embodiments, the cancer is a neuroendocrine tumor, such as a carcinoid tumor in the lung, appendix, digestive tract, prostate, thymus or rectum or a pancreatic neuroendocrine tumor. In further embodiments, the cancer is a neuroendocrine tumor such as a gastrinoma, insulinoma or non-functioning islet cell tumor.

In some embodiments, the cancer is selected from the group consisting of epithelial ovarian cancer, ovarian carcinoma, osteosarcoma, pancreatic adenocarcinoma, colorectal cancer, lung cancer, non-small cell lung cancer, gastric cancer, endometrial carcinoma, pancreatic adenocarcinoma, medullary thyroid carcinoma, differentiated thyroid cancer, breast cancer, invasive ductal carcinoma of the breast, oral squamous cell carcinoma, esophageal cancer, renal cell cancer, insulinoma, prostate cancer, neuroendocrine differentiated prostate cancer, pheochromocytoma, adenoid cystic cancer, hepatocellular carcinoma, cervical cancer, small intestine cancer, neuroendocrine tumor, anal cancer, chordoma, desmoid tumor, head and neck cancer, thymus cancer, pancreatic cancer, cholangiocellular carcinoma, esophageal cancer, salivary gland cancer, sarcoma and carcinoma of unknown primary cancer.

As used herein, the term “expression” in relation to SSTR2 relates to the presentation of the receptor on the surface of the tumor or cancer cell. Healthy tissue may also express SSTR2, whereas cancerous or tumor cells and/or tissues may show upregulation or “overexpression” of SSTR2 meaning that the abundance of the receptor on cancerous cells is greater than when compared to healthy tissue.

In some embodiments, a compound of Formula (I), (IIa), (IIb), (IIc), (IIIa), (IIIb), (IIIc), (IVa), (IVb), (IVc), (Va), (Vb), (Vc), (VIa), (VIb), (VIc), (VIIa), (VIIb), (VIIc), (VIIIa), (VIIIb), (VIIIc), (IXa), (IXb), or (IXc), complexed with a radionuclide may be used for the treatment of a cancer that is associated with expression of SSTR2. In other embodiments, a compound of the present invention complexed with a radionuclide is used for the treatment of a cancer selected from the group consisting of pituitary tumors, neuroendocrine tumors, renal cell cancer, breast cancer, meningioma, glioma, neuroblastoma, colorectal cancer, pheochromocytoma, medullary thyroid cancer, ovarian cancer, head and/or neck cancer, gastric cancer, adrenal cancer, brain cancer, and a hematologic malignancy such as lymphoma or leukemia. In alternative embodiment, a compound of the present invention complexed with a radionuclide, metal ion, or metal-halogen ion is used for the treatment of a cancer selected from the group consisting of pituitary tumors, renal cell cancer, breast cancer, meningioma, glioma, glioblastoma multiforme (GBM), neuroblastoma, colorectal cancer, pheochromocytoma, paraganglioma, medullary thyroid cancer, small cell lung cancer, ovarian cancer, head and/or neck cancer, gastric cancer, adrenal cancer, brain cancer, and a hematologic malignancy. In particular embodiments, the cancer is a neuroendocrine tumor, such as a carcinoid tumor in the lung, appendix, digestive tract, prostate, thymus or rectum or a pancreatic neuroendocrine tumor. In another particular embodiment, the neuroendocrine tumor is selected from the group consisting of gastroenteropancreatic neuroendocrine tumor, carcinoid tumor, pheochromocytoma, paraganglioma, medullary thyroid cancer, pulmonary neuroendocrine tumor, thymic neuroendocrine tumor, a carcinoid tumor or a pancreatic neuroendocrine tumor, pituitary adenoma, adrenal gland tumors, Merkel cell carcinoma, breast cancer, non-Hodgkin lymphoma, Hodgkin lymphoma, head & neck tumor, urothelial carcinoma (bladder), renal cell carcinoma, hepatocellular carcinoma, GIST, neuroblastoma, bile duct tumor, cervix tumor, Ewing sarcoma, osteosarcoma, small cell lung cancer (SCLC), prostate cancer, melanoma, meningioma, glioma, medulloblastoma, hemangioblastoma, supratentorial primitive, neuroectodermal tumor, esthesioneuroblastoma functional carcinoid tumor, insulinoma, gastrinoma, vasoactive intestinal peptide (VIP) oma, glucagonoma, serotoninoma, histaminoma, ACTHoma, pheocromocytoma, and somatostatinoma. In further embodiments, the cancer is a neuroendocrine tumor such as a gastrinoma, insulinoma or non-functioning islet cell tumor.

In another embodiment, a compound of Formula (I), (IIa), (IIb), (IIc), (IIIa), (IIIb), (IIIc), (IVa), (IVb), (IVc), (Va), (Vb), (Vc), (VIa), (VIb), (VIc), (VIIa), (VIIb), (VIIc), (VIIIa), (VIIIb), (VIIIc), (IXa), (IXb), or (IXc), complexed with a radionuclide may be used in combination with a second anti-cancer therapeutic for the treatment of a diseased tissue, wherein the diseased tissue express somatostatin receptors cancer that is associated with expression of SSTR2. In some embodiments, the “second anti-cancer therapeutic” may be selected from chemotherapeutic agents such as capecitabine, temozolomide, streptozocin, 5-fluorouracil, cisplatin, carboplatin, etoposide, and doxorubicin; external beam radiotherapy; or immunotherapies, including immune checkpoint inhibitors (e.g., PD-1, PD-L1, and CTLA-4 inhibitors). Additional combination options include targeted agents that enhance therapeutic efficacy, such as mTOR inhibitors (e.g., everolimus), PARP inhibitors (e.g. olaparib), NAMPT inhibitors (e.g., GMX1778), Hedgehog pathway inhibitors (e.g., sonidegib), and demethylating agents (e.g., ASTX727). These agents may enhance the efficacy of the radionuclide complex through mechanisms such as radiosensitization, enhanced tumor perfusion, SSTR2 upregulation, or complementary DNA damage pathways, therapy improving tumor targeting and minimizing off-target toxicity. In some embodiments, a compound of the present invention complexed with a radionuclide is used in combination with a second anti-cancer agent for the treatment of a cancer selected from the group consisting of pituitary tumors, neuroendocrine tumors, renal cell cancer, breast cancer, small cell lung cancer (SCLC), meningioma, glioma, neuroblastoma, colorectal cancer, pheochromocytoma, medullary thyroid cancer, ovarian cancer, head and/or neck cancer, gastric cancer, adrenal cancer, brain cancer, and a hematologic malignancy such as lymphoma or leukemia.

The present invention also discloses the use of the compounds of Formula (I), (IIa), (IIb), (IIc), (IIIa), (IIIb), (IIIc), (IVa), (IVb), (IVc), (Va), (Vb), (Vc), (VIa), (VIb), (VIc), (VIIa), (VIIb), (VIIc), (VIIIa), (VIIIb), (VIIIc), (IXa), (IXb), or (IXc), complexed with a suitable radionuclide for radioimaging a subject. In certain embodiments, the compounds of Formula (I), (IIa), (IIb), (IIc), (IIIa), (IIIb), (IIIc), (IVa), (IVb), (IVc), (Va), (Vb), (Vc), (VIa), (VIb), (VIc), (VIIa), (VIIb), (VIIc), (VIIIa), (VIIIb), (VIIIc), (IXa), (IXb), or (IXc), complexed with a suitable radionuclide is used for radioimaging a cancer that is associated with expression of SSTR2. In other embodiments, a compound of the present invention is used for the radioimaging of a cancer selected from the group consisting of pituitary tumors, neuroendocrine tumors, renal cell cancer, breast cancer, meningioma, glioma, neuroblastoma, colorectal cancer, pheochromocytoma, medullary thyroid cancer, ovarian cancer, head and/or neck cancer, gastric cancer, adrenal cancer, brain cancer, and a hematologic malignancy such as lymphoma or leukemia. In particular embodiments, the cancer is a neuroendocrine tumor, such as a carcinoid tumor in the lung, appendix, digestive tract, prostate, thymus or rectum or a pancreatic neuroendocrine tumor. In further embodiments, the cancer is a neuroendocrine tumor such as a gastrinoma, insulinoma or non-functioning islet cell tumor.

Another embodiment of the present invention includes the compounds of Formula (I), (IIa), (IIb), (IIc), (IIIa), (IIIb), (IIIc), (IVa), (IVb), (IVc), (Va), (Vb), (Vc), (VIa), (VIb), (VIc), (VIIa), (VIIb), (VIIc), (VIIIa), (VIIIb), (VIIIc) (IXa), (IXb), or (IXc), or a pharmaceutically acceptable salt thereof, the metal complex of any of the compounds disclosed herein, or the pharmaceutical composition comprising the compounds disclosed herein for use as a medicament.

In another embodiment, the present invention includes the compounds of Formula (I), (IIa), (IIb), (IIc), (IIIa), (IIIb), (IIIc), (IVa), (IVb), (IVc), (Va), (Vb), (Vc), (VIa), (VIb), (VIc), (VIIa), (VIIb), (VIIc), (VIIIa), (VIIIb), (VIIIc), (IXa), (IXb), or (IXc), or a pharmaceutically acceptable salt thereof, the metal complex of any of the compounds disclosed herein, or the pharmaceutical composition comprising the compounds disclosed herein for use in a method of treating cancer.

In yet another embodiment, the present invention includes the compounds of Formula (I), (IIa), (IIb), (IIc), (IIIa), (IIIb), (IIIc), (IVa), (IVb), (IVc), (Va), (Vb), (Vc), (VIa), (VIb), (VIc), (VIIa), (VIIb), (VIIc), (VIIIa), (VIIIb), (VIIIc), (IXa), (IXb), or (IXc), or a pharmaceutically acceptable salt thereof, the metal complex of any of the compounds disclosed herein, or the pharmaceutical composition comprising the compounds disclosed herein for use in a method of diagnosing cancer.

In yet another embodiment, the present invention includes the compounds of Formula (I), (IIa), (IIb), (IIc), (IIIa), (IIIb), (IIIc), (IVa), (IVb), (IVc), (Va), (Vb), (Vc), (VIa), (VIb), (VIc), (VIIa), (VIIb), (VIIc), (VIIIa), (VIIIb), (VIIIc), (IXa), (IXb), or (IXc), or a pharmaceutically acceptable salt thereof, the metal complex of any of the compounds disclosed herein, or the pharmaceutical composition comprising the compounds disclosed herein for use in a method of imaging a patient suspected to have cancer.

In an exemplary embodiment, the cancer is a neuroendocrine tumor (NET). In another exemplary embodiment, the neuroendocrine tumor is selected from the group consisting of gastroenteropancreatic neuroendocrine tumor, carcinoid tumor, pheochromocytoma, paraganglioma, medullary thyroid cancer, pulmonary neuroendocrine tumor, thymic neuroendocrine tumor, a carcinoid tumor or a pancreatic neuroendocrine tumor, pituitary adenoma, adrenal gland tumors, Merkel cell carcinoma, breast cancer, non-Hodgkin lymphoma, Hodgkin lymphoma, head & neck tumor, urothelial carcinoma (bladder), renal cell carcinoma, hepatocellular carcinoma, GIST, neuroblastoma, bile duct tumor, cervix tumor, Ewing sarcoma, osteosarcoma, small cell lung cancer (SCLC), prostate cancer, melanoma, meningioma, glioma, medulloblastoma, hemangioblastoma, supratentorial primitive, neuroectodermal tumor, esthesioneuroblastoma functional carcinoid tumor, insulinoma, gastrinoma, vasoactive intestinal peptide (VIP) oma, glucagonoma, serotoninoma, histaminoma, ACTHoma, pheocromocytoma, and somatostatinoma.

A “subject” is a mammal in need of medical treatment or diagnosis, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The terms “administer”, “administering”, “administration”, and the like, as used herein, refer to methods that may be used to enable delivery of compositions to the desired site of biological action. The disclosed compounds or pharmaceutically acceptable salts thereof (including chelation with a radionuclide) or pharmaceutical compositions thereof, may be administered orally or via a parenteral route, usually injection or infusion. A “parenteral administration route” means modes of administration other than enteral and topical administration, usually by injection, and includes, without limitation, intravenous, intramuscular, intraarterial, intrathecal, intracapsular, intraorbital, intracardiac, intradermal, intraperitoneal, intratumoral, transtracheal, subcutaneous, subcuticular, intraarticular, subcapsular, subarachnoid, intraspinal and intrasternal injection and infusion.

“Effective amount” of the disclosed compounds or pharmaceutically acceptable salts thereof (including chelation with a radionuclide) means that amount of therapeutic agent alone, or in combination with other therapies, that provides a therapeutic benefit in the treatment of prevention of a disease, that improves overall therapy, reduces or avoids symptoms or causes of disease, or enhances the therapeutic efficacy of or synergies with another therapeutic agent. With respect to imaging, “diagnostically effective amount” refers to the amount which will result in a useful image for diagnosing, e.g., the presence of a tumor. “Effective amount” of the disclosed compounds or pharmaceutically acceptable salt thereof, is determined by the physician on the basis of the patient-specific parameters, such as age, weight, sex, severity of the disease, etc. The dosage is preferably from 0.0001 mg/kg to 100 mg/kg body weight.

Corresponding to the kind of administration, the medicament is suitably formulated, e.g. in the form of solutions or suspensions, simple tablets or dragees, hard or soft gelatine capsules, suppositories, ovules, preparations for injection, which are prepared according to common galenic methods.

When solutions for infusion or injection are used, they are preferably aqueous solutions or suspensions, it being possible to produce them prior to use, e.g. from lyophilized preparations which contain the active substance as such or together with a carrier, such as mannitol, lactose, glucose, albumin and the like. The ready-made solutions are sterilized and, where appropriate, mixed with excipients, e.g. with preservatives, stabilizers, emulsifiers, solubilizers, buffers and/or salts for regulating the osmotic pressure. The sterilization can be obtained by sterile filtration using filters having a small pore size according to which the composition can be lyophilized, where appropriate. Small amounts of antibiotics can also be added to ensure the maintenance of sterility.

According to another aspect, a pharmaceutical composition is provided, which is suitable for in vivo imaging and/or radiotherapy of a target tissue. Suitable pharmaceutical compositions may contain a radioimaging agent that has a radionuclide either as an element, (i.e., ¹⁸F), or a diagnostic radioactive metal chelate complex (e.g., with ⁶⁴Cu or ⁶⁸Ga), or a radiotherapeutic agent which is radioactive metal chelate complex, in an amount sufficient for binding to the target tissue, together with a pharmaceutically acceptable radiological vehicle. The radiological vehicle should be suitable for injection or aspiration, such as human serum albumin; aqueous buffer solutions, e.g., tris(hydromethyl) aminomethane (and its salts), phosphate, citrate, bicarbonate, etc.; sterile water physiological saline; and balanced ionic solutions containing chloride and or dicarbonate salts or normal blood plasma cautions such as calcium potassium, sodium and magnesium.

The concentration of the radiopharmaceutical agent in the radiological vehicle should be sufficient to provide reasonable binding to the target tissue, such as about 4% to 40% ID/gram. For example, when using an aqueous solution, the human dosage can range from about 1.0 to 500 millicuries of activity. The actual dose administered to a patient for imaging or therapeutic purposes, however, is determined by the physician administering treatment. The imaging agent or therapeutic agent should be administered so as to remain in the patient for about 1 hour to 10 days, although both longer and shorter time periods are acceptable. Therefore, convenient ampoules containing 1 to 15 mL of aqueous solution may be prepared.

Imaging may be carried out in the normal manner, for example by injecting a sufficient amount of the imaging composition to provide adequate imaging and then scanning with a suitable imaging or scanning machine, such as a tomograph or gamma camera. In certain embodiments, a method of imaging a region in a patient includes the steps of: (i) administering to a patient a diagnostically effective amount of a compound complexed with a radionuclide; exposing a region of the patient to the scanning device; and (ii) obtaining an image of the region of the patient. Accordingly, the invention provides a method for obtaining an image of a mammalian subject following administration of the compound. Likewise, imaging can be performed after administration of a therapeutic drug or radiotherapy cycle to assess efficacy. Thus, obtaining an image after administration of the radiotherapeutic may occur after about 1 hour, about 4 hours, about 9 hours, about 12 hours, about 16 hours, about 20 hours, about 24 hours, about 36 hours, about 48 hours, about 72 hours, about 96 hours, about one week, about two weeks, about four weeks, or after completion of a cycle of therapeutic treatments. Thus, in some embodiments, a method of imaging tissue such as SSTR expressing tumor tissue is provided including contacting the tissue with a complex synthesized by contacting an imaging radionuclide with a disclosed compound.

According to another aspect, complexes of the disclosed compounds or pharmaceutically acceptable salts thereof may contain one or more radionuclides which are suitable for use as radio-imaging agents in the field of image guided radiation therapy (IGRT). As described in U.S. patent No. U.S. Pat. No. 10,688,320 B2, IGRT uses images acquired before a treatment session to guide the application of therapeutic radiation during a treatment session. The concentration of the imaging agent or the therapeutic agent in the radiological vehicle should be sufficient to provide satisfactory imaging. For example, when using an aqueous solution, the dosage is about 1.0 to 100 millicuries. Imaging can be performed to provide a fiducial, for guidance for the target region to receive a calculated radiation fluence from a therapeutic radiation source. Similar uses of the compounds as fiducials can be used in guided surgery applications.

The amount of the compound of the present invention, or a formulation comprising a complex of a metal and a compound or pharmaceutically acceptable salt thereof that is administered to a patient depends on several physiological factors that are routinely used by the physician, including the nature of the procedure to be carried out, the volume of tissue to be targeted for imaging or therapy and the body weight and medical history of the patient to be imaged or treated using the compounds.

The examples herein are provided to illustrate advantages of the present technology and to further assist a person of ordinary skill in the art with preparing or using the compounds of the present technology or salts, pharmaceutical compositions, derivatives, prodrugs, or tautomeric forms thereof. The examples herein are also presented in order to more fully illustrate the preferred aspects of the present technology. The examples should in no way be construed as limiting the scope of the present technology, as defined by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### EXEMPLIFICATION

### General Information

Solvents and reagents were purchased from Sigma-Aldrich, VWR, or Fisher Scientific, and used without further purification. Reactions were monitored either by thin-layer chromatography (TLC) or by analytical liquid chromatography-mass spectrometry (LC-MS) employing a Waters Acquity Ultra Performance LC system and a Synapt high-definition mass spectrometer. ¹H NMR spectra were recorded on an Oxford AS400 magnet connected to a Varian Mercury console (300 MHz). All chemical shifts are reported in ppm and coupling constants, J, are reported in hertz (Hz). NMR solvent peaks were referenced as follows: (¹H NMR) CDCl₃: 7.27 ppm, DMSO-d₆: 2.50 ppm. Compounds were purified by flash column chromatography on a Teledyne ISCO Combi-Flash system using normal phase silica gel (SiliCycle Inc.) or reverse phase (Teledyne Gold-C18 or C18Aq) pre-packed columns. The purity of compounds was determined by analytical HPLC (Waters Acquity Ultra Performance) using an Acquity UPLC CSH C18 1.7 μm (50 mm×2.1 mm) column and flow rate of 0.3 mL/min. Gradient conditions: solvent A (0.1% formic acid in water) and solvent B (0.1% formic acid in acetonitrile): 0-0.1 min 95% A, 0.1-4.0 min 5-95% B (linear gradient), 4.0-5.0 min 95% B, UV detection at 254 nm and 220 nm.

### Example 1—Preparation of the Intermediates

### Preparation of Intermediate Cyclo-D-Phe-[Cys(Trt)-Tyr(O-t-Bu)-D-Trp(Boc)-Lys(Me, Boc)-Thr(O-t-Bu)-Cys(Trt)]-Thr(O-t-Bu)-2-Cl-trityl Resin (Int-1)

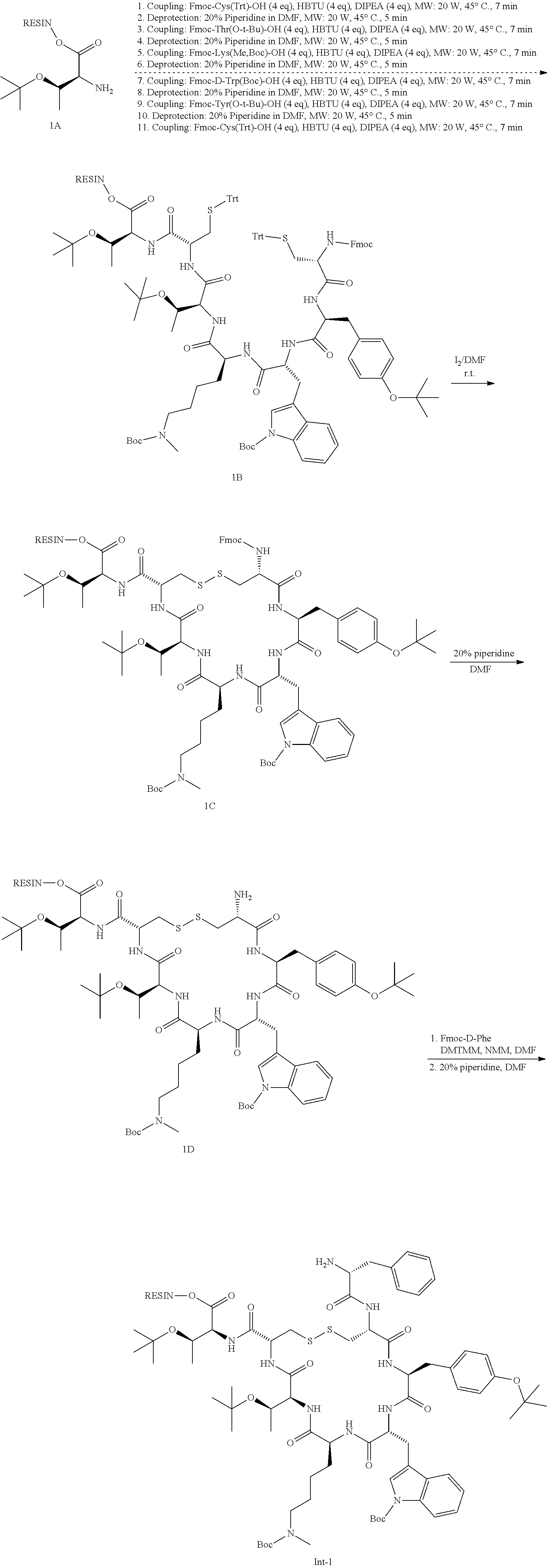

### Fmoc-Cys(Trt)-Tyr(O-t-Bu)-D-Trp(Boc)-Lys(Me, Boc)-Thr(O-t-Bu)-Cys(Trt)-Thr(O-t-Bu)-2-Cl-trityl Resin (1B)

Thr(O-t-Bu) 2-Cl-trityl resin (1A, 1 mmol, Chem Impex Cat #: 06803) was swelled in DMF (20 ml) for 30 minutes, then filtered. The Fmoc group was removed by treating the resin with 20% piperidine in DMF (20 ml) at 25° C. for 1 h, then washed resin with DMF (20 ml×5). SPPS was carried out as follows. Coupling: Pre-activate Fmoc-AA-OH (4 eq.) with HBTU (4 eq.) and DIPEA (4 eq.) in 20 ml of DMF for 1 min. Added to resin and heated in microwave reactor at 20 W, 45° C. for 7 min with gentle stirring. Washed resin with DMF (10 ml×5). Deprotection: 20% piperidine in DMF (20 ml) was added to resin and heated in microwave reactor at 20 W, 45° C. for 5 min with gentle stirring. Washed resin with DMF (10 ml×5). (assume quant. yield of 1B).

### Cyclo-[Fmoc-Cys(Trt)-Tyr(O-t-Bu)-D-Trp(Boc)-Lys(Me, Boc)-Thr(O-t-Bu)-Cys(Trt)]-Thr(O-t-Bu)-2-Cl-trityl Resin (1C)

Resin-peptide 1B (1 mmol) was suspended in DMF (20 ml). Iodine (5 eq) was added, and the mixture was gently stirred at 25° C. for 2 h, filtered and washed resin with DMF (10 ml×5) and DCM (10 ml×5). (assume quant. yield of 1C).

### Cyclo-[Cys(Trt)-Tyr(O-t-Bu)-D-Trp(Boc)-Lys(Me, Boc)-Thr(O-t-Bu)-Cys(Trt)]-Thr(O-t-Bu)-2-CL-trityl Resin (1D)

Resin-peptide 1C (1 mmol) was treated with a solution of 20% piperidine in DMF (20 ml) and heated in microwave reactor at 20 W, 45° C. for 5 min with gentle stirring. Washed resin with DMF (10 ml×5). (assume quant. yield of 1D).

### Cyclo-D-Phe-[Cys(Trt)-Tyr(O-t-Bu)-D-Trp(Boc)-Lys(Me, Boc)-Thr(O-t-Bu)-Cys(Trt)]-Thr(O-t-Bu)-2-Cl-trityl Resin (Int-1)

Resin-peptide 1D (0.2 mmol) was suspended in DMF (5 ml). Fmoc-D-Phe-OH (0.3 mmol, 1.5 eq), NMM (0.4 mmol, 2 eq) and DMTMM (0.3 mmol, 1.5 eq) were added and the reaction was stirred gently at 25° C. for 1 h. The resin-peptide was filtered and washed with DMF (5 ml×5). 20% piperidine in DMF (5 ml) was added to resin-peptide and stirred at 25° C. for 30 min with gentle stirring. Washed resin-peptide with DMF (5 ml×5) then DCM (5 ml×5). (assume quant. yield of Int-1).

### Cyclo-D-Phe-[Cys(Trt)-Tyr(O-t-Bu)-D-Trp(Boc)-Lys(Me, Boc)-Thr(O-t-Bu)-Cys(Trt)]-Thr(O-t-Bu)-OH (Int-2)

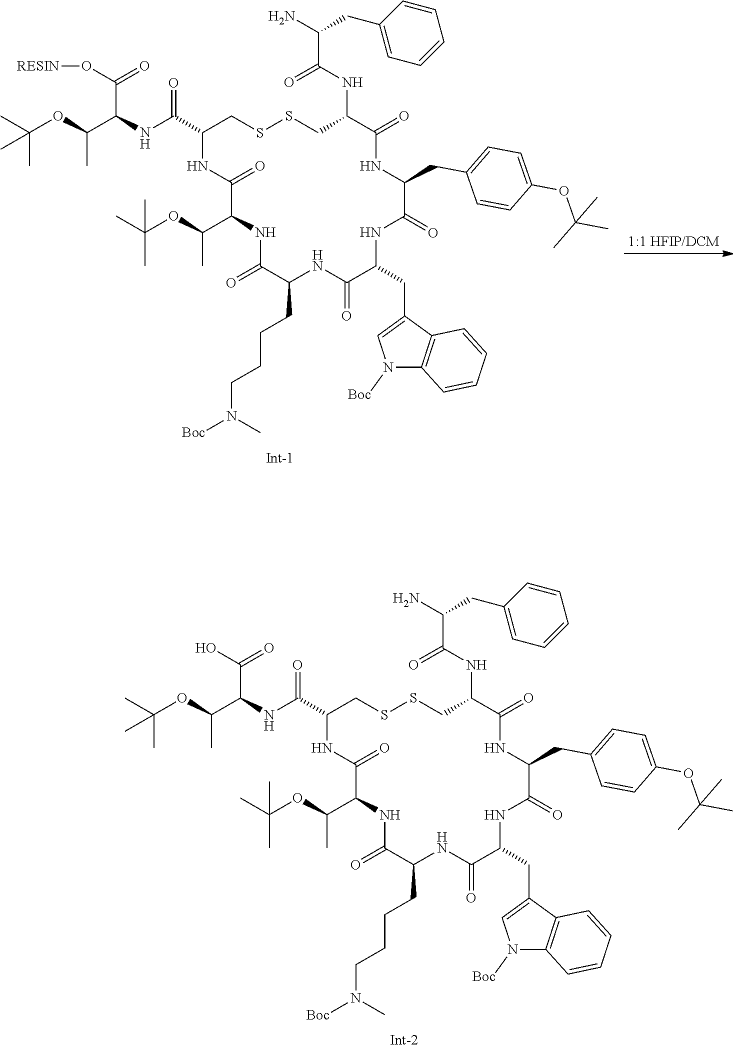

Resin-peptide Int-1 (0.2 mmol) was cleaved using a solution of 1:1 HFIP/DCM (2 ml) at 25° C. for 5 min. Filtered and extracted resin with DCM (5 ml). The resin was cleaved two more times using the same sequence. The combined filtrates were concentrated in vacuo to give a sticky solid that was dissolved in 3:1 MeCN/water (5 ml) and lyophilized to give Int-2 as a white solid (240 mg). MS (ESI) m/z: 1432.1 (M+H)⁺.

The following intermediates were prepared using procedures similar to those for Int-1 and Int-2 with the corresponding amino acids:

| Int No. | Structure |
|---|---|
| Int-26 | 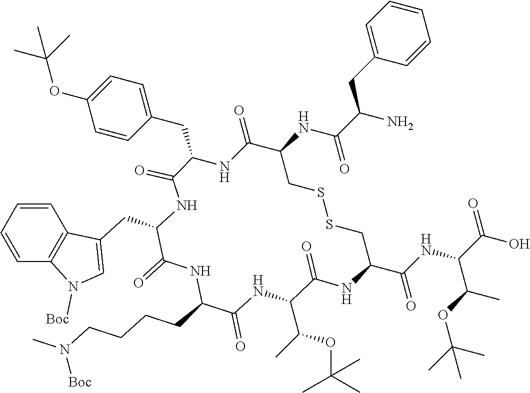 [SMILES: C[C@@H](OC(C)(C)C)[C@H](NC(=O)[C@@H]1CSSC[C@H](NC(=O)[C@H](N)Cc2ccccc2)C(=O)N[C@@H](Cc2ccc(OC(C)(C)C)cc2)C(=O)N[C@@H](Cc2cn(C(=O)OC(C)(C)C)c3ccccc23)C(=O)N[C@H](CCCCN(C)C(=O)OC(C)(C)C)C(=O)N[C@@H]([C@@H](C)OC(C)(C)C)C(=O)N1)C(=O)O] |
| Int-27 | 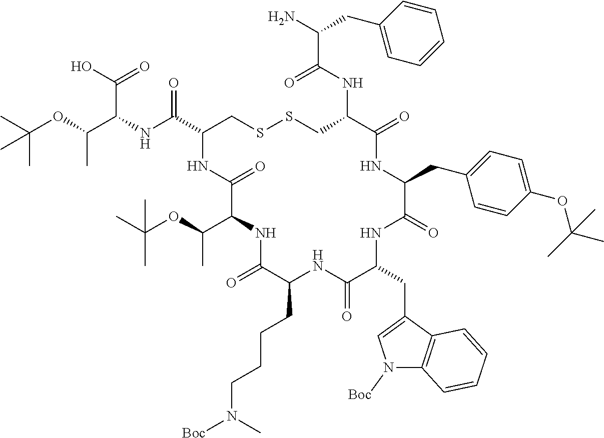 |
| Int-28 | 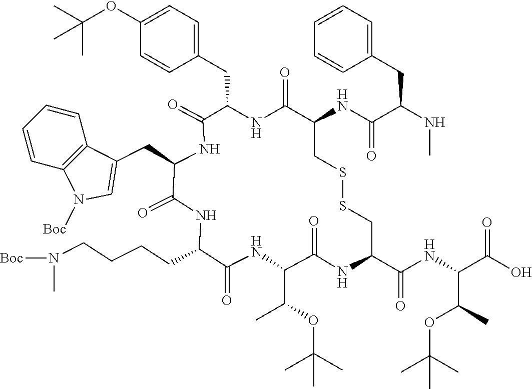 [SMILES: CN[C@H](Cc1ccccc1)C(=O)N[C@H]1CSSC[C@@H](C(=O)N[C@H](C(=O)O)[C@@H](C)OC(C)(C)C)NC(=O)[C@H]([C@@H](C)OC(C)(C)C)NC(=O)[C@H](CCCCN(C)C(=O)OC(C)(C)C)NC(=O)[C@@H](Cc2cn(C(=O)OC(C)(C)C)c3ccccc23)NC(=O)[C@H](Cc2ccc(OC(C)(C)C)cc2)NC1=O] |

### Preparation of Cyclo-ID-1-Nal-[Cys(Trt)-Tyr(O-t-Bu)-D-Trp(Boc)-Lys(Me, Boc)-Thr(O-t-Bu)-Cys(Trt)]-Thr(O-t-Bu)-OH (Int-4)

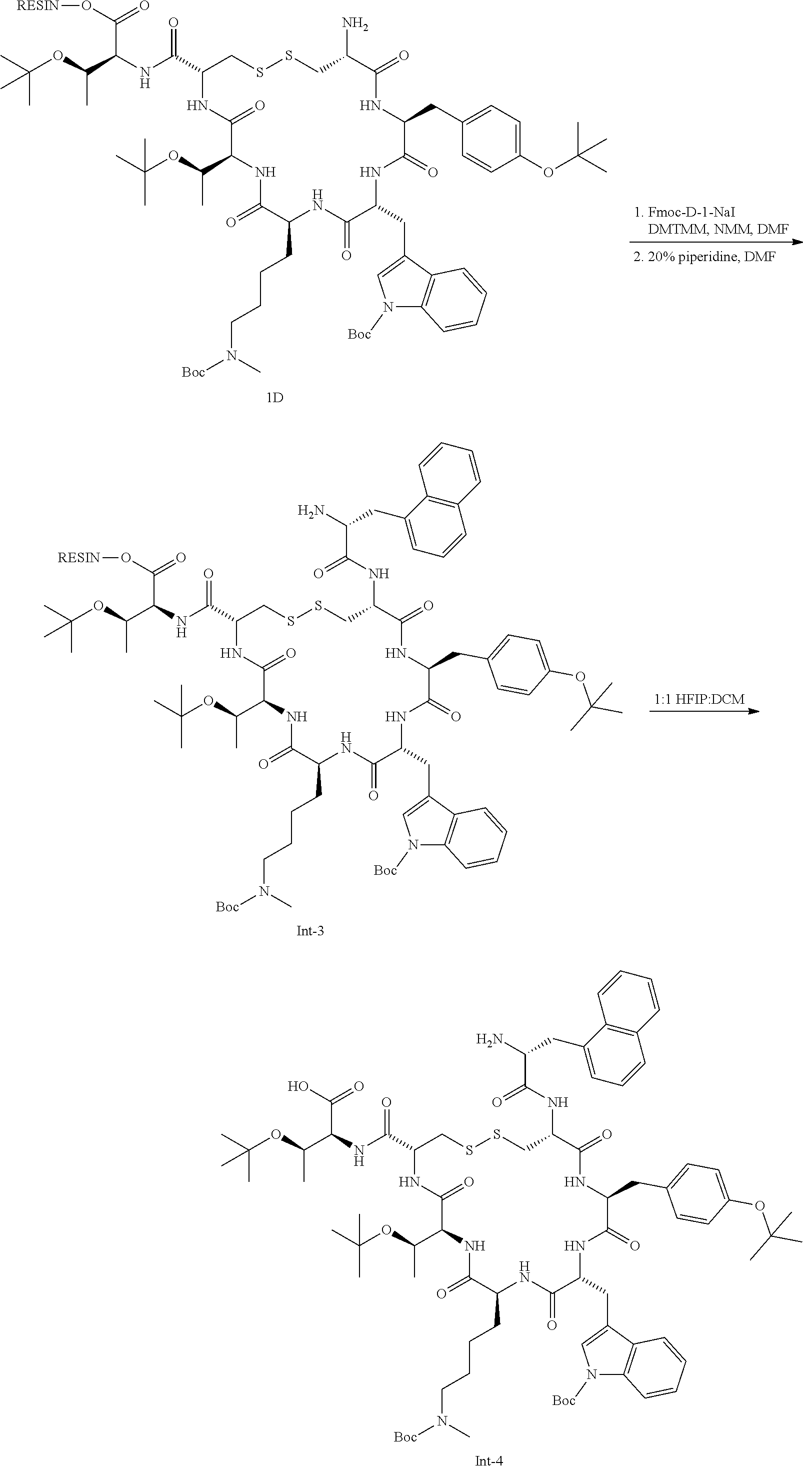

### Cyclo-D-1-Nal-[Cys(Trt)-Tyr(O-t-Bu)-D-Trp(Boc)-Lys(Me, Boc)-Thr(O-t-Bu)-Cys(Trt)]-Thr(O-t-Bu)-2-Cl-trityl Resin (Int-3)

Resin-peptide ID (0.2 mmol) was suspended in DMF (5 ml). Fmoc-D-1-Nal-OH (0.3 mmol, 1.5 eq), NMM (0.4 mmol, 2 eq) and DMTMM (0.3 mmol, 1.5 eq) were added and the reaction was stirred gently at 25° C. for 1 h. The resin-peptide was filtered and washed with DMF (5 ml×5). 20% piperidine in DMF (5 ml) was added to resin-peptide and stirred at 25° C. for 30 min with gentle stirring. Washed resin-peptide with DMF (5 ml×5) then DCM (5 ml×5). (assume quant. yield of Int-3).

### Cyclo-D-1-Nal-[Cys(Trt)-Tyr(O-t-Bu)-D-Trp(Boc)-Lys(Me, Boc)-Thr(O-t-Bu)-Cys(Trt)]-Thr(O-t-Bu)-OH (Int-4)

Resin-peptide Int-3 (0.1 mmol) was cleaved using a solution of 1:1 HFIP/DCM (2 ml) at 25° C. for 5 min. Filtered and extracted resin with DCM (5 ml). The resin was cleaved two more times using the same sequence. The combined filtrates were concentrated in vacuo to give a sticky solid that was dissolved in 3:1 MeCN/water (5 ml) and lyophilized to afford Int-4 as a white solid (127 mg). MS (ESI) m/z: 1482.0 (M+H)⁺.

### Preparation of Cyclo-D-Phe(4-Cl)-[Cys(Trt)-Tyr(O-t-Bu)-D-Trp(Boc)-Lys(Me, Boc)-Thr(O-t-Bu)-Cys(Trt)]-Thr(O-t-Bu)-OH (Int-6)

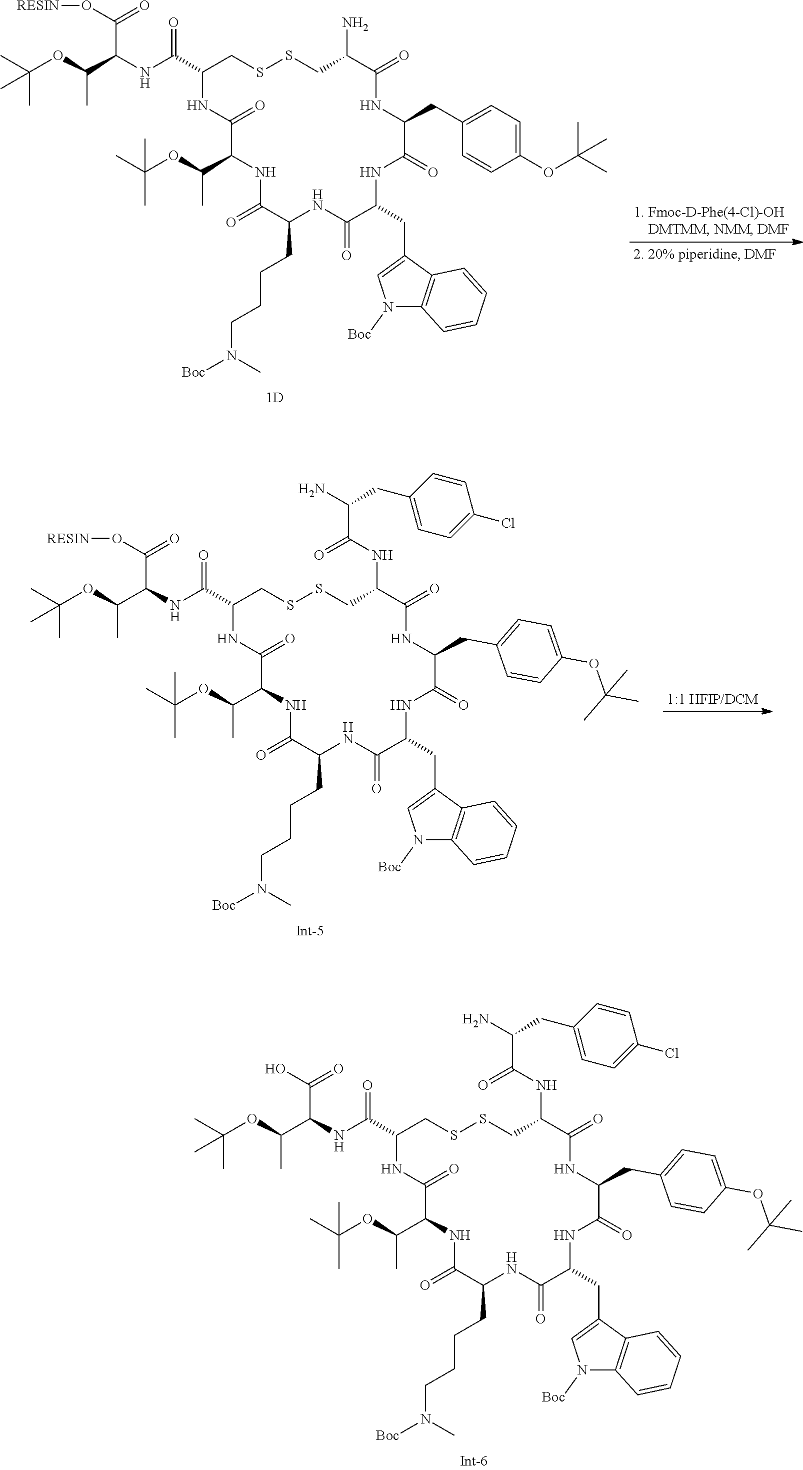

### Cyclo-D-Phe(4-Cl)-[Cys(Trt)-Tyr(O-t-Bu)-D-Trp(Boc)-Lys(Me, Boc)-Thr(O-t-Bu)-Cys(Trt)]-Thr(O-t-Bu)-2-Cl-trityl Resin (Int-5)

Resin-peptide ID (0.2 mmol) was suspended in DMF (5 ml). Fmoc-D-Phe(4-Cl) (0.3 mmol, 1.5 eq), NMM (0.4 mmol, 2 eq) and DMTMM (0.3 mmol, 1.5 eq) were added and the reaction was stirred gently at 25° C. for 1 h. The resin-peptide was filtered and washed with DMF (5 ml×5). 20% piperidine in DMF (5 ml) was added to resin-peptide and stirred at 25° C. for 30 min with gentle stirring. Washed resin-peptide with DMF (5 ml×5) then DCM (5 ml×5). (assume quant. yield of Int-5).

### Cyclo-D-Phe(4-Cl)-[Cys(Trt)-Tyr(O-t-Bu)-D-Trp(Boc)-Lys(Me, Boc)-Thr(O-t-Bu)-Cys(Trt)]-Thr(O-t-Bu)-OH (Int-6)

Resin-peptide Int-5 (0.1 mmol) was cleaved using a solution of 1:1 HFIP/DCM (2 ml) at 25° C. for 5 min. Filtered and extracted resin with DCM (5 ml). The resin was cleaved two more times using the same sequence. The combined filtrates were concentrated in vacuo to give a sticky solid that was dissolved in 3:1 MeCN/water (5 ml) and lyophilized to give Int-6 as a white solid (127 mg). MS (ESI) m/z: 1466.0 (M+H)⁺.

### Preparation of Cyclo-D-Phe(3-I)-[Cys(Trt)-Tyr(O-t-Bu)-D-Trp(Boc)-Lys(Me, Boc)-Thr(O-t-Bu)-Cys(Trt)]-Thr(O-t-Bu)-OH (Int-8)

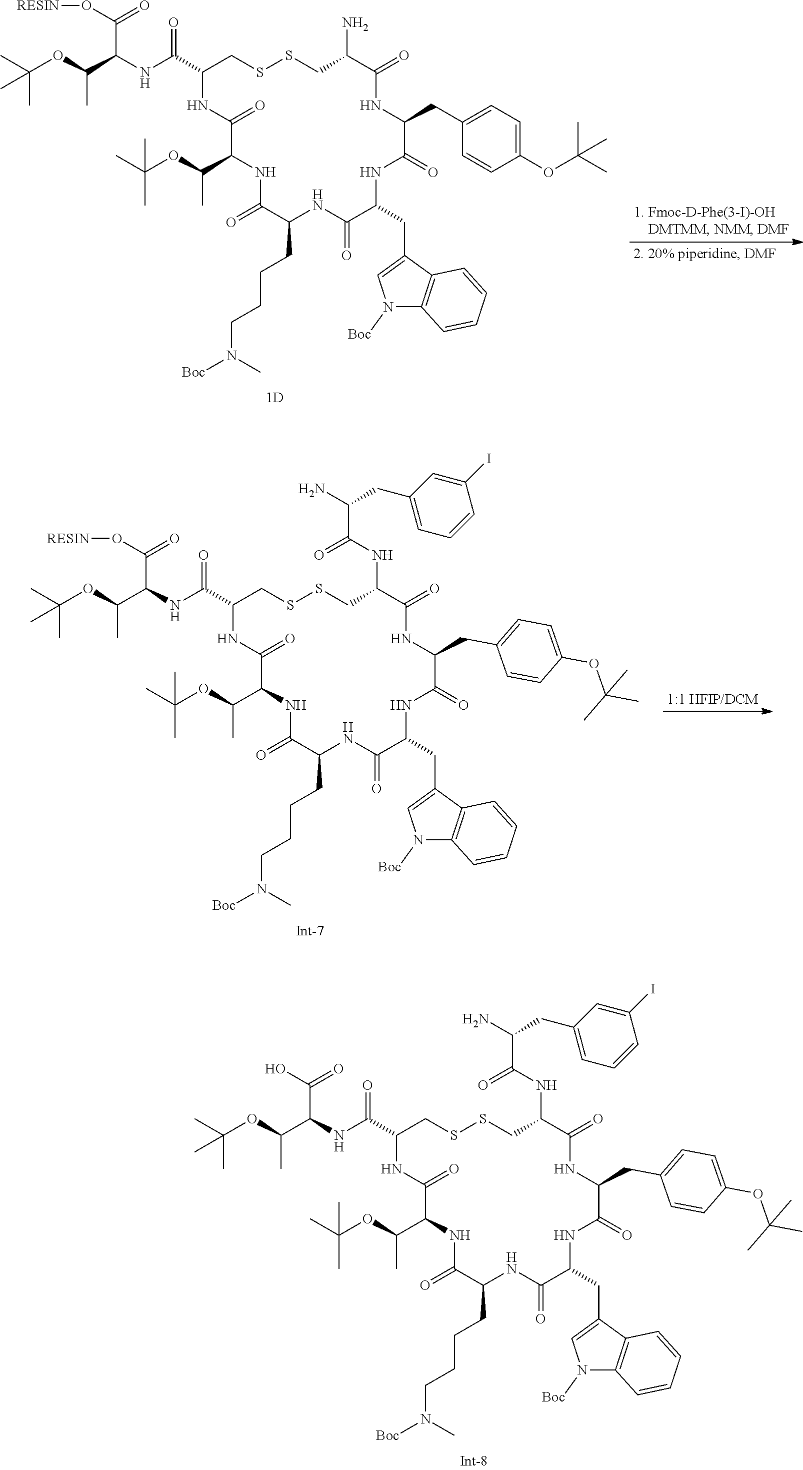

### Cyclo-D-Phe(3-I)-[Cys(Trt)-Tyr(O-t-Bu)-D-Trp(Boc)-Lys(Me, Boc)-Thr(O-t-Bu)-Cys(Trt)]-Thr(O-t-Bu)-2-Cl-trityl Resin (Int-7)

Resin-peptide ID (0.1 mmol) was suspended in DMF (5 ml). Fmoc-D-Phe(3-I) (0.15 mmol, 1.5 eq), NMM (0.2 mmol, 2 eq) and DMTMM (0.15 mmol, 1.5 eq) were added and the reaction was stirred gently at 25° C. for 1 h. The resin-peptide was filtered and washed with DMF (5 ml×5). 20% piperidine in DMF (5 ml) was added to resin-peptide and stirred at 25° C. for 30 min with gentle stirring. Washed resin-peptide with DMF (5 ml×5) then DCM (5 ml×5). (assume quant. yield of Int-7)

### Cyclo-D-Phe(3-I)-[Cys(Trt)-Tyr(O-t-Bu)-D-Trp(Boc)-Lys(Me, Boc)-Thr(O-t-Bu)-Cys(Trt)]-Thr(O-t-Bu)-OH (Int-8)

Resin-peptide Int-7 (0.05 mmol) was cleaved using a solution of 1:1 HFIP/DCM (2 ml) at 25° C. for 5 min. Filtered and extracted resin with DCM (5 ml). The resin was cleaved two more times using the same sequence. The combined filtrates were concentrated in vacuo to give a sticky solid that was dissolved in 3:1 MeCN/water (5 ml) and lyophilized to give Int-8 as a white solid (62 mg). MS (ESI) m/z: 1557.9 (M+H)⁺.

### Preparation of Cyclo-D-Phe-[Cys(Trt)-Tyr(O-t-Bu)-D-Trp(Boc)-Lys(Dimethyl)-Thr(O-t-Bu)-Cys(Trt)]-Thr(O-t-Bu) (Int-9)

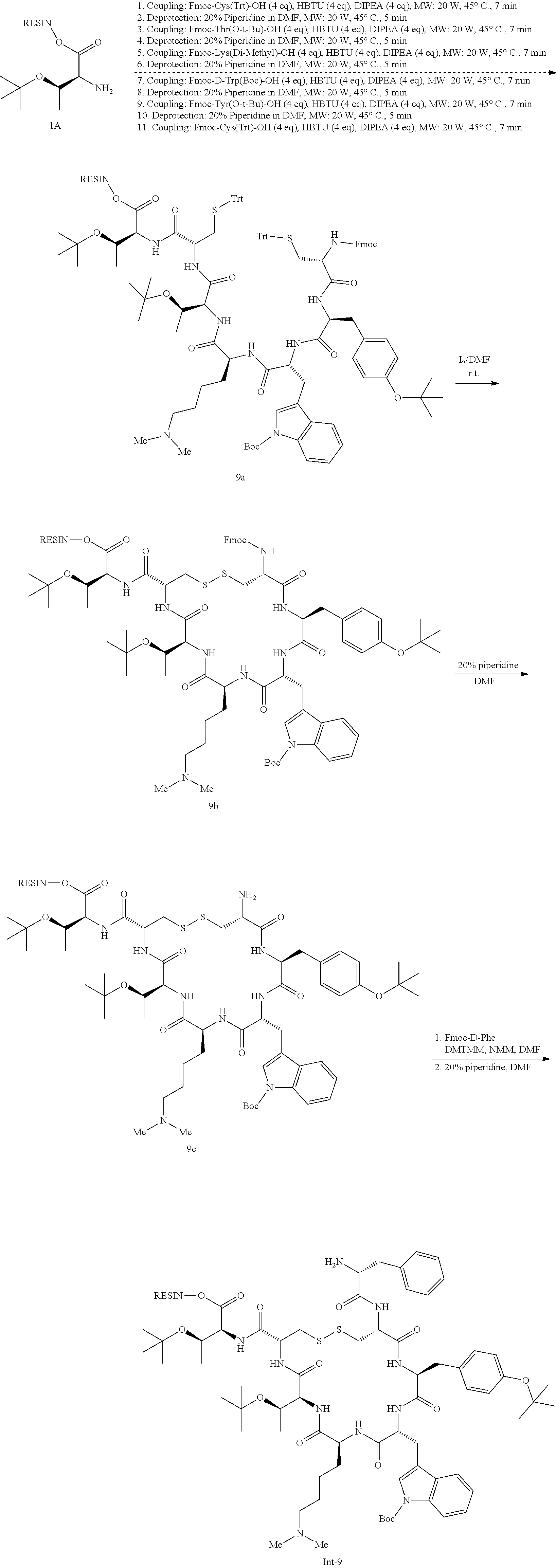

### Fmoc-Cys(Trt)-Tyr(O-t-Bu)-D-Trp(Boc)-Lys(Dimethyl)-Thr(O-t-Bu)-Cys(Trt)-Thr(O-t-Bu)-2-Cl-trityl Resin (9a)

Thr(O-t-Bu) 2-Cl-trityl resin (1A, 0.1 mmol, Chem Impex #) was swelled in DMF (2 ml) for 30 minutes, then filtered. SPPS was carried out as follows. Coupling: Pre-activate Fmoc-AA-OH (4 eq.) with HBTU (4 eq.) and DIPEA (4 eq.) in 20 ml of DMF for 1 min. Added to resin and heated in microwave reactor at 20 W, 45° C. for 7 min with gentle stirring. Washed resin with DMF (10 ml×5). Deprotection: 20% piperidine in DMF (20 ml) was added to resin and heated in microwave reactor at 20 W, 45° C. for 5 min with gentle stirring. Washed resin with DMF (10 ml×5).

### Cyclo-[Fmoc-Cys(Trt)-Tyr(O-t-Bu)-D-Trp(Boc)-Lys(Dimethyl)-Thr(O-t-Bu)-Cys(Trt)]-Thr(O-t-Bu)-2-Cl-trityl Resin (9b)

Resin-peptide 9a (0.1 mmol) was suspended in DMF (5 ml). Iodine (10 eq) was added, and the mixture was gently stirred at room temperature for 2 h, filtered and washed resin with DMF (10 ml×5) and DCM (10 ml×2).

### Cyclo-[Cys(Trt)-Tyr(O-t-Bu)-D-Trp(Boc)-Lys(Dimethyl)-Thr(O-t-Bu)-Cys(Trt)]-Thr(O-t-Bu)-2-CL-trityl Resin (9c)

Resin-peptide 9b (0.1 mmol) was treated with a solution of 20% piperidine in DMF (20 ml) and heated in microwave reactor at 20 W, 45° C. for 5 min with gentle stirring. Washed resin with DMF (10 ml×5).

### Cyclo-D-Phe-[Cys(Trt)-Tyr(O-t-Bu)-D-Trp(Boc)-Lys(Dimethyl)-Thr(O-t-Bu)-Cys(Trt)]-Thr(O-t-Bu)-2-Cl-trityl Resin (Int-9)

Resin-peptide 9c (0.1 mmol) was suspended in DMF (2 ml). Pre-activate Fmoc-D-Phe-OH (4 eq.) with HBTU (4 eq.) and DIPEA (4 eq.) in 4 ml of DMF for 1 min. Added to resin and the reaction was stirred gently at room temperature for 2 h. The resin-peptide was filtered and washed with DMF (5 ml×5). 20% piperidine in DMF (5 ml) was added to resin-peptide and stirred at room temperature for 2 h min. Washed resin-peptide with DMF (5 ml×5) then DCM (5 ml×2).

### Cyclo-D-Phe-[Cys(Trt)-Tyr(O-t-Bu)-D-Trp(Boc)-Lys(Dimethyl)-Thr(O-t-Bu)-Cys(Trt)]-Thr(O-t-Bu) (Int-10)

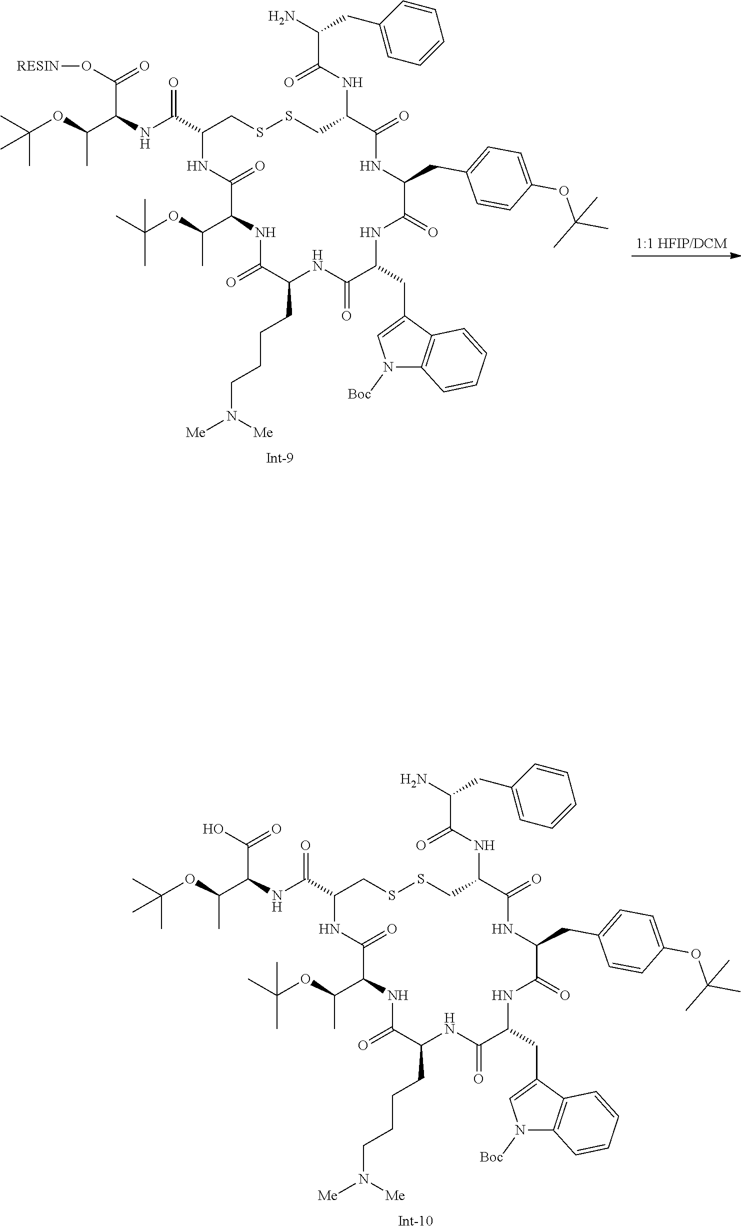

To resin-peptide Int-9 (0.1 mmol) was added a mixture of HFIP:DCM (1:1, 4 mL) and stirred for 20 minutes. The filtrate was collected in a glass vial. The resin was extracted with DCM (5 ml) and collected in the vial. The solvent was evaporated under reduced pressure. The crude was purified by reverse phase chromatography (C18) eluting with a gradient of MeCN/H₂O. The gradient started at 5% MeCN, reaching 50% MeCN over 20 minutes, with the product eluting at 20% MeCN. Pure fractions were collected and evaporated to yield Int-10 as a clear oil (11 mg, 7%).

### Preparation of Cyclo-D-2-Nal-[Cys(Trt)-Tyr(O-t-Bu)-D-Trp(Boc)-Lys(Me, Boc)-Thr(O-t-Bu)-Cys(Trt)]-Thr(O-t-Bu)-2-Cl-trityl Resin (Int-11)

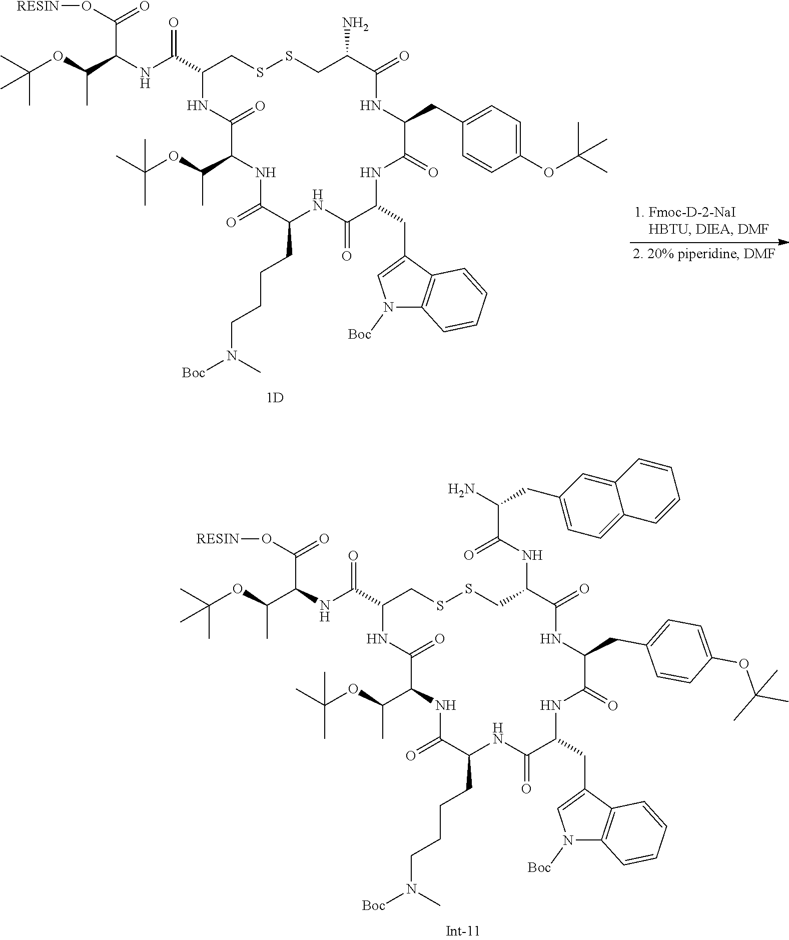

HBTU (0.6 mmol, 1.5 eq) was added to a solution of Fmoc-D-2-Nal-OH (0.6 mmol, 1.5 eq) and DIPEA (0.8 mmol, 2 eq) in DMF and vortex mixed for 2 min. The resin-peptide 1D (0.4 mmol, 1 eq) was added and stirred at 25° C. for 1 h. The resin was filtered and washed with DMF (5 ml×5). 20% piperidine in DMF (5 ml) was added to resin-peptide and stirred at 25° C. for 30 min with gentle stirring. The resin-peptide was washed with DMF (5 ml×5) then DCM (5 ml×5). (assume quant. yield of Int-11).

### Preparation of Cyclo-D-Phe(4-I)-[Cys(Trt)-Tyr(O-t-Bu)-D-Trp(Boc)-Lys(Me, Boc)-Thr(O-t-Bu)-Cys(Trt)]-Thr(O-t-Bu)-2-Cl-trityl Resin (Int-12)

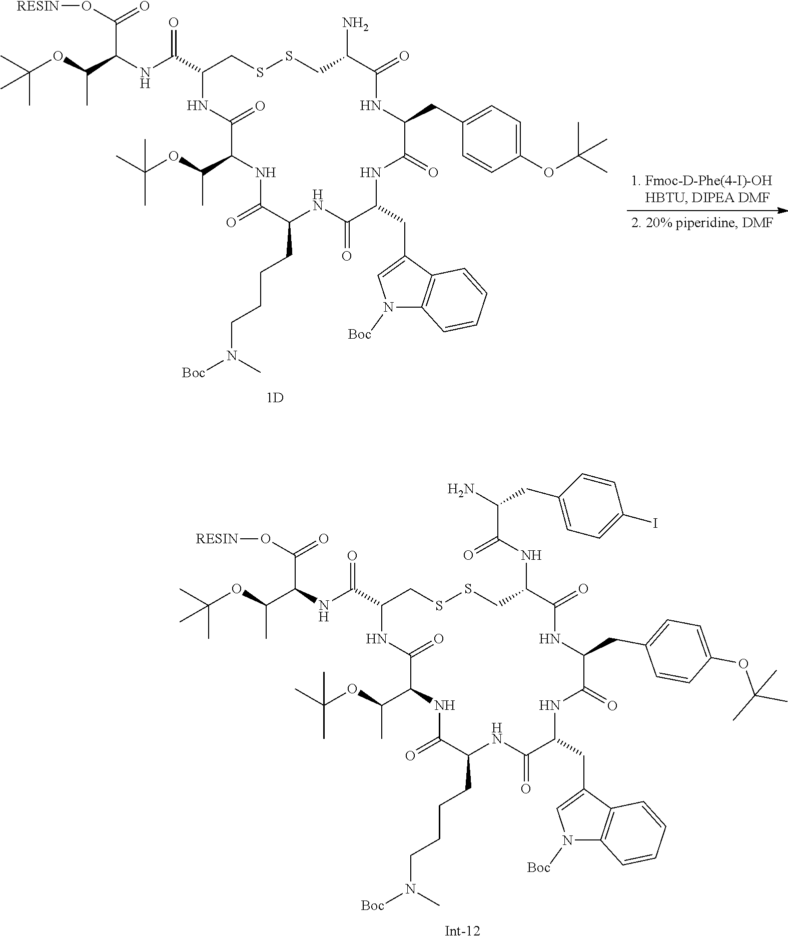

HBTU (0.6 mmol, 1.5 eq) was added to a solution of Fmoc-D-Phe(4-I)—OH (0.6 mmol, 1.5 eq) and DIPEA (0.8 mmol, 2 eq) in DMF and vortex mixed for 2 min. The resin-peptide 1D (0.4 mmol, 1 eq) was added and stirred at 25° C. for 1 h. The resin was filtered and washed with DMF (5 ml×5). 20% piperidine in DMF (5 ml) was added to resin-peptide and stirred at 25° C. for 30 min with gentle stirring. The resin-peptide was washed with DMF (5 ml×5), then DCM (5 ml×5). (assume quant. yield of Int-12).

### Preparation of D-Phe-Cyclo-[Cys(Trt)-Tyr(O-t-Bu)-D-Trp(Boc)-Arg(Pbf)-Thr(O-t-Bu)-Cys(Trt)]-Thr(O-t-Bu)-2-Cl-trityl Resin (Int-13)

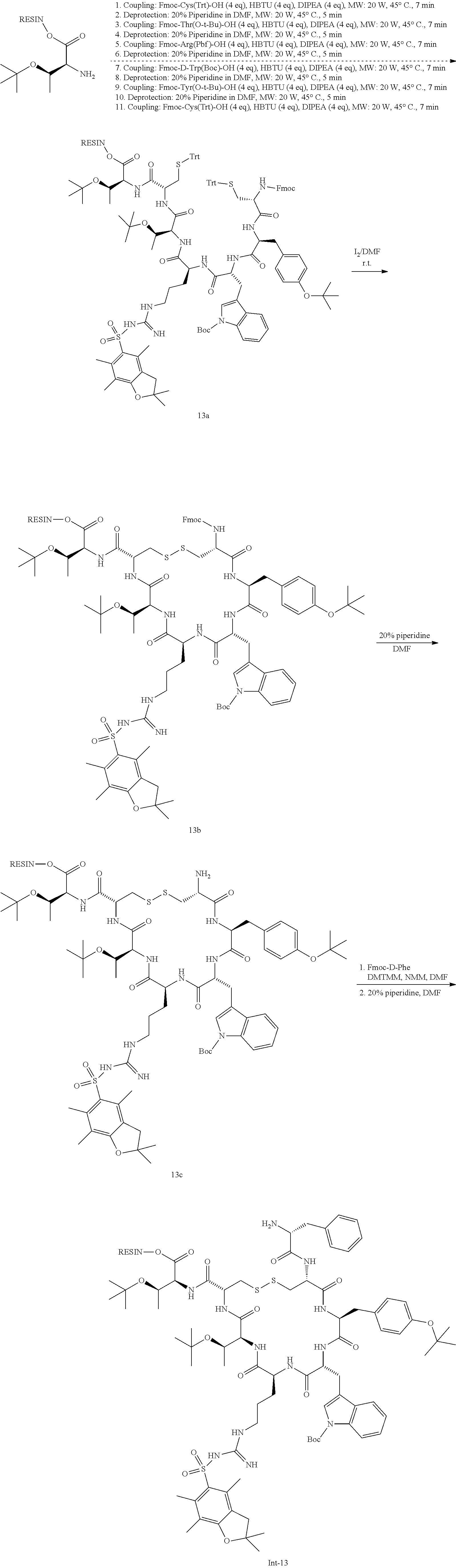

### Fmoc-Cys(Trt)-Tyr(O-t-Bu)-D-Trp(Boc)-Arg(Pbf)-Thr(O-t-Bu)-Cys(Trt)-Thr(O-t-Bu)-2-Cl-trityl Resin (13a)

Fmoc-Thr(O-t-Bu)-2-Chlorotrityl resin (0.1 mmol, Chem Impex Cat #: 06803) was swelled in DMF (5 ml) for 30 minutes, then filtered. The Fmoc group was removed by treating the resin with 20% piperidine in DMF (5 ml) at 25° C. for 1 h, then the resin was washed with DMF (5 ml×5).

SPPS was carried out as follows. Coupling: Pre-activate Fmoc-AA-OH (4 eq.) with HBTU (4 eq.) and DIPEA (4 eq.) in 5 ml of DMF for 1 min. Added to resin and heated in microwave reactor at 20 W, 45° C. for 7 min with gentle stirring. The resin was washed with DMF (5 ml×5). Deprotection: 20% piperidine in DMF (10 ml) was added to resin and heated in microwave reactor at 20 W, 45° C. for 5 min with gentle stirring. The resin was washed with DMF (5 ml×5) to afford 13a (assume quant.).

### Cyclo-[Fmoc-Cys(Trt)-Tyr(O-t-Bu)-D-Trp(Boc)-Arg(Pbf)-Thr(O-t-Bu)-Cys(Trt)]-Thr(O-t-Bu)-2-Cl-trityl Resin (13b)

Resin-peptide 13a (0.1 mmol) was suspended in DMF (5 ml). Iodine (5 eq) was added, and the mixture was gently stirred at 25° C. for 2 h, filtered and the resin was washed with DMF (5 ml×5) and DCM (5 ml×5) to afford 13b (assume quant.).

### Cyclo-[Cys(Trt)-Tyr(O-t-Bu)-D-Trp(Boc)-Arg(Pbf)-Thr(O-t-Bu)-Cys(Trt)]-Thr(O-t-Bu)-2-Cl-trityl Resin (13c)

A solution of 20% piperidine in DMF (5 ml) was added to a vessel containing 13b and stirred at 25° C. for 30 min. The resin was washed with DMF (5 ml×5) to afford 13c (assume quant.).

### D-Phe-Cyclo-[Cys(Trt)-Tyr(O-t-Bu)-D-Trp(Boc)-Arg(Pbf)-Thr(O-t-Bu)-Cys(Trt)]-Thr(O-t-Bu)-2-CL-trityl Resin (Int-13)

Resin bound 13c (0.05 mmol) was suspended in DMF (0.5 mL). To this was added Fmoc-D-Phe-OH (77.5 mg, 0.2 mmol, 4 eq), DMTMM (53.5 mg, 0.2 mmol, 4 eq), and N-methyl morpholine (22 μL, 0.024 mmol, 4 eq). The reaction mixture was stirred for 1 h at 25° C. The resin was washed with DMF (2 mL×5), then treated with 20% piperidine in DMF (5 ml) at 25° C. for 1 h, then the resin was washed with DMF (5 ml×5) afford Int-13 (assume quant. yield).

### Preparation D-Phe-Cyclo-[Cys(Trt)-Tyr(O-t-Bu)-D-Trp(Boc)-nor-Arg(Pbf)-Thr(O-t-Bu)-Cys(Trt)]-Thr(O-t-Bu)-2-Cl-trityl Resin (Int-14)

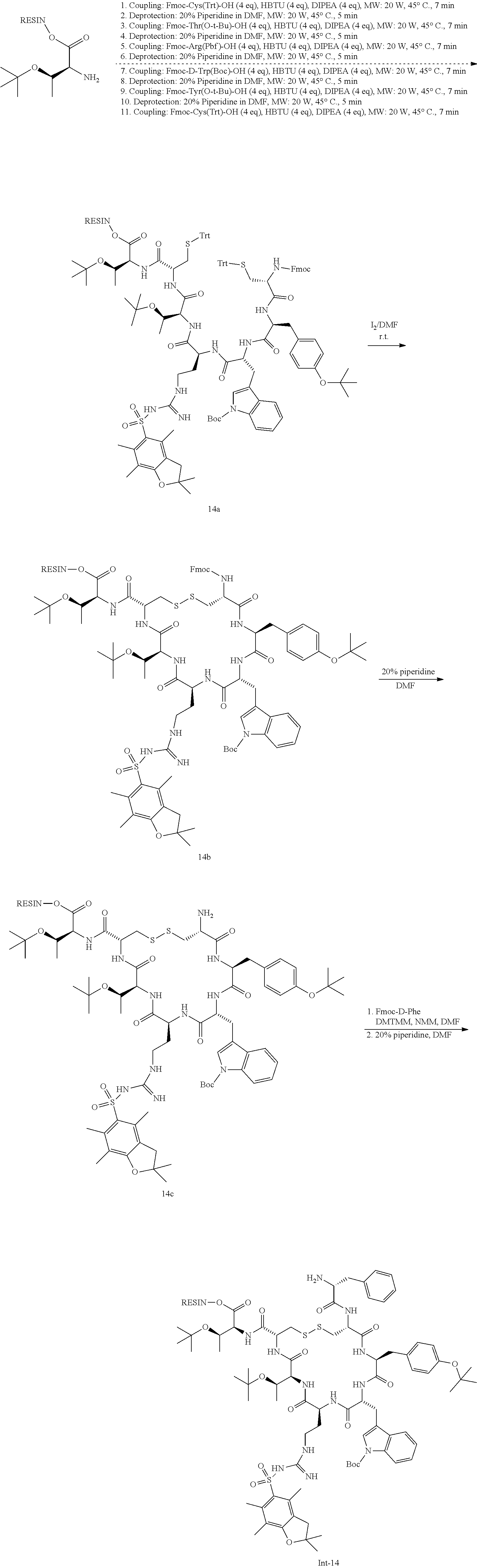

### Fmoc-Cys(Trt)-Tyr(O-t-Bu)-D-Trp(Boc)-nor-Arg(Pbf)-Thr(O-t-Bu)-Cys(Trt)-Thr(O-t-Bu)-2-Cl-trityl Resin (14a)

Fmoc-Thr(O-t-Bu)-2-Chlorotrityl resin (0.1 mmol, Chem Impex Cat #: 06803) was swelled in DMF (5 ml) for 30 min, then filtered. The Fmoc group was removed by treating the resin with 20% piperidine in DMF (5 ml) at 25° C. for 1 h, then the resin was washed with DMF (5 ml×5) to afford 14a (assume quant.).

SPPS was carried out as follows. Coupling: Fmoc-AA-OH (4 eq.) was activated with HBTU (4 eq.) and DIPEA (4 eq.) in 5 ml of DMF for 1 min and then added to the resin and heated in microwave reactor at 20 W, 45° C. for 7 min with gentle stirring. The resin was washed with DMF (5 ml×5). Deprotection: 20% piperidine in DMF (10 ml) was added to resin and heated in microwave reactor at 20 W, 45° C. for 5 min with gentle stirring. The resin was washed with DMF (5 ml×5).

### Cyclo-[Fmoc-Cys(Trt)-Tyr(O-t-Bu)-D-Trp(Boc)-nor-Arg(Pbf)-Thr(O-t-Bu)-Cys(Trt)]-Thr(O-t-Bu)-2-Cl-trityl Resin (14b)

Resin-peptide 14a (0.1 mmol) was suspended in DMF (5 ml). Iodine (5 eq) was added, and the mixture was gently stirred at 25° C. for 2 h, filtered and the resin was washed with DMF (5 ml×5) and DCM (5 ml×5).

### Cyclo-[Cys(Trt)-Tyr(O-t-Bu)-D-Trp(Boc)-nor-Arg(Pbf)-Thr(O-t-Bu)-Cys(Trt)]-Thr(O-t-Bu)-2-CL-trityl Resin (14c)

A solution of 20% piperidine in DMF (5 ml) was added to a vessel containing 14b and stirred at 25° C. for 30 min. The resin was washed with DMF (5 ml×5) to afford 14c (assume quant.).

### D-Phe-Cyclo-[Cys(Trt)-Tyr(O-t-Bu)-D-Trp(Boc)-nor-Arg(Pbf)-Thr(O-t-Bu)-Cys(Trt)]-Thr(O-t-Bu)-2-Cl-trityl Resin (Int-14)

Resin bound 14c (0.05 mmol) was suspended in DMF (0.5 mL). To this was added Fmoc-D-Phe-OH (77.5 mg, 0.2 mmol, 4 eq), DMTMM (53.5 mg, 0.2 mmol, 4 eq), and N-methyl morpholine (22 μL, 0.024 mmol, 4 eq). The reaction mixture was stirred for 1 h at 25° C. The resin was washed with DMF (2 mL×5), then treated with 20% piperidine in DMF (5 ml) at 25° C. for 1 h, then the resin was washed with DMF (5 ml×5) afford Int-14 (assume quant. yield).

### Preparation of Cpa-cyclo-[D-Cys(Trt)-Aph(Hor)-D-Aph(Cbm)-Lys(Me,Boc)-Thr(O-t-Bu)-Cys(Trt)]-D-Tyr(O-t-Bu)-RINKAAMIDE Resin (Int-15)

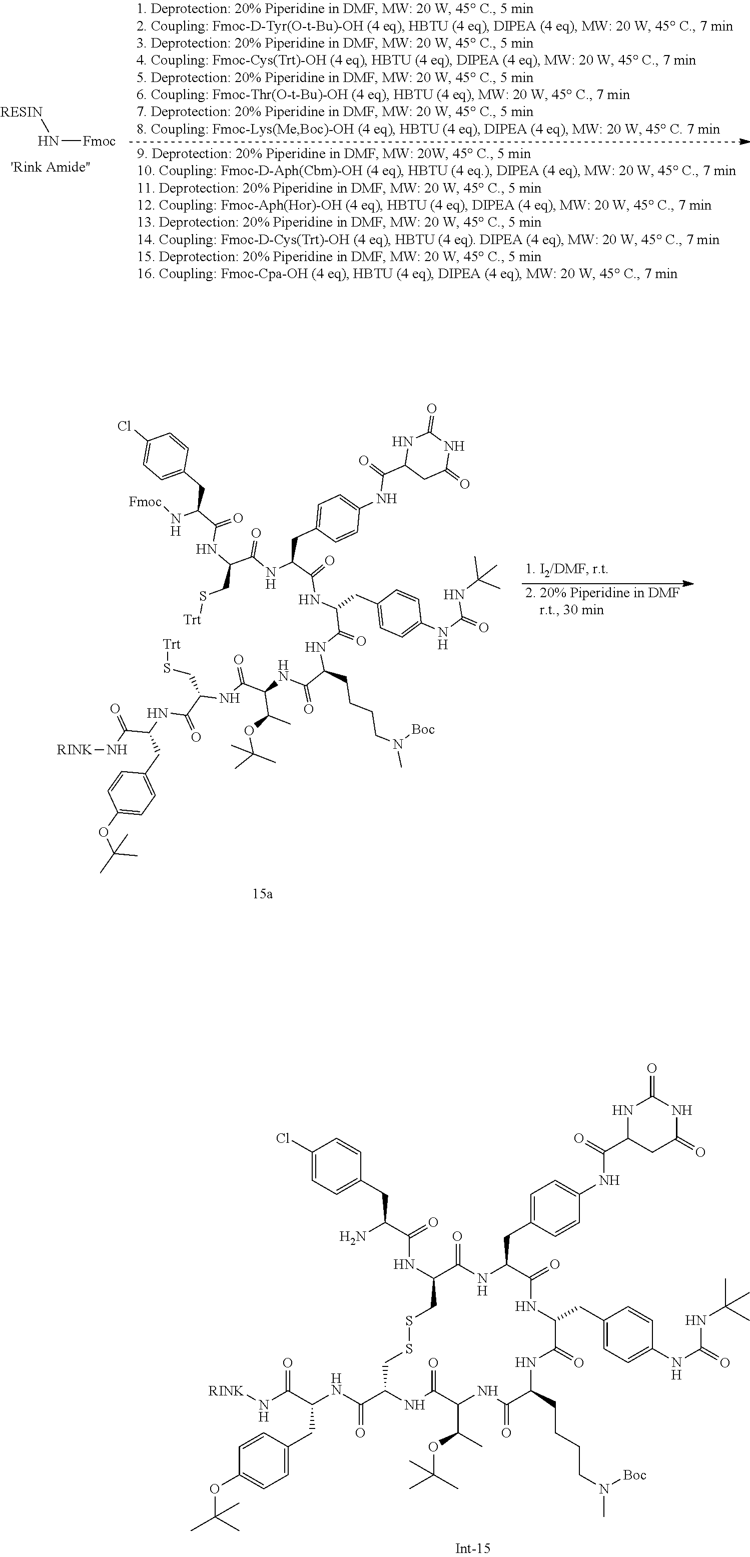

### Fmoc-Cpa-D-Cys(Trt)-Aph(Hor)-D-Aph(Cbm)-Lys(Me,Boc)-Thr(O-t-Bu)-Cys(Trt)-D-Tyr(O-t-Bu)-RINK AMIDE Resin (15a)

RINK AMIDE resin (0.2 mmol, Ambeed Cat #: A451407) was swelled in DMF (5 ml) for 30 minutes, then filtered. The Fmoc group was removed by treating the resin with 20% piperidine in DMF (5 ml) at 25° C. for 1 h, then the resin was washed with DMF (20 ml×5).

### SPPS was Carried Out as Follows:

Coupling: Fmoc-AA-OH (4 eq.) was preactivated with HBTU (4 eq.) and DIPEA (4 eq.) in 10 ml of DMF for 1 min and then added to resin and heated in microwave reactor at 20 W, 45° C. for 7 min with gentle stirring. The resin was washed with DMF (5 ml×5). Deprotection: 20% piperidine in DMF (10 ml) was added to resin and heated in microwave reactor at 20 W, 45° C. for 5 min with gentle stirring. The resin was washed with DMF (5 ml×5).

### Cpa-cyclo-[D-Cys(Trt)-Aph(Hor)-D-Aph(Cbm)-Lys(Me,Boc)-Thr(O-t-Bu)-Cys(Trt)]-D-Tyr(O-t-Bu)-RINK AMIDE Resin (Int-15)

Resin-peptide 15a (0.2 mmol) was suspended in DMF (10 ml). Iodine (5 eq) was added, and the mixture was gently stirred at 25° C. for 2 h, filtered and the resin was washed with DMF (5 ml×5) and DCM (5 ml×5). A solution of 20% piperidine in DMF (10 ml) was added and stirred at 25° C. for 30 min. The resin was washed with DMF (5 ml×5) to afford Int-15 (assume quant.).

### Preparation of 1-Nal-cyclo-[D-Cys(Trt)-Aph(Hor)-D-Aph(Cbm)-Lys(Me,Boc)-Thr(O-t-Bu)-Cys(Trt)]-D-Tyr(O-t-Bu)-RINK AMIDE Resin (Int-16)

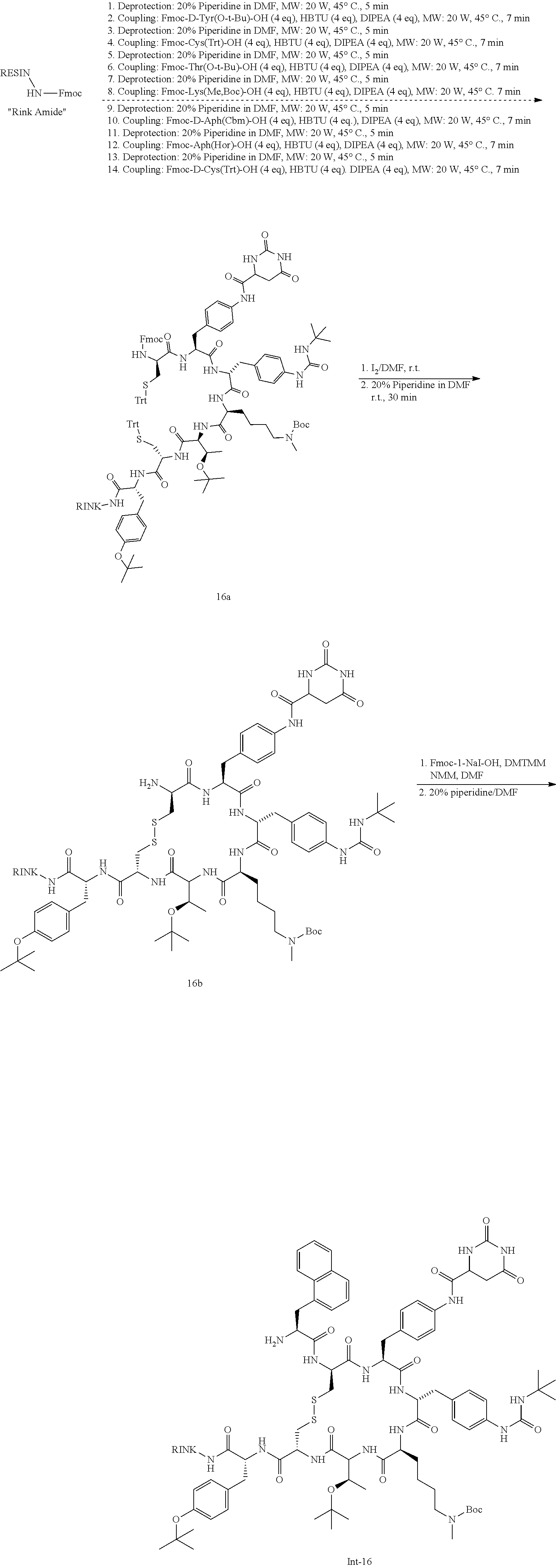

### Fmoc-D-Cys(Trt)-Aph(Hor)-D-Aph(Cbm)-Lys(Me,Boc)-Thr(O-t-Bu)-Cys(Trt)-D-Tyr(O-t-Bu)-RINK AMIDE Resin (16a)

RINK AMIDE resin (0.1 mmol, Ambeed Cat #: A451407) was swelled in DMF (3 ml) for 30 minutes, then filtered. The Fmoc group was removed by treating the resin with 20% piperidine in DMF (3 ml) at 25° C. for 1 h, then the resin was washed with DMF (10 ml×5).

SPPS was carried out as follows. Coupling: Fmoc-AA-OH (4 eq.) was preactivated with HBTU (4 eq.) and DIPEA (4 eq.) in 5 ml of DMF for 1 min and added to resin and heated in microwave reactor at 20 W, 45° C. for 7 min with gentle stirring. The resin was washed with DMF (5 ml×5). Deprotection: 20% piperidine in DMF (5 ml) was added to resin and heated in microwave reactor at 20 W, 45° C. for 5 min with gentle stirring. The resin was washed with DMF (5 ml×5).

### Cyclo-[D-Cys(Trt)-Aph(Hor)-D-Aph(Cbm)-Lys(Me,Boc)-Thr(O-t-Bu)-Cys(Trt)]-D-Tyr(O-t-Bu)-RINK AMIDE Resin (16b)

Resin-peptide 16a (0.1 mmol) was suspended in DMF (5 ml). Iodine (5 eq) was added, and the mixture was gently stirred at 25° C. for 2 h, filtered and the resin was washed with DMF (5 ml×5) and DCM (5 ml×5). A solution of 20% piperidine in DMF (5 ml) was added and stirred at 25° C. for 30 min. The resin was washed with DMF (5 ml×5) to afford 16b (assume quant.).

### 1-Nal-cyclo-[D-Cys(Trt)-Aph(Hor)-D-Aph(Cbm)-Lys(Me,Boc)-Thr(O-t-Bu)-Cys(Trt)]-D-Tyr(O-t-Bu)-RINK AMIDE Resin (Int-16)

Resin bound 16b (0.012 mmol) was suspended in DMF (0.5 mL). To this was added Fmoc-1-Nal-OH (10.5 mg, 0.024 mmol, 2 eq), DMTMM (6.4 mg, 0.024 mmol, 2 eq), and N-methyl morpholine (2.6 μL, 0.024 mmol, 2 eq). The reaction mixture was stirred for 1 h at 25° C. The resin was washed with DMF (2 mL×5), then treated with 20% piperidine in DMF (5 ml) at 25° C. for 1 h, then the resin was washed with DMF (5 ml×5) afford Int-16 (assume quant.).

### Preparation of Phe(3-I)-cyclo-[D-Cys(Trt)-Aph(Hor)-D-Aph(Cbm)-Lys(Me,Boc)-Thr(O-t-Bu)-Cys(Trt)]-D-Tyr(O-t-Bu)-RINK AMIDE Resin (Int-17)

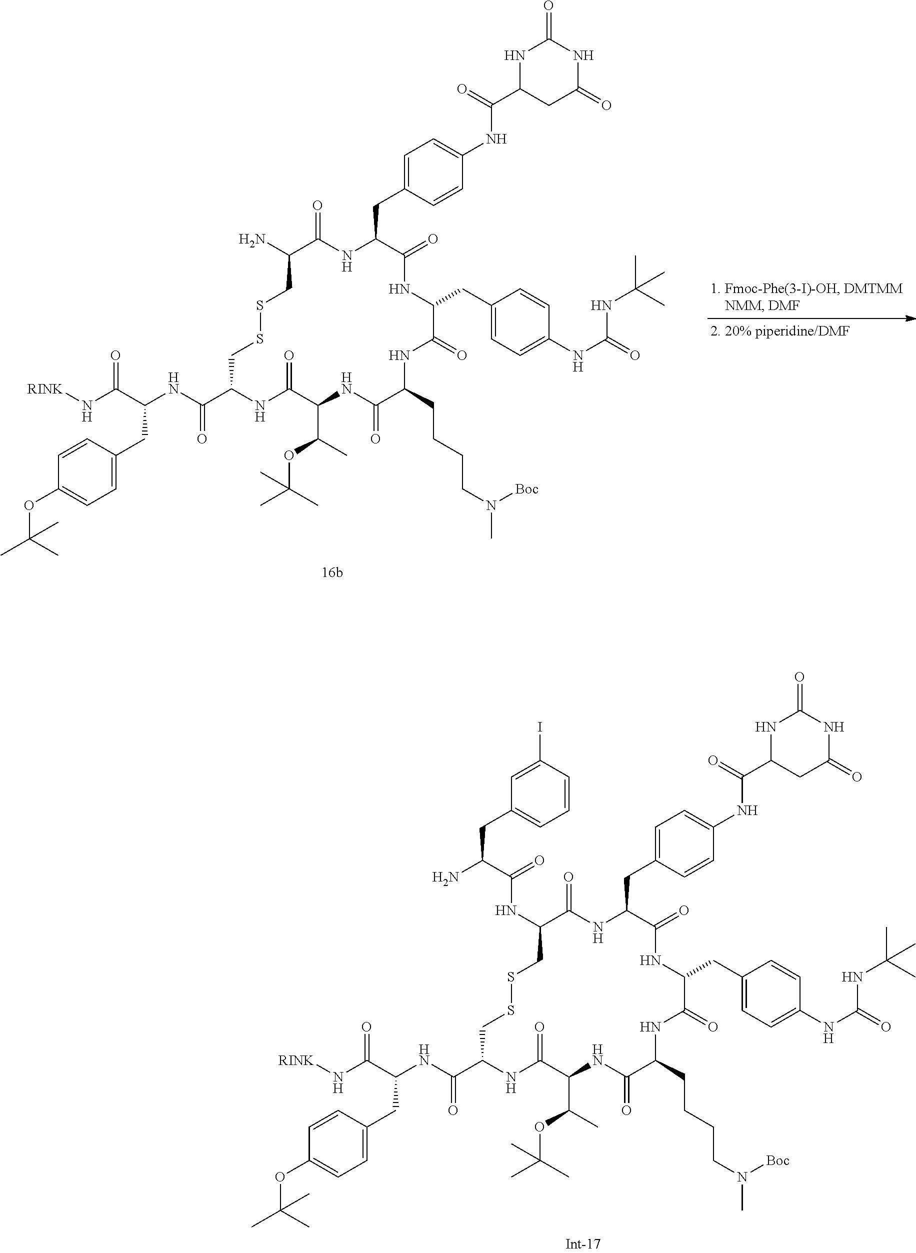

Resin bound 16b (0.012 mmol) was suspended in DMF (0.5 mL). To this was added Fmoc-Phe(3-I)—OH (12.3 mg, 0.024 mmol, 2 eq), DMTMM (6.4 mg, 0.024 mmol, 2 eq), and N-methyl morpholine (2.6 μL, 0.024 mmol, 2 eq). The reaction mixture was stirred for 1 h at 25° C. The resin was washed with DMF (2 mL×5), then treated with 20% piperidine in DMF (5 ml) at 25° C. for 1 h, then the resin was washed with DMF (5 ml×5) to afford Int-17 (assume quant.).

### Preparation of Cyclo-CPA-[D-Cys(Trt)-Tyr(O-t-Bu)-D-Aph(Cbm)-Lys(Me, Boc)-Thr(O-t-Bu)-Cys(Trt)-D-Tyr(O-t-Bu)]-Rink Amide Resin (Int-18)

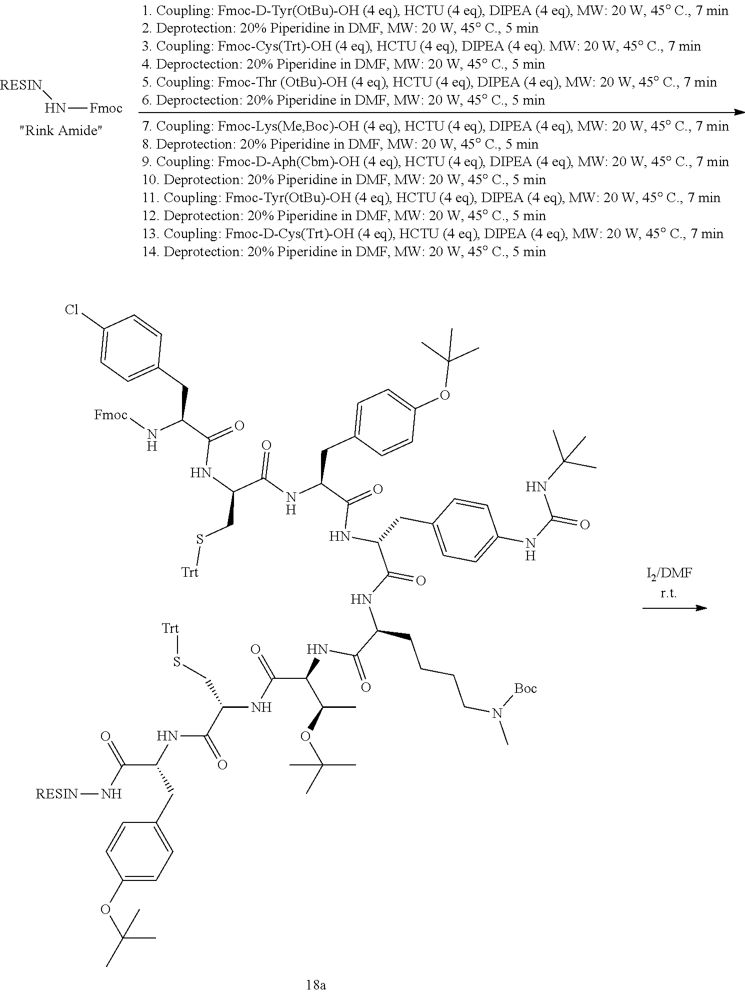

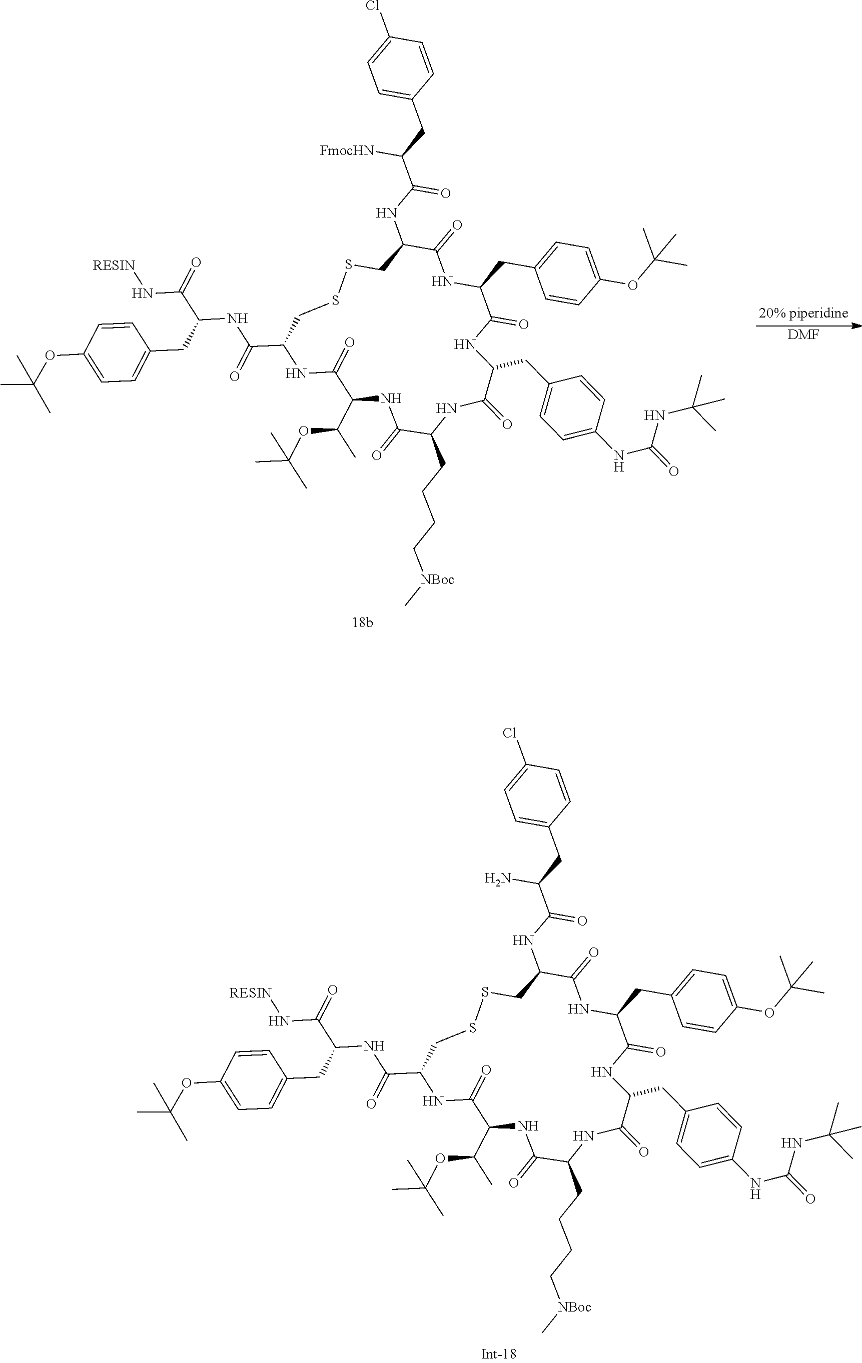

### Fmoc-CPA-D-Cys(Trt)-Tyr(O-t-Bu)-D-Aph(Cbm)-Lys(Me, Boc)-Thr(O-t-Bu)-Cys(Trt)-D-Tyr(O-t-Bu)-Rink Amide Resin (18a)

Rink amide resin (0.2 mmol, Ambeed cat #A451407-10 g) was swelled in DMF (10 ml) for 30 minutes, then filtered. The Fmoc group was removed by treating the resin with 20% piperidine in DMF (10 ml) at 25° C. for 30 min, then washed resin with DMF (10 ml×5).

### SPPS was Carried Out as Follows:

Coupling: Fmoc-AA-OH (4 eq.) was preactivated with HCTU (4 eq.) and DIPEA (4 eq.) in (10 ml) of DMF for 2 min and added to resin and heated in microwave reactor at 20 W, 45° C. for 7 min with gentle stirring. The resin was washed with DMF (10 ml×5).

Deprotection: 20% piperidine in DMF (20 ml) was added to resin and heated in microwave reactor at 20 W, 45° C. for 5 min with gentle stirring. The resin was washed with DMF (10 ml×5).

### Cyclo-Fmoc-CPA-[D-Cys(Trt)-Tyr(O-t-Bu)-D-Aph(Cbm)-Lys(Me, Boc)-Thr(O-t-Bu)-Cys(Trt)-D-Tyr(O-t-Bu)]-Rink Amide Resin (18b)

18a (0.2 mmol) was suspended in DMF (10 ml). Iodine (5 eq) was added, and the mixture was gently stirred at 25° C. for 2 h, filtered and the resin was washed with DMF (10 ml×5) and DCM (10 ml×5).

### Cyclo-CPA-[D-Cys(Trt)-Tyr(O-t-Bu)-D-Aph(Cbm)-Lys(Me, Boc)-Thr(O-t-Bu)-Cys(Trt)-D-Tyr(O-t-Bu)]-Rink Amide Resin (Int-18)

18b (0.2 mmol) was treated with a solution of 20% piperidine in DMF (5 ml) and heated in microwave reactor at 20 W, 45° C. for 5 min with gentle stirring. The resin was washed with DMF (10 ml×5) to afford Int-18 (assume quant.).

The following intermediates were prepared using procedures similar to those for Int-18 with the corresponding amino acids:

| Int No. | Structure |
|---|---|
| Int-29 | 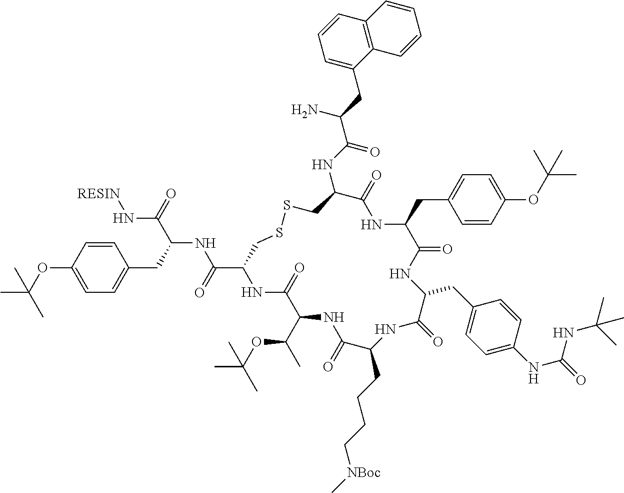 |
| Int-30 | 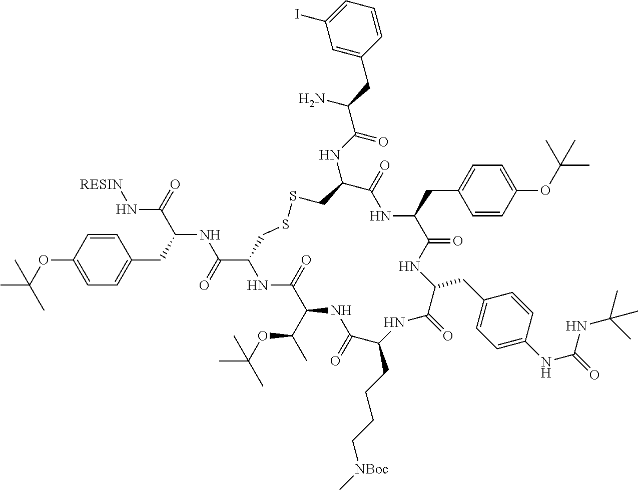 |

### Preparation of Cyclo-NH₂-4-Cl-Phe-[D-Cys(Trt)-Aph(Hor)-D-Aph(Cbm)-Lys(Me, Boc)-Thr(O-t-Bu)-Cys(Trt)]-D-4-Cl-Phe-Rink Amide Resin (Int-19)

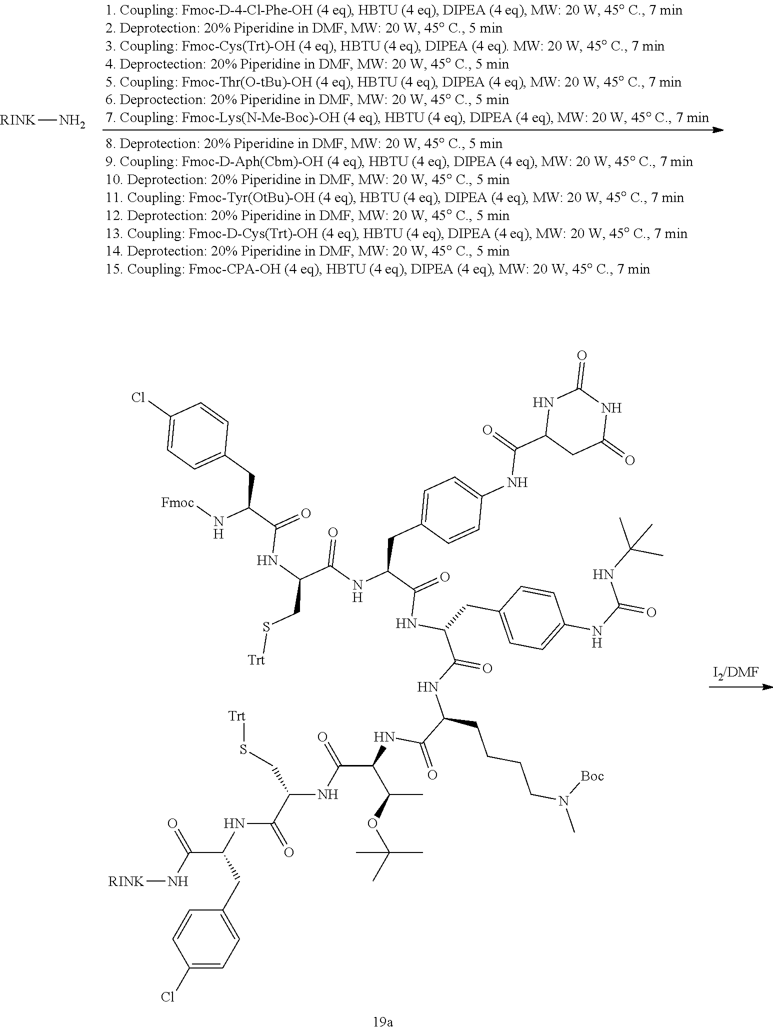

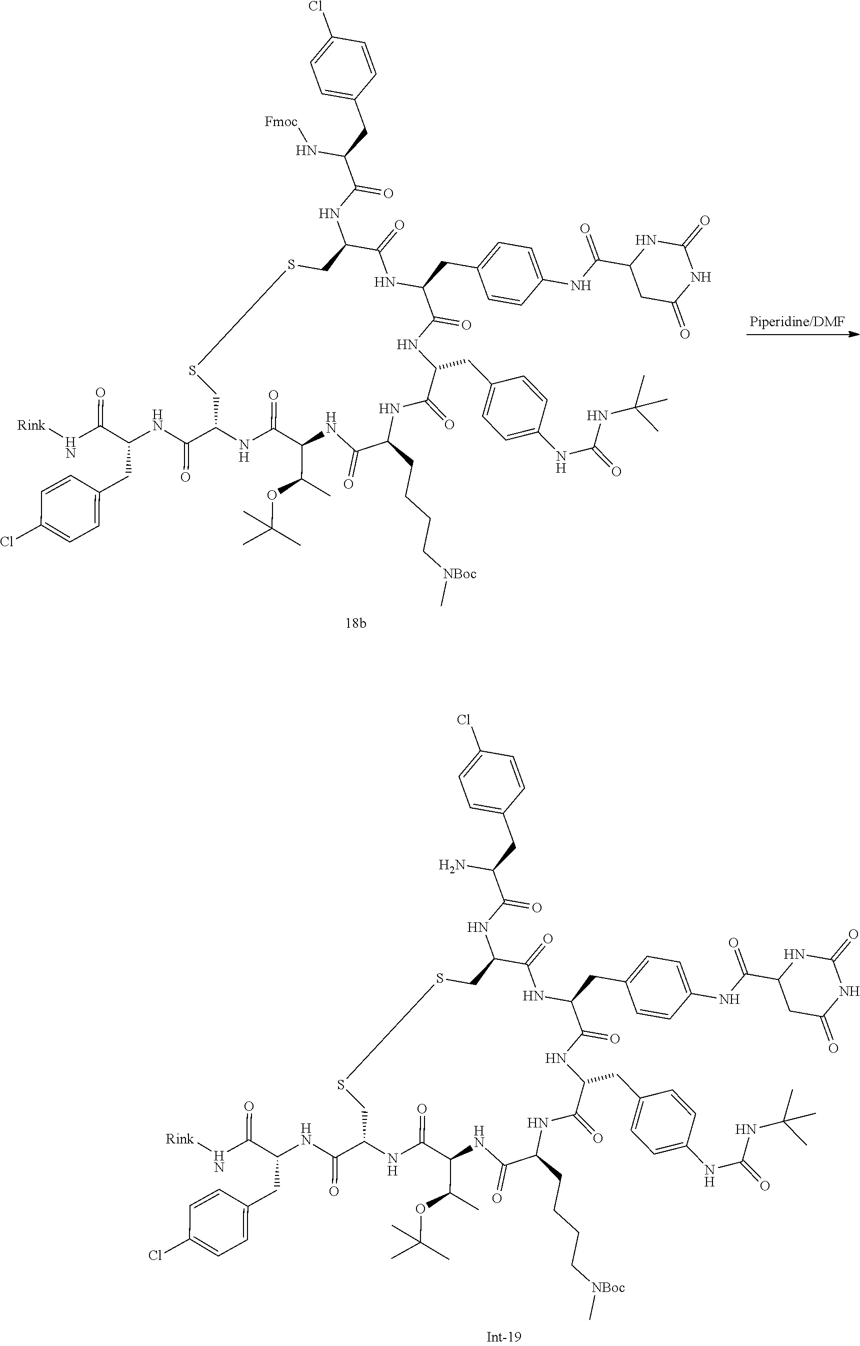

### Fmoc-4-Cl-Phe-D-Cys(Trt)-Aph(Hor)-D-Aph(Cbm)-Lys(Me, Boc)-Thr(O-t-Bu)-Cys(Trt)-D-4-Cl-Phe-Rink Amide Resin (19a)

Rink Amide resin (1 mmol, Ambeed Cat #: A451407) was swelled in DMF (20 ml) for 30 minutes, then filtered. The Fmoc group was removed by treating the resin with 20% piperidine in DMF (20 ml) at 25° C. for 1 h, then the resin was washed with DMF (20 ml×5).

### SPPS was Carried Out as Follows:

Coupling: Fmoc-AA-OH (4 eq.) was preactivated with HBTU (4 eq.) and DIPEA (4 eq.) in 20 ml of DMF for 1 min and added to the resin and heated in microwave reactor at 20 W, 45° C. for 7 min with gentle stirring. The resin was washed with DMF (10 ml×5).

Deprotection: 20% piperidine in DMF (20 ml) was added to resin and heated in microwave reactor at 20 W, 45° C. for 5 min with gentle stirring. The resin was washed with DMF (10 ml×5).

### Cyclo-Fmoc-4-Cl-Phe-[D-Cys(Trt)-Aph(Hor)-D-Aph(Cbm)-Lys(Me, Boc)-Thr(O-t-Bu)-Cys(Trt)]-D-4-Cl-Phe-Rink Amide Resin (19b)

Resin-peptide 19a (1 mmol) was suspended in DMF (20 ml). Iodine (5 eq) was added, and the mixture was gently stirred at 25° C. for 2 h, filtered and the resin was washed with DMF (10 ml×5) and DCM (10 ml×5).

### Cyclo-NH₂-4-Cl-Phe-[D-Cys(Trt)-Aph(Hor)-D-Aph(Cbm)-Lys(Me, Boc)-Thr(O-t-Bu)-Cys(Trt)]-D-4-Cl-Phe-Rink Amide Resin (Int-19)

Resin bound 19b (0.025 mmol) was suspended in 20% piperidine in DMF (5 ml) and stirred at 25° C. for 30 min. Resin-peptide was washed with DMF (5 ml×5) to afford Int-19 (assume quant.).

### Preparation of Cyclo-NH₂-4-Cl-Phe-[D-Cys(Trt)-Aph(Hor)-D-Aph(Cbm)-Lys(Me, Boc)-Thr(O-t-Bu)-Cys(Trt)]-D-4-F-Phe-Rink Amide Resin (Int-20)

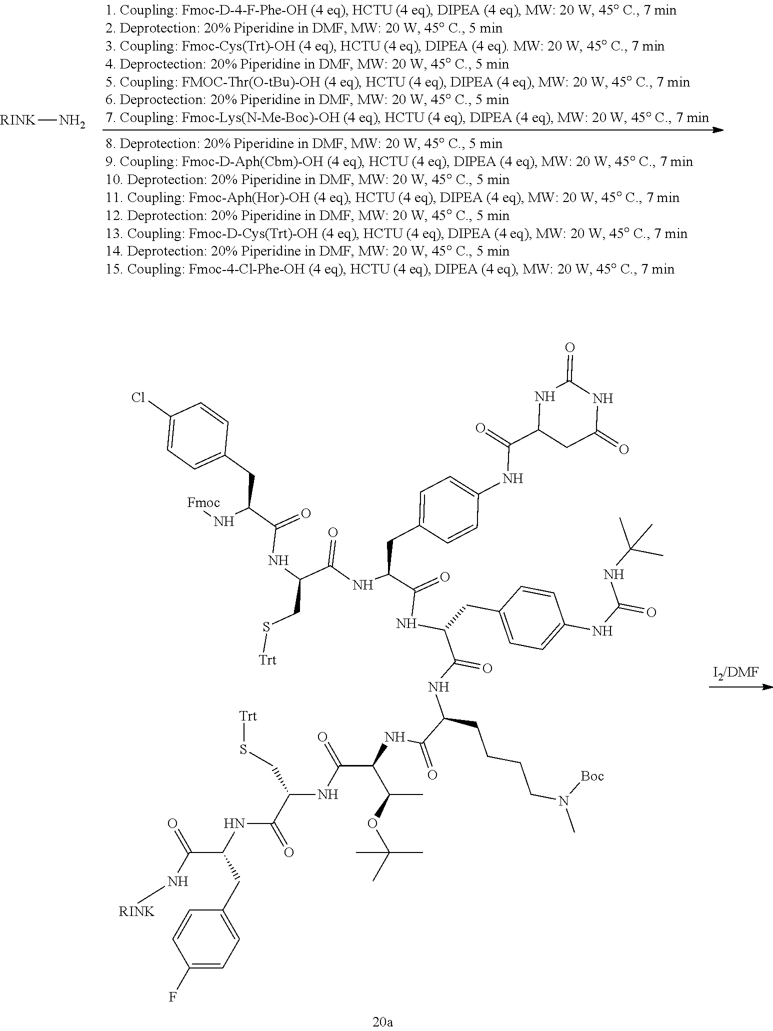

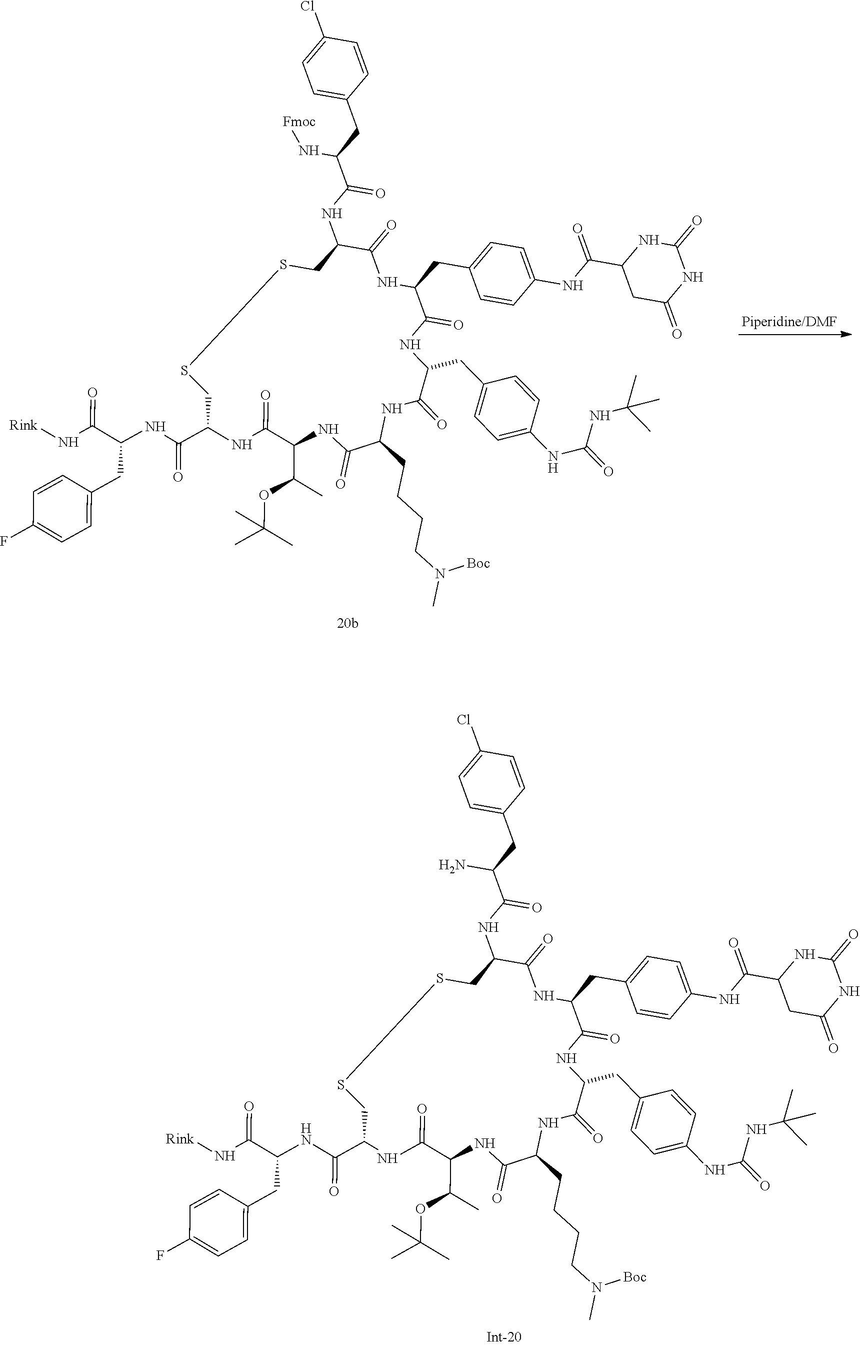

### Fmoc-4-Cl-Phe-D-Cys(Trt)-Aph(Hor)-D-Aph(Cbm)-Lys(Me, Boc)-Thr(O-t-Bu)-Cys(Trt)-D-4-F-Phe-Rink Amide Resin (20a)

Rink Amide resin (1 mmol, Ambeed Cat #: A451407) was swelled in DMF (20 ml) for 30 minutes, then filtered. The Fmoc group was removed by treating the resin with 20% piperidine in DMF (20 ml) at 25° C. for 1 h, then the resin was washed with DMF (20 ml×5).

### SPPS was Carried Out as Follows:

Coupling: Fmoc-AA-OH (4 eq.) was preactivated with HBTU (4 eq.) and DIPEA (4 eq.) in (20 ml) of DMF for 1 min and added to resin and heated in microwave reactor at 20 W, 45° C. for 7 min with gentle stirring. The resin was washed with DMF (10 ml×5).

Deprotection: 20% piperidine in DMF (20 ml) was added to resin and heated in microwave reactor at 20 W, 45° C. for 5 min with gentle stirring. The resin was washed with DMF (10 ml×5).

### Cyclo-Fmoc-4-Cl-Phe-[D-Cys(Trt)-Aph(Hor)-D-Aph(Cbm)-Lys(Me, Boc)-Thr(O-t-Bu)-Cys(Trt)]-D-4-F-Phe-Rink Amide Resin (20b)

20a (1 mmol) was suspended in DMF (20 ml). Iodine (5 eq) was added, and the mixture was gently stirred at 25° C. for 2 h, filtered and the resin was washed with DMF (10 ml×5) and DCM (10 ml×5).

### Cyclo-NH₂-4-Cl-Phe-[D-Cys(Trt)-Aph(Hor)-D-Aph(Cbm)-Lys(Me, Boc)-Thr(O-t-Bu)-Cys(Trt)]-D-4-F-Phe-Rink Amide Resin (Int-20)

20b (0.01 mmol) was suspended in 20% piperidine in DMF (5 ml) and stirred at 25° C. for 30 min. The Resin-peptide was washed with DMF (5 ml×5) to afford Int-20 (assume quant.).

### Preparation of Cyclo-Fmoc-4-Cl-Phe-[D-Cys(Trt)-Aph(Hor)-D-Aph(Cbm)-Lys(α-N-Me)(Me, Boc)-Thr(O-t-Bu)-Cys(Trt)]-D-Tyr(O-t-Bu)-Rink Amide Resin (Int-21)

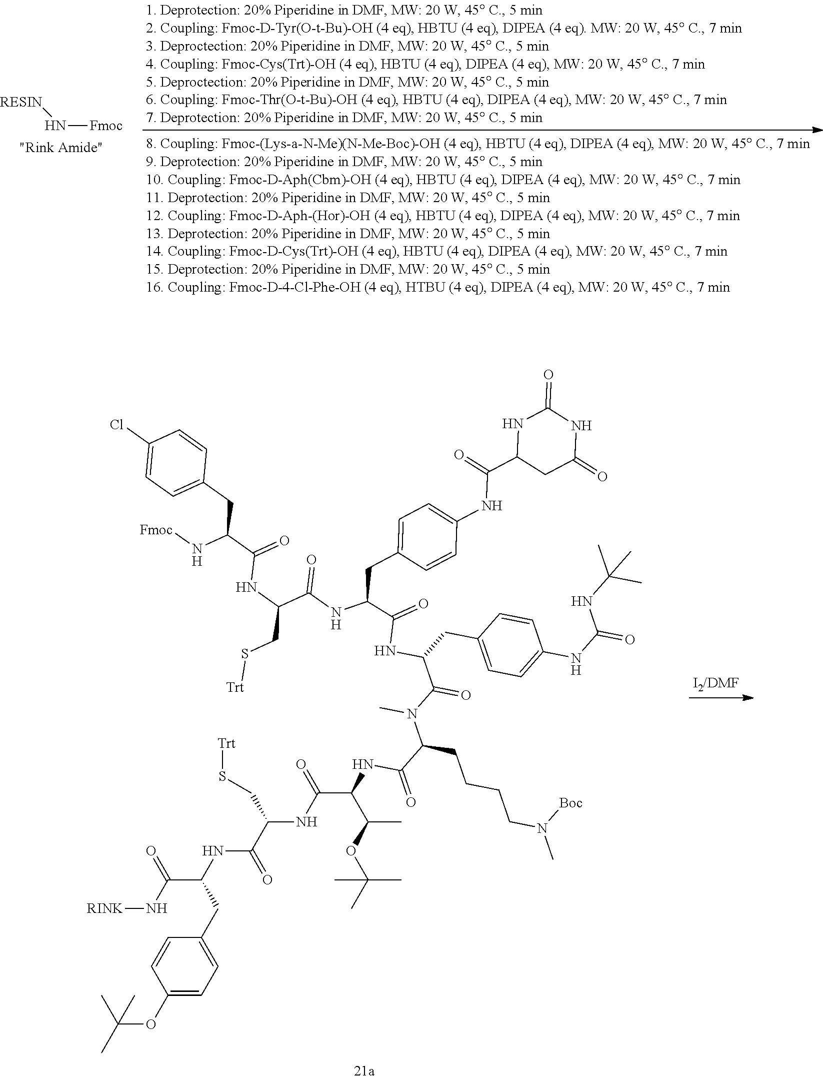

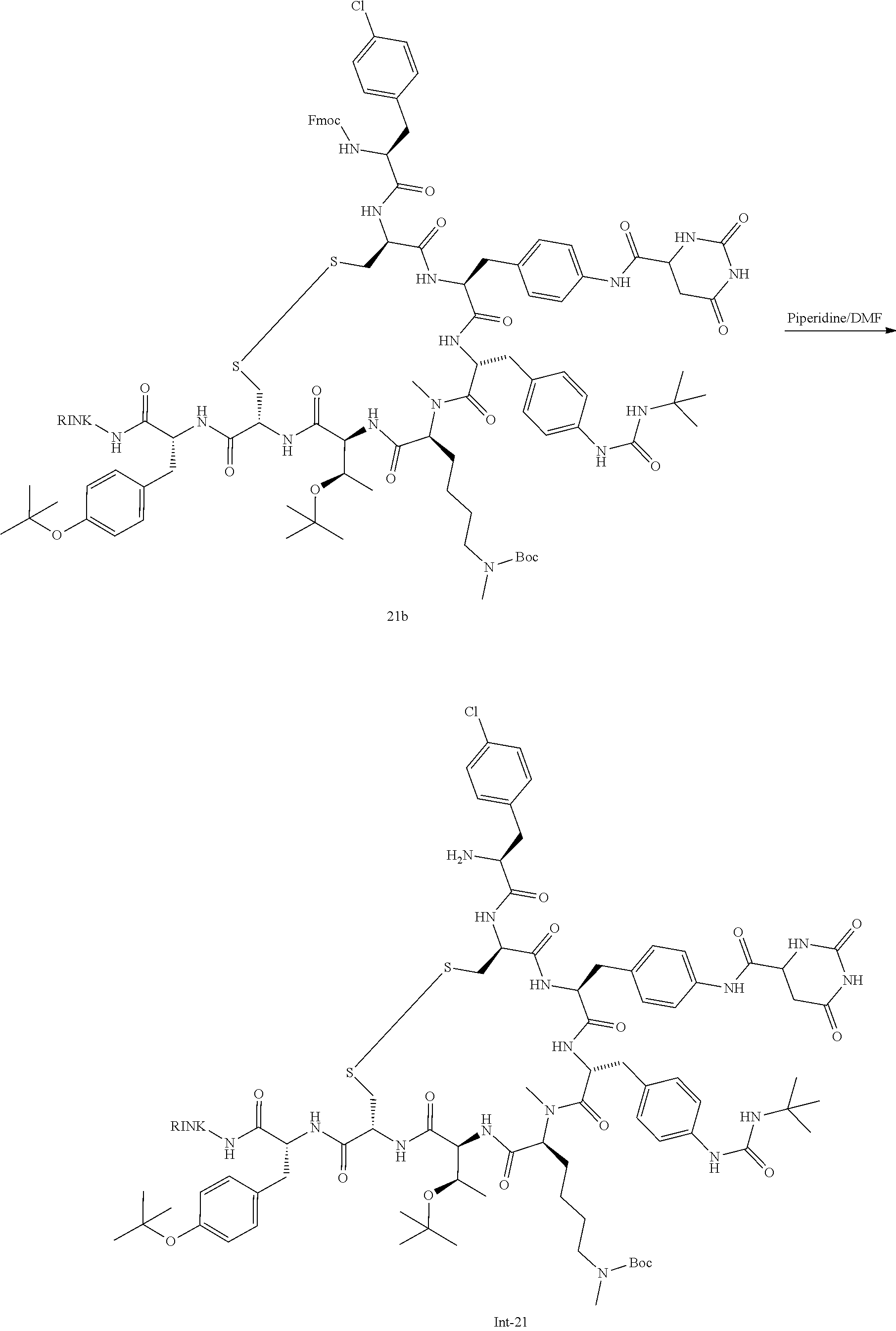

### Fmoc-4-Cl-Phe-D-Cys(Trt)-Aph(Hor)-D-Aph(Cbm)-Lys(α-N-Me)(Me, Boc)-Thr(O-t-Bu)-Cys(Trt)-D-Tyr(O-t-Bu)-Rink Amide Resin (21a)

RINK AMIDE resin (0.2 mmol, Ambeed Cat #: A451407) was swelled in DMF (5 ml) for 30 minutes, then filtered. The Fmoc group was removed by treating the resin with 20% piperidine in DMF (5 ml) at 25° C. for 1 h, then the resin was washed with DMF (20 ml×5).

### SPPS was Carried Out as Follows:

Coupling: Fmoc-AA-OH (4 eq.) was preactivated with HBTU (4 eq.) and DIPEA (4 eq.) in 10 ml of DMF for 1 min and added to resin and heated in microwave reactor at 20 W, 45° C. for 7 min with gentle stirring. The resin was washed with DMF (5 ml×5).

Deprotection: 20% piperidine in DMF (10 ml) was added to resin and heated in microwave reactor at 20 W, 45° C. for 5 min with gentle stirring. The resin was washed with DMF (5 ml×5) and afforded resin-bound peptide 21a (assume quant. yield).

### Cyclo-Fmoc-4-Cl-Phe-[D-Cys(Trt)-Aph(Hor)-D-Aph(Cbm)-Lys(α-N-Me)(Me, Boc)-Thr(O-t-Bu)-Cys(Trt)]-D-Tyr(O-t-Bu)-Rink Amide Resin (21b)

Resin-peptide 21a (0.05 mmol) was suspended in DMF (20 ml). Iodine (5 eq) was added, and the mixture was gently stirred at 25° C. for 2 h, filtered and the resin was washed with DMF (10 ml×5) and DCM (10 ml×5).

### Cyclo-Fmoc-4-Cl-Phe-[D-Cys(Trt)-Aph(Hor)-D-Aph(Cbm)-Lys(α-N-Me)(Me, Boc)-Thr(O-t-Bu)-Cys(Trt)]-D-Tyr(O-t-Bu)-Rink Amide Resin (Int-21)

Resin bound 21b (0.025 mmol) was suspended in 20% piperidine in DMF (5 ml) and stirred at 25° C. for 30 min. The resin-peptide was washed with DMF (5 ml×5) to afford Int-21 (assume quant.).

### Preparation of Cyclo-3-I-D-Phe-[Cys(Trt)-Tyr(O-t-Bu)-D-Trp(Boc)-Lys(α-N-Me, ε-N-Me-Boc)-Thr(O-t-Bu)-Cys(Trt)]-Thr(O-t-Bu)-2-Cl-trityl Resin (Int-22)

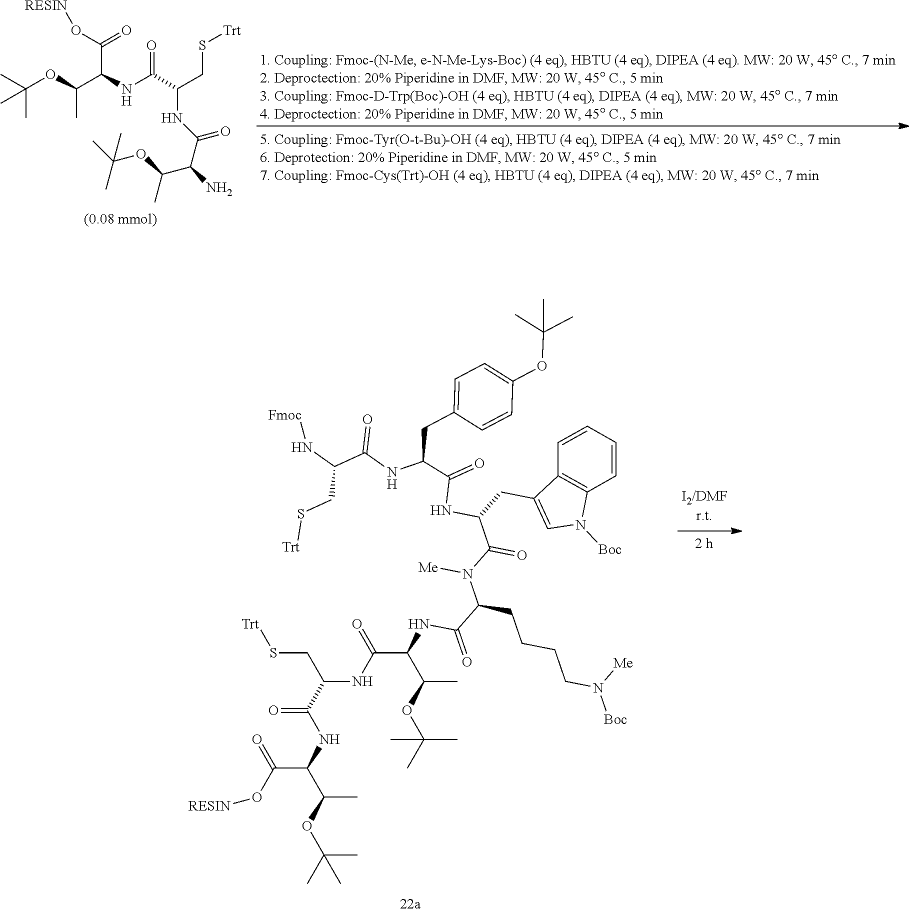

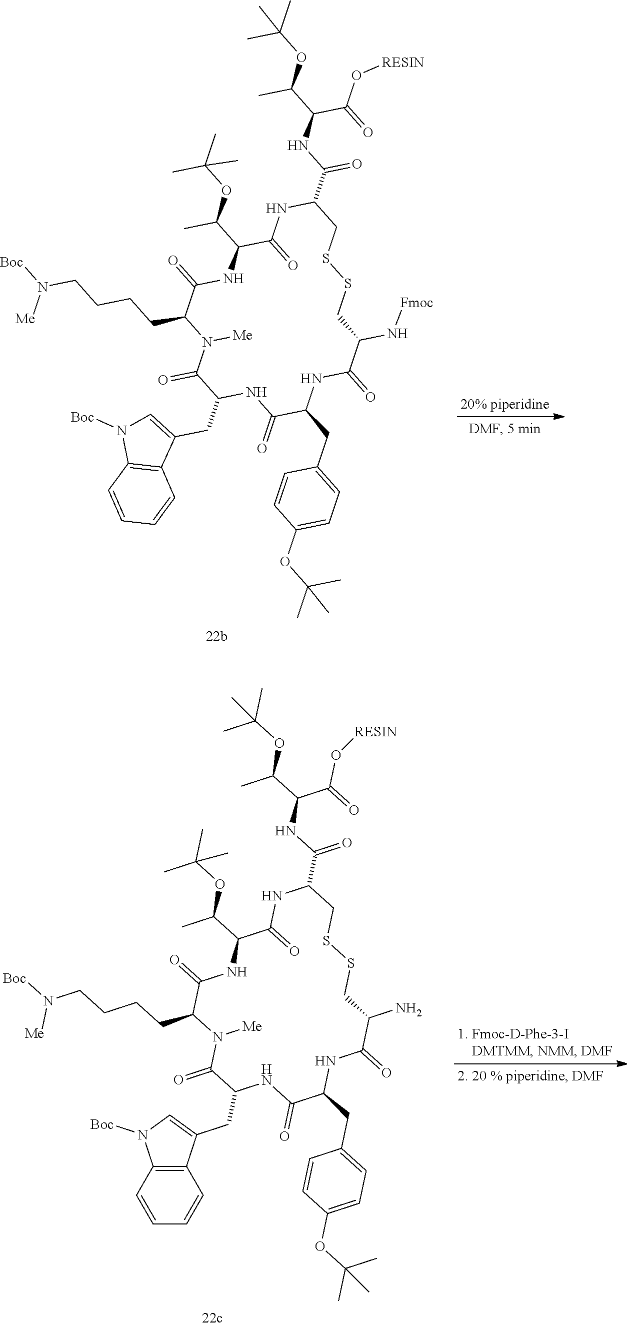

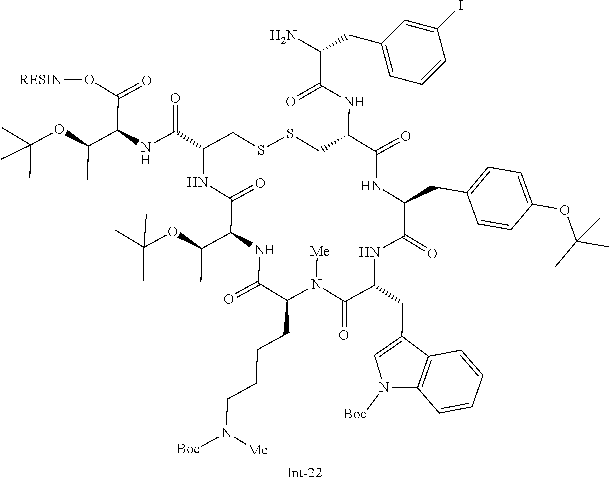

### Fmoc-Cys(Trt)-Tyr(O-t-Bu)-D-Trp(Boc)-Lys(α-N-Me, ε-N-Me-Boc)-Thr(O-t-Bu)-Cys(Trt)-Thr(O-t-Bu)-2-Cl-trityl Resin (22a)

### SPPS was Carried Out as Follows:

Coupling: Fmoc-AA-OH (4 eq.) was preactivated with HBTU (4 eq.) and DIPEA (5 eq.) in (5 ml) of DMF for 1 min and added to the resin and heated in microwave reactor at 20 W, 45° C. for 7 min with gentle stirring. The resin was washed with DMF (10 ml×5).

Deprotection: 20% piperidine in DMF (4 ml) was added to resin and heated in microwave reactor at 20 W, 45° C. for 5 min with gentle stirring. The resin was washed with DMF (10 ml×5).

### Cyclo-[Fmoc-Cys(Trt)-Tyr(O-t-Bu)-D-Trp(Boc)-Lys(α-N-Me,ε-N-Me-Boc)-Thr(O-t-Bu)-Cys(Trt)]-Thr(O-t-Bu)-2-Cl-trityl Resin (22b)

Resin-peptide 22a (0.08 mmol) was suspended in DMF (5 ml). Iodine (5 eq) was added, and the mixture was gently stirred at 25° C. for 2 h, filtered and washed resin with DMF (10 ml×5) and DCM (10 ml×5).

### Cyclo-[Cys(Trt)-Tyr(O-t-Bu)-D-Trp(Boc)Lys(α-N-Me,ε-N-Me-Boc)-Thr(O-t-Bu)-Cys(Trt)]-Thr(O-t-Bu)-2-Cl-trityl Resin (22c)

Resin-peptide 22b (0.03 mmol) was treated with a solution of 20% piperidine in DMF (5 ml) and heated in microwave reactor at 20 W, 45° C. for 5 min with gentle stirring. The resin was washed with DMF (10 ml×5).

### Cyclo-3-I-D-Phe-[Cys(Trt)-Tyr(O-t-Bu)-D-Trp(Boc)-Lys(α-N-Me, ε-N-Me-Boc)-Thr(O-t-Bu)-Cys(Trt)]-Thr(O-t-Bu)-2-Cl-trityl Resin (Int-22)

Resin-peptide 22c (0.03 mmol) was suspended in DMF (5 ml). Fmoc-3-I-D-Phe-OH (0.12 mmol, 4 eq), NMM (0.1 mL) and DMTMM (0.12 mmol, 4 eq) was added and the reaction was stirred gently at 25° C. for 1 h. The resin-peptide was filtered and washed with DMF (5 ml×5). 20% piperidine in DMF (5 ml) was added to resin-peptide and stirred at 25° C. for 30 min with gentle stirring. The resin-peptide was washed with DMF (5 ml×5) then DCM (5 ml×5). Resin-peptide 22c (0.03 mmol) was treated with a solution of 20% piperidine in DMF (5 ml) and heated in microwave reactor at 20 W, 45° C. for 5 min with gentle stirring. The resin was washed with DMF (10 ml×5) to afford resin bound Int-22 (assume quant.).

### Example 2—Preparation of Compounds of the Invention

### Preparation of Compound

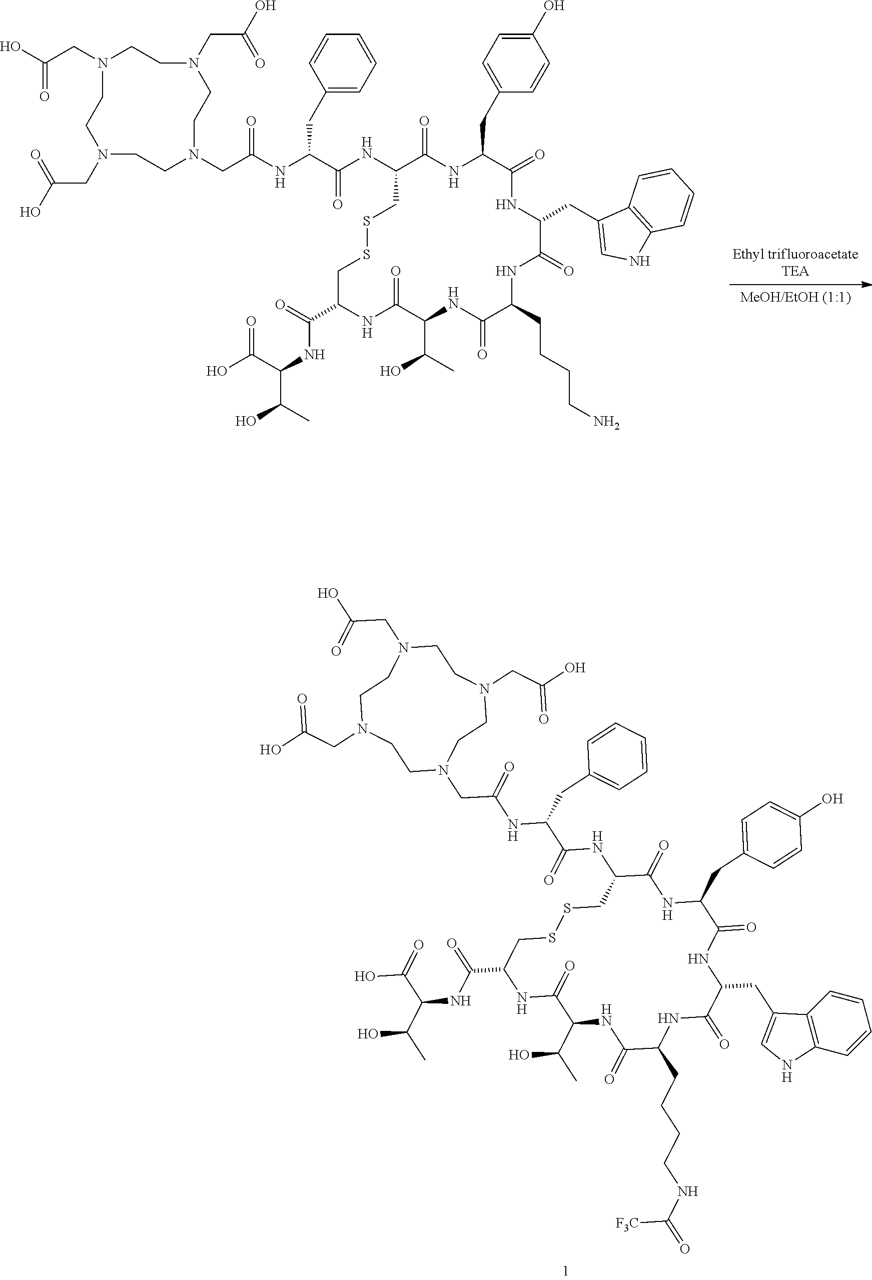 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O ; C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCNC(=O)C(F)(F)F)C(=O)N[C@@H]([C@@H](C)O)C(=O)N1)C(=O)O]

Ethyl trifluoroacetate (0.2 μL, 0.0018 mmol, 3.0 equiv.) was added to a stirring solution of dotatate octreotate (0.8 mg, 0.0006 mmol, 1.0 equiv.) and triethylamine (0.3 μL, 0.0022 mmol, 4.0 equiv.) in 1:1 MeOH/EtOH (1.0 mL) at rt. The reaction was stirred at rt for 3 h, and the solvent evaporated under reduced pressure. The resulting crude product was purified by reverse phase HPLC, eluting with MeCN/H₂O+0.05% formic acid modifier. Fractions containing pure product were combined and lyophilized to afford Compound 1 as a white solid (0.6 mg, 70%); MS (ESI) m/z: 1531.9 (M+H)⁺.

### Preparation of Compound 2

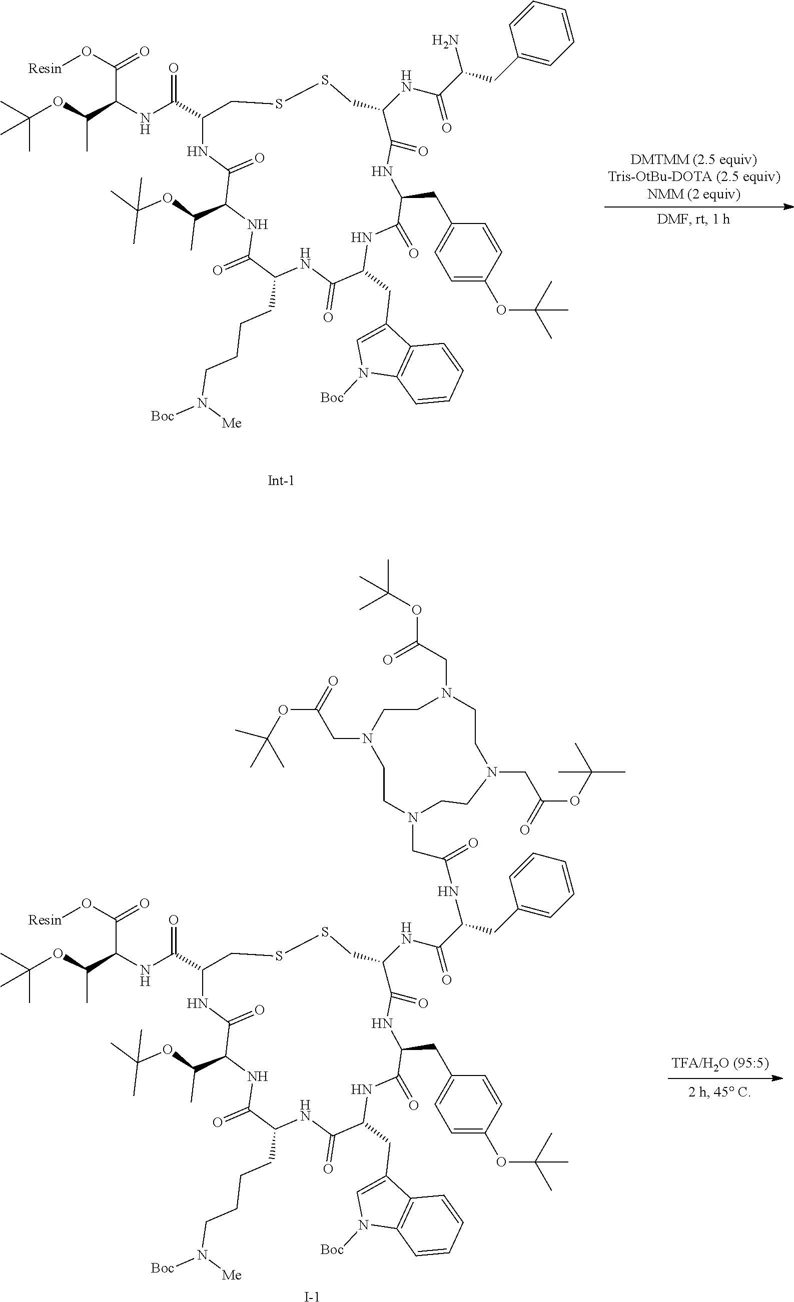

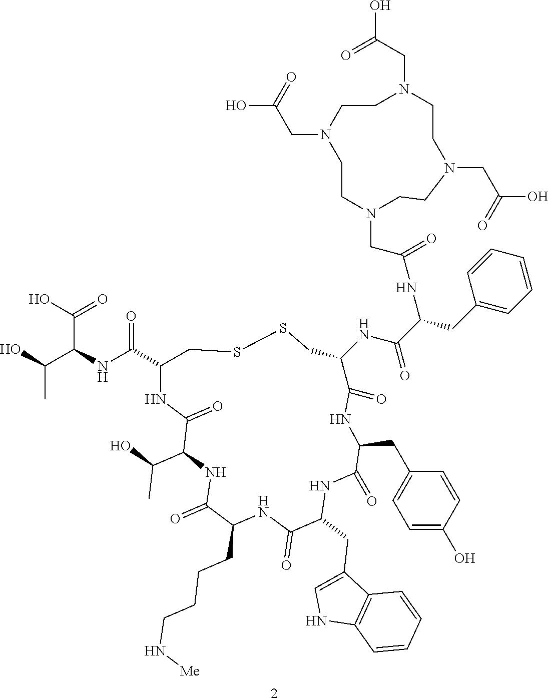

Int-1 (0.25 mmol) was placed in a fritted funnel and suspended in DMF (5 mL). To this suspension, dota-tris(tert-butyl ester) (72 mg, 0.025 mmol, 2.5 equiv), DMTMM/(35 mg, 0.063 mmol, 2.5 equiv), and N-methylmorpholine (5 drops) were added. The reaction mixture was stirred at room temperature for 2 hours. Afterward, the mixture was filtered to isolate resin bound I-1, which was washed sequentially with DMF (10 mL×5) and DCM (10 mL×2). Resin bound material I-1 was utilized without further purification in the subsequent step.

Resin bound I-1 (0.025 mmol) was suspended in a mixture of trifluoroacetic acid and water (95:5, 4 mL) and stirred for 1 minute. The reaction mixture was then filtered into a glass vial, and the remaining resin was washed with an additional 1 mL of the 95:5 trifluoroacetic acid-water mixture and filtered again. The filtrate was stirred at 45° C. and monitored by LC/MS for 2 hours. Afterward, the reaction mixture was concentrated under reduced pressure and purified by reverse-phase C18 column chromatography, eluting with a gradient of MeCN/H₂O (0.05% formic acid modifier). The gradient started at 5% MeCN, reaching 95% MeCN over 20 minutes, with the product eluting at 35% MeCN. Pure fractions were collected and lyophilized to yield compound 2 as a white solid (4.4 mg, 16% yield). MS (ESI) m/z: 1450.0 (M+H)*; HPLC purity: 100% (Diode Array), t_{R}: 0.71 min.

The following compounds were prepared using procedures similar to those for Compound 2:

| Compound | | Starting |
|---|---|---|
| No. | Structure | Material |
| 3 | 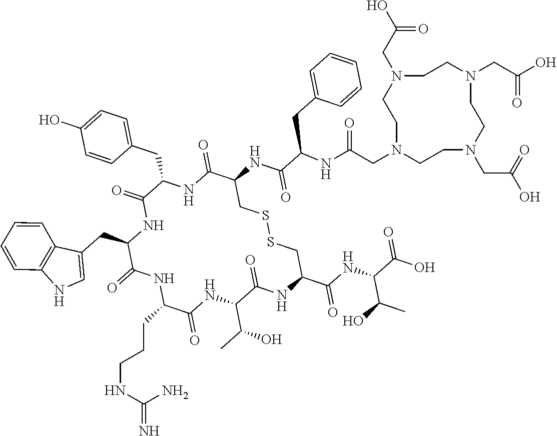 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCNC(=N)N)C(=O)N[C@@H]([C@@H](C)O)C(=O)N1)C(=O)O] | Int-13 |
| 4 | 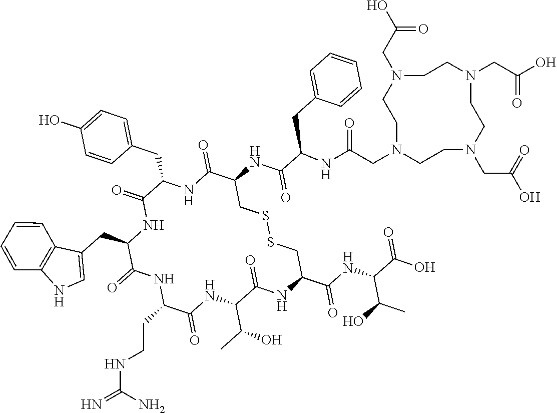 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCNC(=N)N)C(=O)N[C@@H]([C@@H](C)O)C(=O)N1)C(=O)O] | Int-14 |
| 5 | 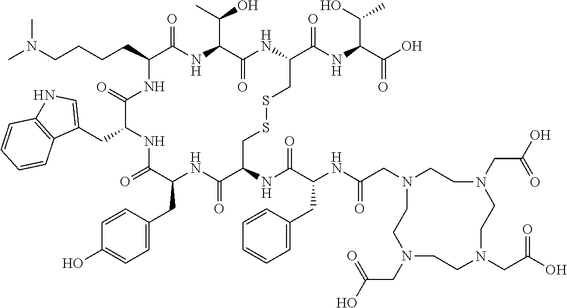 [SMILES: C[C@@H](O)[C@H](NC(=O)[C@@H]1CSSC[C@@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN(C)C)C(=O)N[C@@H]([C@@H](C)O)C(=O)N1)C(=O)O] | Int-10 |
| 6 | 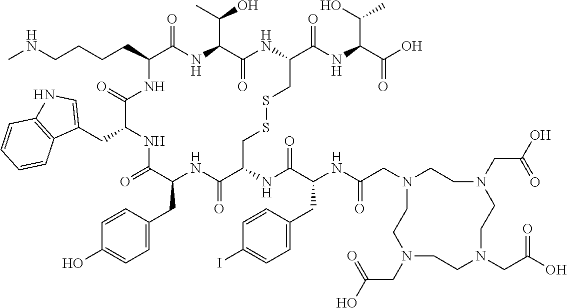 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccc(I)cc2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] | Int-12 |
| 7 | 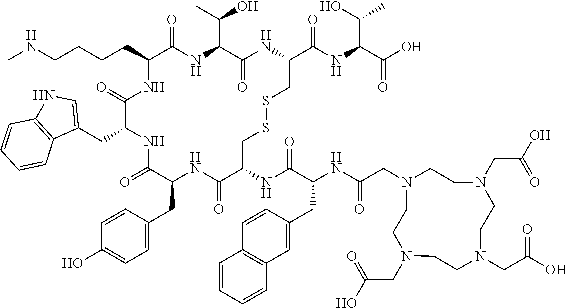 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccc3ccccc3c2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] | Int-11 |
| 8 | 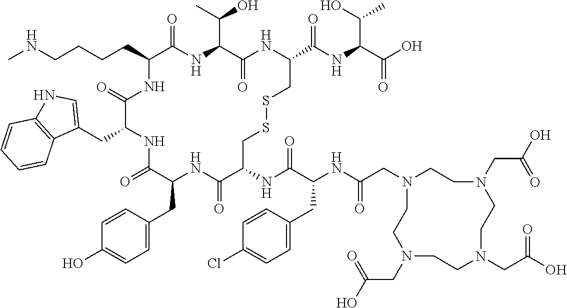 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccc(Cl)cc2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] | Int-5 |
| 11 | 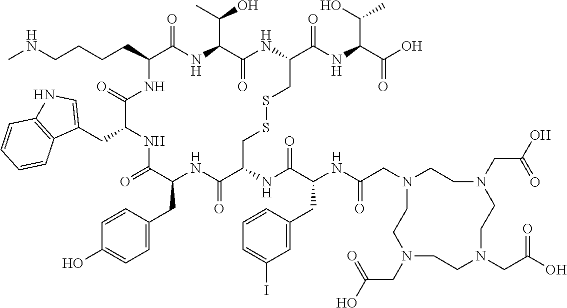 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2cccc(I)c2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] | Int-7 |
| 12 | 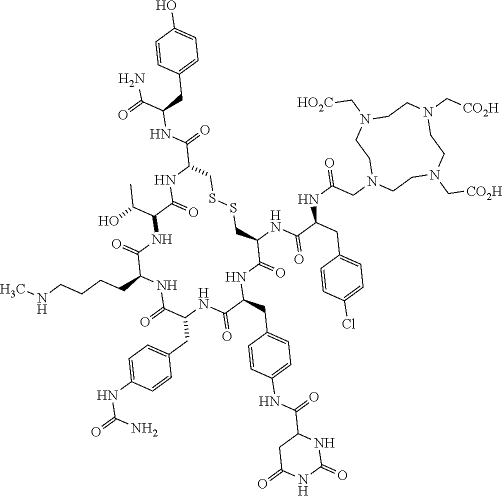 | Int-15 |
| 13 | 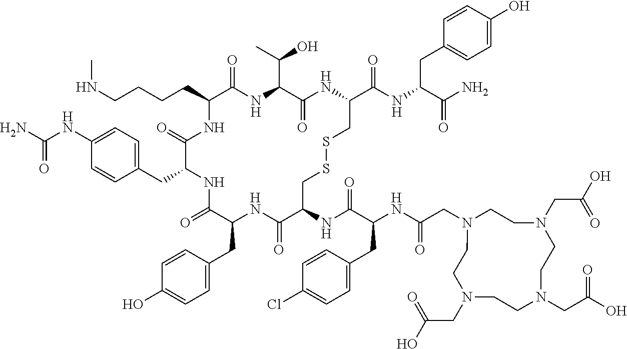 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2ccc(Cl)cc2)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] | Int-18 |
| 15 | 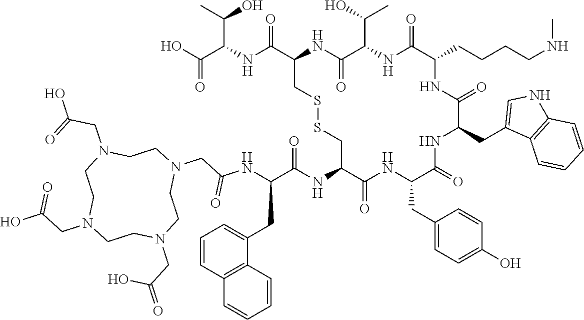 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2cccc3ccccc23)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] | Int-3 |
| 23 | 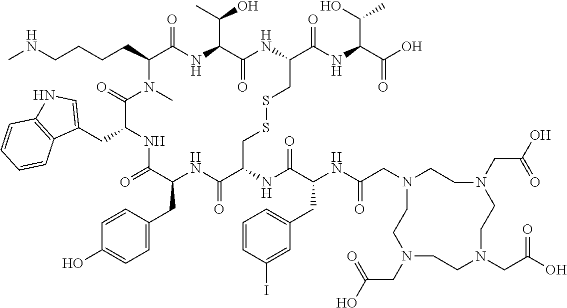 [SMILES: CNCCCC[C@H]1C(=O)N[C@@H]([C@@H](C)O)C(=O)N[C@H](C(=O)N[C@H](C(=O)O)[C@@H](C)O)CSSC[C@H](NC(=O)[C@@H](Cc2cccc(I)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N1C] | Int-22 |
| 24 | 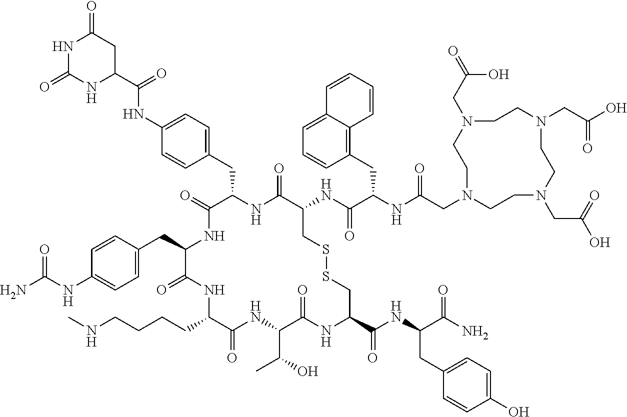 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2cccc3ccccc23)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] | Int-16 |
| 25 | 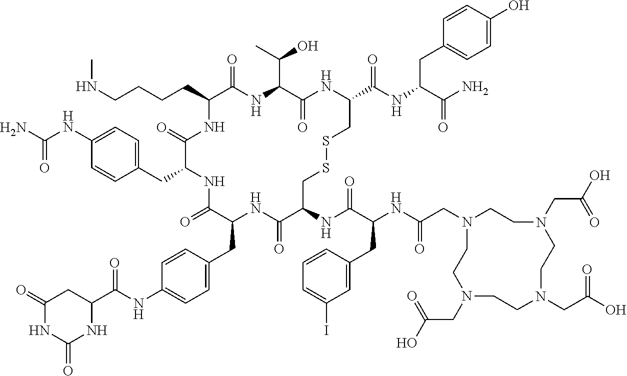 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2cccc(I)c2)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] | Int-17 |
| 26 | 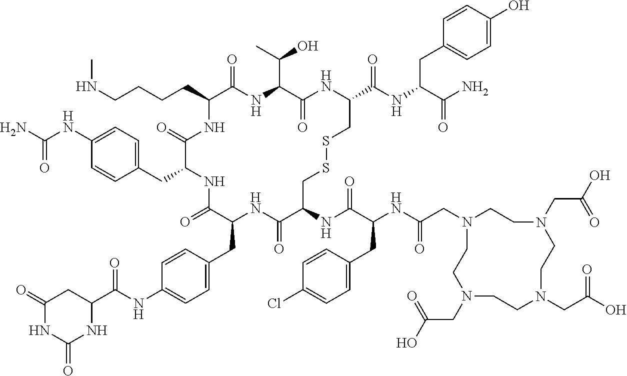 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2ccc(Cl)cc2)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] | Int-19 |
| 27 | 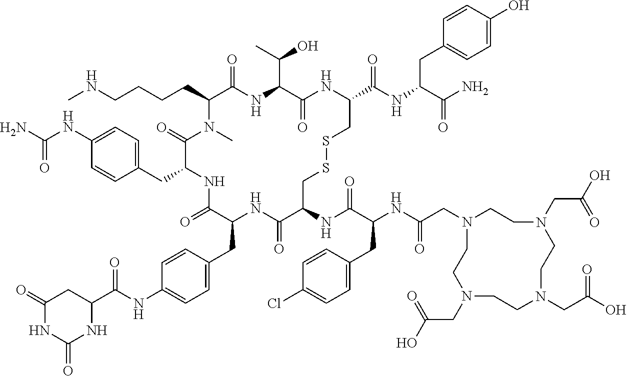 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)CN2CCN(CC(=O)O)CCN(CC(=O)O)CCN(CC(=O)O)CC2)C(=O)N[C@@H](Cc2ccc(NC(=O)C3CC(=O)NC(=O)N3)cc2)C(=O)N[C@H](Cc2ccc(NC(N)=O)cc2)C(=O)N1C] | Int-21 |
| 31 | 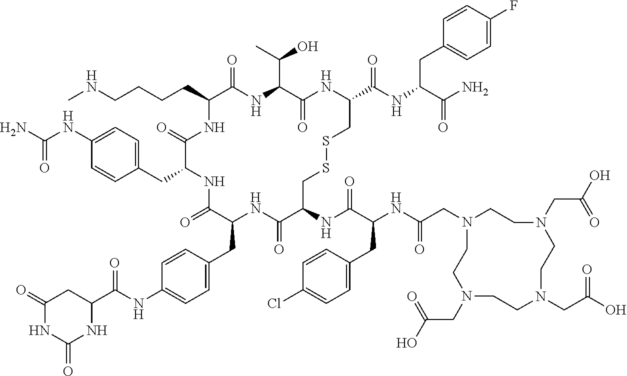 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2ccc(Cl)cc2)NC(=O)CN2CCN(CC(=O)O)CCN(CC(=O)O)CCN(CC(=O)O)CC2)CSSC[C@@H](C(=O)N[C@H](Cc2ccc(F)cc2)C(N)=O)NC(=O)[C@H]([C@@H](C)O)NC1=O] | Int-20 |
| 32 | 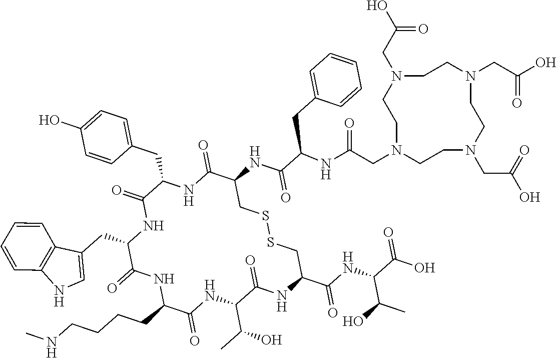 [SMILES: CNCCCC[C@H]1NC(=O)[C@H](Cc2c[nH]c3ccccc23)NC(=O)[C@H](Cc2ccc(O)cc2)NC(=O)[C@@H](NC(=O)[C@@H](Cc2ccccc2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] | Int-26 |
| 38 | 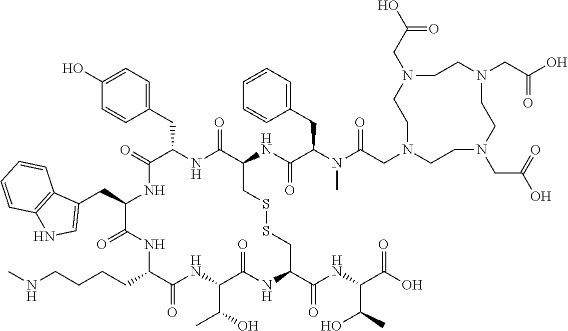 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccccc2)N(C)C(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] | Int-28 |
| 39 | 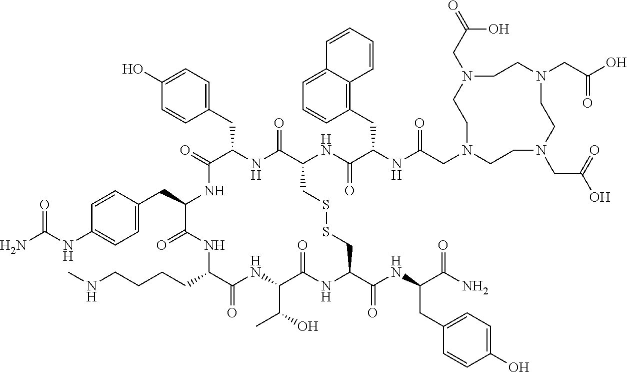 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2cccc3ccccc23)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] | Int-29 |
| 40 | 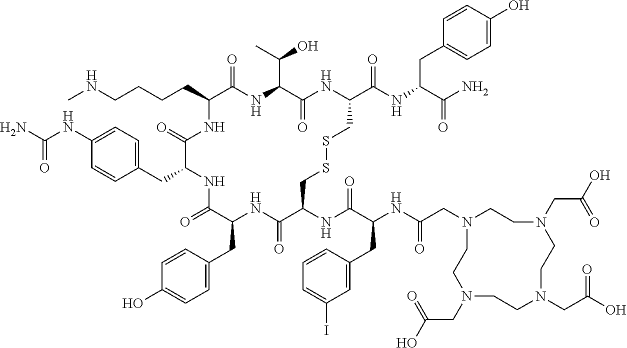 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2cccc(I)c2)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] | Int-30 |

### Preparation of Compound 9

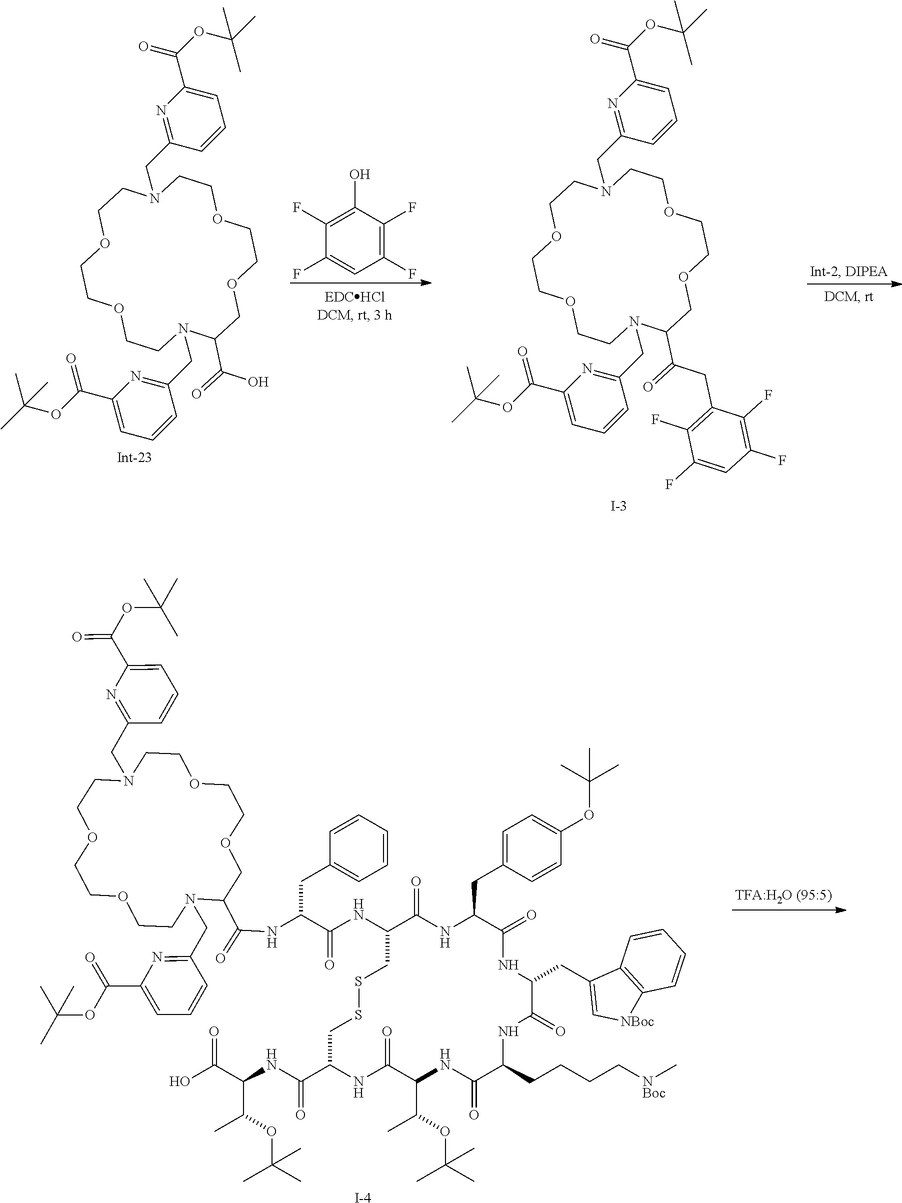

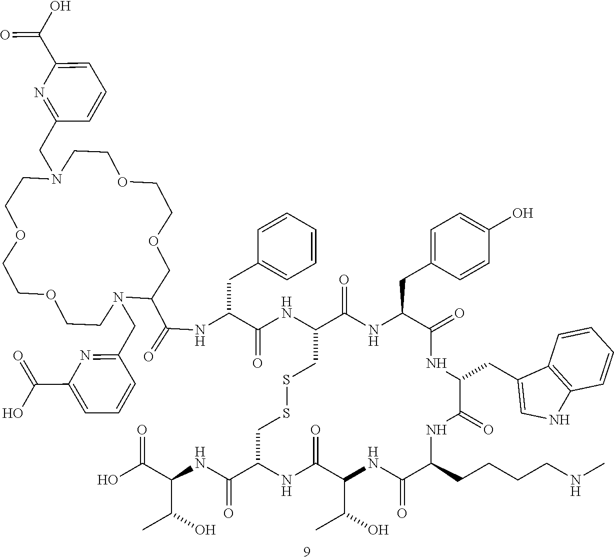

EDC·HCl (10.2 mg, 0.053 mmol, 1.5 equiv.) was added to a stirring solution of Int-23 (24.5 mg, 0.036 mmol, 1.0 equiv.), DIPEA (11.7 μL, 0.107 mmol, 3 equiv.) and 2,3,5,6-tetrafluorophenol (8.9 mg, 0.053 mmol, 1.5 equiv.) in DCM (1.5 mL) at rt, and the resulting solution was stirred at rt for 3 h. The solvent was evaporated under reduced pressure, and the resulting crude product was purified by reverse phase HPLC, eluting with MeCN/H₂O+0.05% formic acid modifier. Fractions containing pure product were combined and lyophilized to afford I-3 as a white solid (15 mg, 50%).

A solution of I-3 (8.5 mg, 0.010 mmol), Int-2 (14.5 mg, 0.010 mmol) and DIPEA (5.3 μL, 0.031 mmol) was stirred at rt for 18 h. The excess solvent was evaporated under reduced pressure and the resulting crude product was purified by reverse phase HPLC, eluting with MeCN/H₂O+0.05% formic acid modifier. The pure fractions were concentrated under reduced pressure to afford I-4 as a white solid (10 mg, 47%); MS (ESI) m/z: 2103.2 (M+H)⁺.

I-4 (10 mg, 0.005 mmol) was dissolved in a solution of 95:5 mixture of trifluroacetic acid:water (1.5 mL) and was stirred at 40° C. for 1 h. The solvent was concentrated under reduced pressure, and the resulting crude product was purified by reverse phase HPLC, eluting with MeCN/H₂O+0.05% formic acid modifier. Fractions containing pure product were combined and concentrated under reduced pressure to afford compound 9 as a white solid (4.0 mg, 52%); MS (ESI) m/z: 1621.8 (M+H)⁺.

### Preparation of Compound 10

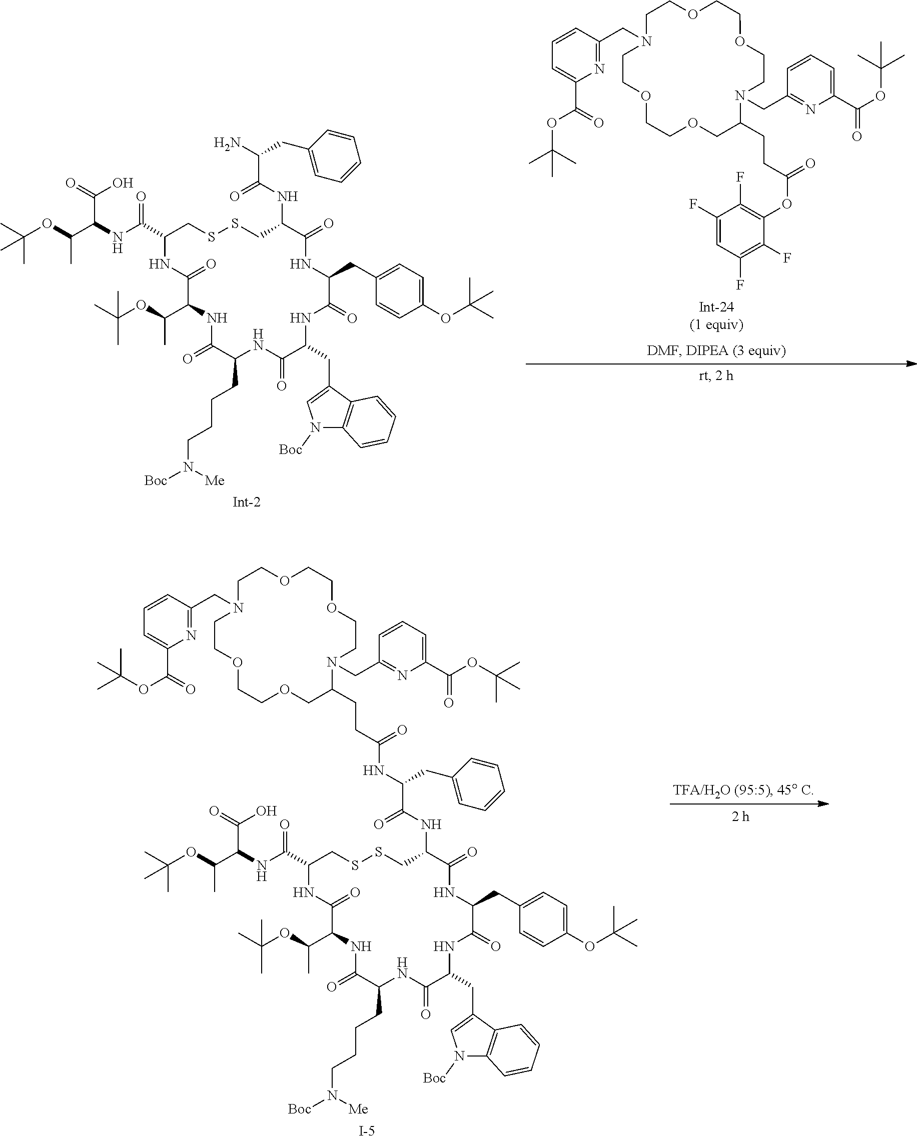

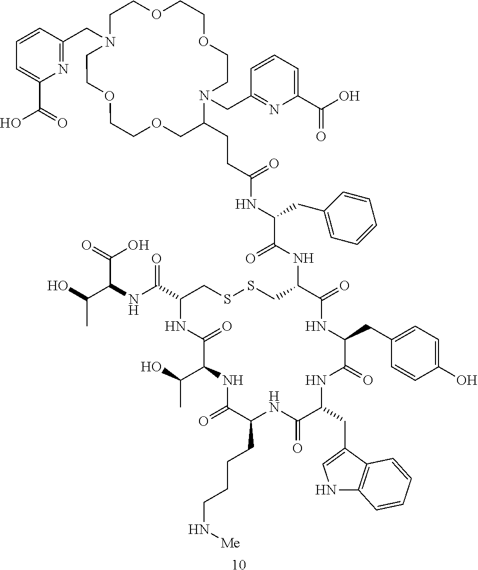

Int-2 (0.010 mmol, 15 mg, 1 equiv) diluted in 3 mL of dry DMF was reacted with Int-24 (0.013 mmol, 11 mg, 1.2 equiv) under basic conditions using DIPEA (3 equiv) at room temperature for 2 h. Subsequently, the solvent was evaporated and the crude was purified by reverse-phase C18 column chromatography, eluting with a gradient of MeCN/H₂O. The gradient started at 30% MeCN, reaching 100% MeCN and then to 50% MeCN—50% MeOH over 15 minutes, with the product eluting at 50% MeCN—50% MeOH. Pure fractions were collected and evaporated to yield Compound I-5 as a colorless oil (8.2 mg, 37% yield).

Macropa-Peptide I-5 (8.2 mg) was suspended in a mixture of trifluoroacetic acid and water (95:5, 4 mL), stirred for 1.5 h at 45° C. while monitored by LC/MS. Afterward, the reaction mixture was concentrated under reduced pressure and purified by reverse-phase C18 column chromatography, eluting with a gradient of MeCN/H₂O (0.05% formic acid modifier). The gradient started at 5% MeCN, reaching 50% MeCN over 20 minutes, with the product eluting at 30% MeCN. Pure fractions were collected and lyophilized to yield compound 10 as a white solid (2.6 mg, 41% yield). MS (ESI) m/z: 1649.8 (M+H)⁺

The following compounds were prepared using procedures similar to those for Compound 16:

| Compound | | Starting |
|---|---|---|
| No. | Structure | Material |
| 17 | 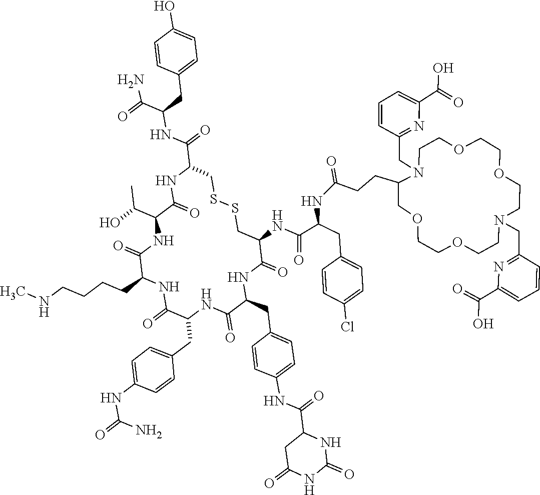 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2ccc(Cl)cc2)NC(=O)CCC2COCCOCCN(Cc3cccc(C(=O)O)n3)CCOCCOCCN2Cc2cccc(C(=O)O)n2)CSSC[C@@H](C(=O)N[C@H](Cc2ccc(O)cc2)C(N)=O)NC(=O)[C@H]([C@@H](C)O)NC1=O] | Int-15 |
| 20 | 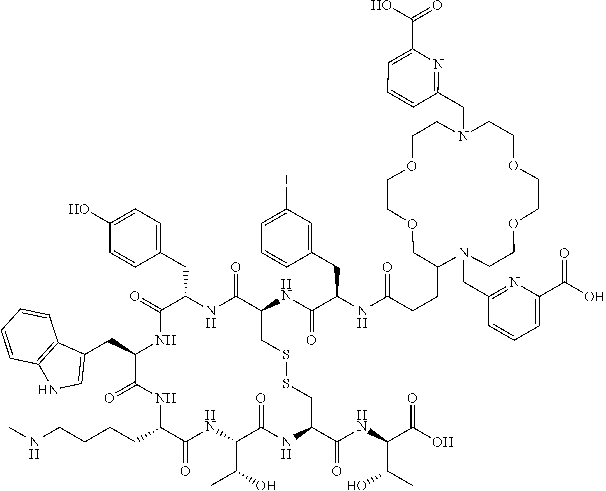 | Int-7 |
| 21 | 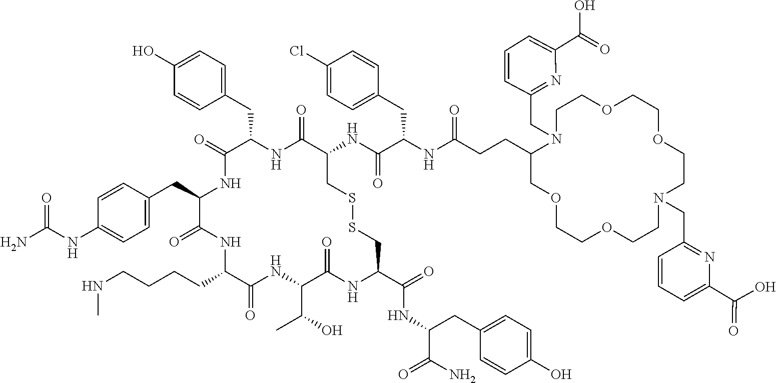 | Int-18 |
| 22 | 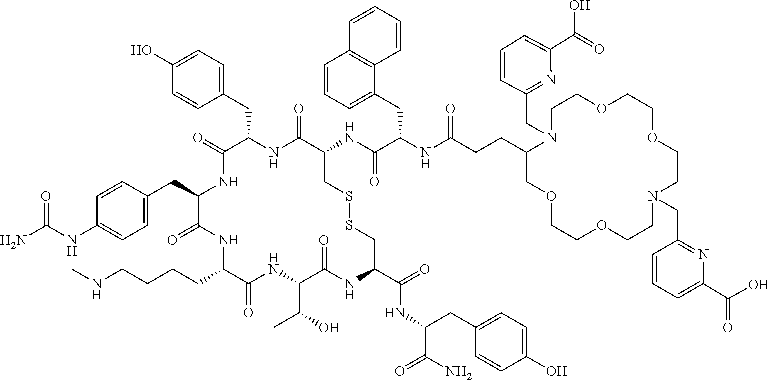 | Int-4 |

### Preparation of Compound 16

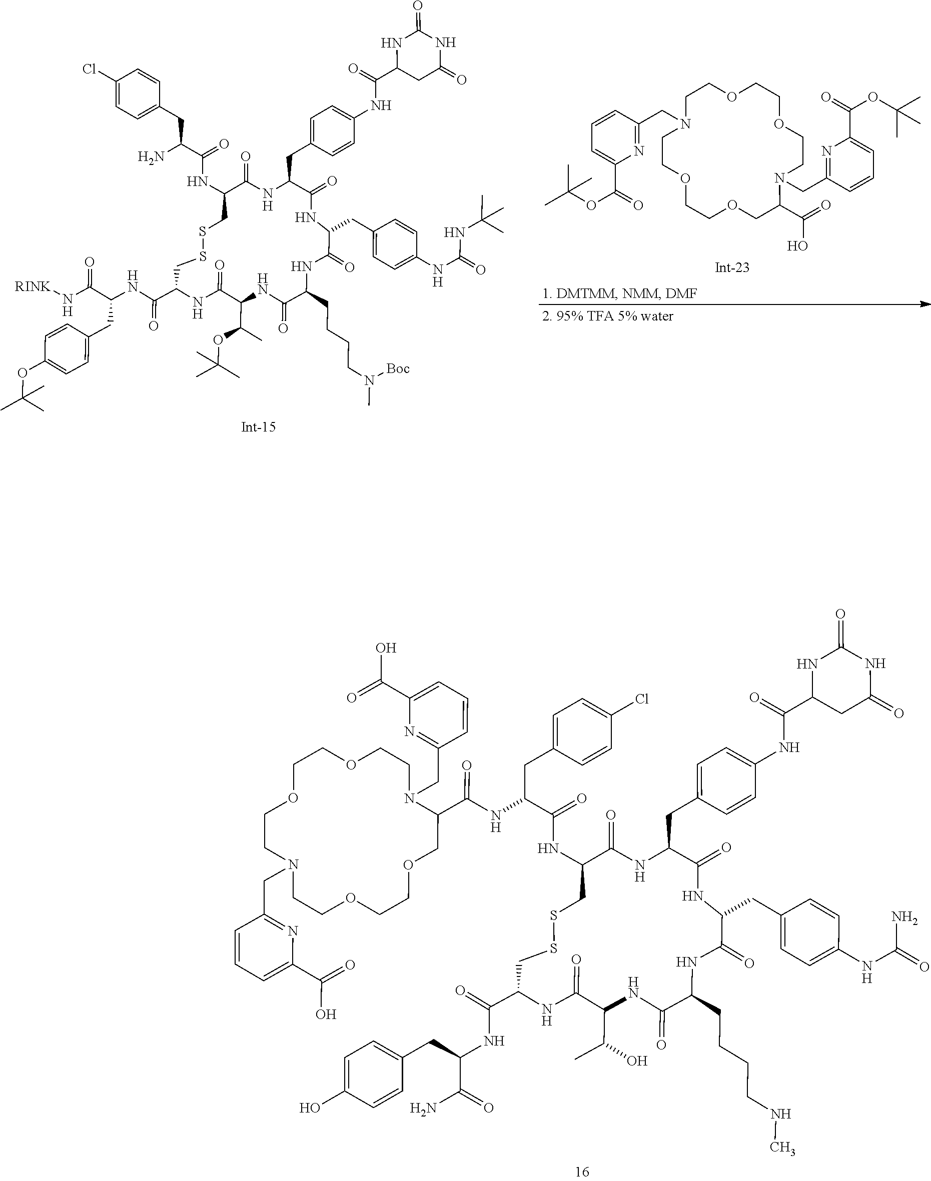

Int-15 (0.005 mmol) was suspended in DMF (1 mL). To this was added Int-23 (6.9 mg, 0.01 mmol, 2 eq), DMTMM (2.7 mg, 0.01 mmol, 2 eq), and N-methyl morpholine (1 μL, 0.01 mmol, 2 eq). The reaction mixture was stirred overnight at 25° C. The resin was washed with DMF (1 mL×5), and DCM (1 mL×5) then treated with (95:5) (TFA:water) (1.5 ml) at 45° C. for 1.5 h, filtered, and filtrate concentrated to dryness. The crude solid was dissolved in (3:1) (water/MeCN) (2 ml) and purified by reverse phase C18 flash chromatography (gradient elution; 10% MeCN/water—50% MeCN/water with 0.05% HCO₂H as mobile phase additive) to afford compound 16 (1.0 mg, 0.00053 mmol, 10.7%) as a white solid. MS (ESI) m/z: 937.7 (M+2H⁺)/2.

The following compounds were prepared using procedures similar to those for Compound 22:

| Compound | | Starting |
|---|---|---|
| No. | Structure | Material |
| 14 | 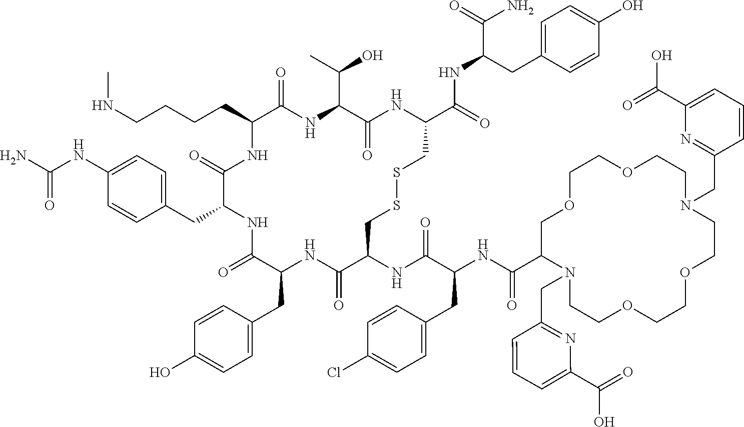 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2ccc(Cl)cc2)NC(=O)C2COCCOCCN(Cc3cccc(C(=O)O)n3)CCOCCOCCN2Cc2cccc(C(=O)O)n2)CSSC[C@@H](C(=O)N[C@H](Cc2ccc(O)cc2)C(N)=O)NC(=O)[C@H]([C@@H](C)O)NC1=O] | Int-18 |
| 18 | 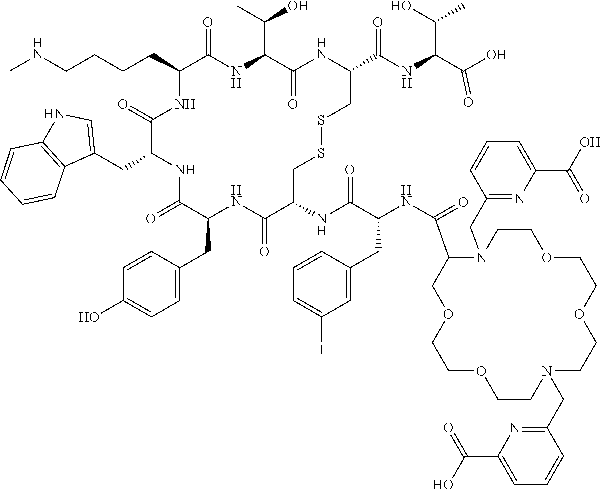 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2cccc(I)c2)NC(=O)C2COCCOCCN(Cc3cccc(C(=O)O)n3)CCOCCOCCN2Cc2cccc(C(=O)O)n2)CSSC[C@@H](C(=O)N[C@H](C(=O)O)[C@@H](C)O)NC(=O)[C@H]([C@@H](C)O)NC1=O] | Int-7 |
| 19 | 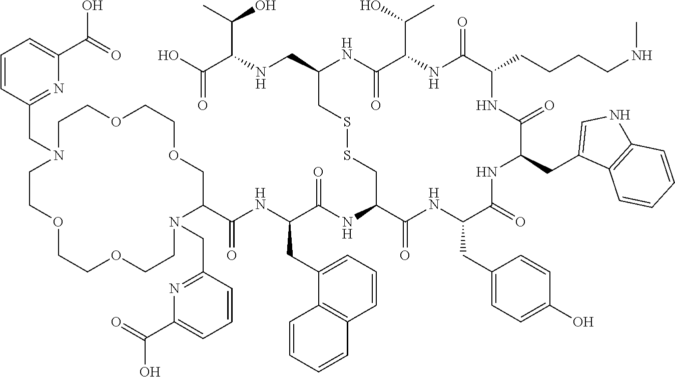 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2cccc3ccccc23)NC(=O)C2COCCOCCN(Cc3cccc(C(=O)O)n3)CCOCCOCCN2Cc2cccc(C(=O)O)n2)CSSC[C@@H](CN[C@H](C(=O)O)[C@@H](C)O)NC(=O)[C@H]([C@@H](C)O)NC1=O] | Int-4 |
| 28 | 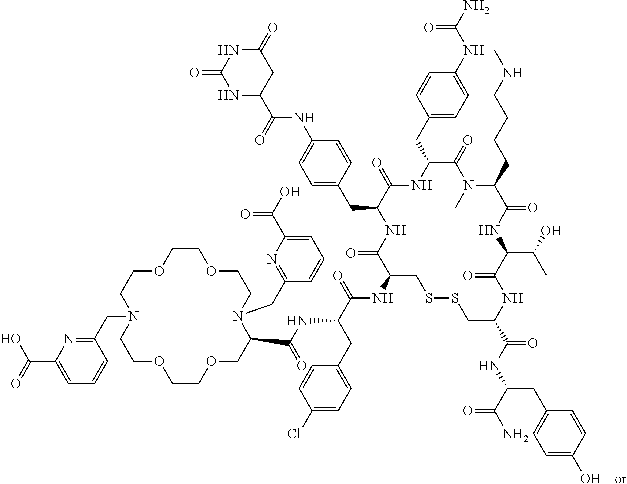 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)[C@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] | Int-21, peak 1 |
| | 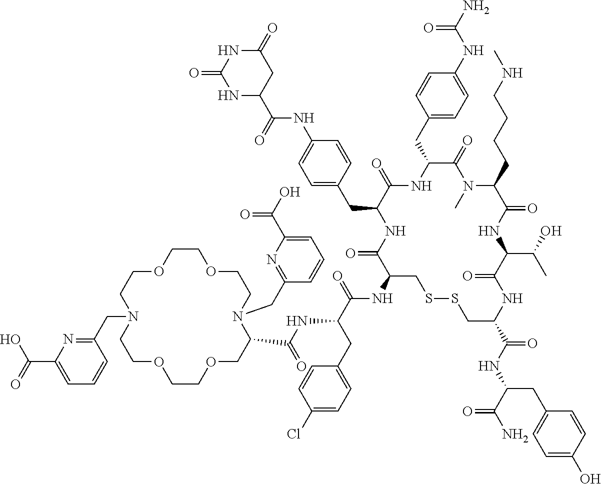 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)[C@@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] | |
| 29 | 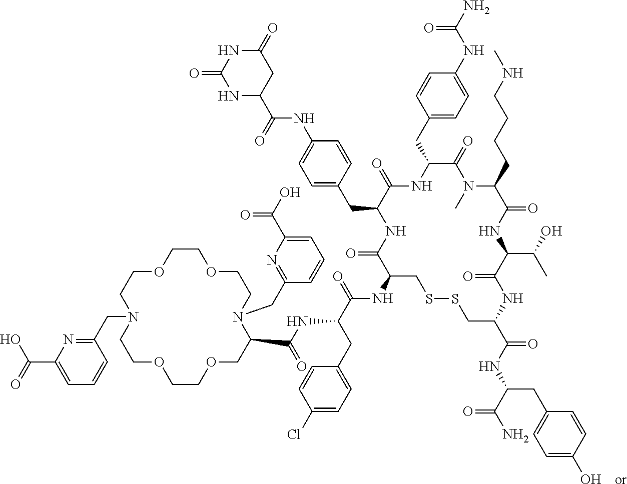 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)[C@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] | Int-21, peak 2 |
| | 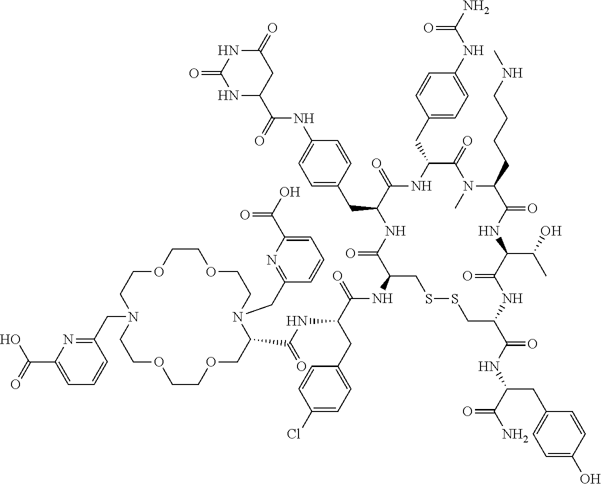 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)[C@@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] | |

### Preparation of Compound 24

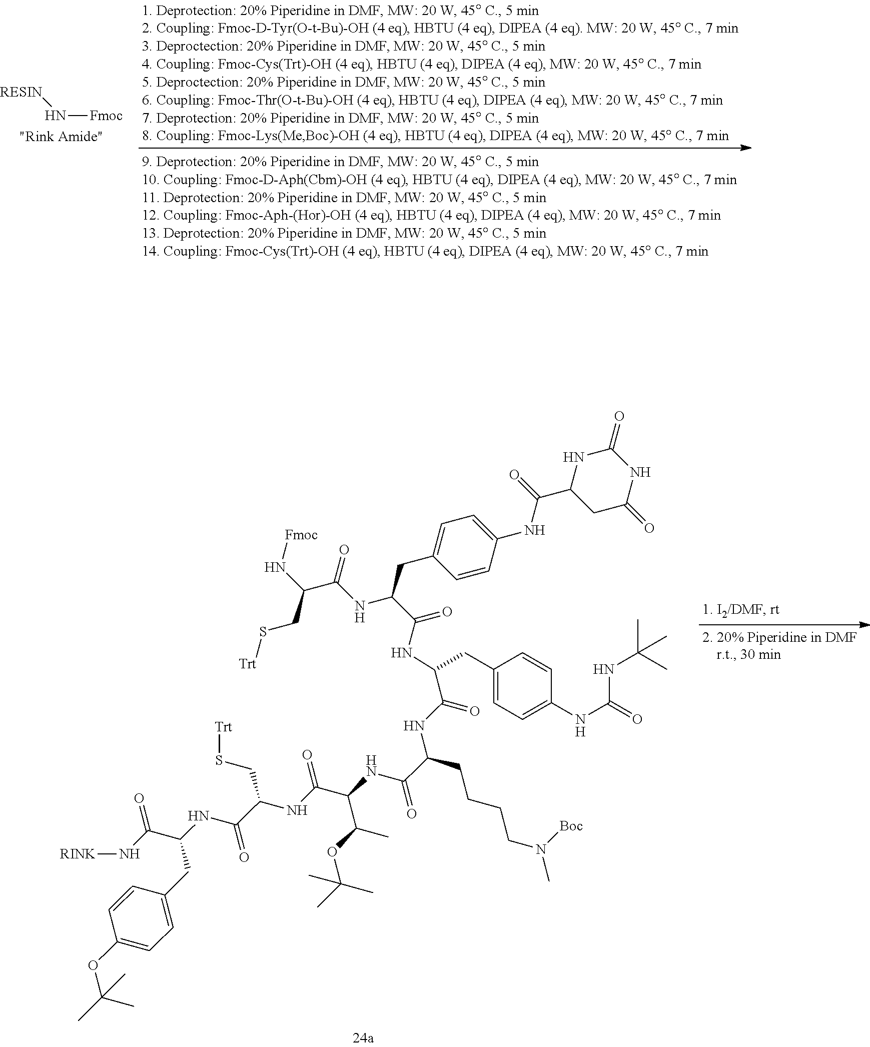

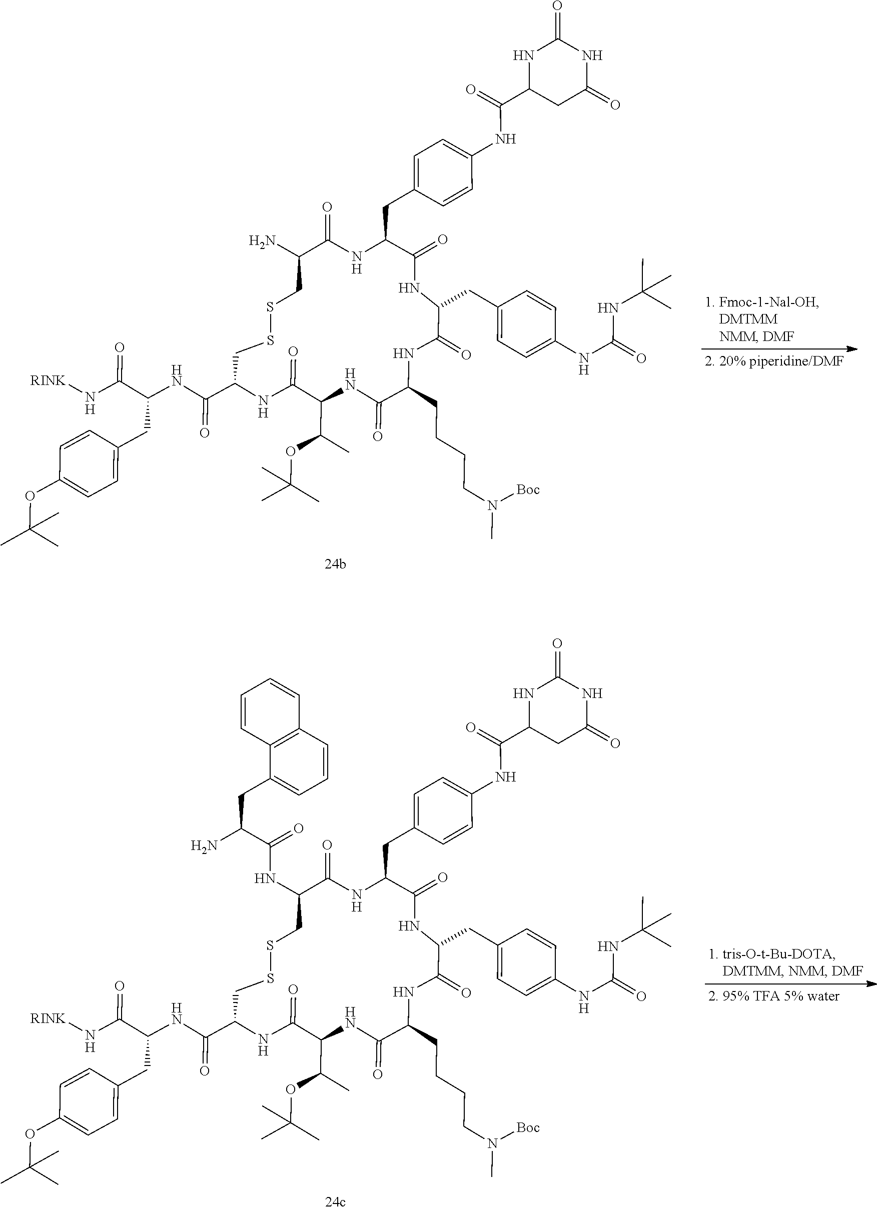

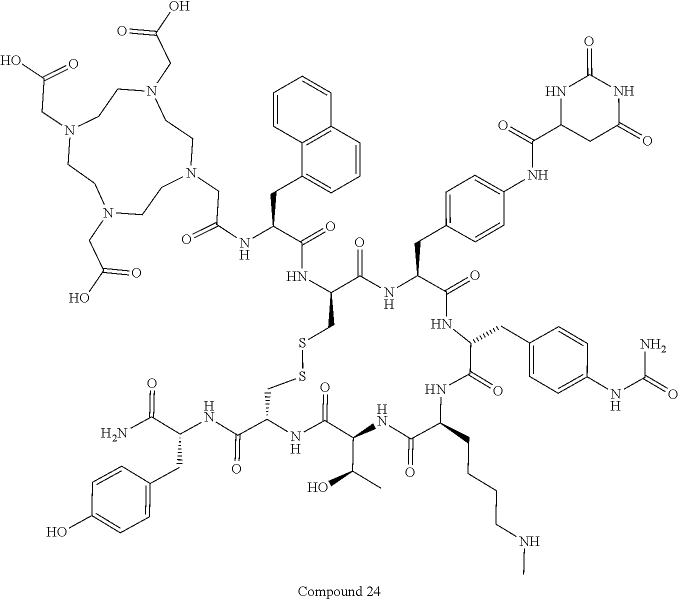 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2cccc3ccccc23)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O]

### Fmoc-D-Cys(Trt)-Aph(Hor)-D-Aph(Cbm)-Lys(Me,Boc)-Thr(O-t-Bu)-Cys(Trt)-D-Tyr(O-t-Bu)-RINK AM/IDE Resin (24a)

RINK AMIDE resin (0.1 mmol, Ambeed Cat #: A451407) was swelled in DMF (3 ml) for 30 minutes, then filtered. The Fmoc group was removed by treating the resin with 20% piperidine in DMF (3 ml) at 25° C. for 1 h, then the resin was washed with DMF (10 ml×5).

SPPS was carried out as follows. Coupling: Fmoc-AA-OH (4 eq.) was preactivated with HBTU (4 eq.) and DIPEA (4 eq.) in 5 ml of DMF for 1 min and added to resin and heated in microwave reactor at 20 W, 45° C. for 7 min with gentle stirring. The resin was washed with DMF (5 ml×5). Deprotection: 20% piperidine in DMF (5 ml) was added to resin and heated in microwave reactor at 20 W, 45° C. for 5 min with gentle stirring. The resin was washed with DMF (5 ml×5).

### Cyclo-[D-Cys(Trt)-Aph(Hor)-D-Aph(Cbm)-Lys(Me,Boc)-Thr(O-t-Bu)-Cys(Trt)]-D-Tyr(O-t-Bu)-RINK AMIDE Resin (24b)

Resin-peptide 24a (0.1 mmol) was suspended in DMF (5 ml). Iodine (5 eq) was added, and the mixture was gently stirred at 25° C. for 2 h, filtered and the resin was washed with DMF (5 ml×5) and DCM (5 ml×5). A solution of 20% piperidine in DMF (5 ml) was added and stirred at 25° C. for 30 min. The resin was washed with DMF (5 ml×5) to afford 24b (assume quant.). 1-Nal-cyclo-[D-Cys(Trt)-Aph(Hor)-D-Aph(Cbm)-Lys(Me,Boc)-Thr(O-t-Bu)-Cys(Trt)]-D-Tyr(O-t-Bu)-RINK AMIDE Resin (24c):

Resin bound 24b (0.012 mmol) was suspended in DMF (0.5 mL). To this was added Fmoc-1-Nal-OH (10.5 mg, 0.024 mmol, 2 eq), DMTMM (6.4 mg, 0.024 mmol, 2 eq), and N-methyl morpholine (2.6 μL, 0.024 mmol, 2 eq). The reaction mixture was stirred for 1 h at 25° C. The resin was washed with DMF (2 mL×5), then treated with 20% piperidine in DMF (5 ml) at 25° C. for 1 h, then the resin was washed with DMF (5 ml×5) afford 24c (assume quant.). DOTA-1-Nal-cyclo-[D-Cys(Trt)-Aph(Hor)-D-Aph(Cbm)-Lys(Me,Boc)-Thr(O-t-Bu)-Cys(Trt)]-D-Tyr(O-t-Bu)-NH₂ (Compound 24):

Resin bound 24c (0.012 mmol) was suspended in DMF (1 mL). To this were added dota-tris-(tert-butyl ester) (68.6 mg, 0.12 mmol, 10 eq), DMTMM (32.1 mg, 0.12 mmol, 10 eq), and N-methyl morpholine (13.2 μL, 0.12 mmol, 10 eq). The reaction mixture was stirred at 25° C. for 6 h. The resin was washed with DMF (2 mL×5), and DCM (2 mL×5) then treated with (95:5) (TFA:water) (2 ml) and heated at 45° C. for 1 h, filtered, and filtrate concentrated to dryness. The crude solid was dissolved in (3:1) (water:MeCN) (2 ml) and purified by reverse phase C18 flash chromatography (gradient elution; 10% MeCN/water—50% MeCN/water with 0.05% HCO₂H as mobile phase additive) to afford Compound 24 (2.0 mg, 0.0012 mmol, 10%) as a white solid. MS (ESI) m/z: 860.7(M+2H⁺)/2.

### Preparation of Compound 25

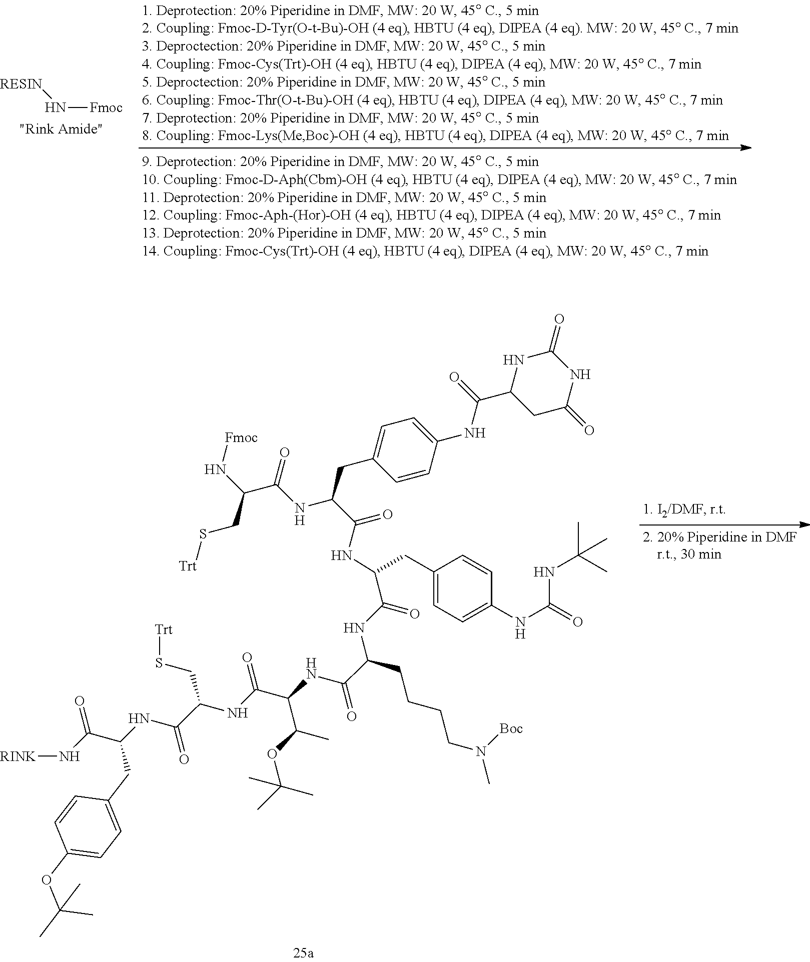

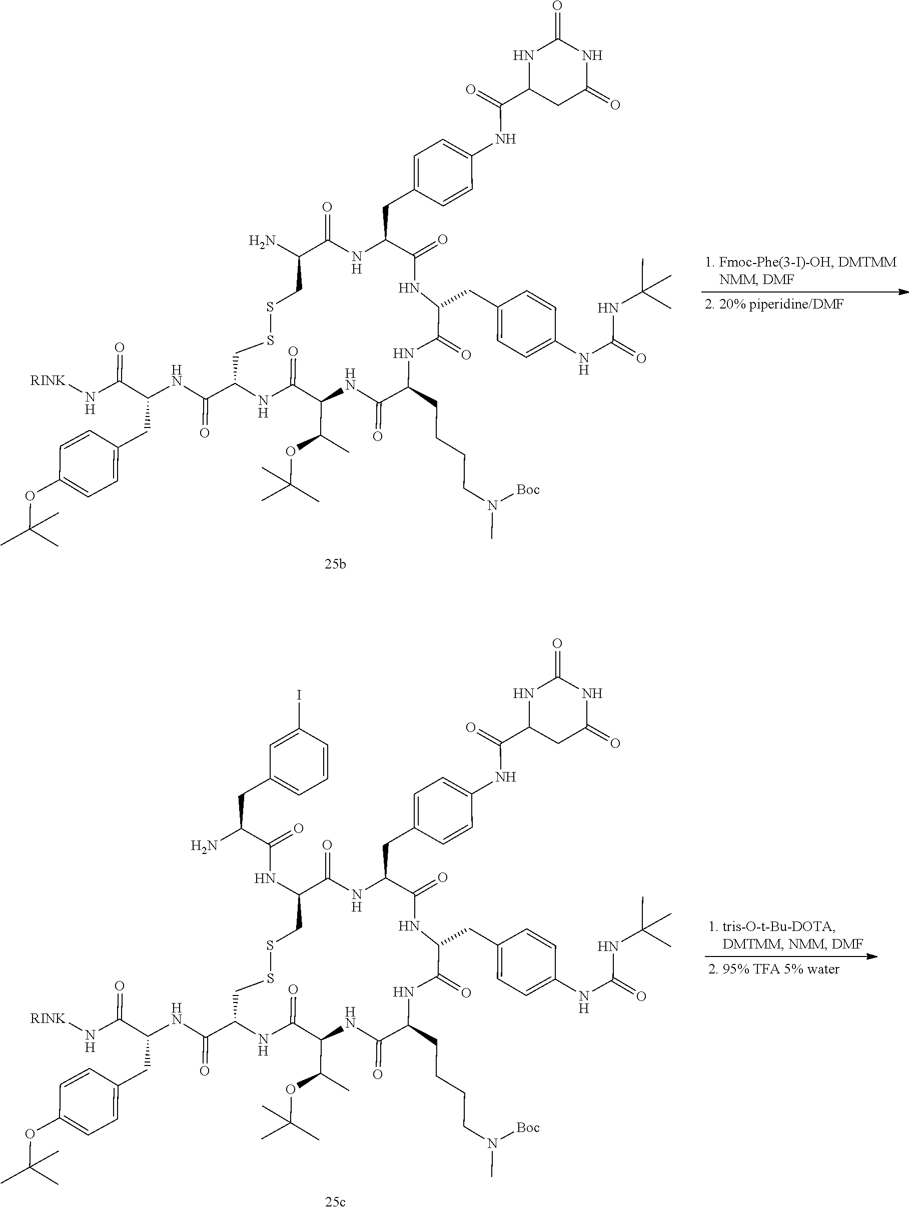

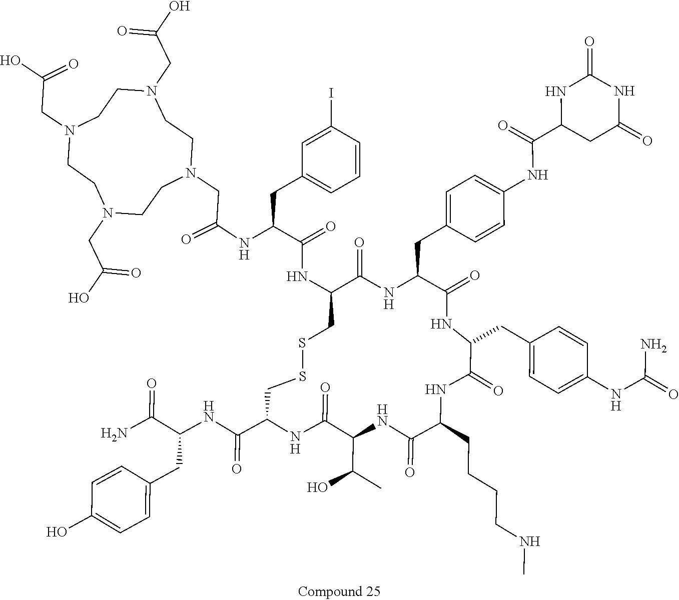 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2cccc(I)c2)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O]

### Fmoc-D-Cys(Trt)-Aph(Hor)-D-Aph(Cbm)-Lys(Me,Boc)-Thr(O-t-Bu)-Cys(Trt)-D-Tyr(O-t-Bu)-RINK AMIDE Resin (25a)

RINK AMIDE resin (0.1 mmol, Ambeed Cat #: A451407) was swelled in DMF (3 ml) for 30 minutes, then filtered. The Fmoc group was removed by treating the resin with 20% piperidine in DMF (3 ml) at 25° C. for 1 h, then the resin was washed with DMF (10 ml×5).

SPPS was carried out as follows. Coupling: Fmoc-AA-OH (4 eq.) was preactivated with HBTU (4 eq.) and DIPEA (4 eq.) in 5 ml of DMF for 1 min and added to resin and heated in microwave reactor at 20 W, 45° C. for 7 min with gentle stirring. The resin was washed with DMF (5 ml×5). Deprotection: 20% piperidine in DMF (5 ml) was added to resin and heated in microwave reactor at 20 W, 45° C. for 5 min with gentle stirring. The resin was washed with DMF (5 ml×5).

### Cyclo-[D-Cys(Trt)-Aph(Hor)-D-Aph(Cbm)-Lys(Me,Boc)-Thr(O-t-Bu)-Cys(Trt)]-D-Tyr(O-t-Bu)-RINK AMIDE Resin (25b)

Resin-peptide 25a (0.1 mmol) was suspended in DMF (5 ml). Iodine (5 eq) was added, and the mixture was gently stirred at 25° C. for 2 h, filtered and the resin was washed with DMF (5 ml×5) and DCM (5 ml×5). A solution of 20% piperidine in DMF (5 ml) was added and stirred at 25° C. for 30 min. The resin was washed with DMF (5 ml×5) to afford 25b (assume quant.).

### Phe(3-I)-cyclo-[D-Cys(Trt)-Aph(Hor)-D-Aph(Cbm)-Lys(Me,Boc)-Thr(O-t-Bu)-Cys(Trt)]-D-Tyr(O-t-Bu)-RINK AMIDE Resin (25c)

Resin bound 25b (0.012 mmol) was suspended in DMF (0.5 mL). To this was added Fmoc-Phe(3-I)—OH (12.3 mg, 0.024 mmol, 2 eq), DMTMM (6.4 mg, 0.024 mmol, 2 eq), and N-methyl morpholine (2.6 μL, 0.024 mmol, 2 eq). The reaction mixture was stirred for 1 h at 25° C. The resin was washed with DMF (2 mL×5), then treated with 20% piperidine in DMF (5 ml) at 25° C. for 1 h, then the resin was washed with DMF (5 ml×5) afford 25c (assume quant.).

### DOTA-Phe(3-I)-cyclo-[D-Cys(Trt)-Aph(Hor)-D-Aph(Cbm)-Lys(Me,Boc)-Thr(O-t-Bu)-Cys(Trt)]-D-Tyr(O-t-Bu)-NH₂ (Compound 25)

Resin bound 25c (0.012 mmol) was suspended in DMF (1 mL). To this were added dota-tris-(tert-butyl ester) (68.6 mg, 0.12 mmol, 10 eq), DMTMM (32.1 mg, 0.12 mmol, 10 eq), and N-methyl morpholine (13.2 μL, 0.12 mmol, 10 eq). The reaction mixture was stirred at 25° C. for 6 h. The resin was washed with DMF (2 mL×5), and DCM (2 mL×5) then treated with (95:5) (TFA:water) (2 ml) and heated at 45° C. for 1 h, filtered, and filtrate concentrated to dryness. The crude solid was dissolved in (3:1) (water:MeCN) (2 ml) and purified by reverse phase C18 flash chromatography (gradient elution; 10% MeCN/water—50% MeCN/water with 0.05% HCO₂H as mobile phase additive) to afford Compound 25 (0.8 mg, 0.00045 mmol, 4%) as a white solid. MS (ESI) m/z: 898.1 (M+2H⁺)/2.

### Preparation of Compound 28 and 29

Compound 28 and 29 were prepared according to the above procedure. The crude solid was dissolved in (3:1) (water:MeCN) (2 ml) and purified by reverse phase C18 flash chromatography (gradient elution; 10% MeCN/water—50% MeCN/water with 0.05% HCO₂H as mobile phase additive) to afford two species, compound 28 (0.4 mg, 2%, MS (ESI) m/z: 945.8 (M+2H⁺)/2, peak 1, 0.68 min rt) as a white solid and compound 29 (0.6 mg, 3%, MS (ESI) m/z: 945.8 (M+2H⁺)/2, peak 2, 0.75 min rt) as a white solid. rt).

### Preparation of Compound 30

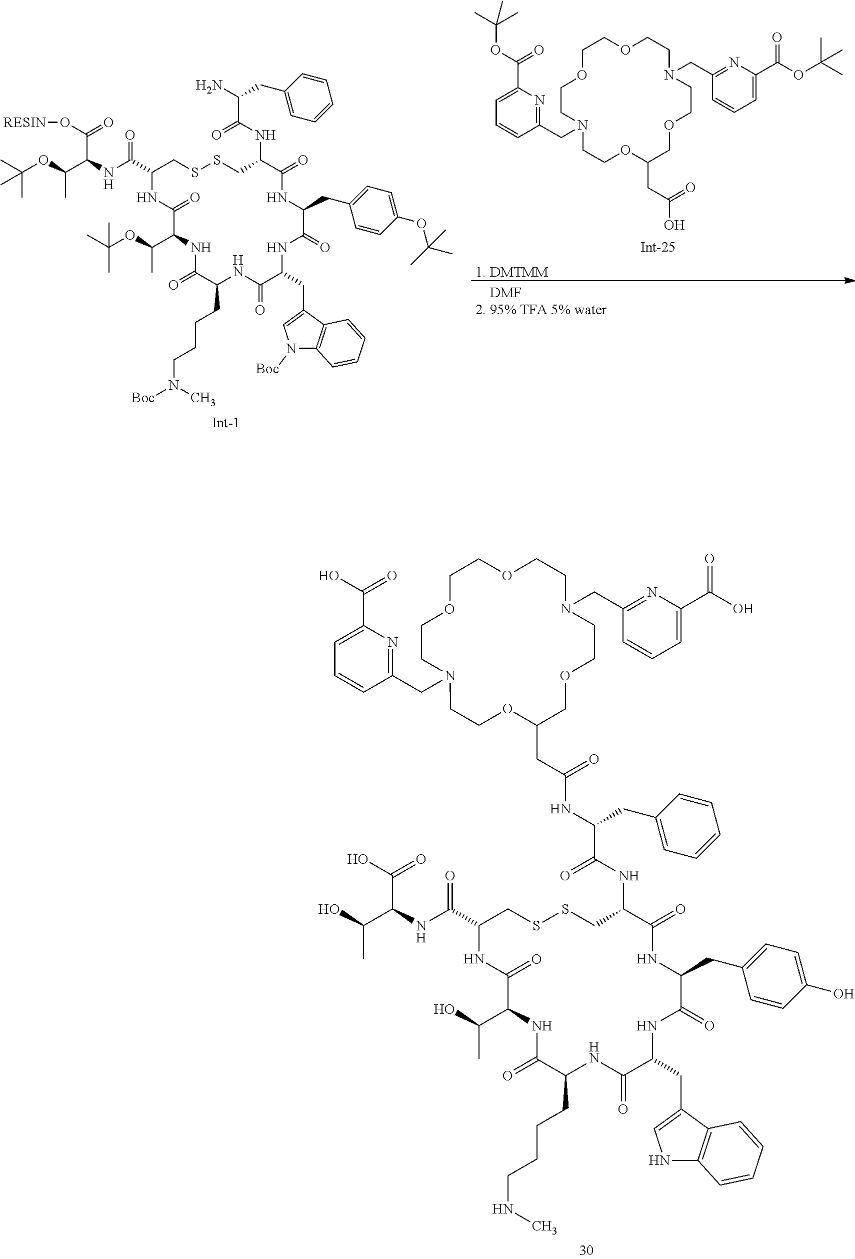

Int-1 (0.005 mmol) was suspended in DMF (1 mL). To this was added Int-25 (5.3 mg, 0.008 mmol, 1.5 eq), DMTMM (2.0 mg, 0.008 mmol, 1.5 eq), and N-methyl morpholine (0.8 μL, 0.008 mmol, 1.5 eq). The reaction mixture was stirred overnight at 25° C. The resin was washed with DMF (1 mL×5), and DCM (1 mL×5) then treated with (95:5) TFA/water (1.5 ml) at 45° C. for 1.5 h, filtered, and filtrate concentrated to dryness. The crude solid was dissolved in (3:1) (water:MeCN) (2 ml) and purified by reverse phase C18 flash chromatography (gradient elution; 1000 MeCN/water—5000 MeCN/water with 0.05% HCO₂H as mobile phase additive) to afford compound 30 (2.0 mg, 0.0012 mmol, 24%) as a white solid. MS (ESI) m/z: 818.2(M+2H⁺)/2.

The following compounds were prepared using procedures similar to those for Compound 30:

| Compound | | Starting |
|---|---|---|
| No. | Structure | Material |
| 34 | 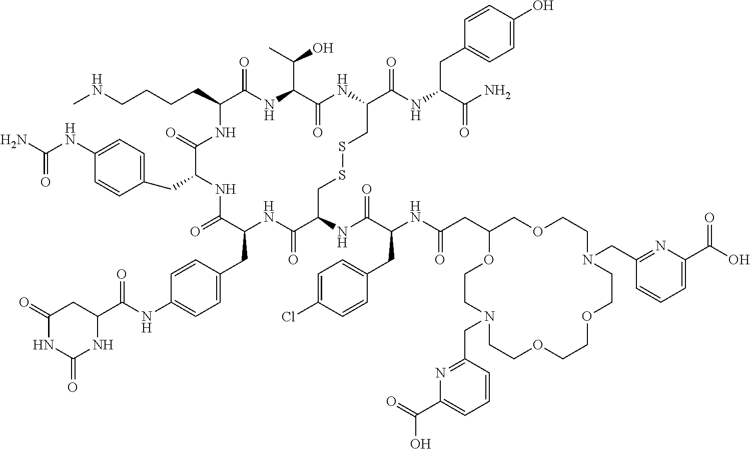 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2ccc(Cl)cc2)NC(=O)CC2COCCN(Cc3cccc(C(=O)O)n3)CCOCCOCCN(Cc3cccc(C(=O)O)n3)CCO2)CSSC[C@@H](C(=O)N[C@H](Cc2ccc(O)cc2)C(N)=O)NC(=O)[C@H]([C@@H](C)O)NC1=O] | Int-15 |
| 37 | 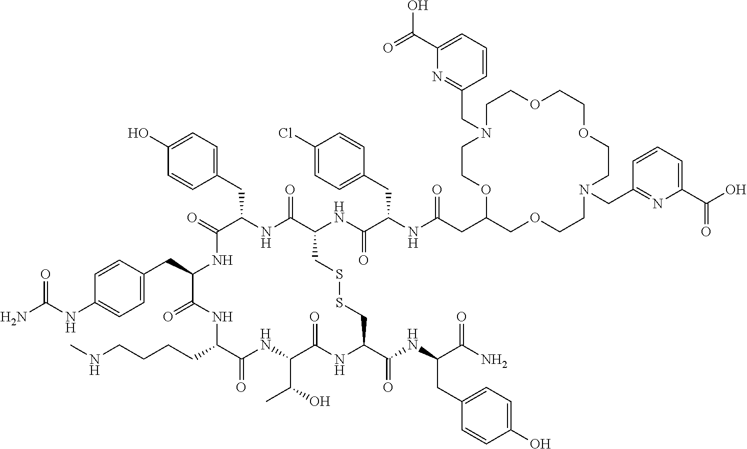 | Int-18 |

### Preparation of Compound 33

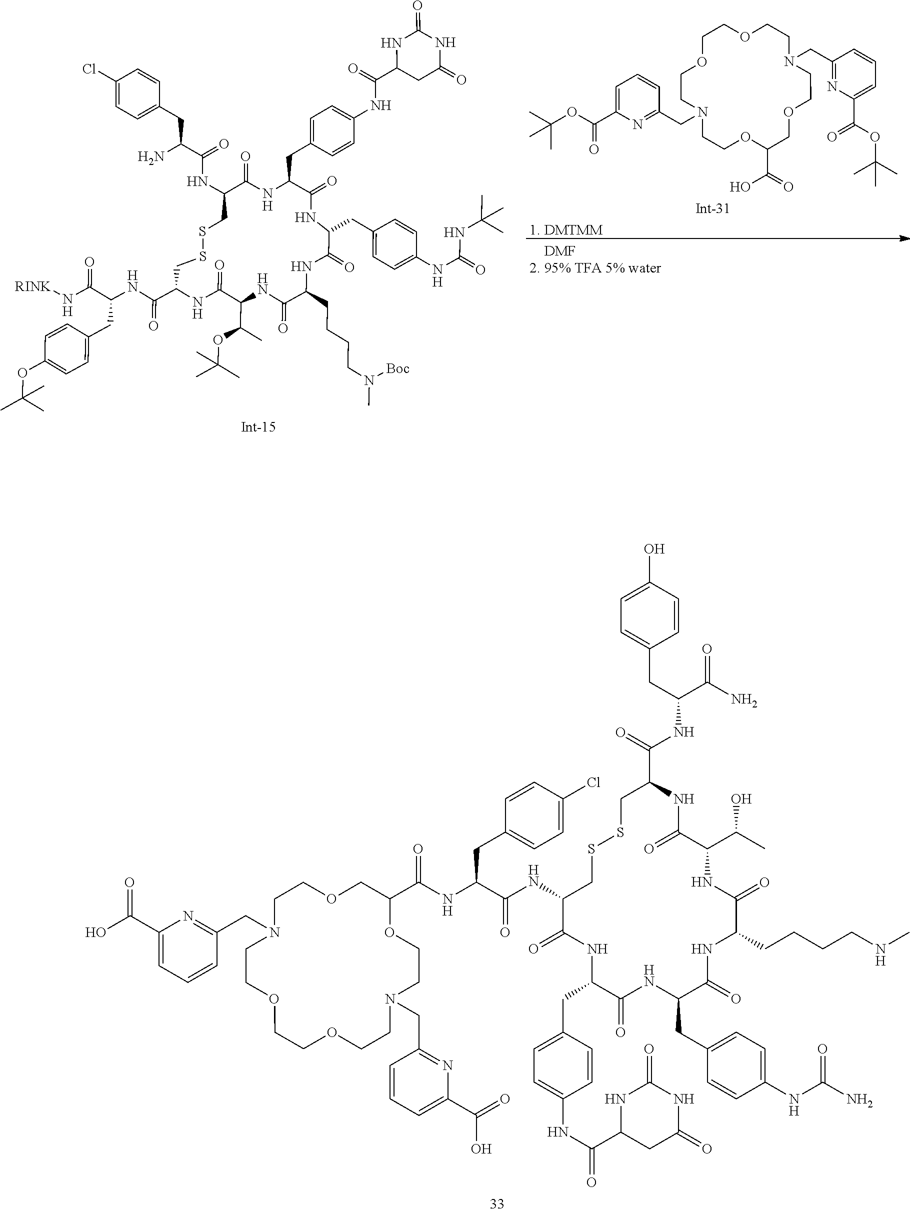

Int-15 (0.005 mmol) was suspended in DMF (1 mL). To this was added Int-31 (5.3 mg, 0.008 mmol, 1.5 eq), DMTMM (2.0 mg, 0.008 mmol, 1.5 eq), and N-methyl morpholine (0.8 μL, 0.008 mmol, 1.5 eq). The reaction mixture was stirred overnight at 25° C. The resin was washed with DMF (1 mL×5), and DCM (1 mL×5) then treated with (95:5) TFA/water (1.5 ml) at 45° C. for 1.5 h, filtered, and filtrate concentrated to dryness. The crude solid was dissolved in (3:1) (water:MeCN) (2 ml) and purified by reverse phase C18 flash chromatography (gradient elution; 1000 MeCN/water—50% MeCN/water with 0.05% HCO₂H as mobile phase additive) to afford compound 33.

The following compounds were prepared using procedures similar to those for Compound 33:

| Compound | | Starting |
|---|---|---|
| No. | Structure | Material |
| 35 | 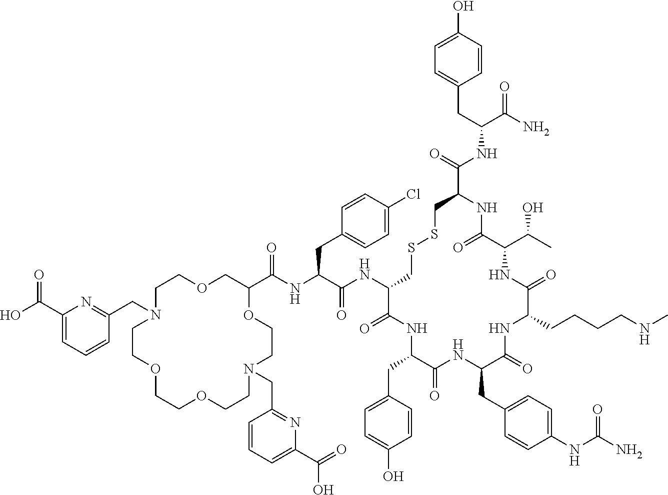 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2ccc(Cl)cc2)NC(=O)C2COCCN(Cc3cccc(C(=O)O)n3)CCOCCOCCN(Cc3cccc(C(=O)O)n3)CCO2)CSSC[C@@H](C(=O)N[C@H](Cc2ccc(O)cc2)C(N)=O)NC(=O)[C@H]([C@@H](C)O)NC1=O] | Int-18 |
| 36 | 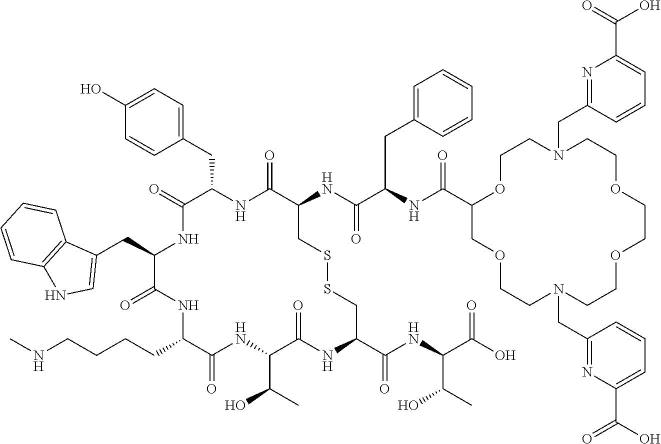 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)C2COCCN(Cc3cccc(C(=O)O)n3)CCOCCOCCN(Cc3cccc(C(=O)O)n3)CCO2)CSSC[C@@H](C(=O)N[C@@H](C(=O)O)[C@H](C)O)NC(=O)[C@H]([C@@H](C)O)NC1=O] | Int-27 |

Compounds 1a-40a can be prepared according to the above procedures using the corresponding amino acids. Compounds 41-62 are prepared according to the above procedures using standard deprotection and resin cleavage techniques. Compounds 41a to 62a can be prepared according to the above procedures using the corresponding amino acids and suitable deprotection and resin cleavage techniques.

### Example 3: Compounds of the Invention Demonstrate Low IC50 Values in Cell-Based Assays Displacing Radiolabeled DOTATATE

AR42J cells were cultured on poly-D-lysine coated 24-well plates at a density of 175,000 cells per well and incubated for 1-2 days. Prior to the experiment, the growth media (DMEM supplemented with 10% FBS and penicillin/streptomycin) was replaced with serum-free DMEM. Test compounds were prepared as serial 10-fold dilutions in DMSO, with final concentrations ranging from 0.01 nM to 10 μM. Radiolabeled compounds ([¹⁷⁷Lu]DOTATATE or [⁶⁸Ga]DOTATATE) were used at a final concentration of 0.2 nM, mixed with the respective dilutions of unlabeled compounds in pre-warmed DMEM, and added to the cells in a volume of 500 μL per well in triplicate. Control wells received the same volume of DMSO without unlabeled compounds. After a one hour incubation at 37° C., the medium was removed, and the cells were washed twice with ice-cold PBS. A solution of 500 μL of 1M NaOH was added to each well and incubated at room temperature for at least 5 minutes to lyse the cells. The contents of each well were transferred to pre-labeled gamma counter tubes, followed by an additional wash with 500 μL of PBS, which was also collected in the same tubes. Radioactivity was measured in counts per minute (CPM) using a gamma counter, with data corrected for decay and background. Results were normalized to wells without unlabeled compound. Data analysis was performed using GraphPad Prism.

These values are compiled in Table 3 below.

TABLE 3
Results for Binding Affinity Studies

| Compound No. | IC₅₀ (nM) | Compound No. | IC₅₀ (nM) |
|---|---|---|---|
| DOTA-JR11 | 2.0 | DOTATATE | — |
| 1 | 556 | 2 | 15 |
| 3 | >1000 | 4 | >1000 |
| 5 | 31 | 6 | 35 |
| 7 | 24 | 8 | 11 |
| 9 | 1.8 | 10 | 3.9 |
| 11 | 3.0 | 12 | 0.19 |
| 13 | 0.93 | 14 | 2.0 |
| 15 | 18 | 16 | 10 |
| 17 | 10 | 18 | 13 |
| 19 | 5.0 | 20 | 7.0 |
| 21 | 11 | 22 | 2.8 |
| 23 | 9.5 | 24 | 1.8 |
| 25 | 2.7 | 26 | 0.48 |
| 27 | 0.70 | 28 | 1.3 |
| 29 | 1.6 | 30 | 1.5 |
| 31 | 0.55 | 32 | >1000 |
| 33 | 0.79 | 34 | 1.3 |
| 35 | 0.56 | 36 | 1.9 |
| 37 | 0.9 | 38 | 7.9 |
| 39 | 0.6 | 40 | 2.0 |

### Example 4—Mouse Biodistribution Studies of the Compounds of the Invention

Female Nu/J mice were inoculated subcutaneously on the right flank with AR42J cells in F12 media. When the tumors reached a volume of 100-300 mm³, radiolabeled ligand at a mass dose of approximately 0.003 g to 0.3 g was administered intravenously (IV) via tail vein. At various time points post-injection, mice were humanely euthanized via CO₂ asphyxiation and tissue samples (blood, bone (femur), heart, lungs, liver, spleen, pancreas, both kidneys, adrenals, stomach, small intestines (including contents), large intestines (including contents), muscle (quadriceps), tumor, and tail) were resected, weighed and counted with a gamma counter. The activity of each collected tissue was measured in units of counts per minute (CPM). Triplicate aliquots of the radiotracer were also assayed in the gamma counter to calculate a factor for converting counts to units of activity (μCi/CPM). Values were decay corrected to the time of injection and corrected for background radiation.

The values for kidney and tumor accumulation are shown in Table 4 below. Compounds 1-8, 11-13, 23-27 and 38-40 were radiolabeled using ¹⁷⁷Lu. Compounds 9, 10, 14, 16-22, 28-30, and 33-37 were radiolabeled using ²²⁵Ac. DOTA-JR11 and DOTATATE are state of the art compounds having the structures shown below:

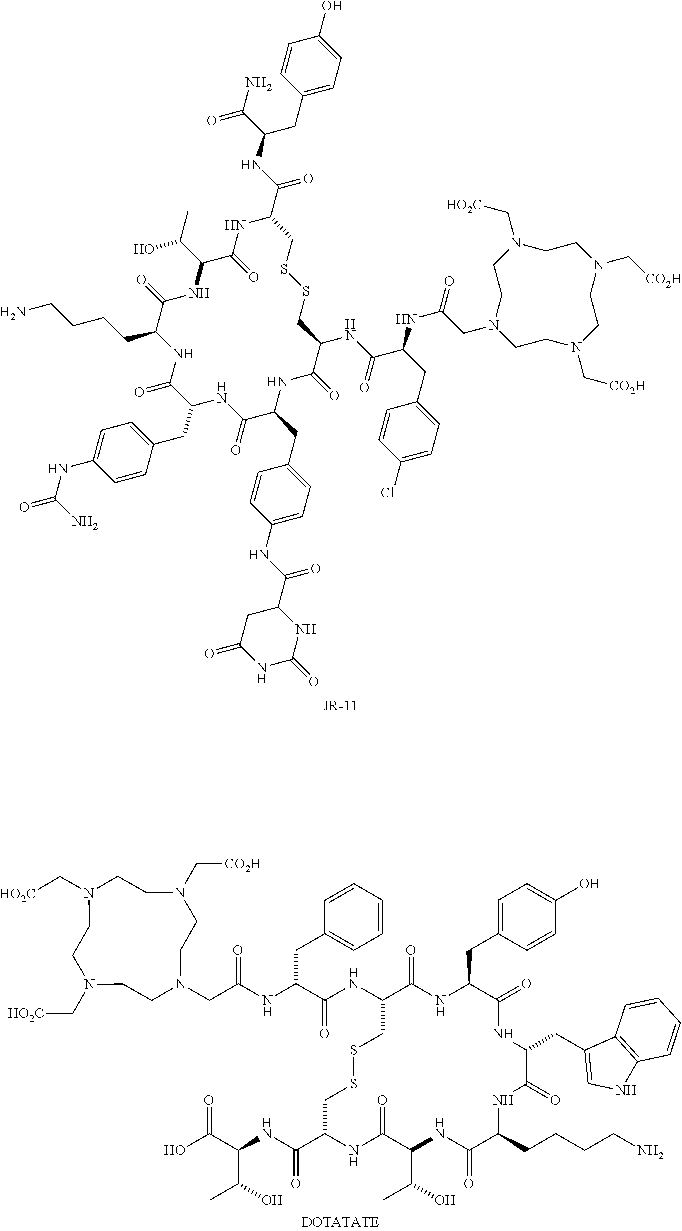

TABLE 4
Results for Biodistribution Studies

| | | | | | Tumor/ | Tumor/ |
|---|---|---|---|---|---|---|
| | Tumor | Tumor | Kidney | Kidney | Kidney | Kidney |
| Compound | Activity | Activity | Activity | Activity | Ratio | Ratio |
| No. | (1 h) | (24 h) | (1 h) | (24 hr) | (1h) | (24 h) |
| DOTA-JR11 | 31.73 | 31.51 | 8.26 | 6.76 | 3.84 | 4.66 |
| DOTATATE | 20.34 | 11.67 | 7.22 | 4.40 | 2.82 | 2.65 |
| 1 | 0.43 | 0.16 | 3.16 | 1.57 | 0.14 | 0.10 |
| 2 | 23.59 | 10.68 | 2.34 | 1.26 | 10.08 | 8.48 |
| 3 | 0.33 | 0.11 | 3.23 | 1.78 | 0.10 | 0.06 |
| 4 | 0.19 | 0.06 | 2.62 | 1.40 | 0.07 | 0.04 |
| 5 | 2.11 | 0.69 | 5.41 | 3.07 | 0.39 | 0.22 |
| 6 | 23.91 | 9.30 | 3.03 | 1.46 | 7.89 | 6.37 |
| 7 | 20.90 | 8.61 | 2.95 | 1.66 | 7.08 | 5.19 |
| 8 | 30.72 | 12.92 | 3.33 | 2.11 | 9.23 | 6.12 |
| 9 | 17.64 | 9.51 | 4.91 | 0.88 | 3.59 | 10.81 |
| 10 | 18.65 | 6.76 | 38.4 | 0.51 | 0.49 | 13.25 |
| 11 | 26.43 | 14.34 | 3.19 | 1.93 | 8.29 | 7.43 |
| 12 | 35.80 | 25.91 | 2.92 | 2.13 | 12.26 | 12.16 |
| 13 | 36.70 | 22.70 | 3.11 | 1.66 | 11.80 | 13.67 |
| 14 | 32.22 | 17.36 | 5.09 | 2.46 | 6.33 | 7.06 |
| 16 | 30.85 | 22.44 | 6.16 | 3.16 | 5.01 | 7.10 |
| 17 | 35.96 | 35.78 | 6.95 | 2.32 | 5.17 | 15.42 |
| 18 | 21.26 | 10.71 | 7.35 | 1.82 | 2.89 | 5.88 |
| 19 | 23.00 | 15.80 | 7.00 | 1.01 | 3.29 | 15.64 |
| 20 | 21.90 | 9.47 | 10.65 | 0.65 | 2.06 | 14.57 |
| 21 | 21.09 | 26.28 | 5.70 | 1.46 | 3.70 | 18.00 |
| 22 | 22.40 | 10.87 | 17.69 | 0.91 | 1.27 | 11.95 |
| 23 | 15.62 | 8.84 | 3.05 | 1.95 | 5.12 | 4.53 |
| 24 | 54.26 | 40.91 | 3.50 | 1.75 | 15.50 | 23.38 |
| 25 | 43.92 | 37.91 | 3.58 | 1.82 | 12.27 | 20.83 |
| 26 | 72.85 | 43.08 | 4.27 | 2.42 | 17.06 | 17.80 |
| 27 | 55.46 | 38.34 | 5.07 | 3.46 | 10.94 | 11.08 |
| 28 | 22.06 | 17.12 | 7.01 | 3.99 | 3.15 | 4.29 |
| 29 | 26.35 | 16.89 | 6.40 | 3.95 | 4.12 | 4.28 |
| 30 | 21.93 | 14.81 | 3.94 | 0.77 | 5.57 | 19.23 |
| 33 | 23.82 | 19.03 | 4.60 | 3.00 | 5.18 | 6.34 |
| 34 | 30.36 | 14.96 | 5.00 | 2.37 | 6.07 | 6.31 |
| 35 | 34.62 | 28.01 | 5.70 | 2.53 | 6.07 | 11.07 |
| 36 | 21.37 | 11.48 | 3.55 | 0.92 | 6.02 | 12.48 |
| 37 | 20.89 | 9.65 | 5.92 | 2.28 | 3.53 | 4.23 |
| 38 | 12.74 | 6.05 | 1.93 | 1.03 | 6.60 | 5.87 |
| 39 | 51.93 | 40.73 | 2.90 | 1.78 | 17.91 | 22.88 |
| 40 | 34.80 | 34.18 | 3.95 | 2.44 | 8.81 | 14.01 |

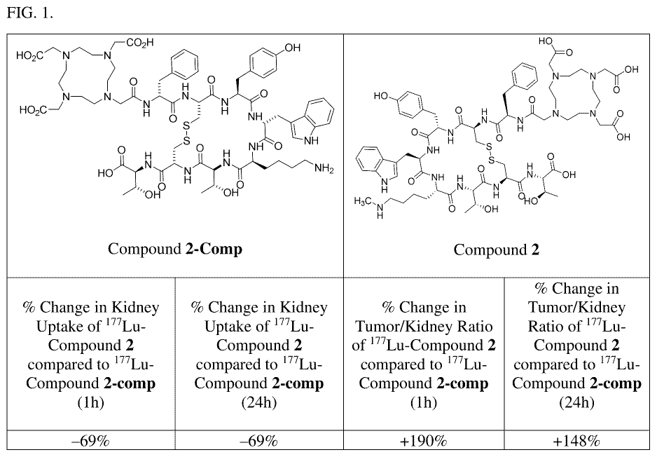

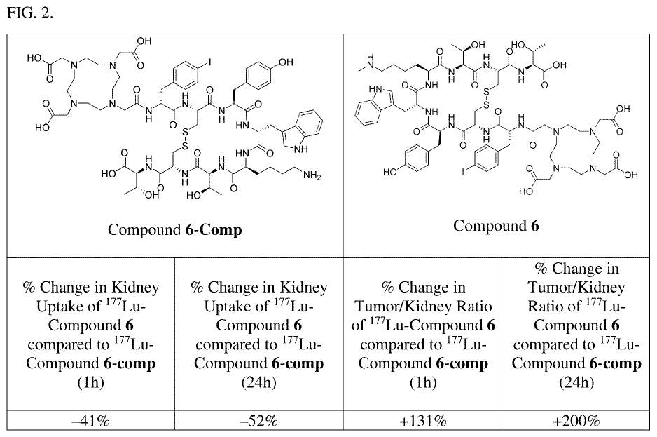

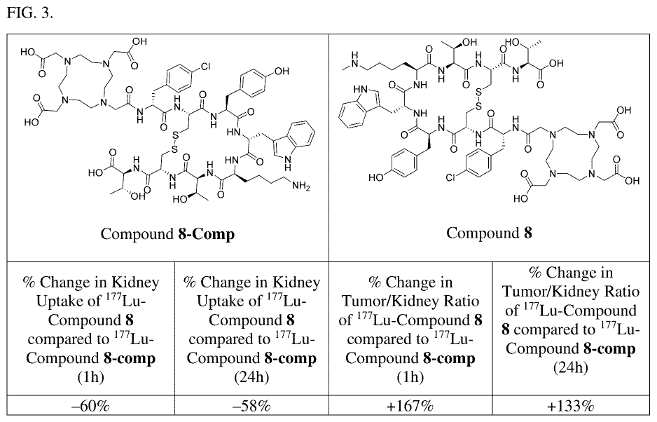

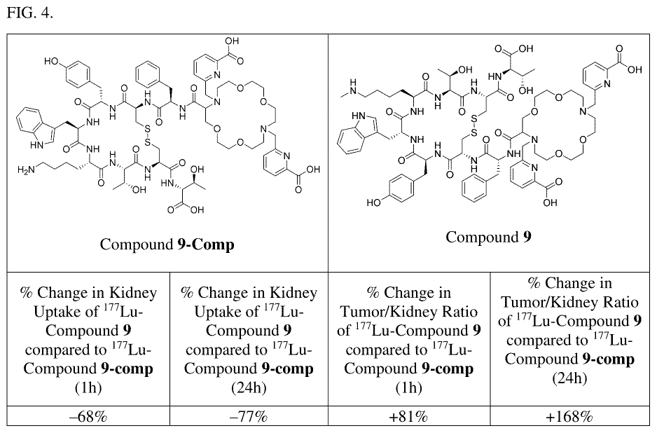

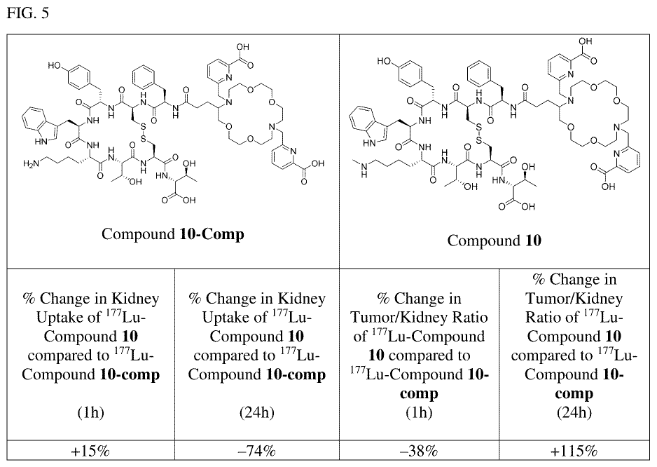

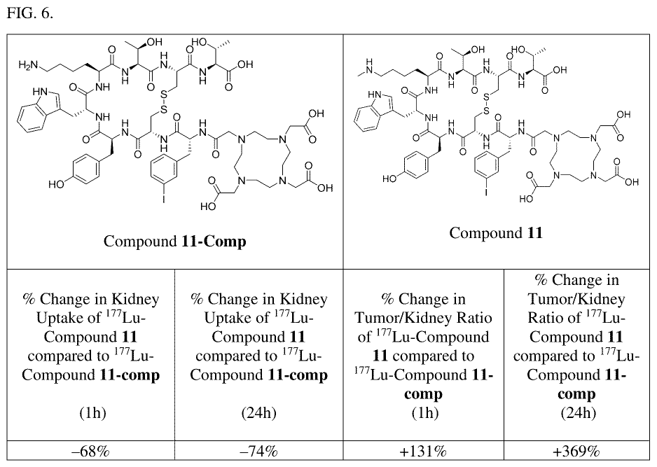

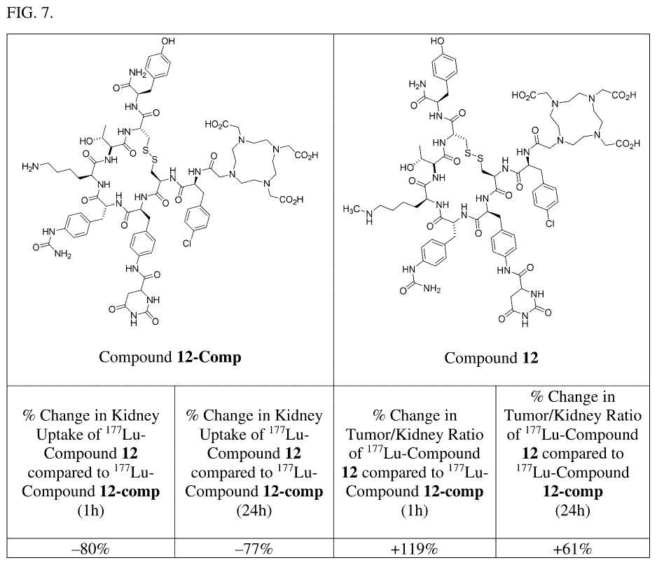

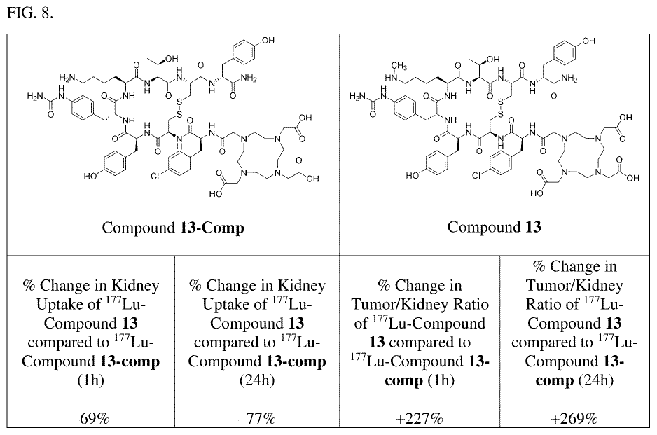

All of the tested compounds except for 1, 3-5, 9, 10, 18-22, 28 and 37 demonstrate improved tumor to kidney ratios compared to DOTATATE and DOTA-JR11 at 1 hour. Meanwhile, all of the tested compounds except for compounds 3-5, 23, 28, 29, and 37 demonstrate improved tumor to kidney ratios compared to DOTATATE and DOTA-JR11 at 24 hours. Additionally, as shown in FIGS. 1-8, the biodistribution of compounds 2, 6, and 8-13 showed reduced kidney uptake and improved tumor to kidney ratios compared to control compounds having an unmethylated lysine moiety. This biodistribution data demonstrates improved selectivity for the tumor relative to other organs, such as the kidneys.

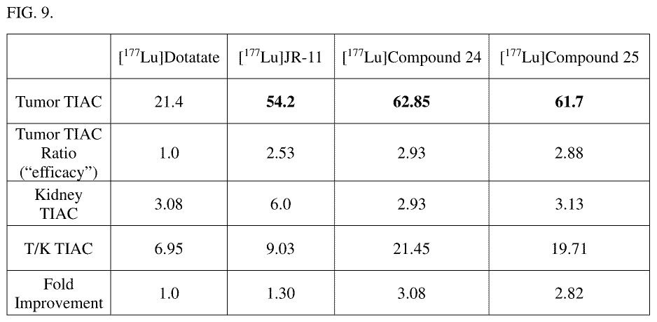

Moreover, the tissue time-activity coefficients described in Table 4 were fit with exponential models (mono-exponential, bi-exponential, and rise-fall). The model yielding the highest R² value was selected to compute the time-integrated activity coefficient (TIAC) which are proportional to the total radiation dose per gram to the tissues of interest. As shown in FIG. 9, the present compounds have demonstrated an increase in tumor uptake and/or a reduction in kidney uptake as compared to DOTATATE and DOTA-JR11. Specifically, [¹⁷⁷Lu]Compound 24 and [¹⁷⁷Lu]Compound 25 demonstrate about a 3-fold improvement over [¹⁷⁷Lu]DOTATATE in tumor uptake and [¹⁷⁷Lu]Compound 24 and [¹⁷⁷Lu]Compound 25 demonstrate a modest improvement in improved tumor uptake and about a 2-fold reduction in kidney uptake over [¹⁷⁷Lu]DOTA-JR11. This combined improvement of increased tumor uptake and reduced kidney uptake affords about a 3-fold improvement over the current state of the art compounds, [¹⁷⁷Lu]DOTATATE and [¹⁷⁷Lu]DOTA-JR11.

### Example 5—Mouse Efficacy Studies of the Compounds of the Invention

Female Nu/J mice were inoculated subcutaneously on the right flank with AR42J cells in F12 media. When the tumors reached a volume of 100-300 mm³, [¹⁷⁷Lu]DOTATATE, [¹⁷⁷Lu]DOTA-JR11, and [¹⁷⁷Lu]Compound 24 were dosed at 7.5, 15.0, and 30.0 MBq and administered intravenously (IV) via tail vein. The mice were then monitored for survivability daily and tumor measurements were taken every 2-3 days. When tumors reached 2000 mm³, mice were humanely euthanized via CO2 asphyxiation.

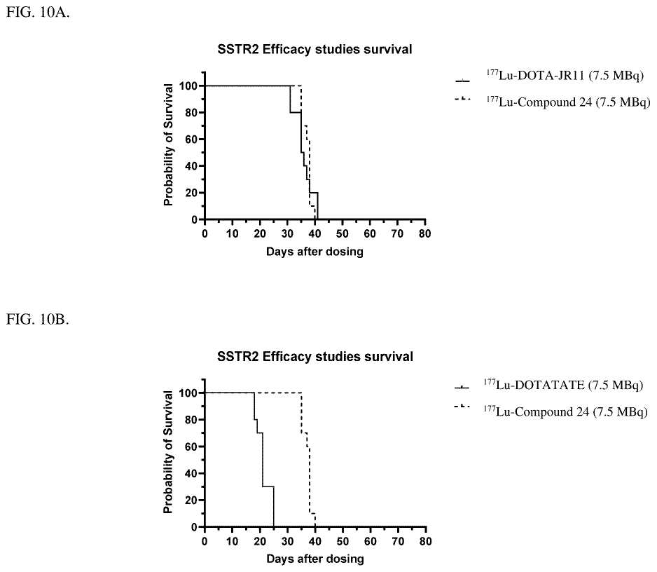

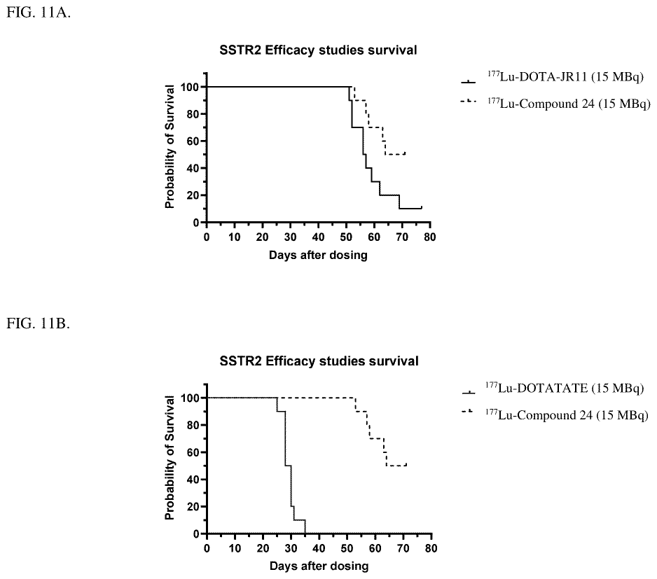

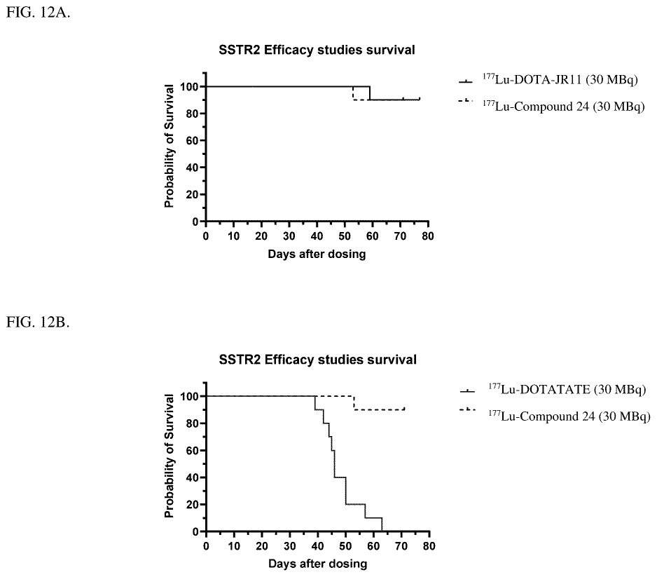

As shown in FIGS. 10-12, [¹⁷⁷Lu]Compound 24 demonstrated improved survivability of the mice at all dosages compared to [¹⁷⁷Lu]DOTATATE. In addition, [¹⁷⁷Lu]Compound 24 demonstrated similar or modest improvements in survivability of the mice at all dosages compared to [¹⁷Lu]DOTA-JR11.

### EQUIVALENTS

While certain embodiments have been illustrated and described a person with ordinary the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only and is not intended to be limiting. Thus, it is intended that the specification be considered as exemplary only with the breadth, scope and spirit of the present technology indicated only by the appended claims, definitions therein and any equivalents thereof.

All publications, patents, and other documents referred to in this specification are herein incorporated by reference in its entirety.

1. A compound represented by structural formula (I) 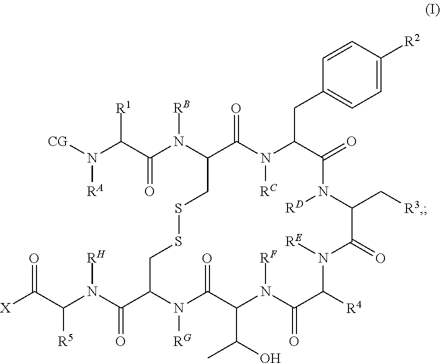
or a pharmaceutically acceptable salt thereof, wherein:
CG is a chelating group, an optical dye or fluorophore, a cytotoxic agent, or an immune stimulant;
X is OH or NH₂;
R^{A}, R^{B}, R^{C}, R^{D}, R^{E}, R^{F}, R^{G}, and R^{H} are each independently selected from hydrogen and C₁₋₄ alkyl;
R¹ is C₁₋₆ alkyl or phenyl, wherein said C₁₋₆ alkyl represented by R¹ is substituted with one R¹⁰;
R¹⁰ is independently selected from phenyl and naphthyl, wherein said phenyl or naphthyl represented by R¹⁰ is optionally substituted with one or more groups selected from halogen;
R² is OH or 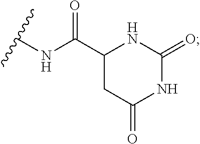 [SMILES: *NC(=O)C1CC(=O)NC(=O)N1]
R³ is 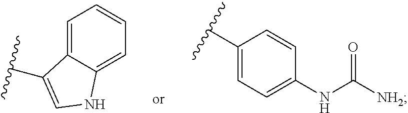 [SMILES: *c1c[nH]c2ccccc12 ; *c1ccc(NC(N)=O)cc1]
R⁴ is C₁₋₆ alkyl-NR^{4a}R^{4b} or C₁₋₆ alkyl-NHC(═NH)NH₂, wherein said C₁₋₆ alkyl in the group represented by R⁴ is optionally substituted with one or more halogen or C₁₋₃ alkyl;
R^{4a} is hydrogen or C₁₋₃ alkyl and R^{4b} is hydrogen, C₁₋₃ alkyl, C(O)C₁₋₃ alkyl and C(O)C₁₋₃ haloalkyl, provided that R^{4a} and R^{4b} are not both hydrogen;
R⁵ is C₁₋₆ alkyl or C₁₋₆ aralkyl, wherein the alkyl represented by R⁵ or the aryl portion of the aralkyl represented by R⁵ are independently substituted with one or more halogen, —OH, or C₁₋₆ alkoxy.

2. The compound of claim 1, wherein the compound is represented by:
(i) structural formula (IIa), (IIb), or (IIc): 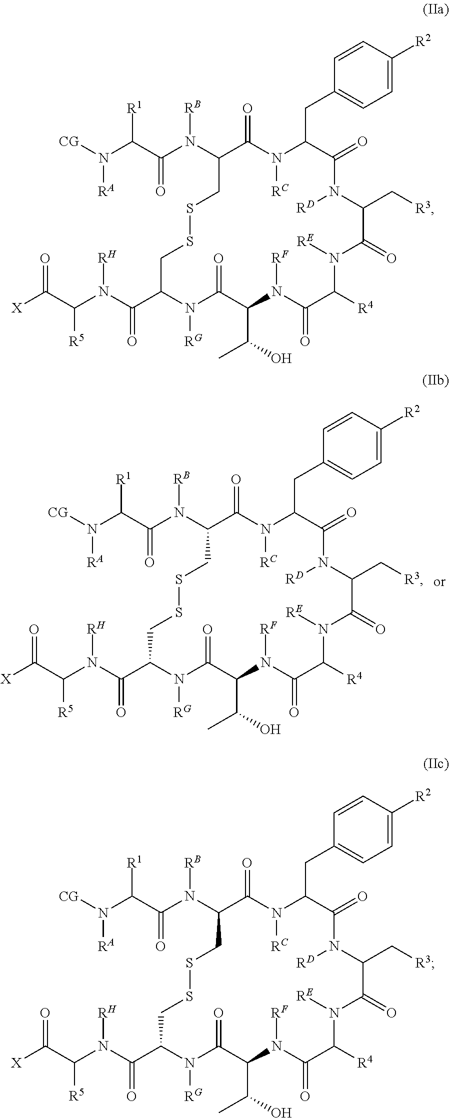
or a pharmaceutically acceptable salt thereof,
(ii) structural formula (IIIa), (IIIb), or (IIIc): 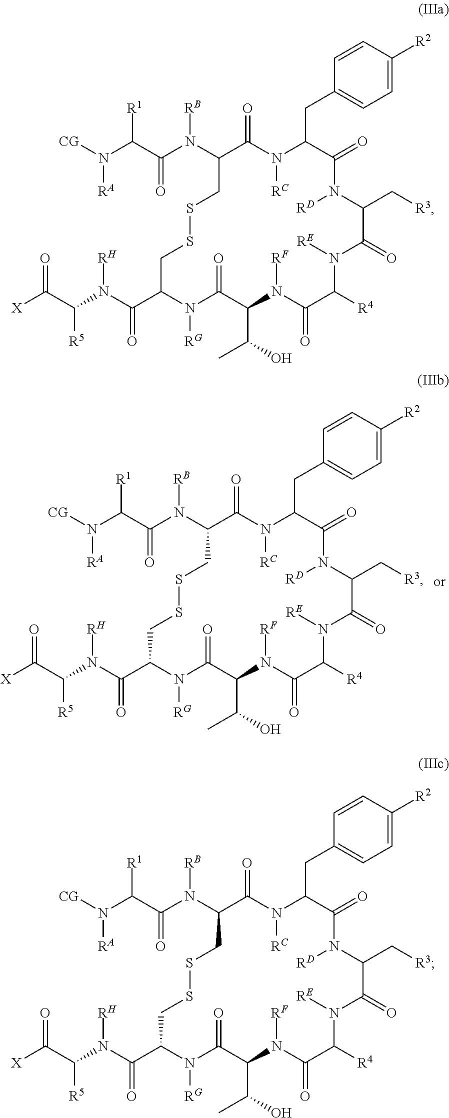
or a pharmaceutically acceptable salt thereof; or
(iii) structural formula (IVa), (IVb), or (IVc): 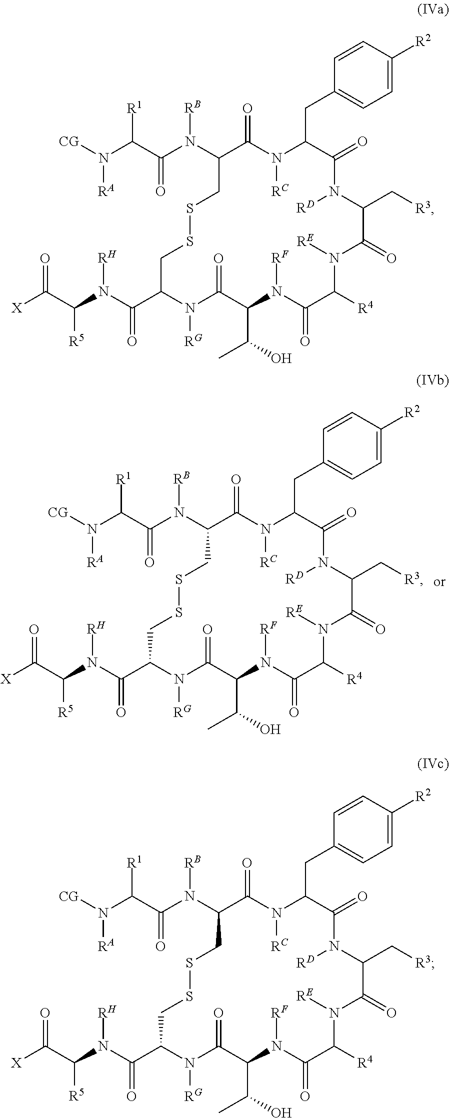
or a pharmaceutically acceptable salt thereof.

3. (canceled)

4. (canceled)

5. The compound of claim 1, or a pharmaceutically acceptable salt thereof, wherein R⁴ is C₁₋₄ alkyl-NR^{4a}R^{4b} and wherein R^{4a} is hydrogen and R^{4b} is C₁₋₃ alkyl.

6. (canceled)

7. (canceled)

8. The compound of claim 1, or a pharmaceutically acceptable salt thereof, wherein R¹ is:
(i) C₁₋₃ alkyl substituted with one R¹⁰ and wherein R¹⁰ is phenyl or naphthyl, wherein said phenyl is optionally substituted with one to five halogen,
(ii) selected from the group consisting of 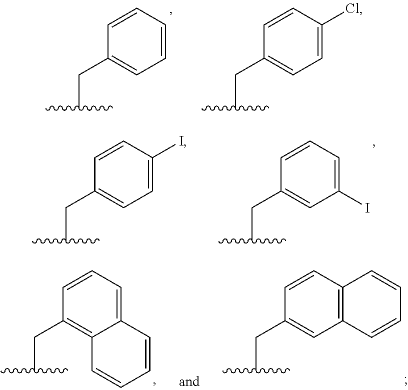
or
(iii) selected from the group consisting of

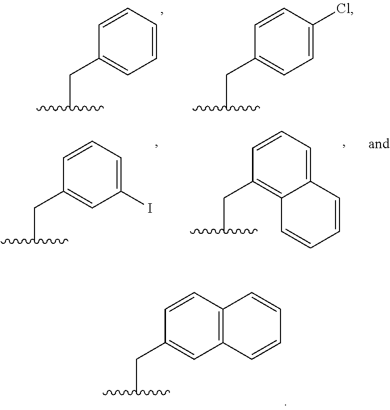

9-15. (canceled)

16. The compound of claim 1, wherein R⁵ is:
(i) C₁₋₃ alkyl or C₁₋₃ aralkyl, wherein the alkyl represented by R⁵ is substituted with —OH and the aryl portion of the aralkyl represented by R⁵ is substituted with one F, Cl, or —OH;

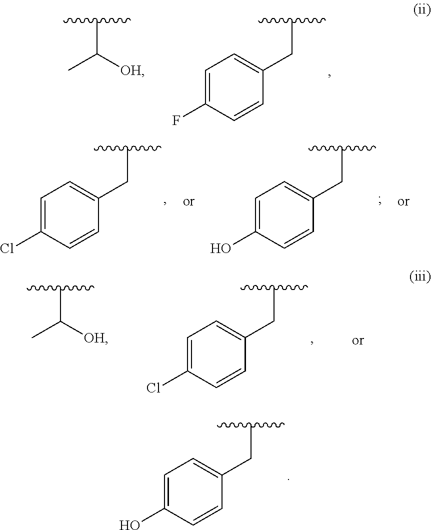

17. (canceled)

18. (canceled)

19. The compound of claim 1, wherein:
(i) R^{A}, R^{B}, R^{C}, R^{D}, R^{E}, R^{F}, R^{G}, and R^{H} are each independently selected from hydrogen or CH₃; or
(ii) R^{E} is hydrogen or CH₃ and R^{A}, R^{B}, R^{C}, R^{D}, R^{F}, R^{G}, and R^{H} are each hydrogen.

20. (canceled)

21. The compound of claim 1, or a pharmaceutically acceptable salt thereof, wherein CG is a fluorophore or an optical dye, wherein the fluorophore is 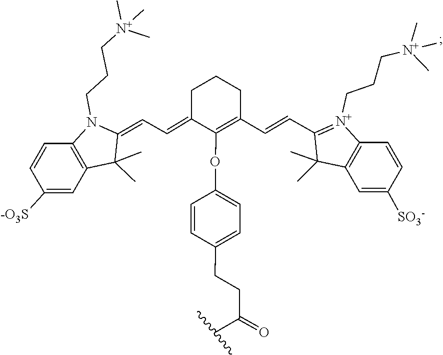
and the optical dye is selected from the group consisting of: a carbocyanine, indocarbocyanine, oxacarbocyanine, thiacarbocyanine, merocyanine, polymethine, coumarin, rhodamine, xanthene, fluorescein, Borodipyrromethane (BODIPY), VivoTag-680, VivoTag-S750, AlexaFluor dyes (e.g., AlexaFluor660, AlexaFluor680, AlexaFluor700, AlexaFluor750, AlexaFluor790) and DylightFluor dyes.

22. (canceled)

23. The compound of claim 1 or a pharmaceutically acceptable salt thereof, wherein CG is a chelating group that is the residue of a chelating agent, wherein the residue of a chelating agent selected from 1,4,7-triazacyclononane-1,4,7-triacetic acid (NOTA), p-SCN-Bn-NOTA, NODAGA (2-(4,7-bis(carboxymethyl)-1,4,7-triazonin-1-yl)pentanedioic acid), 1,4,7,10-tetraazacyclododecane-1,4,7,10-tetraacetic acid (DOTA), DOTAGA (2-(4,7,10-tris(carboxymethyl)-1,4,7,10-tetraazacyclododecan-1-yl)pentanedioic acid), p-SCN-Bn-DOTA (also known as 2B-DOTA-NCS),PIP-DOTA, diethylenetriaminepentaacetic acid (DTPA), PIP-DTPA, AZEP-DTPA, ethylenediamine tetraacetic acid (EDTA), triethylenetetraamine-N,N,N′,N″,N′″,N′″-hexa-acetic acid (TTHA), 7-[2-(bis-carboxymethylamino)-ethyl]-4,10-bis-carboxymethyl-1,4,7,10-tetraaza-cyclododec-1-yl-acetic acid (DEPA), 2,2′,2″-(10-(2-(bis(carboxymethyl)amino)-5-(4-isothiocyanatophenyl) pentyl)-1,4,7,10-tetraazacyclododecane-1,4,7-triyl)triacetic acid (3p-C-DEPA-NCS), NETA,{4-carboxymethyl-7-[2-(carboxymethylamino)-ethyl]-perhydro-1,4,7-triazonin-1-yl}-acetic acid (NPTA), diacetylpyridinebis(benzoylhydrazone), 1,4,7,10,13,16-hexaazacyclooctadecane N,N′,N″,N′″,N″″,N′″″-hexaaceticacid (HEHA), octadentate terephthalamide ligands, 2,2′-(4-(2-(bis(carboxymethyl)amino)-5-(4-isothiocyanatophenyl)pentyl)-10-(2-(bis(carboxymethyl)amino)ethyl)-1,4,7,10-tetraazacyclododecane-1,7-diyl)diacetic acid, N,N′-bis[(6-carboxy-2-pyridyl)methyl]-4,13-diaza-18-crown-6 (H2macropa), 6-((16-((6-carboxypyridin-2-yl)methyl)-1,4,10,13-tetraoxa-7,16-diazacyclooctadecan-7-yl)methyl)-4-isocyanatopicolinic acid (macropa-NCO), 6-((16-((6-carboxypyridin-2-yl)methyl)-1,4,10,13-tetraoxa-7,16-diazacyclooctadecan-7-yl)methyl)-4-isothiocyanatopicolinic acid (macropa-NCS), 3,9-carboxymethyl-6-(2-methoxy-5-isothiocyanatophenyl)carboxymethyl-3,6,9,15-tetraazabicyclo-[9.3.1]pentadeca-1(15),11,13-triene and 2-[4,7,10-tris(2-amino-2-oxoethyl)-1,4,7,10-tetrazacyclododec-1-yl]acetamide (TCMC or DOTAM).

24. (canceled)

25. The compound of claim 23 or a pharmaceutically acceptable salt thereof, wherein the chelating group is
i) represented by the structural formula 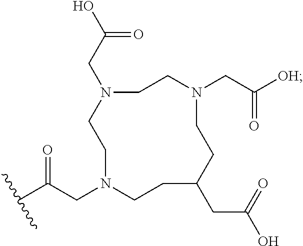 [SMILES: *C(=O)CN1CCC(CC(=O)O)CCN(CC(=O)O)CCN(CC(=O)O)CC1]
or
ii) represented by one of the following structural formulae 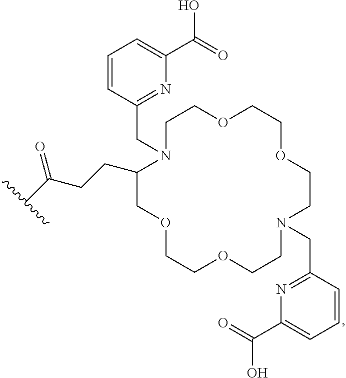 [SMILES: *C(=O)CCC1COCCOCCN(Cc2cccc(C(=O)O)n2)CCOCCOCCN1Cc1cccc(C(=O)O)n1] 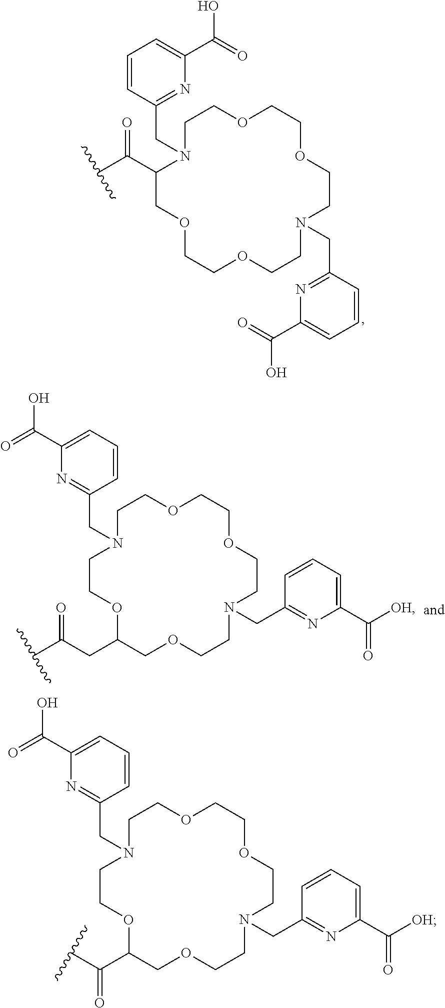 [SMILES: *C(=O)C1COCCOCCN(Cc2cccc(C(=O)O)n2)CCOCCOCCN1Cc1cccc(C(=O)O)n1 ; *C(=O)CC1COCCN(Cc2cccc(C(=O)O)n2)CCOCCOCCN(Cc2cccc(C(=O)O)n2)CCO1 ; *C(=O)C1COCCN(Cc2cccc(C(=O)O)n2)CCOCCOCCN(Cc2cccc(C(=O)O)n2)CCO1]
iii) represented by the structural formula

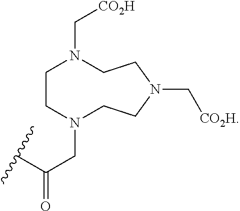 [SMILES: *C(=O)CN1CCN(CC(=O)O)CCN(CC(=O)O)CC1]

26-28. (canceled)

29. The compound of claim 1, or a pharmaceutically acceptable salt thereof, wherein the compound is selected from one of the following structural formula:

| Compound | |
|---|---|
| No. | Structure |
| 1 | 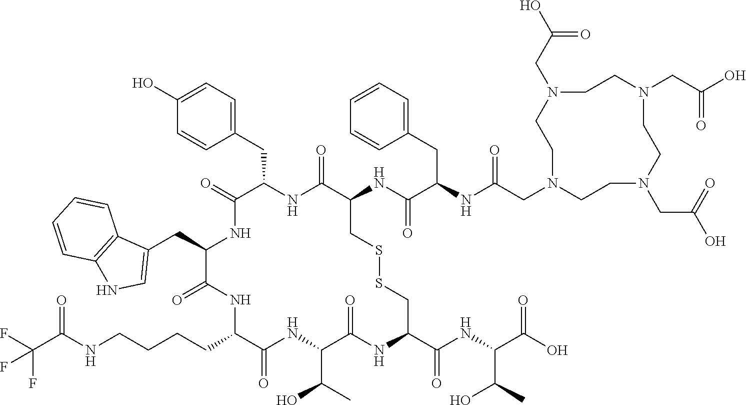 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCNC(=O)C(F)(F)F)C(=O)N[C@@H]([C@@H](C)O)C(=O)N1)C(=O)O] |
| 2 | 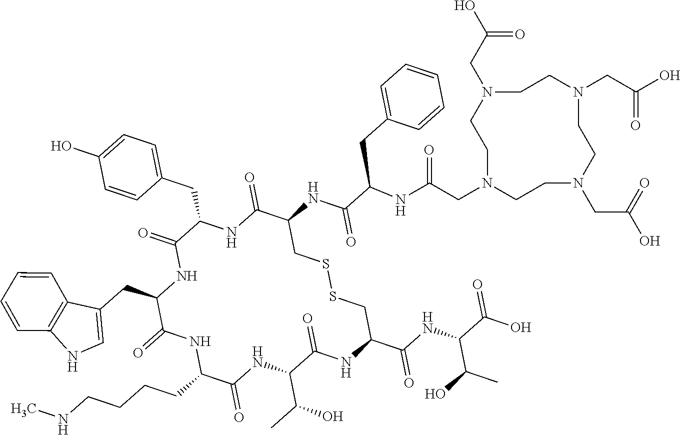 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 3 | 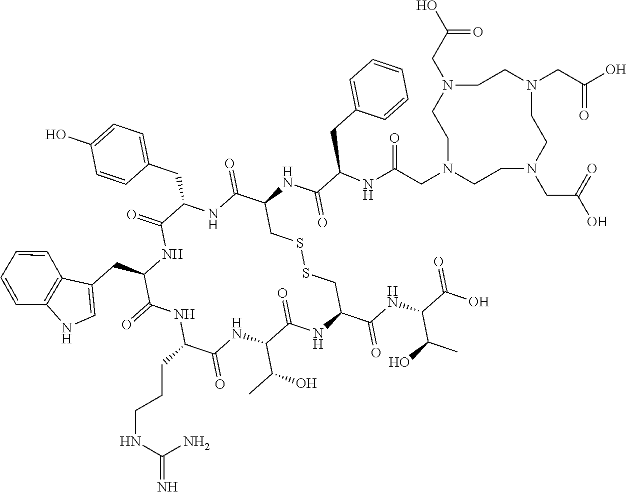 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCNC(=N)N)C(=O)N[C@@H]([C@@H](C)O)C(=O)N1)C(=O)O] |
| 4 | 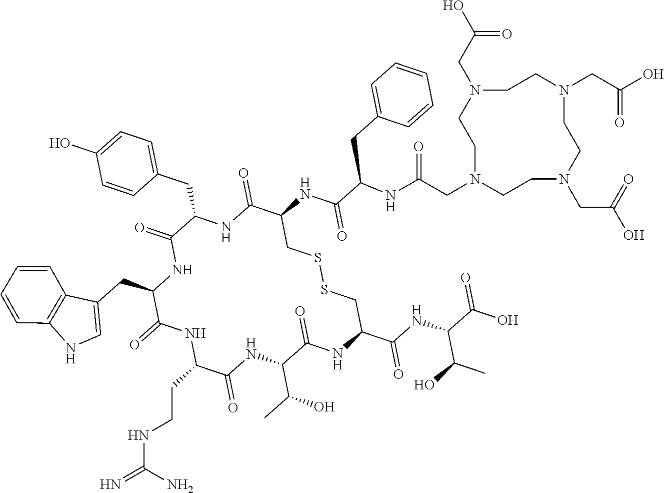 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCNC(=N)N)C(=O)N[C@@H]([C@@H](C)O)C(=O)N1)C(=O)O] |
| 5 | 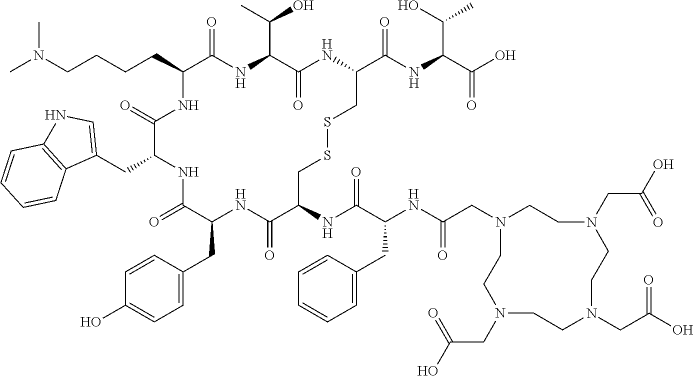 [SMILES: C[C@@H](O)[C@H](NC(=O)[C@@H]1CSSC[C@@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN(C)C)C(=O)N[C@@H]([C@@H](C)O)C(=O)N1)C(=O)O] |
| 6 | 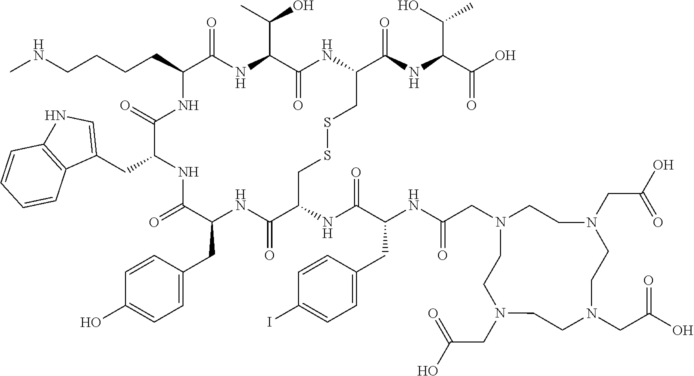 |
| 7 | 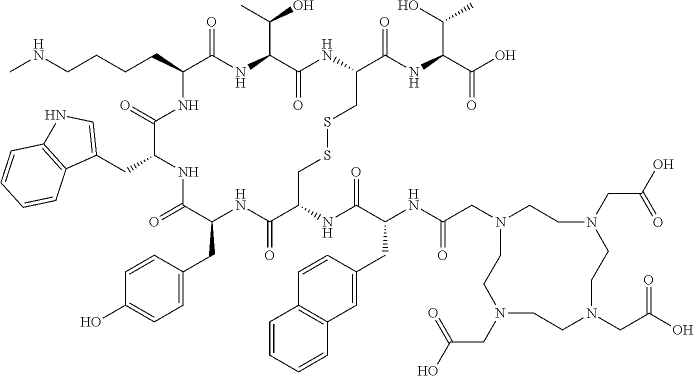 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccc3ccccc3c2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 8 | 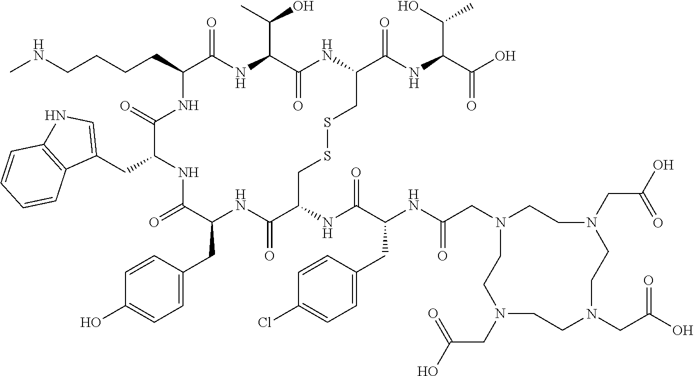 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccc(Cl)cc2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 9 | 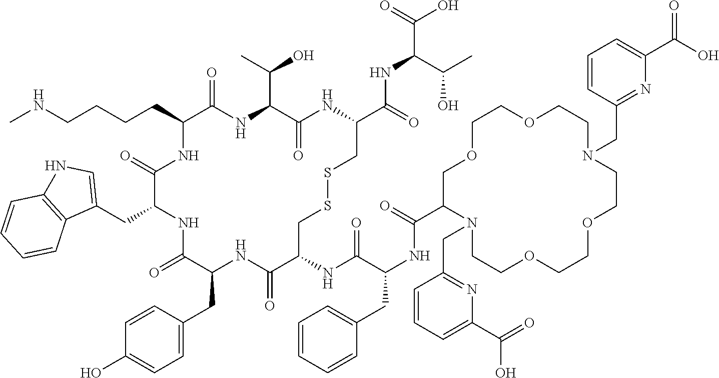 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)C2COCCOCCN(Cc3cccc(C(=O)O)n3)CCOCCOCCN2Cc2cccc(C(=O)O)n2)CSSC[C@@H](C(=O)N[C@@H](C(=O)O)[C@H](C)O)NC(=O)[C@H]([C@@H](C)O)NC1=O] |
| 10 | 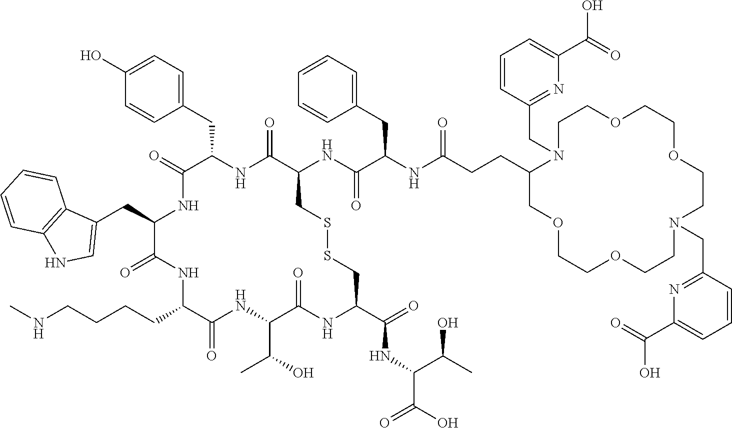 |
| 11 | 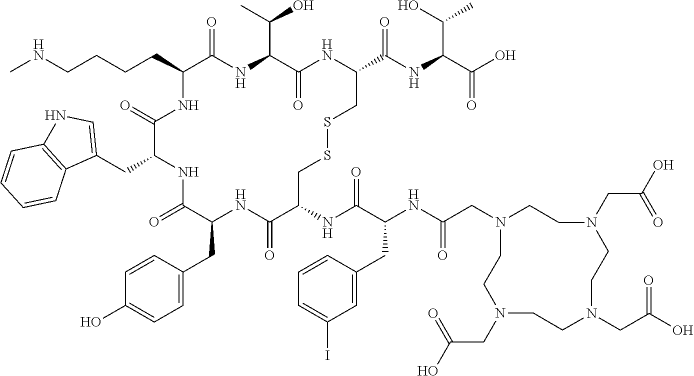 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2cccc(I)c2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 12 | 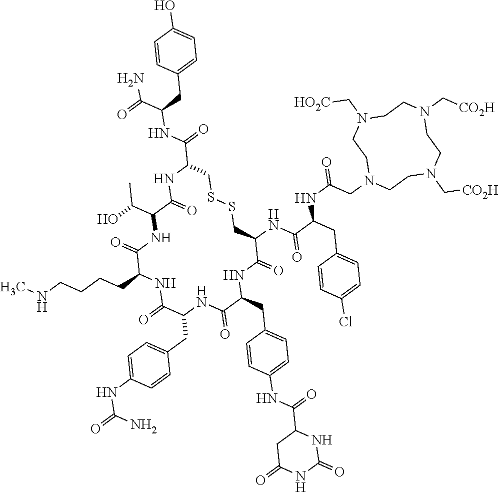 |
| 13 | 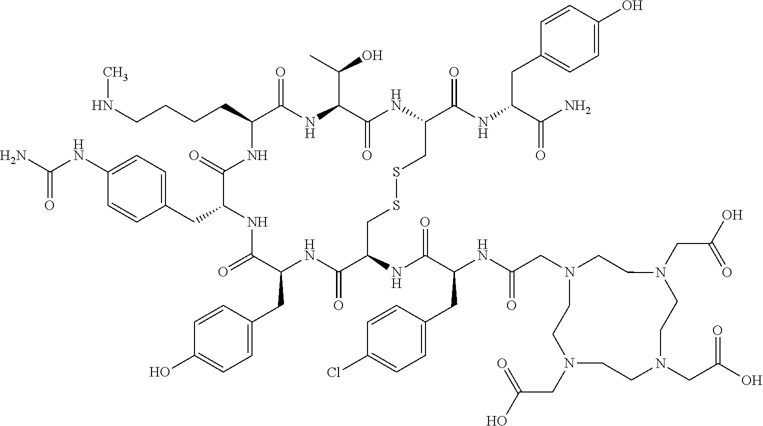 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2ccc(Cl)cc2)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |
| 14 | 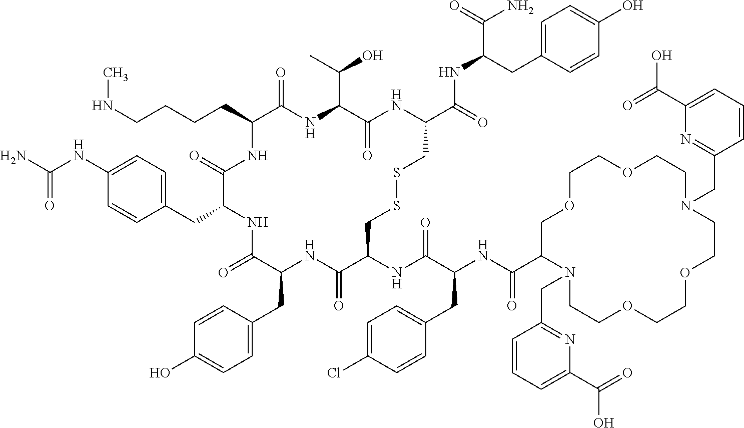 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2ccc(Cl)cc2)NC(=O)C2COCCOCCN(Cc3cccc(C(=O)O)n3)CCOCCOCCN2Cc2cccc(C(=O)O)n2)CSSC[C@@H](C(=O)N[C@H](Cc2ccc(O)cc2)C(N)=O)NC(=O)[C@H]([C@@H](C)O)NC1=O] |
| 15 | 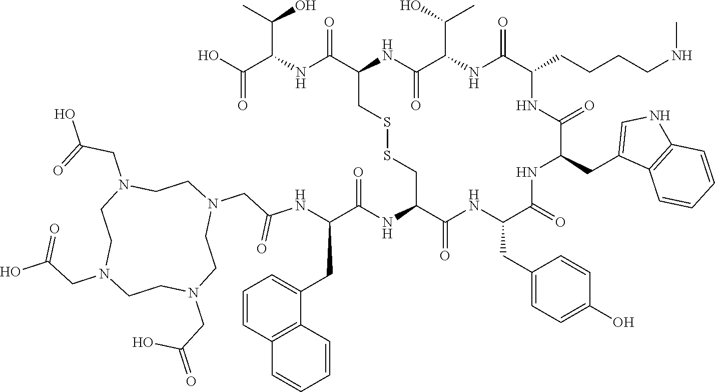 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2cccc3ccccc23)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 16 | 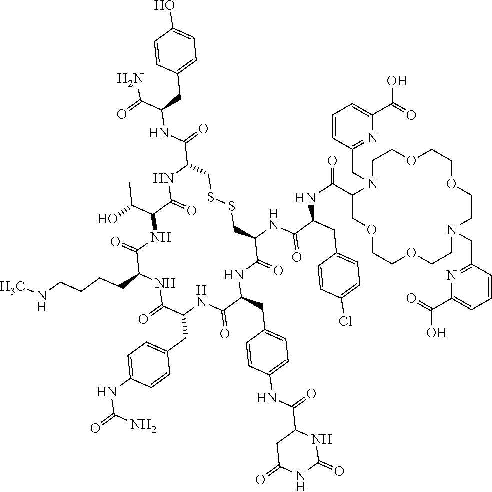 |
| 17 | 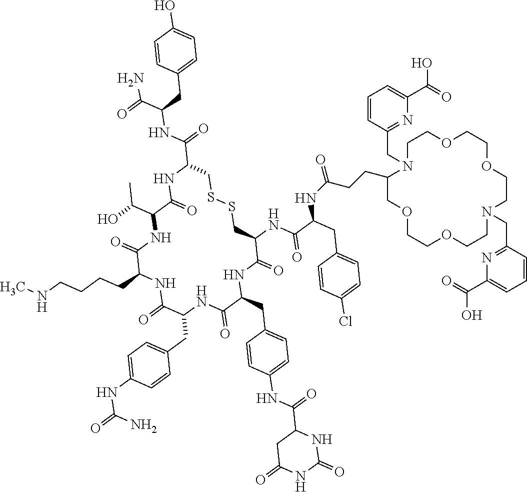 |
| 18 | 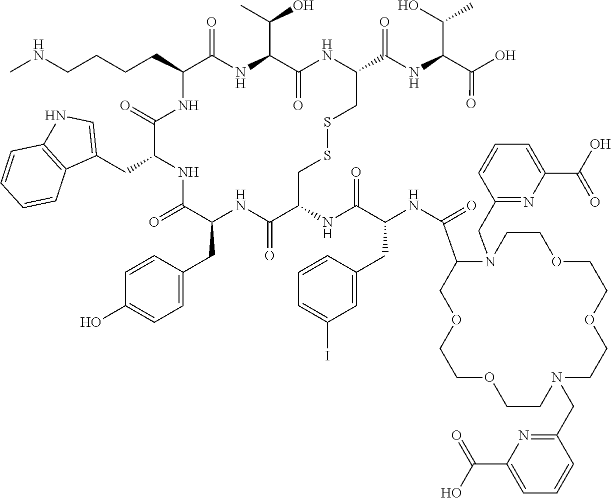 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2cccc(I)c2)NC(=O)C2COCCOCCN(Cc3cccc(C(=O)O)n3)CCOCCOCCN2Cc2cccc(C(=O)O)n2)CSSC[C@@H](C(=O)N[C@H](C(=O)O)[C@@H](C)O)NC(=O)[C@H]([C@@H](C)O)NC1=O] |
| 19 | 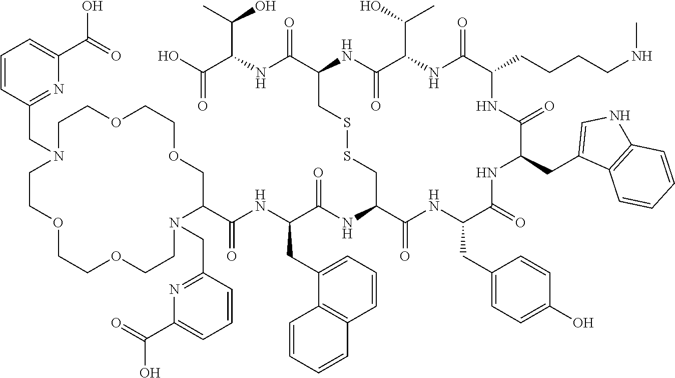 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2cccc3ccccc23)NC(=O)C2COCCOCCN(Cc3cccc(C(=O)O)n3)CCOCCOCCN2Cc2cccc(C(=O)O)n2)CSSC[C@@H](C(=O)N[C@H](C(=O)O)[C@@H](C)O)NC(=O)[C@H]([C@@H](C)O)NC1=O] |
| 20 | 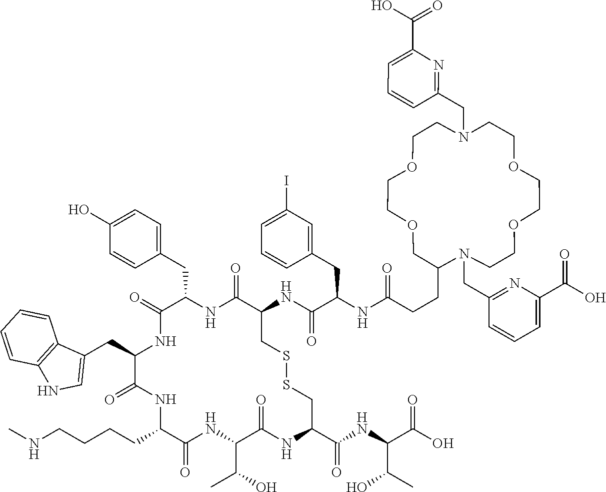 |
| 21 | 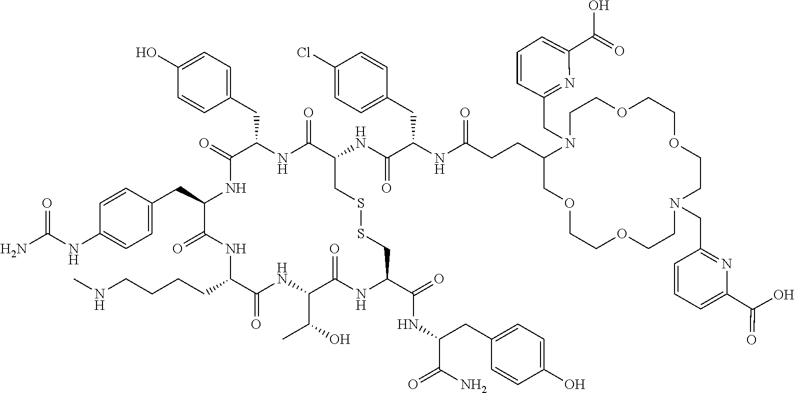 |
| 22 | 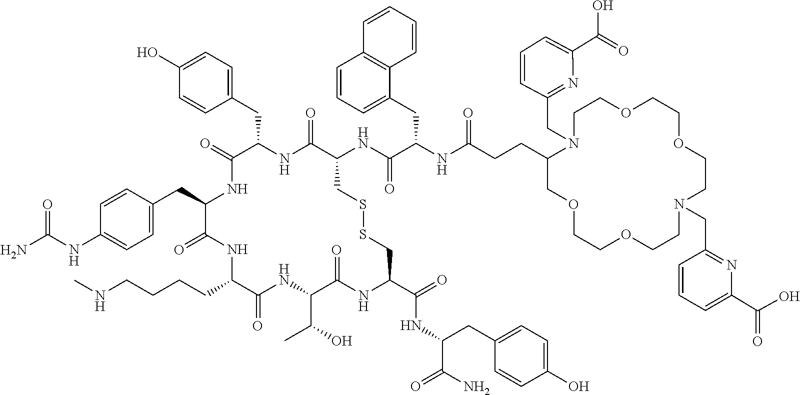 |
| 23 | 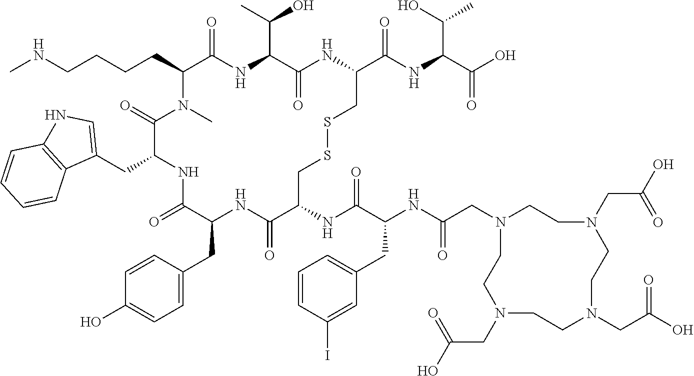 [SMILES: CNCCCC[C@H]1C(=O)N[C@@H]([C@@H](C)O)C(=O)N[C@H](C(=O)N[C@H](C(=O)O)[C@@H](C)O)CSSC[C@H](NC(=O)[C@@H](Cc2cccc(I)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N1C] |
| 24 | 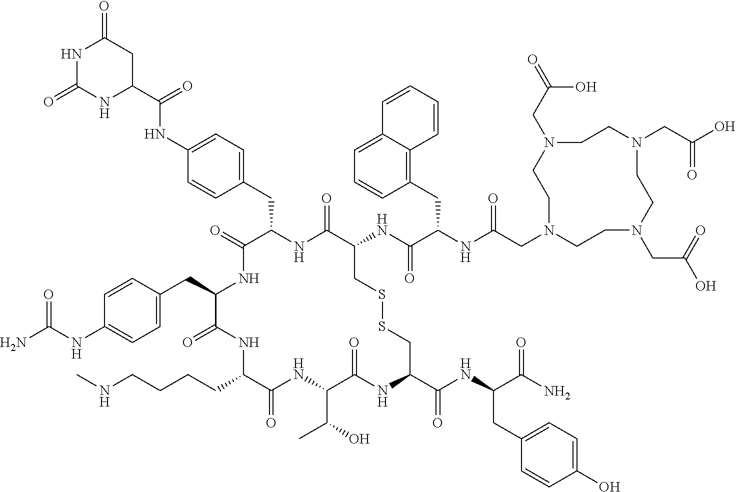 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2cccc3ccccc23)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |
| 25 | 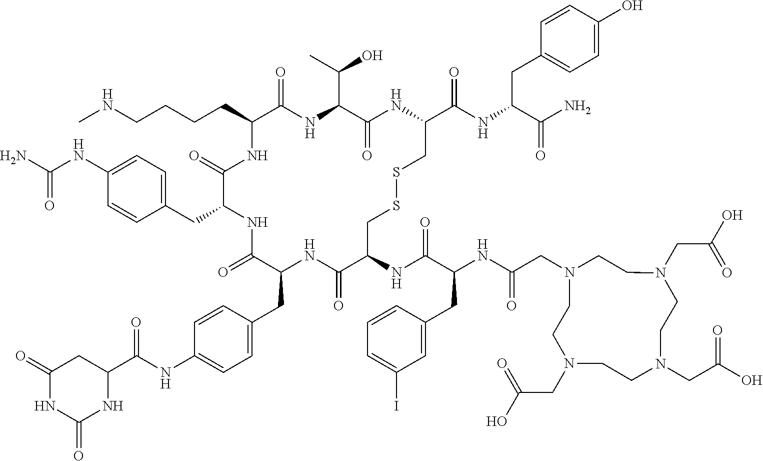 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2cccc(I)c2)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |
| 26 | 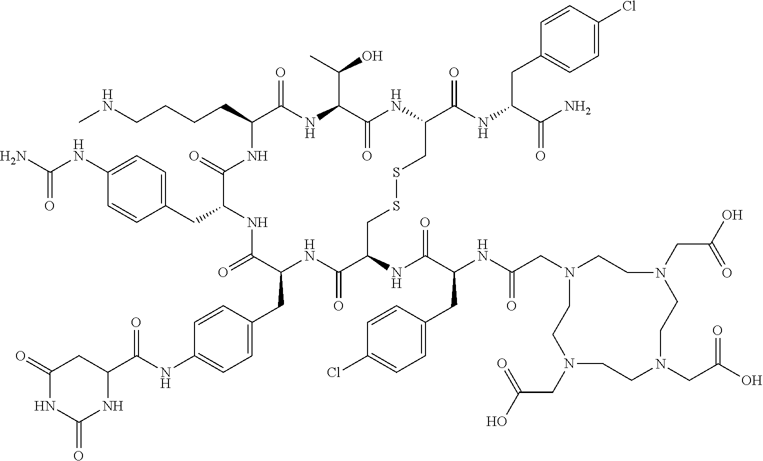 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2ccc(Cl)cc2)NC(=O)CN2CCN(CC(=O)O)CCN(CC(=O)O)CCN(CC(=O)O)CC2)CSSC[C@@H](C(=O)N[C@H](Cc2ccc(Cl)cc2)C(N)=O)NC(=O)[C@H]([C@@H](C)O)NC1=O] |
| 27 | 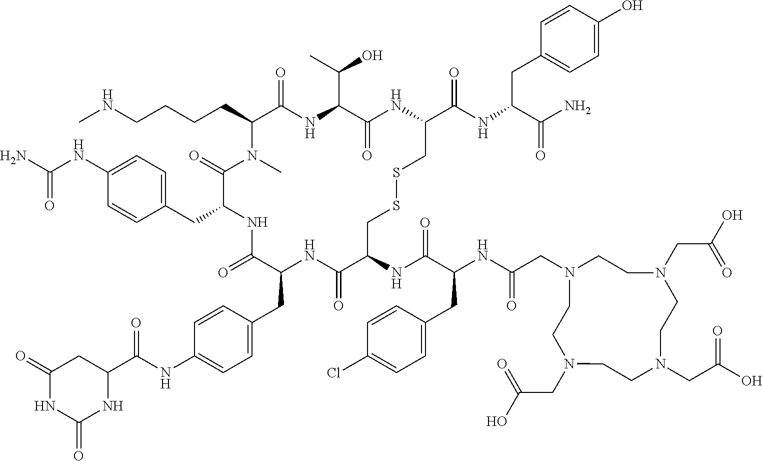 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)CN2CCN(CC(=O)O)CCN(CC(=O)O)CCN(CC(=O)O)CC2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| 28 | 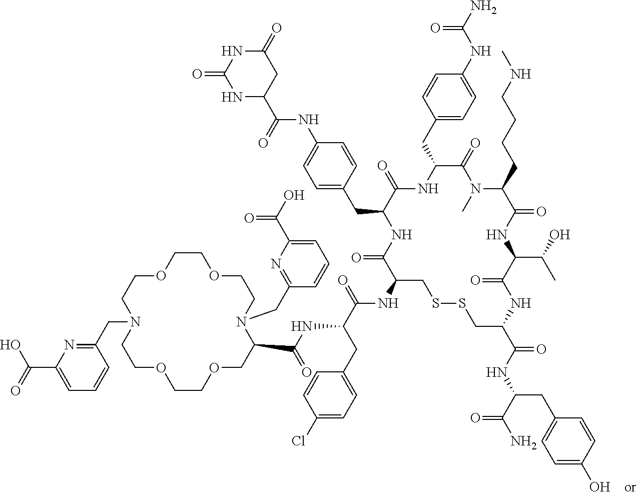 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)[C@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| | 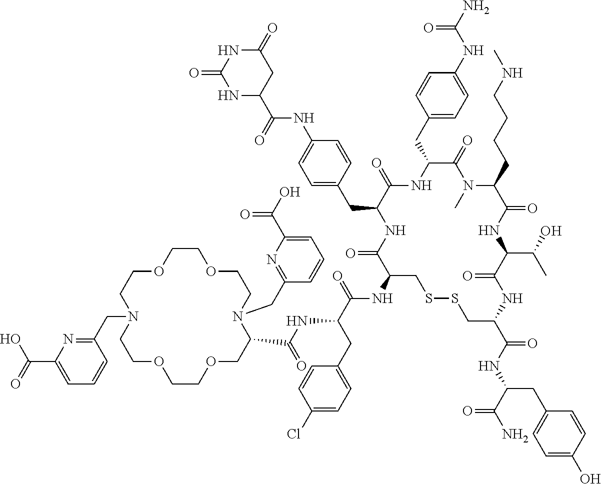 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)[C@@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| 29 | 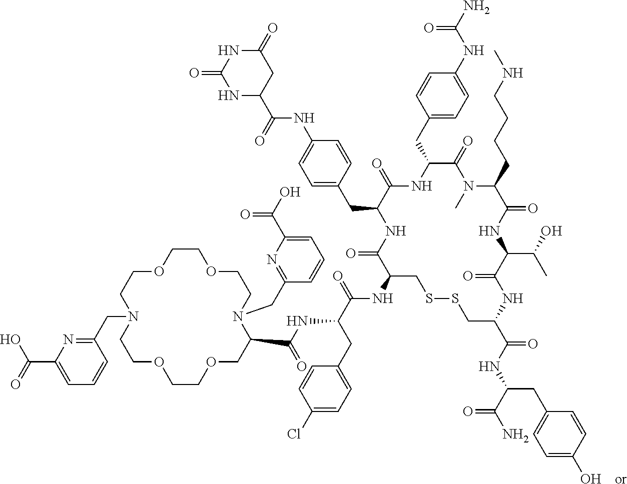 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)[C@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| | 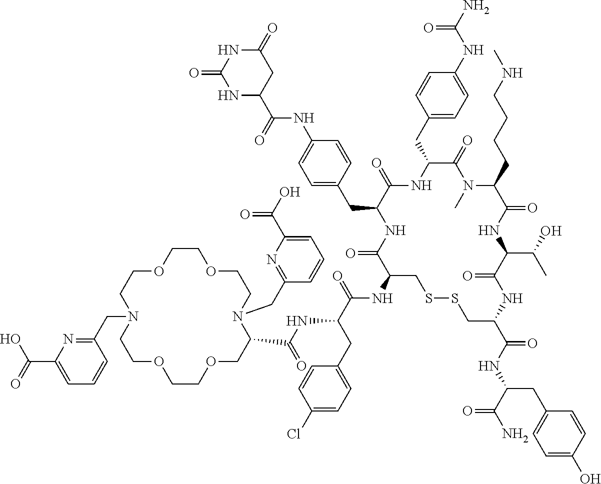 [SMILES: CNCCCC[C@H]1C(=O)N[C@@H]([C@@H](C)O)C(=O)N[C@H](C(=O)N[C@H](Cc2ccc(O)cc2)C(N)=O)CSSC[C@@H](NC(=O)[C@H](Cc2ccc(Cl)cc2)NC(=O)[C@@H]2COCCOCCN(Cc3cccc(C(=O)O)n3)CCOCCOCCN2Cc2cccc(C(=O)O)n2)C(=O)N[C@@H](Cc2ccc(NC(=O)C3CC(=O)NC(=O)N3)cc2)C(=O)N[C@H](Cc2ccc(NC(N)=O)cc2)C(=O)N1C] |
| 30 | 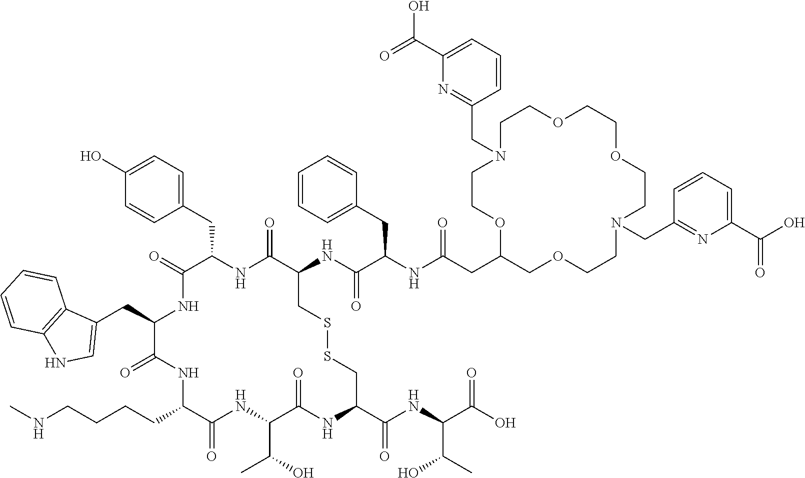 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)CC2COCCN(Cc3cccc(C(=O)O)n3)CCOCCOCCN(Cc3cccc(C(=O)O)n3)CCO2)CSSC[C@@H](C(=O)N[C@@H](C(=O)O)[C@H](C)O)NC(=O)[C@H]([C@@H](C)O)NC1=O] |
| 31 | 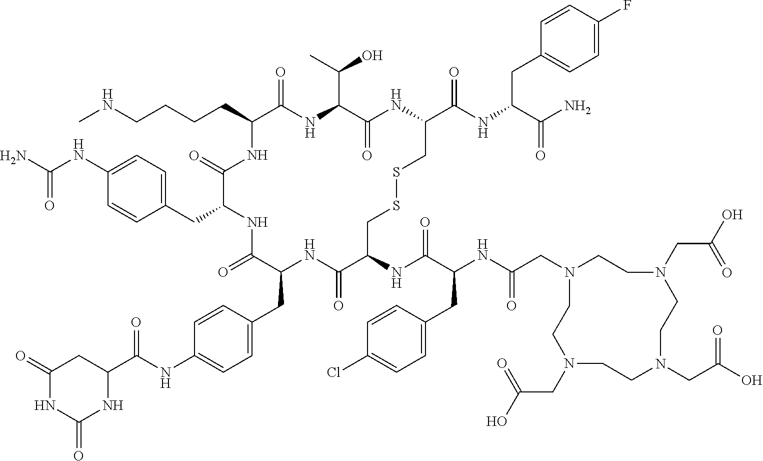 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2ccc(Cl)cc2)NC(=O)CN2CCN(CC(=O)O)CCN(CC(=O)O)CCN(CC(=O)O)CC2)CSSC[C@@H](C(=O)N[C@H](Cc2ccc(F)cc2)C(N)=O)NC(=O)[C@H]([C@@H](C)O)NC1=O] |
| 32 | 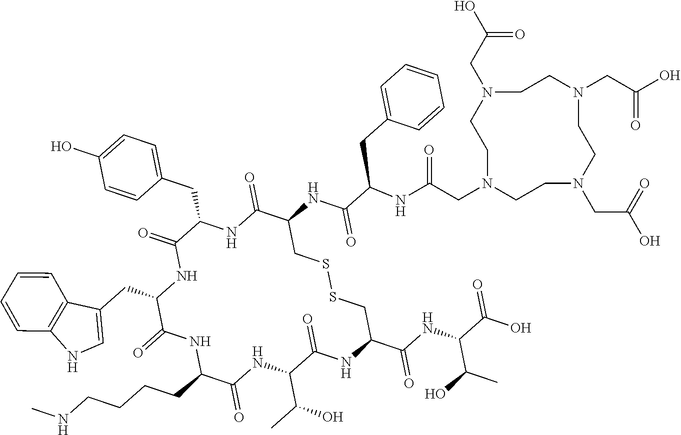 [SMILES: CNCCCC[C@H]1NC(=O)[C@H](Cc2c[nH]c3ccccc23)NC(=O)[C@H](Cc2ccc(O)cc2)NC(=O)[C@@H](NC(=O)[C@@H](Cc2ccccc2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 33 | 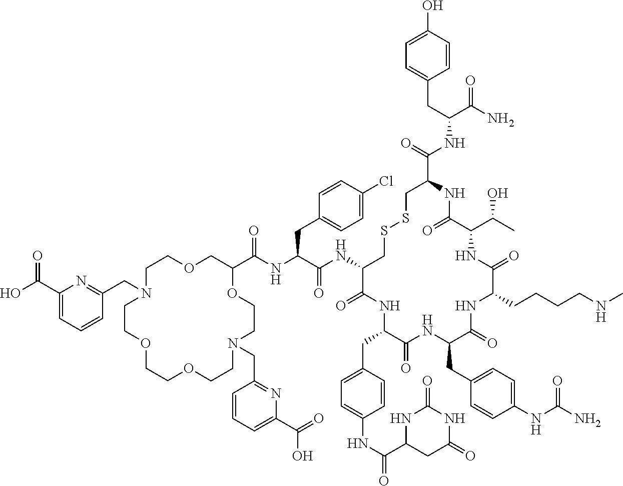 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2ccc(Cl)cc2)NC(=O)C2COCCN(Cc3cccc(C(=O)O)n3)CCOCCOCCN(Cc3cccc(C(=O)O)n3)CCO2)CSSC[C@@H](C(=O)N[C@H](Cc2ccc(O)cc2)C(N)=O)NC(=O)[C@H]([C@@H](C)O)NC1=O] |
| 34 | 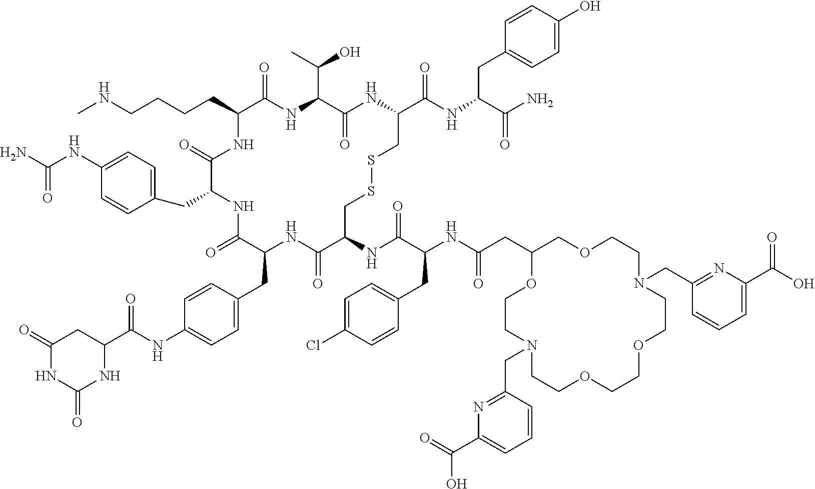 [SMILES: CNCCCC[C@@H]1NC(=O)[C@@H](Cc2ccc(NC(N)=O)cc2)NC(=O)[C@H](Cc2ccc(NC(=O)C3CC(=O)NC(=O)N3)cc2)NC(=O)[C@H](NC(=O)[C@H](Cc2ccc(Cl)cc2)NC(=O)CC2COCCN(Cc3cccc(C(=O)O)n3)CCOCCOCCN(Cc3cccc(C(=O)O)n3)CCO2)CSSC[C@@H](C(=O)N[C@H](Cc2ccc(O)cc2)C(N)=O)NC(=O)[C@H]([C@@H](C)O)NC1=O] |
| 35 | 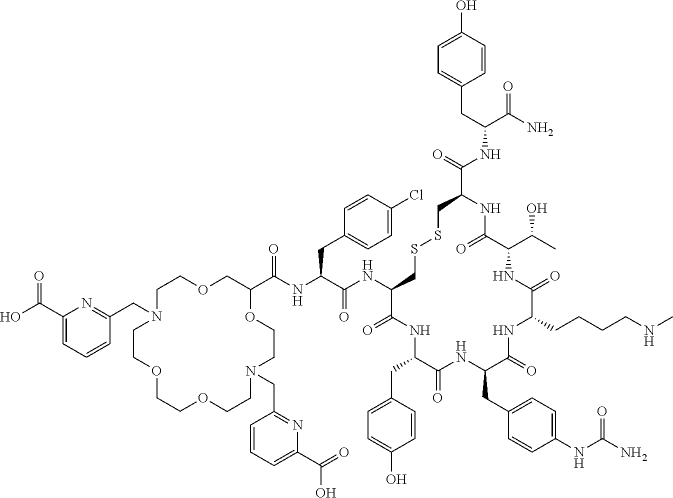 [SMILES: CNCCCC[C@@H]1NC(=O)[C@@H](Cc2ccc(NC(N)=O)cc2)NC(=O)[C@H](Cc2ccc(O)cc2)NC(=O)[C@@H](NC(=O)[C@H](Cc2ccc(Cl)cc2)NC(=O)C2COCCN(Cc3cccc(C(=O)O)n3)CCOCCOCCN(Cc3cccc(C(=O)O)n3)CCO2)CSSC[C@@H](C(=O)N[C@H](Cc2ccc(O)cc2)C(N)=O)NC(=O)[C@H]([C@@H](C)O)NC1=O] |
| 36 | 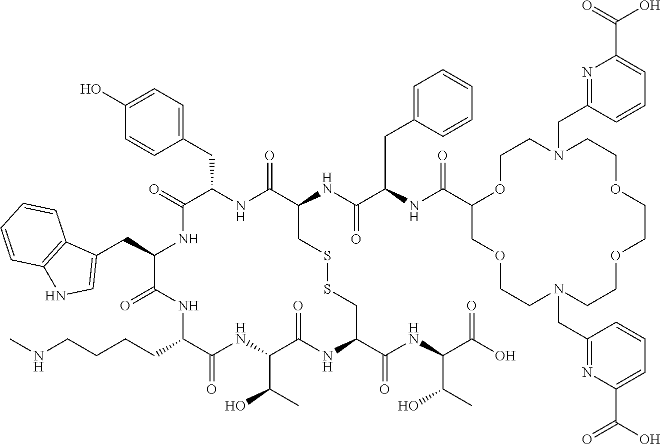 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)C2COCCN(Cc3cccc(C(=O)O)n3)CCOCCOCCN(Cc3cccc(C(=O)O)n3)CCO2)CSSC[C@@H](C(=O)N[C@@H](C(=O)O)[C@H](C)O)NC(=O)[C@H]([C@@H](C)O)NC1=O] |
| 37 | 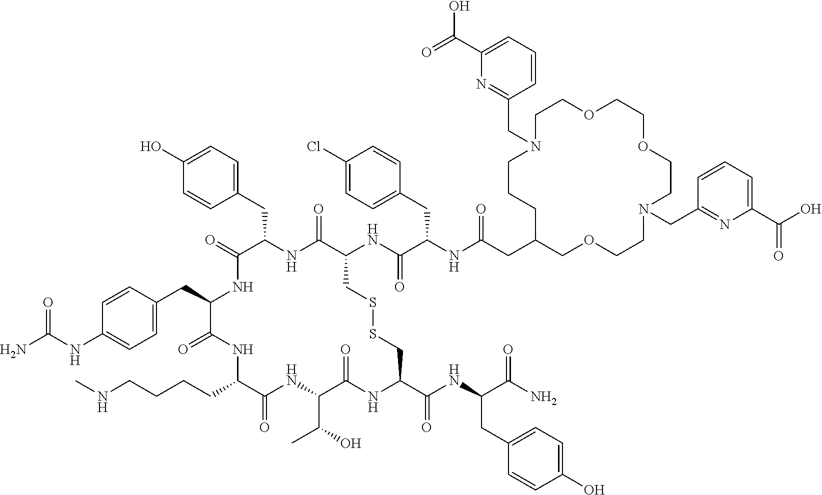 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2ccc(Cl)cc2)NC(=O)CC2CCCN(Cc3cccc(C(=O)O)n3)CCOCCOCCN(Cc3cccc(C(=O)O)n3)CCOC2)CSSC[C@@H](C(=O)N[C@H](Cc2ccc(O)cc2)C(N)=O)NC(=O)[C@H]([C@@H](C)O)NC1=O] |
| 38 | 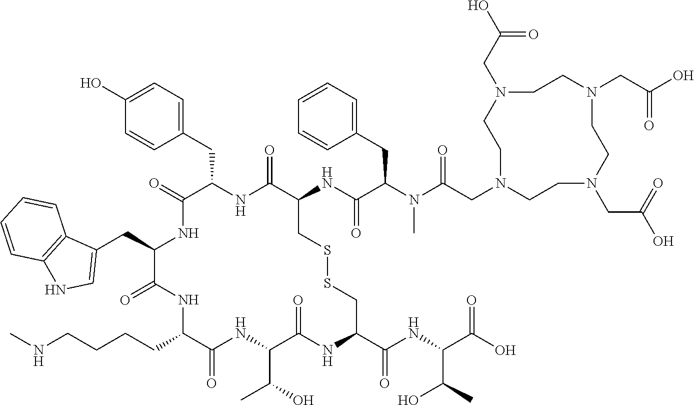 [SMILES: CNCCCC[C@@H]1NC(=O)[C@@H](Cc2c[nH]c3ccccc23)NC(=O)[C@H](Cc2ccc(O)cc2)NC(=O)[C@@H](NC(=O)[C@@H](Cc2ccccc2)N(C)C(=O)CN2CCN(CC(=O)O)CCN(CC(=O)O)CCN(CC(=O)O)CC2)CSSC[C@@H](C(=O)N[C@H](C(=O)O)[C@@H](C)O)NC(=O)[C@H]([C@@H](C)O)NC1=O] |
| 39 | 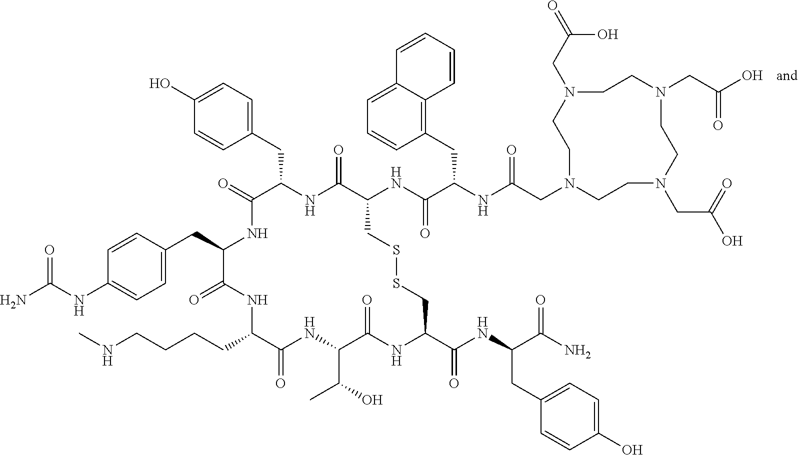 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2cccc3ccccc23)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |
| 40 | 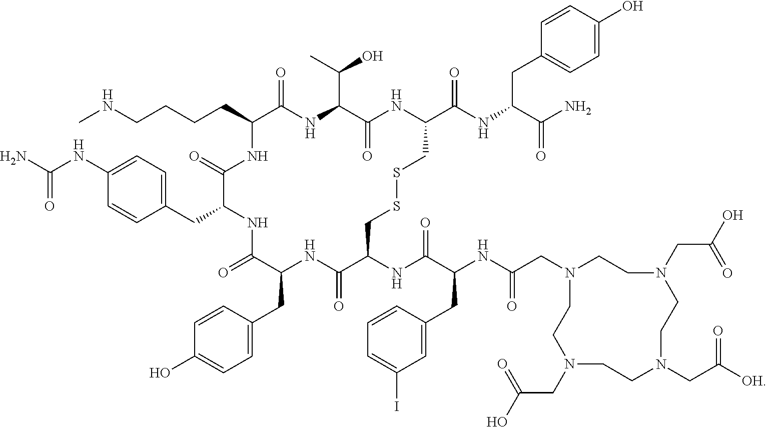 [SMILES: CNCCCC[C@@H]1NC(=O)[C@@H](Cc2ccc(NC(N)=O)cc2)NC(=O)[C@H](Cc2ccc(O)cc2)NC(=O)[C@H](NC(=O)[C@H](Cc2cccc(I)c2)NC(=O)CN2CCN(CC(=O)O)CCN(CC(=O)O)CCN(CC(=O)O)CC2)CSSC[C@@H](C(=O)N[C@H](Cc2ccc(O)cc2)C(N)=O)NC(=O)[C@H]([C@@H](C)O)NC1=O] |

30. The compound of claim 1, or a pharmaceutically acceptable salt thereof, wherein the compound is represented by structural formula (Va), (Vb), (Vc), (VIa), (VIb), (VIc), (VIIa), (VIIb), or (VIIc): 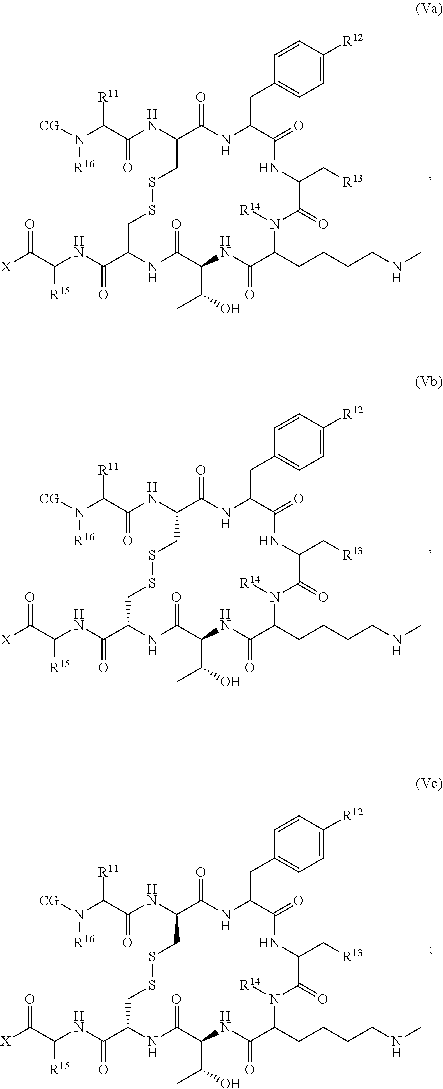 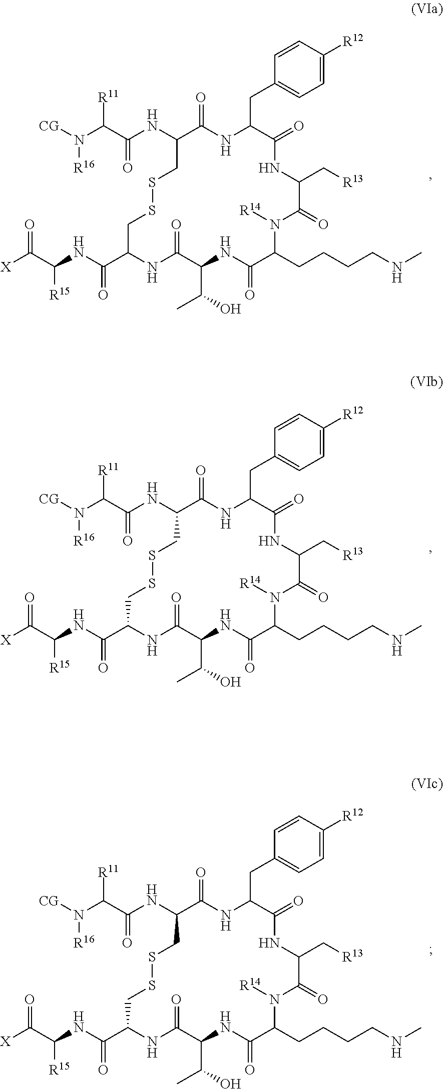 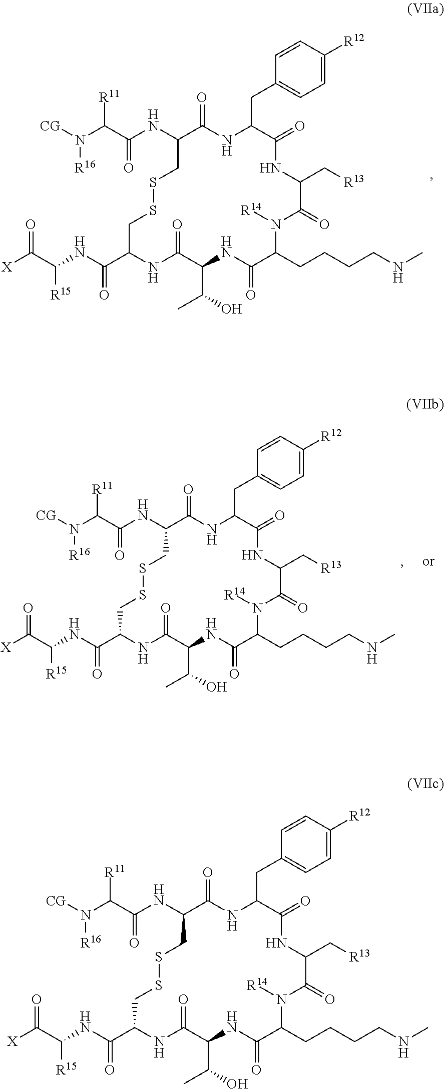
wherein:
X is OH or NH₂,
CG is a chelating group selected from: 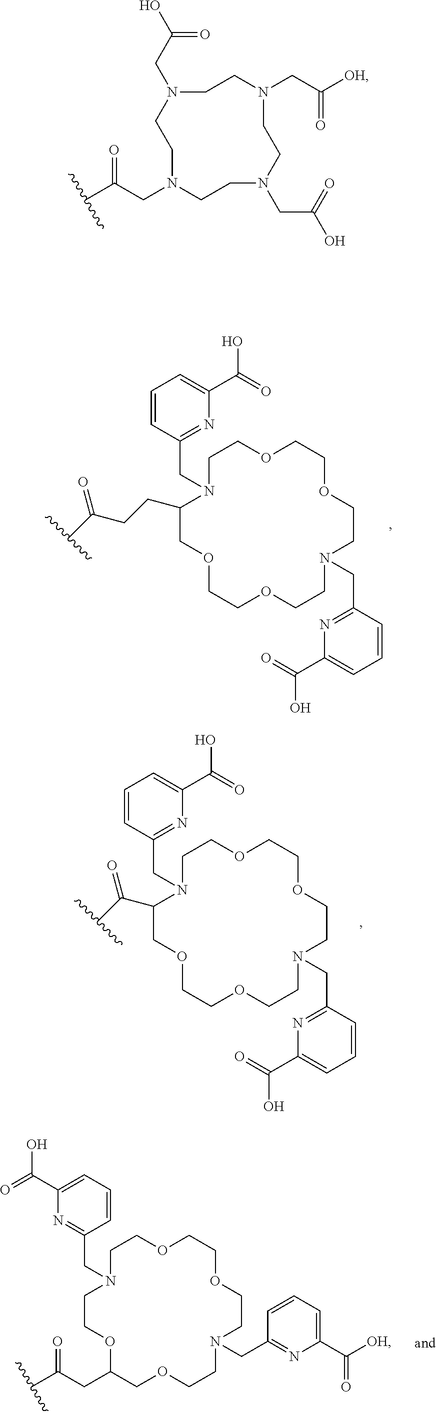 [SMILES: *C(=O)CN1CCN(CC(=O)O)CCN(CC(=O)O)CCN(CC(=O)O)CC1 ; *C(=O)CCC1COCCOCCN(Cc2cccc(C(=O)O)n2)CCOCCOCCN1Cc1cccc(C(=O)O)n1 ; *C(=O)C1COCCOCCN(Cc2cccc(C(=O)O)n2)CCOCCOCCN1Cc1cccc(C(=O)O)n1 ; *C(=O)CC1COCCN(Cc2cccc(C(=O)O)n2)CCOCCOCCN(Cc2cccc(C(=O)O)n2)CCO1] 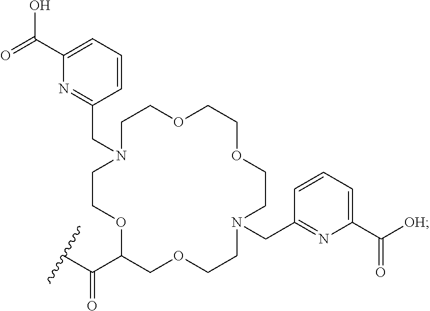 [SMILES: *C(=O)C1COCCN(Cc2cccc(C(=O)O)n2)CCOCCOCCN(Cc2cccc(C(=O)O)n2)CCO1]
R¹¹ is selected from 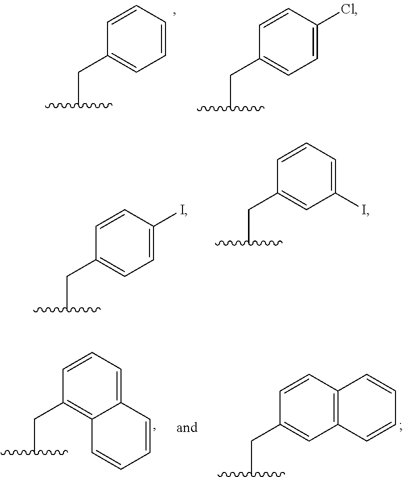 [SMILES: *Cc1ccccc1 ; *Cc1ccc(Cl)cc1 ; *Cc1ccc(I)cc1 ; *Cc1cccc(I)c1 ; *Cc1cccc2ccccc12 ; *Cc1ccc2ccccc2c1]
R¹² is OH or 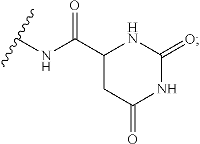 [SMILES: *NC(=O)C1CC(=O)NC(=O)N1]
R¹³ is 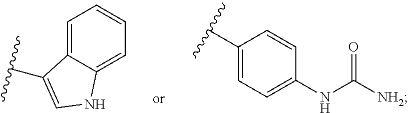 [SMILES: *c1c[nH]c2ccccc12 ; *c1ccc(NC(N)=O)cc1]
R¹⁴ and R¹⁶ are each independently H or CH₃; and
R¹⁵ is

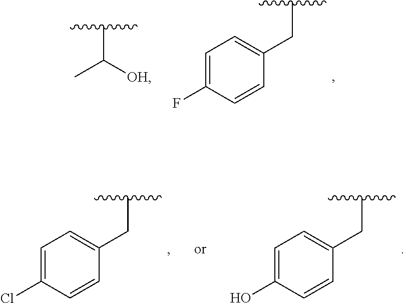

31. (canceled)

32. (canceled)

33. The compound of claim 30, or a pharmaceutically acceptable salt thereof, wherein the compound is represented by structural formula (VIIIa), (VIIIb), or (VIIIc): 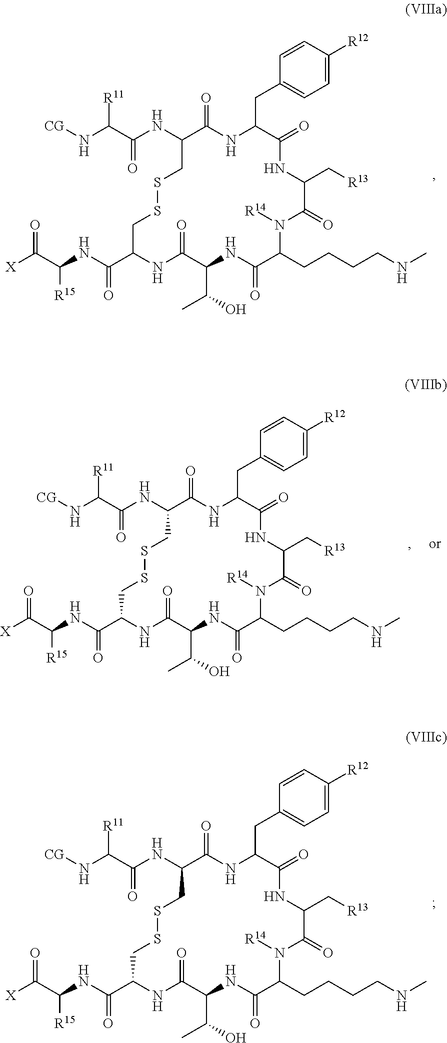
or a pharmaceutically acceptable salt thereof wherein:
CG is a chelating group selected from: 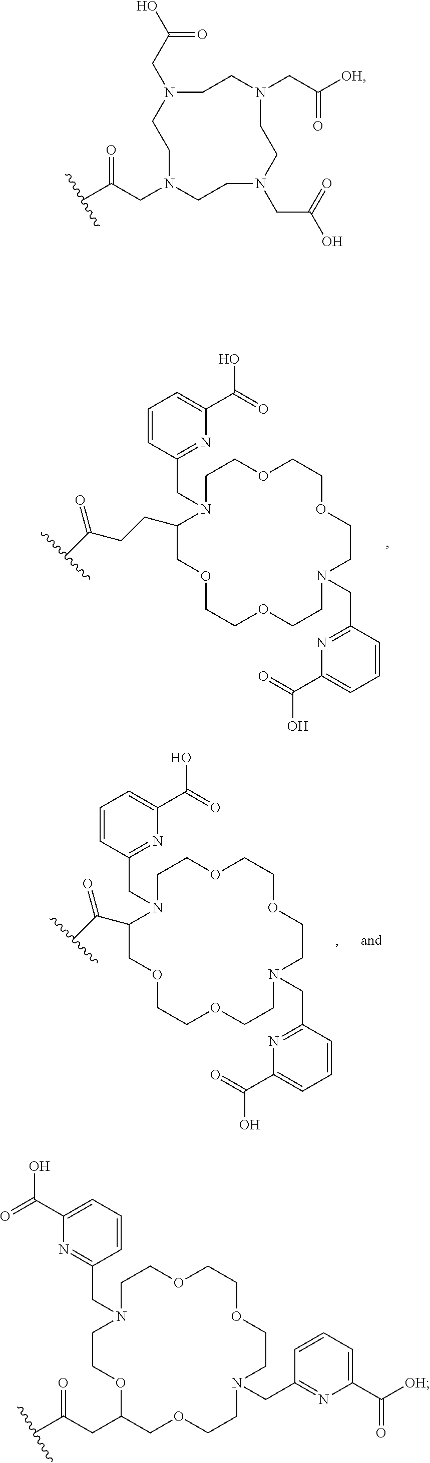 [SMILES: *C(=O)CN1CCN(CC(=O)O)CCN(CC(=O)O)CCN(CC(=O)O)CC1 ; *C(=O)CCC1COCCOCCN(Cc2cccc(C(=O)O)n2)CCOCCOCCN1Cc1cccc(C(=O)O)n1 ; *C(=O)C1COCCOCCN(Cc2cccc(C(=O)O)n2)CCOCCOCCN1Cc1cccc(C(=O)O)n1 ; *C(=O)CC1COCCN(Cc2cccc(C(=O)O)n2)CCOCCOCCN(Cc2cccc(C(=O)O)n2)CCO1]
R¹¹ is selected from 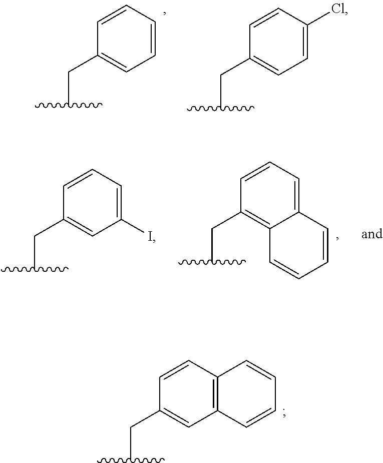 [SMILES: *Cc1ccccc1 ; *Cc1ccc(Cl)cc1 ; *Cc1cccc(I)c1 ; *Cc1cccc2ccccc12 ; *Cc1ccc2ccccc2c1]
and
R¹⁵ is

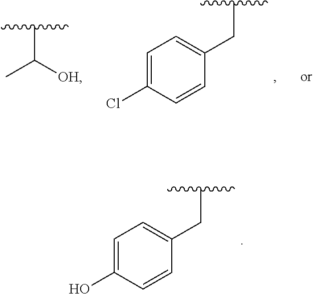

34. The compound of claim 1, or a pharmaceutically acceptable salt thereof, wherein the compound is represented by structural formula (IXa), (IXb), or (IXc): 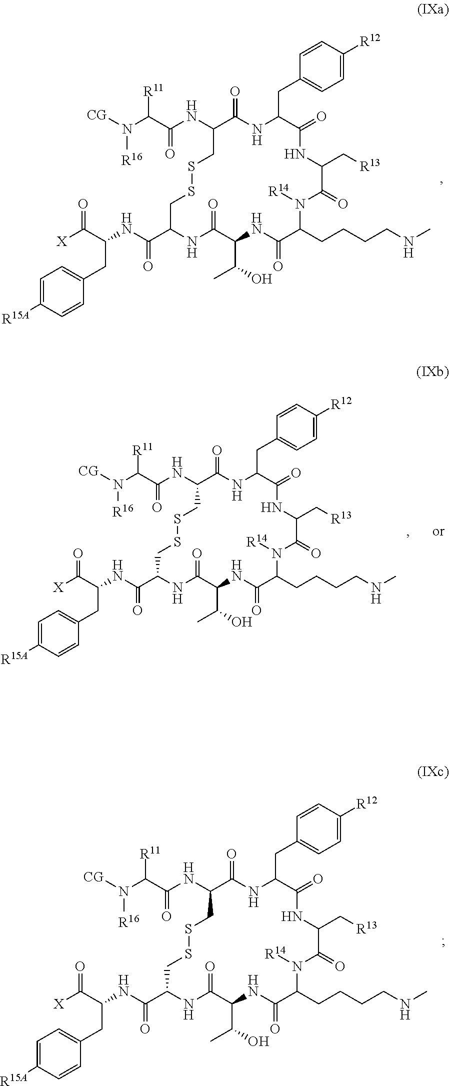
wherein:
X is OH or NH₂;
CG is a chelating group selected from: 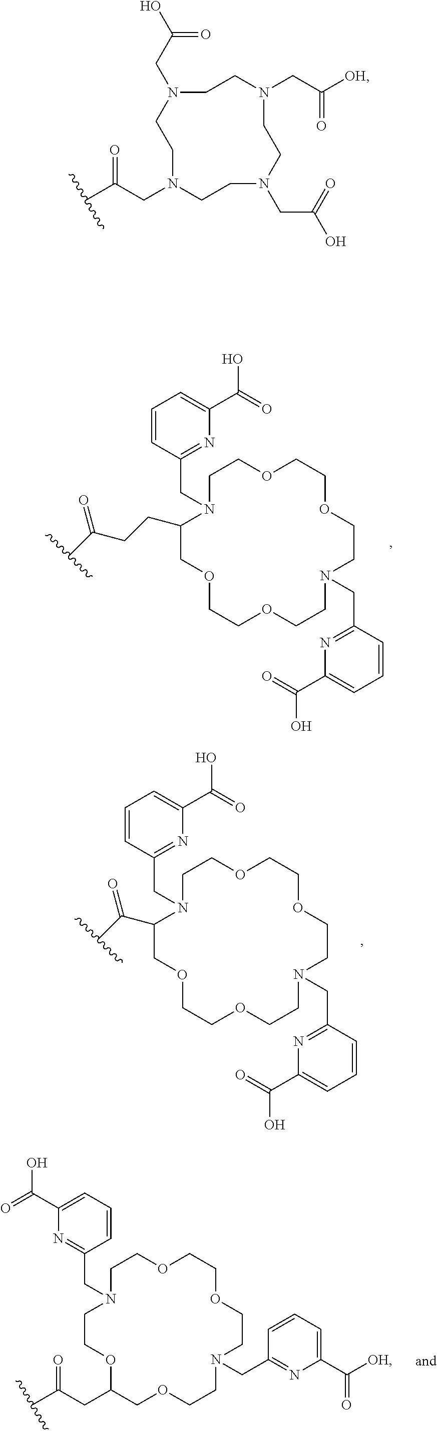 [SMILES: *C(=O)CN1CCN(CC(=O)O)CCN(CC(=O)O)CCN(CC(=O)O)CC1 ; *C(=O)CCC1COCCOCCN(Cc2cccc(C(=O)O)n2)CCOCCOCCN1Cc1cccc(C(=O)O)n1 ; *C(=O)C1COCCOCCN(Cc2cccc(C(=O)O)n2)CCOCCOCCN1Cc1cccc(C(=O)O)n1 ; *C(=O)CC1COCCN(Cc2cccc(C(=O)O)n2)CCOCCOCCN(Cc2cccc(C(=O)O)n2)CCO1] 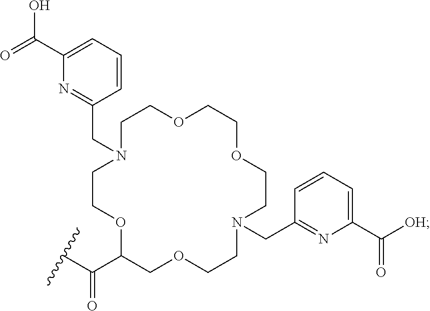 [SMILES: *C(=O)C1COCCN(Cc2cccc(C(=O)O)n2)CCOCCOCCN(Cc2cccc(C(=O)O)n2)CCO1]
R¹¹ is selected from 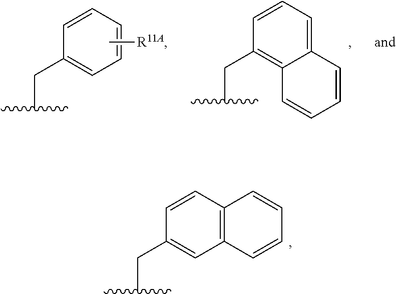
wherein R^{11A} is H or halogen;
R¹² is OH or 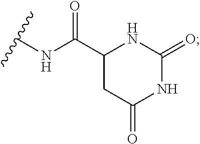 [SMILES: *NC(=O)C1CC(=O)NC(=O)N1]
R¹³ is 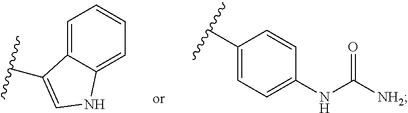 [SMILES: *c1c[nH]c2ccccc12 ; *c1ccc(NC(N)=O)cc1]
R¹⁴ and R¹⁶ are each independently H or CH₃; and
R^{15A} is Cl, OH, or F.

35. (canceled)

36. (canceled)

37. The compound of claim 34, or a pharmaceutically acceptable salt thereof, wherein:
CG is a chelating group selected from: 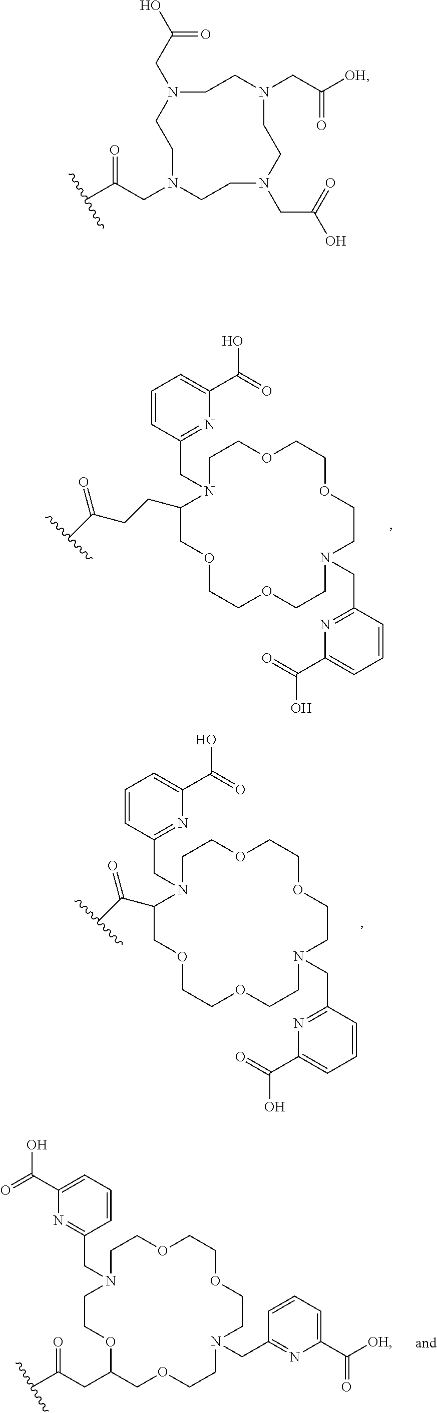 [SMILES: *C(=O)CN1CCN(CC(=O)O)CCN(CC(=O)O)CCN(CC(=O)O)CC1 ; *C(=O)CCC1COCCOCCN(Cc2cccc(C(=O)O)n2)CCOCCOCCN1Cc1cccc(C(=O)O)n1 ; *C(=O)C1COCCOCCN(Cc2cccc(C(=O)O)n2)CCOCCOCCN1Cc1cccc(C(=O)O)n1 ; *C(=O)CC1COCCN(Cc2cccc(C(=O)O)n2)CCOCCOCCN(Cc2cccc(C(=O)O)n2)CCO1] 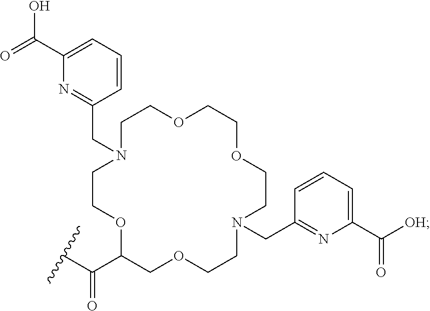 [SMILES: *C(=O)C1COCCN(Cc2cccc(C(=O)O)n2)CCOCCOCCN(Cc2cccc(C(=O)O)n2)CCO1]
R¹¹ is selected from 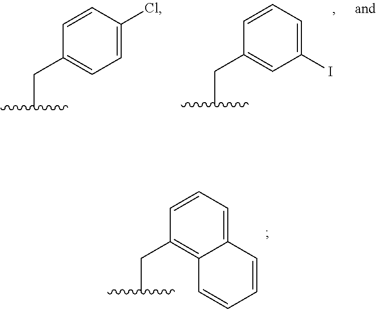 [SMILES: *Cc1ccc(Cl)cc1 ; *Cc1cccc(I)c1 ; *Cc1cccc2ccccc12]
R¹² is OH or 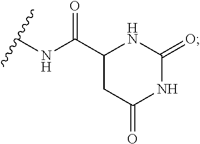 [SMILES: *NC(=O)C1CC(=O)NC(=O)N1]
R¹³ is 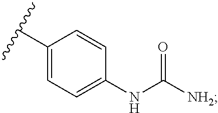 [SMILES: *c1ccc(NC(N)=O)cc1]
R¹⁴ is H or CH₃ and R¹⁶ are H; and
R^{15A} is Cl, OH, or F.

38-41. (canceled)

42. The compound of claim 1, or a pharmaceutically acceptable salt thereof, wherein the chelating group is chelated with a radionuclide, metal ion or metal-halogen ion, wherein the radionuclide, metal ion or metal-halogen ion is selected from ¹⁷⁷Lu, ¹⁷⁵Lu, ⁴⁴Sc, ⁴⁵Sc, ⁴⁷Sc, ⁶⁴Cu, ⁶⁷Cu, ⁶⁸Cu, ¹⁸F, ⁶⁶Ga, ⁶⁷Ga, ⁶⁸Ga, ⁶⁹Ga, ⁷¹Ga, ⁹⁰Y, ⁸⁹Y, ⁸⁶Y, ⁸⁹Zr, ⁹⁰Y, ⁹⁹mTc, ¹¹¹In, ¹¹³In, ¹¹⁵In, ¹²³I, ¹²⁵I, ¹³¹I, ¹³⁹La, ¹³⁴Ce, ¹³⁶Ce, ¹³⁸Ce, ¹⁴⁰Ce, ¹⁴²Ce, ¹⁴³Ce, ¹⁵³Sm, ¹⁵¹Eu, ¹⁵³Eu, ¹⁵²Dy, ¹⁴⁹Tb, ¹⁵⁹Tb, ¹⁶¹Tb, ¹⁵⁴Gd, ¹⁵⁵Gd, ¹⁵⁶Gd, ¹⁵⁷Gd, ¹⁵⁸Gd, ¹⁶⁰Gd, ¹⁸⁸Re, ¹⁸⁶Re, ²¹³Bi, ²¹¹At, ²¹⁷At, ²²⁷Th, ²²⁶Th, ²²⁵Ac, ²²³Ra, ²²⁴Ra, ²³³Ra, ¹⁵²Dy, ²¹³Bi, ²¹²Bi, ²¹¹Bi, ²⁰³Pb, ²¹²Pb, ²⁵⁵Fm, and ²³⁰U.

43. The compound of claim 42 or a pharmaceutically acceptable salt thereof, wherein the ¹⁸F is in the form of [¹⁸F]AlF²⁺.

44. The compound of claim 42 or a pharmaceutically acceptable salt thereof, wherein the radionuclide is:
(i) an alpha-emitting radionuclide;
(ii) an Auger electron emitting radionuclide or a beta-minus-emitting radionuclide;
(iii) a beta-plus-emitting radionuclide for positron-emitting tomography (PET) or gamma ray/photon emitting radionuclide for single-photon emission computerized tomography (SPECT); or
(iv) ²²⁵Ac.

45. The compound of claim 44 or a pharmaceutically acceptable salt thereof, wherein the radionuclide is selected from the group consisting of ²²⁵Ac, ²³³Ra, ²¹²Pb, ¹⁷⁷Lu, ⁸⁸Re, ¹⁶¹Tb, ⁹⁰Y, and ⁶⁷Cu.

46-48. (canceled)

49. A metal complex comprising:
the compound of claim 1 or a pharmaceutically acceptable salt thereof, and
the radionuclide, metal ion or metal-halogen ion selected from ¹⁷⁷Lu, ¹⁷⁵Lu, ⁴⁴Sc, ⁴⁵Sc, ⁴⁷Sc, ⁶⁴Cu, ⁶⁷Cu, ⁶⁸Cu, ¹⁸F ⁶⁶Ga, ⁶⁷Ga, ⁶⁸Ga, ⁶⁹Ga, ⁷¹Ga, ⁹⁰Y, ⁸⁹Y, ⁸⁶Y, ⁸⁹Zr, ⁹⁰Y, ⁹⁹mTc, ¹¹¹In, ¹¹³In, ¹¹⁵In, ¹²³I, ¹²⁵I, ¹³¹I, ¹³⁹La, ¹³⁴Ce, ¹³⁶Ce, ¹³⁸Ce, ¹⁴⁰Ce, ¹⁴²Ce, ¹⁴³Ce, ¹⁵³Sm, ¹⁵¹Eu, ¹⁵³Eu, ¹⁵²Dy, ¹⁴⁹Tb, ¹⁵⁹Tb, ¹⁶¹Tb, ¹⁵⁴Gd, ¹⁵⁵Gd, ¹⁵⁶Gd, ¹⁵⁷Gd, ¹⁵⁸Gd, ¹⁶⁰Gd, ¹⁸⁸Re, ¹⁸⁶Re, ²¹³Bi, ²¹¹At, ²¹⁷At, ²²⁷Th, ²²⁶Th, ²²⁵Ac, ²²³Ra, ²²⁴Ra, ²³³Ra, ¹⁵²Dy, ²¹³Bi, ²¹²Bi, ²¹¹Bi, ²⁰³Pb, ²¹²Pb, ²⁵⁵Fm, and ²³⁰U
wherein the radionuclide, metal ion, or metal-halogen ion is complexed to the chelating group.

50. A pharmaceutical composition comprising: i) the compound of claim 1 or a pharmaceutically acceptable salt thereof;
and ii) a pharmaceutically acceptable carrier or diluent.

51. The pharmaceutical composition of claim 50, comprising the compound of claim 1 chelated to a first radionuclide, metal ion, or metal-halogen ion, and further comprising the compound of claim 1 chelated to a second radionuclide, metal ion, or metal-halogen ion, wherein the first and second radionuclide, metal ion, or metal-halogen ion are different.

52. The pharmaceutical composition of claim 51, wherein the first radionuclide, metal ion, or metal-halogen ion is ¹⁷⁷Lu and the second radionuclide, metal ion, or metal halogen ion is ²²⁵Ac.

53. A method of treating diseased tissue in a subject, wherein the diseased tissue expresses somatostatin receptors, comprising administering an effective amount of the compound or pharmaceutically acceptable salt of claim 42 to the subject and wherein the radionuclide is a therapeutic radionuclide.

54. (canceled)

55. A method of treating a disease in a subject, wherein the disease is characterized by the expression of somatostatin receptors, comprising administering an effective amount of the compound or pharmaceutically acceptable salt of claim 42 to the subject and wherein the radionuclide is a therapeutic radionuclide.

56. The method of claim 55, wherein the disease is:
i) a cancer selected from the group consisting of pituitary tumors, renal cell cancer, breast cancer, meningioma, glioma, glioblastoma multiforme (GBM), neuroblastoma, colorectal cancer, pheochromocytoma, paraganglioma, medullary thyroid cancer, small cell lung cancer, ovarian cancer, head and/or neck cancer, gastric cancer, adrenal cancer, brain cancer, and a hematologic malignancy;
ii) a neuroendocrine tumor; or
iii) a neuroendocrine tumor selected from the group consisting of gastroenteropancreatic neuroendocrine tumor, carcinoid tumor, pheochromocytoma, paraganglioma, medullary thyroid cancer, pulmonary neuroendocrine tumor, thymic neuroendocrine tumor, a carcinoid tumor or a pancreatic neuroendocrine tumor, pituitary adenoma, adrenal gland tumors, Merkel cell carcinoma, breast cancer, non-Hodgkin lymphoma, Hodgkin lymphoma, head & neck tumor, urothelial carcinoma (bladder), renal cell carcinoma, hepatocellular carcinoma, GIST, neuroblastoma, bile duct tumor, cervix tumor, Ewing sarcoma, osteosarcoma, small cell lung cancer (SCLC), prostate cancer, melanoma, meningioma, glioma, medulloblastoma, hemangioblastoma, supratentorial primitive, neuroectodermal tumor, esthesioneuroblastoma functional carcinoid tumor, insulinoma, gastrinoma, vasoactive intestinal peptide (VIP) oma, glucagonoma, serotoninoma, histaminoma, ACTHoma, pheocromocytoma, and somatostatinoma.

57-59. (canceled)

60. A method of treating diseased tissue in a subject wherein the diseased tissue expresses somatostatin receptors, comprising administering an effective amount of the compound or pharmaceutically acceptable salt of claim 42 in combination with a second anti-cancer therapeutic agent to the subject and wherein the radionuclide is a therapeutic radionuclide.

61. A method of imaging a region in a subject having or suspected of having diseased tissue which expresses somatostatin receptors, comprising:
(i) administering to the subject a diagnostically effective amount of a compound or pharmaceutically acceptable salt thereof of claim 42, or the pharmaceutical composition thereof and wherein the radionuclide is a diagnostic radionuclide;
(ii) exposing the region in the subject to an imaging device; and
(iii) obtaining an image of the diseased tissue in the region;
wherein the region has or is suspected of having diseased tissue that includes a primary cancer or a metastasis of the cancer.

62. (canceled)

63. A method of imaging tumors, the method comprising:
(i) contacting the tumor and/or surrounding tissue with a compound or pharmaceutically acceptable salt thereof of claim 21 in an amount sufficient to bind to the tumor;
(ii) irradiating the tumor and/or surrounding tissue at a wavelength absorbed by the compound;
(iii) and detecting a signal from the compound, thereby imaging the tumor and/or surrounding tissue.

64. A method of treating diseased tissue, comprising:
(i) administering to a subject a compound of claim 1, or a pharmaceutically acceptable salt thereof, in an amount effective to contact and bind to the diseased tissue;
(ii) using the compound as a fiducial, irradiating the region of the bound compound with one or more doses of external beam radiation, thereby treating the diseased tissue with radiation.

65. (canceled)

66. A method of treating diseased tissue, comprising: administering to a subject, a compound of claim 1, or a pharmaceutically acceptable salt thereof, in an amount effective to contact and bind to the diseased tissue; and using the compound as a fiducial for guided surgery applications, to resect the region of the diseased tissue thereby excising the diseased tissue.

67-74. (canceled)
